SEQUENCE LISTING (494 sequences)

SEQ ID NO: 1 - protein, 5 aa, synthetic construct
DYGMA

SEQ ID NO: 2 - protein, 17 aa, synthetic construct
FITSLAYIIYYADSVKG

SEQ ID NO: 3 - protein, 11 aa, synthetic construct
NYGSSPYYFDY

SEQ ID NO: 4 - protein, 120 aa, synthetic construct
EVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSS

SEQ ID NO: 5 - protein, 11 aa, synthetic construct
RASQDINKFLS

SEQ ID NO: 6 - protein, 7 aa, synthetic construct
YTSSLQS

SEQ ID NO: 7 - protein, 8 aa, synthetic construct
LQYDNLYT

SEQ ID NO: 8 - protein, 106 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIK

SEQ ID NO: 9 - protein, 246 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSS

SEQ ID NO: 10 - protein, 5 aa, synthetic construct
TSWIE

SEQ ID NO: 11 - protein, 17 aa, synthetic construct
SILPGSGSTNYAQKFQG

SEQ ID NO: 12 - protein, 13 aa, synthetic construct
GDDGSYVRWYFDH

SEQ ID NO: 13 - protein, 122 aa, synthetic construct
QVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYVRWYFDHWGQGTLVTVSS

SEQ ID NO: 14 - protein, 17 aa, synthetic construct
RASQSLLFSVNQKNYLA

SEQ ID NO: 15 - protein, 7 aa, synthetic construct
WASSLQS

SEQ ID NO: 16 - protein, 9 aa, synthetic construct
QHYYSYPWT

SEQ ID NO: 17 - protein, 113 aa, synthetic construct
DIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIK

SEQ ID NO: 18 - protein, 255 aa, synthetic construct
DIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYVRWYFDHWGQGTLVTVSS

SEQ ID NO: 19 - protein, 737 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYVRWYFDHWGQGTLVTVSS

SEQ ID NO: 20 - protein, 20 aa, synthetic construct
GGGGSGGGGSGGGGSGGGGS

SEQ ID NO: 21 - protein, 7 aa, synthetic construct
GGGGSPS

SEQ ID NO: 22 - protein, 229 aa, synthetic construct
EPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 23 - protein, 17 aa, synthetic construct
SILPGSGSTNYNQKFQG

SEQ ID NO: 24 - protein, 122 aa, synthetic construct
QVQLVQSGAEVKKPGSSVKVSCKASGYTFSTSWIEWIRQAPGQGLEWMGSILPGSGSTNYNQKFQGRVTITADTSTSTAYMELSSLRSEDTAVYYCARGDDGSYVRWYFDHWGQGTLVTVSS

SEQ ID NO: 25 - protein, 255 aa, synthetic construct
DIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYNQKFQGRVTITADTSTSTAYMELSSLRSEDTAVYYCARGDDGSYVRWYFDHWGQGTLVTVSS

SEQ ID NO: 26 - protein, 737 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYNQKFQGRVTITADTSTSTAYMELSSLRSEDTAVYYCARGDDGSYVRWYFDHWGQGTLVTVSS

SEQ ID NO: 27 - protein, 122 aa, synthetic construct
QVQLVQSGAEVKKPGSSVKVSCKASGYTFSTSWIEWIRQAPGQCLEWMGSILPGSGSTNYAQKFQGRVTITADTSTSTAYMELSSLRSEDTAVYYCARGDDGSYVRWYFDHWGQGTLVTVSS

SEQ ID NO: 28 - protein, 113 aa, synthetic construct
DIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGCGTKVEIK

SEQ ID NO: 29 - protein, 255 aa, synthetic construct
DIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGCGTKVEIKGGGGSGGGGSGGGGSGGGGSQVQLVQSGAEVKKPGSSVKVSCKASGYTFSTSWIEWIRQAPGQCLEWMGSILPGSGSTNYAQKFQGRVTITADTSTSTAYMELSSLRSEDTAVYYCARGDDGSYVRWYFDHWGQGTLVTVSS

SEQ ID NO: 30 - protein, 737 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGCGTKVEIKGGGGSGGGGSGGGGSGGGGSQVQLVQSGAEVKKPGSSVKVSCKASGYTFSTSWIEWIRQAPGQCLEWMGSILPGSGSTNYAQKFQGRVTITADTSTSTAYMELSSLRSEDTAVYYCARGDDGSYVRWYFDHWGQGTLVTVSS

SEQ ID NO: 31 - protein, 13 aa, synthetic construct
GDDGSYNRWYFDH

SEQ ID NO: 32 - protein, 122 aa, synthetic construct
QVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYNRWYFDHWGQGTLVTVSS

SEQ ID NO: 33 - protein, 255 aa, synthetic construct
DIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYNRWYFDHWGQGTLVTVSS

SEQ ID NO: 34 - protein, 122 aa, synthetic construct
QVQLVQSGAEVKKPGSSVKVSCKASGYTFSTSWIEWIRQAPGQGLEWMGSILPGSGSTNYNQKFQGRVTITADTSTSTAYMELSSLRSEDTAVYYCARGDDGSYNRWYFDHWGQGTLVTVSS

SEQ ID NO: 35 - protein, 255 aa, synthetic construct
DIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYNQKFQGRVTITADTSTSTAYMELSSLRSEDTAVYYCARGDDGSYNRWYFDHWGQGTLVTVSS

SEQ ID NO: 36 - protein, 737 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYNQKFQGRVTITADTSTSTAYMELSSLRSEDTAVYYCARGDDGSYNRWYFDHWGQGTLVTVSS

SEQ ID NO: 37 - protein, 17 aa, synthetic construct
RASQSLTFSVNQKNYLA

SEQ ID NO: 38 - protein, 113 aa, synthetic construct
DIQMTQSPSSVSASVGDRVTITCRASQSLTFSVNQKNYLAWYQQKPGKAPKLLIYWASSLQSGVPSRFSGSGSGTDFTLTISSLQPEDFATYYCQHYYSYPWTFGQGTKVEIK

SEQ ID NO: 39 - protein, 255 aa, synthetic construct
DIQMTQSPSSVSASVGDRVTITCRASQSLTFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYVRWYFDHWGQGTLVTVSS

SEQ ID NO: 40 - protein, 737 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLTFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYVRWYFDHWGQGTLVTVSS

SEQ ID NO: 41 - protein, 17 aa, synthetic construct
RASQSLNFSVNQKNYLA

SEQ ID NO: 42 - protein, 113 aa, synthetic construct
DIQMTQSPSSVSASVGDRVTITCRASQSLNFSVNQKNYLAWYQQKPGKAPKLLIYWASSLQSGVPSRFSGSGSGTDFTLTISSLQPEDFATYYCQHYYSYPWTFGQGTKVEIK

SEQ ID NO: 43 - protein, 255 aa, synthetic construct
DIQMTQSPSSVSASVGDRVTITCRASQSLNFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYVRWYFDHWGQGTLVTVSS

SEQ ID NO: 44 - protein, 737 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLNFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYVRWYFDHWGQGTLVTVSS

SEQ ID NO: 45 - protein, 17 aa, synthetic construct
SINPGSGSTNYAQKFQG

SEQ ID NO: 46 - protein, 122 aa, synthetic construct
QVQLVQSGAEVKKPGSSVKVSCKASGYTFSTSWIEWIRQAPGQGLEWMGSINPGSGSTNYAQKFQGRVTITADTSTSTAYMELSSLRSEDTAVYYCARGDDGSYVRWYFDHWGQGTLVTVSS

SEQ ID NO: 47 - protein, 255 aa, synthetic construct
DIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSINPGSGSTNYAQKFQGRVTITADTSTSTAYMELSSLRSEDTAVYYCARGDDGSYVRWYFDHWGQGTLVTVSS

SEQ ID NO: 48 - protein, 737 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSINPGSGSTNYAQKFQGRVTITADTSTSTAYMELSSLRSEDTAVYYCARGDDGSYVRWYFDHWGQGTLVTVSS

SEQ ID NO: 49 - protein, 17 aa, synthetic construct
SIQPGSGSTNYAQKFQG

SEQ ID NO: 50 - protein, 122 aa, synthetic construct
QVQLVQSGAEVKKPGSSVKVSCKASGYTFSTSWIEWIRQAPGQGLEWMGSIQPGSGSTNYAQKFQGRVTITADTSTSTAYMELSSLRSEDTAVYYCARGDDGSYVRWYFDHWGQGTLVTVSS

SEQ ID NO: 51 - protein, 255 aa, synthetic construct
DIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSIQPGSGSTNYAQKFQGRVTITADTSTSTAYMELSSLRSEDTAVYYCARGDDGSYVRWYFDHWGQGTLVTVSS

SEQ ID NO: 52 - protein, 737 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSIQPGSGSTNYAQKFQGRVTITADTSTSTAYMELSSLRSEDTAVYYCARGDDGSYVRWYFDHWGQGTLVTVSS

SEQ ID NO: 53 - protein, 271 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERSSSLNDIFEAQKIEWHEDYKDDDDKDYKDDDDKDYKDDDDKHHHHHHHHHH

SEQ ID NO: 54 - protein, 456 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERSSSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 55 - protein, 271 aa, synthetic construct
FTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERSSSLNDIFEAQKIEWHEDYKDDDDKDYKDDDDKDYKDDDDKHHHHHHHHHH

SEQ ID NO: 56 - protein, 456 aa, synthetic construct
FTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERSSSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 57 - protein, 270 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRSSSLNDIFEAQKIEWHEDYKDDDDKDYKDDDDKDYKDDDDKHHHHHHHHHH

SEQ ID NO: 58 - protein, 455 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRSSSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 59 - protein, 272 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 60 - protein, 272 aa, synthetic construct
FTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERTHLVILGAIFLLLGVALTFIFYLRKGRMMDMKKCGIRVTNSKKQRDTQLEET

SEQ ID NO: 61 - protein, 265 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 62 - protein, 248 aa, synthetic construct
DVKLVESGGGLVKPGGSLKLSCAASGFTFSSYTMSWVRQTPEKRLEWVATISSGGNYTYYPDSVKGRFTISRDNAKNTLYLQLSSLKSEDTAMYYCTRDQAYYGNLFTYWGQGTLVTVSAGGGGSGGGGSGGGGSGGGGSDIQMTQSPASLSASVGETVTITCRASENIYSFLAWYQQKQGKSPQLLVYKAKTLTEGVPSRFSGSGSGTQFSLKINSLQPEDFGSYYCQHHYGSPPETFGGGTKVEIK

SEQ ID NO: 63 - protein, 243 aa, synthetic construct
QIQLQQSGPELVKPGASVKISCKASGYTFTDYYINWVKQKPEQGLEWIGWTFPGSINTKYNEKFKGKATLTVDTSSSTAYMQLNSLTSEDTAVYFCARSPYEFDYWGQGTTLTVSSGGGGSGGGGSGGGGSGGGGSDIQMTQTTSSLSASLGDRVTISCSASQGIRNYLNWYQQKPDGTVKLLIYYTSTLHSGVPSRFSGSGSGTDYSLTISNLEPEDIATYYCQQYSKLPWTFGGGTRLEIK

SEQ ID NO: 64 - protein, 246 aa, synthetic construct
EVQLQESGPSLVKPSQTLSLTCSVTGDSITSGYWNWIRKFPGKKLEYMGYISYTGSTYYNPSLKSRISIARDTSKNQYFLQLNSVTTEDTATYYCTRGENWDGGYFDYWGQGTTLTVSSGGGGSGGGGSGGGGSGGGGSDIQMTQTTSSLSASLGDRVTISCSASQGIRNYLNWYQQKPDGTVKLLIYYTSTLHSGVPSRFSGSGSGTDYSLTISNLEPEDIATYYCQQYSKLPWTFGGGTRLEIK

SEQ ID NO: 65 - protein, 246 aa, synthetic construct
EVKLVESGGGLVQPGGSRKLSCAASGFTFSDYGMAWVRQAPGKGPEWVAFITSLAYIIFYSDTVTGRFTISRENAKNTLYLEMSSLRSEDTAVYYCARNYGSSPYYFDYWGQGTTLTVSSGGGGSGGGGSGGGGSGGGGSDIQMTQSPSSLSTSLGGKVTITCKASQDINKFISWYQHKPGKGPRLLIHYTSSLQPGIPSRFSGSGSGRDYSFSISNLEPEDIATYYCLQYDNLYTFGGGTKLEIK

SEQ ID NO: 66 - protein, 17 aa, synthetic construct
FITSLAYIIFYSDTVTG

SEQ ID NO: 67 - protein, 11 aa, synthetic construct
KASQDINKFIS

SEQ ID NO: 68 - protein, 7 aa, synthetic construct
YTSSLQP

SEQ ID NO: 69 - protein, 248 aa, synthetic construct
EVKFEESGGGLVQPGGSMKLSCFASGFTFSYYWMNWVRQSPEKGLEWIAEIRLKSNNYATHYAESVKGRFTISRDDSKSSVYLEMNNLRAEDTGIYHCTRPPIYYGNYEAYWGQGTLVTVSAGGGGSGGGGSGGGGSGGGGSDIQMTQSPSSLSASLGGKVTITCKASQDINKYIAWYQHKPGKGPRLLIHYTSILQPGIPSRFSGSGSGRDYSFSISNLEPEDIATYYCLQYDNLRTFGGGTKLEIK

SEQ ID NO: 70 - protein, 246 aa, synthetic construct
QGQMQQSGAELVKPGASVKLSCKTSGFTFSSSYITWLKQKPGQSLEWIAWIYVGTGGISYNQKFTGKAQLTVDTSSSTAYMQFSSLTTEDSAIYYCARHGRYHWYFDVWGAGTTVTVSSGGGGSGGGGSGGGGSGGGGSDIVMTQSHKFMSTSVGDRVTITCKASQDVGTAVAWYQKKPGQSPKLLISWASTRHTGVPDRFTGSGSGTDFTLTISNVQSEDLADYFCQQYSSYPYTFGGGTNLEIK

SEQ ID NO: 71 - protein, 248 aa, synthetic construct
EVQLQESGPSLVKPSQTLSLTCSVTGDSITSGYWNWIRKFPGNRLEYMGFRSYSGTTYYNPSLKSRISITRDTSKNQYYLQLNSVTTEDTATYYCARFSDFGSSENTMDYWGQGTSVTVSSGGGGSGGGGSGGGGSGGGGSDIVMTQSHKFMSTSVGDRVTITCKASQDVGTAVAWYQKKPGQSPKLLISWASTRHTGVPDRFTGSGSGTDFTLTISNVQSEDLADYFCQQYSSYPYTFGGGTNLEIK

SEQ ID NO: 72 - protein, 248 aa, synthetic construct
EVQLQESGPSLVKPSQTLSLTCSVTGDSITSGYWNWIRKFPGNRLEYMGFRSYSGTTYYNPSLKSRISITRDTSKNQYYLQLNSVTTEDTATYYCARFSDFGSSENTMDYWGQGTSVTVSSGGGGSGGGGSGGGGSGGGGSDVQITQSPSYLAASPGETITINCRASKSISKYLAWYQEKPGKTNKLLIYSGSTLQSGIPSRFSGSGSGTDFTLTISSLEPEDFAMYYCQQHNEYPFTFGAGTKLEIK

SEQ ID NO: 73 - protein, 250 aa, synthetic construct
EVQLQESGPSLVKPSQTLSLTCSVTGDSITSGYWNWIRKFPGKKLEYMGYISYTGSTYYNPSLKSRISIARDTSKNQYFLQLNSVTTEDTATYYCTRGENWDGGYFDYWGQGTTLTVSSGGGGSGGGGSGGGGSGGGGSDIVLTQSPASLAVSLGQRATISCRASKSVSKSGYSYLHWYQQKPGQTPKLLIYLASNLESGVPARFSGSGSGTDFTLNIHPVEEEDAATYYCQHSWELPYTFGGGTKLEIK

SEQ ID NO: 74 - protein, 245 aa, synthetic construct
KVQLQQSGAEVVKPGASVKLSCKASGYNLTEYIIHWVKQRSGQGLEWIGWFYPGSGSIKYNEKFKDKATLTADKSSTTVYMDLSRLTSEDSAVYFCARHGLYYGFPYWGQGTSVTVSSGGGGSGGGGSGGGGSGGGGSDIQMTQTTSSLSASLGDRVTISCRASQDIRNYLNWYQQKPDGTVKLLIYYTSRLQSGVPSRFSGSGSGTDYSLTITTLEQEDFATYFCQQGNMFPYTFGGGTKLEIK

SEQ ID NO: 75 - protein, 481 aa, synthetic construct
EVQLVESGGGLVQPGGSLRLSCAASGFTFSDYGMNWVRQAPGKGPEWVASITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQHKPGKAPKLLIHYTSSLQPGVPSRFSGSGSGRDYTLTISSLQPEDFATYYCLQYDNLYTFGQGTKLEIKSSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 481 aa, synthetic construct
EVQLLESGGGLVQPGGSLRLSCAASGFTFSDYGMSWVRQAPGKGPEWVAAITSLAYIIYYADSVKGRFTISRDNSKNTLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQHKPGKAPKLLIHYTSSLQPGVPSRFSGSGSGRDYTLTISSLQPEDFATYYCLQYDNLYTFGQGTKLEIKSSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 481 aa, synthetic construct
EVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIKSSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 360 aa, synthetic construct
GAGGTACAACTGGTAGAATCAGGGGGCGGACTGGTACAGCCCGGGGGCTCCCTGAGACTGAGTTGCGCCGCCTCTGGCTTTACTTTCTCCGATTACGGTATGGCATGGGTTAGACAAGCCCCCGGAAAAGGGCTCGAATGGGTGTCATTCATCACATCACTGGCCTACATTATCTATTATGCCGACTCCGTTAAGGGGCGGTTCACCATTAGCCGTGACAACGCAAAGAATAGTCTTTACCTGCAAATGAACTCTCTCAGGGCAGAAGATACAGCCGTCTATTACTGTGCCAGAAACTATGGTAGTTCTCCTTATTACTTTGATTACTGGGGACAAGGAACTTTGGTTACCGTGAGCTCA

SEQ ID NO: 79 - protein, 318 aa, synthetic construct
GATATTCAGATGACCCAAAGTCCATCATCCCTTAGTGCCTCCGTGGGGGATAGAGTAACTATCACATGCCGCGCTAGTCAAGATATAAACAAATTTTTGTCCTGGTATCAGCAGAAGCCTGGTAAAGCTCCAAAATTGCTGATCCATTATACCTCTTCCCTGCAATCTGGGGTACCTTCCCGTTTCTCTGGGTCCGGATCAGGTACAGACTTCACTCTTACCATAAGCTCACTCCAACCAGAAGATTTCGCAACCTATTACTGTCTCCAGTACGATAACCTGTATACTTTTGGCCAGGGAACTAAGTTGGAGATTAAG

SEQ ID NO: 80 - protein, 409 aa, synthetic construct
EPPTACREKQYLINSQCCSLCQPGQKLVSDCTEFTETECLPCGESEFLDTWNRETRCHQHKYCDPNLGLRVQQKGTSETDTICTCEEGLHCTSESCESCVPHRSCLPGFGVKQIATGVSDTICEPCPVGFFSNVSSAFEKCRPWTSCETKDLVVQQAGTNKTDVVCGPQDRQRSSSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 81 - protein, 202 aa, synthetic construct
EPPTACREKQYLINSQCCSLCQPGQKLVSDCTEFTETECLPCGESEFLDTWNRETRCHQHKYCDPNLGLRVQQKGTSETDTICTCEEGLHCTSESCESCVPHRSCLPGFGVKQIATGVSDTICEPCPVGFFSNVSSAFEKCRPWTSCETKDLVVQQAGTNKTDVVCGPQDRQRHHHHHHHHHHGLNDIFEAQKIEWHEEPEA

SEQ ID NO: 82 - protein, 408 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLSSSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 83 - protein, 201 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLHHHHHHHHHHGLNDIFEAQKIEWHEEPEA

SEQ ID NO: 84 - protein, 257 aa, Homo sapiens
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 85 - protein, 258 aa, Macaca fascicularis
EPPTACREKQYLINSQCCSLCQPGQKLVSDCTEFTETECLPCGESEFLDTWNRETRCHQHKYCDPNLGLRVQQKGTSETDTICTCEEGLHCTSESCESCVPHRSCLPGFGVKQIATGVSDTICEPCPVGFFSNVSSAFEKCRPWTSCETKDLVVQQAGTNKTDVVCGPQDRQRALVVIPICLGILFVILLLVLVFIKKVAKKPNDKVPHPKQEPQEINFPDDLPGSNPAAPVQETLHGCQPVTQEDGKESRISVQERQ

SEQ ID NO: 86 - protein, 726 aa, synthetic construct
EVQLQQSGPELVKPGASMKISCKASGYSITGYTMNWVKQSHGKNLEWIGLINTYTGGTTYNQKFKGKATLTVDKSSSTAYMELLSLTSEDSAVYYCAGTGTGAMDYWGQGTSVTVSSGGGGSGGGGSGGGGSGGGGSQIVLTQSPAIMSSSPGEKVTMTCSASSSVSYIHWYQQKSGTSPKRWIYDTSKLASGVPARFSGSGSGTSYSLTINSVEAEDAATYYCQQWRTNPYTFGGGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 87 - skipped

SEQ ID NO: 88 - protein, 727 aa, synthetic construct
DVQLVESGGGLVQPGGSRKLSCAASGFTFSTFGMHWVRQAPEKGLEWVAYISSGSNTIYYADTLKGRFTISRDNPRNTLFLQMTSLRSEDTAIYYCTRRPIPGSMDYWGQGTSVTVSSGGGGSGGGGSGGGGSGGGGSQIVLTQSPALISAFPGEKVTMTCSASSSVTYMHWYQQKPRSSPKSWIYLTSNLASGVPTRFSGSGSGTSYSLTISSMEAEDAATYFCQQWSTNSLTFGAGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 89 - skipped

SEQ ID NO: 90 - protein, 738 aa, synthetic construct
QVQLQQSGAELMNPGASVRISCKATGYTFSTSWIEWIKQRPGHGLEWIGEILPGSGSTNYNEKFKGKATFTADTSSNTAYMQLSSLTFEDSAVYYCARGDDGSYVRWYFDVWGAGTTVTVSSGGGGSGGGGSGGGGSGGGGSDIVMSQSPSSLAVSVGEKVTMSCKSSQSLLFSVNQKNYLAWYQQKPGQSPKLLIYWASTRESGVPDRFTGSGSGTDFTLTIGSVKAEDLAVYYCQHYYSYPWTFGGGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 91 - skipped

SEQ ID NO: 92 - protein, 727 aa, synthetic construct
DVQLVESGGGLVQPGGSRKLSCAASGFTFSTFGMHWVRQAPEKGLEWVAYISSGSNTIYYADTLKGRFTISRDNPRNTLFLQMTSLRSEDTAIYYCTRRPIPGAMDYWGQGTSVTVSSGGGGSGGGGSGGGGSGGGGSQIVLTQSPALISAFPGEKVTMTCSASSSVTYMHWYQQKPRSSPKSWIYLTSNLASGVPSRFSGSGSGTSYSLTISSMEAEDAATYFCQQWSTNSLTFGAGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 93 - skipped

SEQ ID NO: 94 - protein, 737 aa, synthetic construct
EVMLVESGGGLVKPGGSLKLSCAASGFTFSSYAMSWVRQTPERRLEWVASISSAGTYIYYPDSVKGRFTISRDNAKNILYLQMNSLRSEDTAMYYCARHGNGYDPLWYFDVWGAGTTVTVSSGGGGSGGGGSGGGGSGGGGSDVVMTQTPLSLPVSLGDQASISCRSSQSLVHYNGYTYLHWYLQKPGQSPKLLIYKVSNRFSGVPDRFSGSGSGTDFTLKISRVEAEDLGVYFCSQTTHVPLTFGAGTKLELREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 95 - skipped

SEQ ID NO: 96 - protein, 727 aa, synthetic construct
DVQLVESGGGLVQPGGSRKLSCAASGFAFSSFGIHWVRQSPEKGLEWVAYISGGSSTIYYADSLKGRFTISRDNPKNTLFLQMTSLRSEDTAIYYCVRRPIPGAMDYWGQGISVTVSSGGGGSGGGGSGGGGSGGGGSQIVLTQSPALMSASPGEKVTMTCRASSRVSYIHWYQQKPRSSPKSWIYLTSNLASGVPSRFSGSGSGTSYSLTISSMEAEDAATYYCQQWSSNSLTFGAGTKLELKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 97 - skipped

SEQ ID NO: 98 - protein, 727 aa, synthetic construct
DVQLVESGGGLVQPGGSRKLSCAASGFTFSSFGMHWVRQAPEKGLEWVAYISSGSITIYYADTLKGRFTISRDNPKNTLFLQMTSLRSEDTAMYYCTRRPIPGAMDYWGQGTSVTVSSGGGGSGGGGSGGGGSGGGGSQIVLTQSPALMSASPGEKVTMTCSASSSVTYMHWYQQKPRSSPKSWIYLTSNLASGVPARFSGSGSGTSYSLTISSMEAEDAATYYCQQWSSDSLTFGAGTKLELKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 99 - skipped

SEQ ID NO: 100 - protein, 731 aa, synthetic construct
EVLLQQSGPELVKPGASVKISCKASGYKFNDYNIDWVKQSQGKSLEWIGNINPDNGGTIYNQKFKGKATLTVDKSSSTPYMELRSLTSEDTAVYFCARERDNRYDRWSAYWGQGTLVTVSAGGGGSGGGGSGGGGSGGGGSDIVMTQSQKFMSTSVGDRVSVTCKASQNVGANVAWYQQKPGQSPKALIYSASYRYSGVPDRFTGSGSGTDFTLTISNVHSDDLAEYFCQQYNTYPFTFGSGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 101 - skipped

SEQ ID NO: 102 - protein, 731 aa, synthetic construct
EVLLQQSGPEVVKPGASVKISCKASGYKFNDYNIDWVKQSHGKSLDWIGNINPNNGGTIYNQKFKGKATLTVDKSSSTPYMDLRSLTSEDTAVYFCSRERDNRYDRWSAYWGQGTLVTVSAGGGGSGGGGSGGGGSGGGGSDIVMTQSQKFMSTSVGDRVSVTCKASQNVGANVAWYQHKPGQSPKALIYSASYWNSGVPDRFTGSGSGTDFTLTISSVHSDDLAEYFCQQYNSYPFAFGSGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 103 - skipped

SEQ ID NO: 104 - protein, 726 aa, synthetic construct
EVQLQQSGPELVKPGASMKISCKASGYSFTGYTMNWVKQSHGKNLEWIGLVNVYHGGTTYNQKFKGKATLTVDKSSSTAYMELLSLTSEDSAVYYCAGTGTGALDYWGQGTSVTVSSGGGGSGGGGSGGGGSGGGGSQIVLTQSPAIMSASPGEKVTMTCSASSSVSYMHWYQQKSGPSPKRWIYDTSKLASGVPARFSGSGSGTSYSLTISSMEAEDAATYYCQQWRSNPYTFGGGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 105 - skipped

SEQ ID NO: 106 - protein, 727 aa, synthetic construct
DVQLVESGGGLVQPGGSRKLSCAASGFTFSTFGMHWVRQAPEKGLEWVAYISSGSNTIYYADTLKGRFTISRDNPRNTLFLQMTSLRSEDTAIYYCTRRPIPGAMDYWGQGTSVTVSSGGGGSGGGGSGGGGSGGGGSQIVLTQSPALISAFPGERVTMTCSASSSVTYIHWYQQKPRSSPKSWIYLTSNLASGVPTRFSGSGSGTSYSLTISSMEAEDAATYFCQQWSTNSLTFGAGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 107 - skipped

SEQ ID NO: 108 - protein, 738 aa, synthetic construct
QVQLQQSGAELMKPGASVKIPCKATGYTFSSYWIEWVKQRPGHGLEWIGEVLPGSGSTNYNEKFKGKATFTADTSSNTAYMQLSSLTSEDSAVYYCARPYYRYDVGAWFAYWGQGTLVTVSAGGGGSGGGGSGGGGSGGGGSDIVMSQSPSSLAVSVGEKVTMSCKSSQSLLYSGNQKNYLAWYQQKPGQSPKLLIYWASTRESGVPDRFTGSGSGTDFTLTISSVKAEDLAVYYCQQYYSYPYTFGGGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 109 - skipped

SEQ ID NO: 110 - protein, 727 aa, synthetic construct
QVQLQQSGAELAKPGASVKMSCKASGYAFTTYWMHWVKQRPGQGLEWIGYINPTTGYTDYNQKFKDKATLTADKSSSTAYMQLSSLTSEDSAVYYCARWGLPPFAYWGQGTLVTVSAGGGGSGGGGSGGGGSGGGGSDIVMTQSHKFMSTSVGDRVSITCKASQDVNTAVAWYQQKPGQSPKLLIYSASYRYTGVPDRFTGSGSGTDFTFTISSVQAEDLAVYYCQQHYSTPWTFGGGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 111 - skipped

SEQ ID NO: 112 - protein, 735 aa, synthetic construct
QVQLKESGPGLVAPSQSLSITCTVSGFSLSRYSIQWVRQPPGKGLEWLGMIWGGGSTDYNSALKSRLSISKDNSKSQVFLKMNSLQTDDTAMYYCARNQGGYDVWFAYWGQGTLVTVSAGGGGSGGGGSGGGGSGGGGSDIVMSQSPSSLAVSVGEKVTMNCKSSQSLLYSGNQKKYLAWYQQKPGQSPKLLIYWASTRESGVPDRFTGSGSGTDFTLTISSVKAEDLAIYYCQQYYSYPFTFGGGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 113 - skipped

SEQ ID NO: 114 - protein, 738 aa, synthetic construct
QVQLQQSGTELMKPGAAVKISCKATGYTISSYWIEWVKQRPGHGLEWIGEILPGSGSINYNEKFKGKATFTADTSSNTAYIQLSSLTSEDSAVYYCARGGIYYGPLYAMDYWGQGTSVTVSSGGGGSGGGGSGGGGSGGGGSDIVMSQSPSSLAVSVGEKVTMSCKSSQSLLYSGNQKNYLAWYQQKPGQSPKLLIYWASTRESGVPDRFTGSGSGTDFTLTISSVKAEDLAVYYCQQYYSYPYTFGGGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 115 - skipped

SEQ ID NO: 116 - protein, 730 aa, synthetic construct
EVKLVESGGGLVQPGGALRLSCATSGFTFTDYYMSWVRQPPGKALEWLGFIRNKANGYTTEYSASVKGRFTISRDNSQSILYLQMNTLRAEDSATYYCASHYYGRAWFAYWGQGTLVTVSAGGGGSGGGGSGGGGSGGGGSQIVLTQSPTIMSASPGEKVTMTCSASSSVSNMHWYQQKSGTSPKRWIYDTSTLASGVPARFSGSGSGTSYSLTISSMEAEDVATYYCQQWSSNPPTFGGGTTLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 117 - skipped

SEQ ID NO: 118 - protein, 738 aa, synthetic construct
DIVMSQSPSSLAVSVGEKVTMSCKSSQSLLFSVNQKNYLAWYQQKPGQSPKLLIYWASTRESGVPDRFTGSGSGTDFTLTIGSVKAEDLAVYYCQHYYSYPWTFGGGTKLEIKGGGGSGGGGSGGGGSGGGGSQVQLQQSGAELMNPGASVRISCKATGYTFSTSWIEWIKQRPGHGLEWIGEILPGSGSTNYNEKFKGKATFTADTSSNTAYMQLSSLTFEDSAVYYCARGDDGSYVRWYFDVWGAGTTVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 119 - protein, 278 aa, synthetic construct
DYKDDDDKDYKDDDDKGGSGGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 120 - protein, 278 aa, synthetic construct
DYKDDDDKDYKDDDDKGGSGGEPPTACSDKQYLHDGQCCDLCQPGSRLTSHCTALEKTQCHPCDSGEFSAQWNREIRCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 121 - protein, 278 aa, synthetic construct
DYKDDDDKDYKDDDDKGGSGGEPPTACREKQYLINSQCCSLCQPGQKLVSDCTEFTETECLPCGESEFLDTWNRETHCHQHRHCEPNQGLRVKKEGTAESDTVCTCKEGQHCTSKDCEACAQHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 122 - protein, 278 aa, synthetic construct
DYKDDDDKDYKDDDDKGGSGGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHTPCIPGFGVMEMATETTDTVCHPCPVGFFSNQSSLFEKCYPWTSCEDKNLEVLQKGTSQTNVICGLKSRMRALVVIPIIFGILFAILLVLVFIKKVAKKPTNKAPHPKQEPQEINFPDDLPGSNTAAPVQETLHGCQPVTQEDGKESRISVQERQ

SEQ ID NO: 123 - protein, 278 aa, synthetic construct
DYKDDDDKDYKDDDDKGGSGGEPPTACSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALVVIPIIFGILFAILLVLVFIKKVAKKPTNKAPHPKQEPQEINFPDDLPGSNTAAPVQETLHGCQPVTQEDGKESRISVQERQ

SEQ ID NO: 124 - protein, 278 aa, synthetic construct
DYKDDDDKDYKDDDDKGGSGGEPPTACREKQYLINSQCCSLCQPGQKLVSDCTEFTETECLPCGESEFLDTWNRETHCHQHKYCDPNLGLRVQQKGTSETDTICTCKEGQHCTSKDCEACAQHRSCSPGFGVKQIATETTDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 125 - protein, 278 aa, synthetic construct
DYKDDDDKDYKDDDDKGGSGGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHTPCIPGFGVKQIATGVSDTICEPCPVGFFSNQSSLFEKCHPWTSCETKDLVVQQAGTSQTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 126 - protein, 278 aa, synthetic construct
DYKDDDDKDYKDDDDKGGSGGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVMEMATETTDTVCHPCPVGFFSNVSSAFEKCYPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 127 - protein, 278 aa, synthetic construct
DYKDDDDKDYKDDDDKGGSGGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCYPWTSCEDKNLEVLQKGTSQTNVICGLKSRMRALVVIPIIFGILFAILLVLVFIKKVAKKPTNKAPHPKQEPQEINFPDDLPGSNTAAPVQETLHGCQPVTQEDGKESRISVQERQ

SEQ ID NO: 128 - protein, 726 aa, synthetic construct
DIVMTQSHKFMSTSVGDRVSITCKASQDVNTAVAWYQQKPGQSPKLLIYSASYRYTGVPDRFTGSGSGTDFTFTISSVQAEDLAVYYCQQHYSTPWTFGGGTKLEIKGGGGSGGGGSGGGGSGGGGSQVQLQQSGAELAKPGASVKMSCKASGYAFTTYWMHWVKQRPGQGLEWIGYINPTTGYTDYNQKFKDKATLTADKSSSTAYMQLSSLTSEDSAVYYCARWGLPPFAYWGQGTLVTVSAEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 129 - protein, 736 aa, synthetic construct
DVVMTQTPLSLPVSLGDQASISCRSSQSLVHYNGYTYLHWYLQKPGQSPKLLIYKVSNRFSGVPDRFSGSGSGTDFTLKISRVEAEDLGVYFCSQTTHVPLTFGAGTKLELRGGGGSGGGGSGGGGSGGGGSEVMLVESGGGLVKPGGSLKLSCAASGFTFSSYAMSWVRQTPERRLEWVASISSAGTYIYYPDSVKGRFTISRDNAKNILYLQMNSLRSEDTAMYYCARHGNGYDPLWYFDVWGAGTTVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 130 - protein, 737 aa, synthetic construct
DIVMSQSPSSLAVSVGEKVTMSCKSSQSLLFSVNQKNYLAWYQQKPGQSPKLLIYWASTRESGVPDRFTGSGSGTDFTLTIGSVKAEDLAVYYCQHYYSYPWTFGGGTKLEIKGGGGSGGGGSGGGGSGGGGSQVQLQQSGAELMNPGASVRISCKATGYTFSTSWIEWIKQRPGHGLEWIGEILPGSGSTNYNEKFKGKATFTADTSSNTAYMQLSSLTFEDSAVYYCARGDDGSYVRWYFDVWGAGTTVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 131 - protein, 726 aa, synthetic construct
EVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIVMTQSHKFMSTSVGDRVSITCKASQDVNTAVAWYQQKPGQSPKLLIYSASYRYTGVPDRFTGSGSGTDFTFTISSVQAEDLAVYYCQQHYSTPWTFGGGTKLEIKGGGGSGGGGSGGGGSGGGGSQVQLQQSGAELAKPGASVKMSCKASGYAFTTYWMHWVKQRPGQGLEWIGYINPTTGYTDYNQKFKDKATLTADKSSSTAYMQLSSLTSEDSAVYYCARWGLPPFAYWGQGTLVTVSA

SEQ ID NO: 132 - protein, 736 aa, synthetic construct
EVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDVVMTQTPLSLPVSLGDQASISCRSSQSLVHYNGYTYLHWYLQKPGQSPKLLIYKVSNRFSGVPDRFSGSGSGTDFTLKISRVEAEDLGVYFCSQTTHVPLTFGAGTKLELRGGGGSGGGGSGGGGSGGGGSEVMLVESGGGLVKPGGSLKLSCAASGFTFSSYAMSWVRQTPERRLEWVASISSAGTYIYYPDSVKGRFTISRDNAKNILYLQMNSLRSEDTAMYYCARHGNGYDPLWYFDVWGAGTTVTVSS

SEQ ID NO: 133 - protein, 737 aa, synthetic construct
EVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIVMSQSPSSLAVSVGEKVTMSCKSSQSLLFSVNQKNYLAWYQQKPGQSPKLLIYWASTRESGVPDRFTGSGSGTDFTLTIGSVKAEDLAVYYCQHYYSYPWTFGGGTKLEIKGGGGSGGGGSGGGGSGGGGSQVQLQQSGAELMNPGASVRISCKATGYTFSTSWIEWIKQRPGHGLEWIGEILPGSGSTNYNEKFKGKATFTADTSSNTAYMQLSSLTFEDSAVYYCARGDDGSYVRWYFDVWGAGTTVTVSS

SEQ ID NO: 134 - protein, 476 aa, synthetic construct
EVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 474 aa, synthetic construct
DIVMTQSHKFMSTSVGDRVSITCKASQDVNTAVAWYQQKPGQSPKLLIYSASYRYTGVPDRFTGSGSGTDFTFTISSVQAEDLAVYYCQQHYSTPWTFGGGTKLEIKGGGGSGGGGSGGGGSGGGGSQVQLQQSGAELAKPGASVKMSCKASGYAFTTYWMHWVKQRPGQGLEWIGYINPTTGYTDYNQKFKDKATLTADKSSSTAYMQLSSLTSEDSAVYYCARWGLPPFAYWGQGTLVTVSAEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 136 - protein, 476 aa, synthetic construct; identical to SEQ ID NO: 134

SEQ ID NO: 137 - protein, 484 aa, synthetic construct
DVVMTQTPLSLPVSLGDQASISCRSSQSLVHYNGYTYLHWYLQKPGQSPKLLIYKVSNRFSGVPDRFSGSGSGTDFTLKISRVEAEDLGVYFCSQTTHVPLTFGAGTKLELRGGGGSGGGGSGGGGSGGGGSEVMLVESGGGLVKPGGSLKLSCAASGFTFSSYAMSWVRQTPERRLEWVASISSAGTYIYYPDSVKGRFTISRDNAKNILYLQMNSLRSEDTAMYYCARHGNGYDPLWYFDVWGAGTTVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 138 - protein, 476 aa, synthetic construct; identical to SEQ ID NO: 134

SEQ ID NO: 139 - protein, 485 aa, synthetic construct
DIVMSQSPSSLAVSVGEKVTMSCKSSQSLLFSVNQKNYLAWYQQKPGQSPKLLIYWASTRESGVPDRFTGSGSGTDFTLTIGSVKAEDLAVYYCQHYYSYPWTFGGGTKLEIKGGGGSGGGGSGGGGSGGGGSQVQLQQSGAELMNPGASVRISCKATGYTFSTSWIEWIKQRPGHGLEWIGEILPGSGSTNYNEKFKGKATFTADTSSNTAYMQLSSLTFEDSAVYYCARGDDGSYVRWYFDVWGAGTTVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 140 - protein, 482 aa, synthetic construct
EPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 141 - protein, 726 aa, synthetic construct
DIVMTQSHKFMSTSVGDRVSITCKASQDVNTAVAWYQQKPGQSPKLLIYSASYRYTGVPDRFTGSGSGTDFTFTISSVQAEDLAVYYCQQHYSTPWTFGGGTKLEIKGGGGSGGGGSGGGGSGGGGSQVQLQQSGAELAKPGASVKMSCKASGYAFTTYWMHWVKQRPGQGLEWIGYINPTTGYTDYNQKFKDKATLTADKSSSTAYMQLSSLTSEDSAVYYCARWGLPPFAYWGQGTLVTVSAEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 142 - protein, 482 aa, synthetic construct; identical to SEQ ID NO: 140

SEQ ID NO: 143 - protein, 736 aa, synthetic construct
DVVMTQTPLSLPVSLGDQASISCRSSQSLVHYNGYTYLHWYLQKPGQSPKLLIYKVSNRFSGVPDRFSGSGSGTDFTLKISRVEAEDLGVYFCSQTTHVPLTFGAGTKLELRGGGGSGGGGSGGGGSGGGGSEVMLVESGGGLVKPGGSLKLSCAASGFTFSSYAMSWVRQTPERRLEWVASISSAGTYIYYPDSVKGRFTISRDNAKNILYLQMNSLRSEDTAMYYCARHGNGYDPLWYFDVWGAGTTVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 144 - protein, 482 aa, synthetic construct; identical to SEQ ID NO: 140

SEQ ID NO: 145 - protein, 737 aa, synthetic construct
DIVMSQSPSSLAVSVGEKVTMSCKSSQSLLFSVNQKNYLAWYQQKPGQSPKLLIYWASTRESGVPDRFTGSGSGTDFTLTIGSVKAEDLAVYYCQHYYSYPWTFGGGTKLEIKGGGGSGGGGSGGGGSGGGGSQVQLQQSGAELMNPGASVRISCKATGYTFSTSWIEWIKQRPGHGLEWIGEILPGSGSTNYNEKFKGKATFTADTSSNTAYMQLSSLTFEDSAVYYCARGDDGSYVRWYFDVWGAGTTVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 146 - protein, 474 aa, synthetic construct
DIVMTQSHKFMSTSVGDRVSITCKASQDVNTAVAWYQQKPGQSPKLLIYSASYRYTGVPDRFTGSGSGTDFTFTISSVQAEDLAVYYCQQHYSTPWTFGGGTKLEIKGGGGSGGGGSGGGGSGGGGSQVQLQQSGAELAKPGASVKMSCKASGYAFTTYWMHWVKQRPGQGLEWIGYINPTTGYTDYNQKFKDKATLTADKSSSTAYMQLSSLTSEDSAVYYCARWGLPPFAYWGQGTLVTVSAEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 147 - protein, 726 aa, synthetic construct; identical to SEQ ID NO: 141

SEQ ID NO: 148 - protein, 484 aa, synthetic construct
DVVMTQTPLSLPVSLGDQASISCRSSQSLVHYNGYTYLHWYLQKPGQSPKLLIYKVSNRFSGVPDRFSGSGSGTDFTLKISRVEAEDLGVYFCSQTTHVPLTFGAGTKLELRGGGGSGGGGSGGGGSGGGGSEVMLVESGGGLVKPGGSLKLSCAASGFTFSSYAMSWVRQTPERRLEWVASISSAGTYIYYPDSVKGRFTISRDNAKNILYLQMNSLRSEDTAMYYCARHGNGYDPLWYFDVWGAGTTVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 149 - protein, 736 aa, synthetic construct; identical to SEQ ID NO: 143

SEQ ID NO: 150 - protein, 485 aa, synthetic construct
DIVMSQSPSSLAVSVGEKVTMSCKSSQSLLFSVNQKNYLAWYQQKPGQSPKLLIYWASTRESGVPDRFTGSGSGTDFTLTIGSVKAEDLAVYYCQHYYSYPWTFGGGTKLEIKGGGGSGGGGSGGGGSGGGGSQVQLQQSGAELMNPGASVRISCKATGYTFSTSWIEWIKQRPGHGLEWIGEILPGSGSTNYNEKFKGKATFTADTSSNTAYMQLSSLTFEDSAVYYCARGDDGSYVRWYFDVWGAGTTVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 151 - protein, 737 aa, synthetic construct; identical to SEQ ID NO: 145

SEQ ID NO: 152 - protein, 476 aa, synthetic construct; identical to SEQ ID NO: 134

SEQ ID NO: 153 - protein, 726 aa, synthetic construct
EVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGGGSPSDIVMTQSHKFMSTSVGDRVSITCKASQDVNTAVAWYQQKPGQSPKLLIYSASYRYTGVPDRFTGSGSGTDFTFTISSVQAEDLAVYYCQQHYSTPWTFGGGTKLEIKGGGGSGGGGSGGGGSGGGGSQVQLQQSGAELAKPGASVKMSCKASGYAFTTYWMHWVKQRPGQGLEWIGYINPTTGYTDYNQKFKDKATLTADKSSSTAYMQLSSLTSEDSAVYYCARWGLPPFAYWGQGTLVTVSA

SEQ ID NO: 154 - protein, 476 aa, synthetic construct; identical to SEQ ID NO: 134

SEQ ID NO: 155 - protein, 736 aa, synthetic construct
EVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGGGSPSDVVMTQTPLSLPVSLGDQASISCRSSQSLVHYNGYTYLHWYLQKPGQSPKLLIYKVSNRFSGVPDRFSGSGSGTDFTLKISRVEAEDLGVYFCSQTTHVPLTFGAGTKLELRGGGGSGGGGSGGGGSGGGGSEVMLVESGGGLVKPGGSLKLSCAASGFTFSSYAMSWVRQTPERRLEWVASISSAGTYIYYPDSVKGRFTISRDNAKNILYLQMNSLRSEDTAMYYCARHGNGYDPLWYFDVWGAGTTVTVSS

SEQ ID NO: 156 - protein, 476 aa, synthetic construct; identical to SEQ ID NO: 134

SEQ ID NO: 157 - protein, 737 aa, synthetic construct
EVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGGGSPSDIVMSQSPSSLAVSVGEKVTMSCKSSQSLLFSVNQKNYLAWYQQKPGQSPKLLIYWASTRESGVPDRFTGSGSGTDFTLTIGSVKAEDLAVYYCQHYYSYPWTFGGGTKLEIKGGGGSGGGGSGGGGSGGGGSQVQLQQSGAELMNPGASVRISCKATGYTFSTSWIEWIKQRPGHGLEWIGEILPGSGSTNYNEKFKGKATFTADTSSNTAYMQLSSLTFEDSAVYYCARGDDGSYVRWYFDVWGAGTTVTVSS

SEQ ID NO: 158 - protein, 480 aa, synthetic construct
EPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSPSDIVMTQSHKFMSTSVGDRVSITCKASQDVNTAVAWYQQKPGQSPKLLIYSASYRYTGVPDRFTGSGSGTDFTFTISSVQAEDLAVYYCQQHYSTPWTFGGGTKLEIKGGGGSGGGGSGGGGSGGGGSQVQLQQSGAELAKPGASVKMSCKASGYAFTTYWMHWVKQRPGQGLEWIGYINPTTGYTDYNQKFKDKATLTADKSSSTAYMQLSSLTSEDSAVYYCARWGLPPFAYWGQGTLVTVSA

SEQ ID NO: 159 - protein, 726 aa, synthetic construct; identical to SEQ ID NO: 153

SEQ ID NO: 160 - protein, 490 aa, synthetic construct
EPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSPSDVVMTQTPLSLPVSLGDQASISCRSSQSLVHYNGYTYLHWYLQKPGQSPKLLIYKVSNRFSGVPDRFSGSGSGTDFTLKISRVEAEDLGVYFCSQTTHVPLTFGAGTKLELRGGGGSGGGGSGGGGSGGGGSEVMLVESGGGLVKPGGSLKLSCAASGFTFSSYAMSWVRQTPERRLEWVASISSAGTYIYYPDSVKGRFTISRDNAKNILYLQMNSLRSEDTAMYYCARHGNGYDPLWYFDVWGAGTTVTVSS

SEQ ID NO: 161 - protein, 736 aa, synthetic construct; identical to SEQ ID NO: 155

SEQ ID NO: 162 - protein, 491 aa, synthetic construct
EPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSPSDIVMSQSPSSLAVSVGEKVTMSCKSSQSLLFSVNQKNYLAWYQQKPGQSPKLLIYWASTRESGVPDRFTGSGSGTDFTLTIGSVKAEDLAVYYCQHYYSYPWTFGGGTKLEIKGGGGSGGGGSGGGGSGGGGSQVQLQQSGAELMNPGASVRISCKATGYTFSTSWIEWIKQRPGHGLEWIGEILPGSGSTNYNEKFKGKATFTADTSSNTAYMQLSSLTFEDSAVYYCARGDDGSYVRWYFDVWGAGTTVTVSS

SEQ ID NO: 163 - protein, 737 aa, synthetic construct; identical to SEQ ID NO: 157

SEQ ID NO: 164 - protein, 725 aa, synthetic construct
EVQLQESGPGLVKPSETLSLTCTVSGYSITSNYYWNWIRQPPGKGLEWMGYIRYDGSNNYNPSLKNRVTISRDTSKNQFSLKLSSVTAADTAVYYCARLDYWGQGTTVTVSSDLSGGGGSGGGGSGGGGSGGGGSTGDAVMTQTPLSLSVTPGQPASISCRSSQSLENTNGNTFLNWYLQKPGQSPQLLIYRVSNRFSGVPDRFSGSGSGTDFTLKISRVEAEDVGVYYCLQVTHVPFTFGQGTKLEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSS

SEQ ID NO: 165 - protein, 472 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 481 aa, synthetic construct; identical to SEQ ID NO: 77

SEQ ID NO: 167 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 901 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSNPQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLGGGGSGGGSQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKLGGGGSGGGSQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKL

SEQ ID NO: 169 - protein, 484 aa, synthetic construct
SSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSSGGGGSGGGGSGGGGSGGGGSDIQMTQSPSSLSASVGDRVTITCRASQDINKFLSWYQQKPGKAPKLLIHYTSSLQPGVPSRFSGSGSGTDFTLTISSLQPEDFATYYCLQYDNLYTFGQGTKLEIK

SEQ ID NO: 170 - protein, 254 aa, synthetic construct
EVVMTQSPGTLSLSPGERATLSCRSSQSLVHYNGYTYLHWYQQKPGQAPRLLIYKVSNRFSGIPARFSGSGSGTDFTLTISRLEPEDLAVYFCSQTTHVPLTFGQGTKVEIKGGGGSGGGGSGGGGSGGGGSEVQLVESGGGLVQPGRSLRLSCAASGFTFSSYAMSWVRQAPGKGLEWVASISSAGTYIYYADSVQGRFTISRDNGKNSLYLQMNSLRAEDTALYYCARHGNGYDPLWYFDVWGQGTMVTVSS

SEQ ID NO: 171 - protein, 254 aa, synthetic construct
EVVMTQSPDTLSVSPGERATLSCRSSQSLVHYNGYTYLHWYQQKPGQVPRLLIYKVSNRFSGVPARFTGSGSGTEFTLTISSLQSEDFAVYFCSQTTHVPLTFGQGTKLEIKGGGGSGGGGSGGGGSGGGGSEVQLVESGGGLVQPGRSLRLSCAASGFTFSSYAMSWVRQAPGKGLEWVASISSAGTYIYYADSVQGRFTISRDNGKNSLYLQMNSLRAEDTALYYCARHGNGYDPLWYFDVWGQGTMVTVSS

SEQ ID NO: 172 - protein, 254 aa, synthetic construct
EVVMTQSPDFQSVTPKEKVTITCRSSQSLVHYNGYTYLHWYQQKPDQSPKLLIYKVSNRFSGVPSRFSGSGSGTDFTLTINSLEAEDAAAYFCSQTTHVPLTFGPGTKVDIKGGGGSGGGGSGGGGSGGGGSEVQLVESGGGLVQPGRSLRLSCAASGFTFSSYAMSWVRQAPGKGLEWVASISSAGTYIYYADSVQGRFTISRDNGKNSLYLQMNSLRAEDTALYYCARHGNGYDPLWYFDVWGQGTMVTVSS

SEQ ID NO: 173 - protein, 254 aa, synthetic construct
DVQMTQSPILLSASVGDRVTITCRSSQSLVHYNGYTYVHWYQQRTNGSPRLLIYKVSNRFSGVPDRFSGSGSGTDFTLTISSLQPEDIADYFCQQTTHVPLTFGAGTKVEIKGGGGSGGGGSGGGGSGGGGSEVQLVESGGGLVQPGRSLRLSCAASGFTFSSYAMSWVRQAPGKGLEWVASISSAGTYIYYADSVQGRFTISRDNGKNSLYLQMNSLRAEDTALYYCARHGNGYDPLWYFDVWGQGTMVTVSS

SEQ ID NO: 174 - protein, 254 aa, synthetic construct
EVVMTQSPDFQSVTPKEKVTITCRSSQSLVHYNGYTYLHWYQQKPDQSPKLLIYKVSNRFSGVPSRFSGSGSGTDFTLTINSLEAEDAAAYFCSQTTHVPLTFGPGTKVDIKGGGGSGGGGSGGGGSGGGGSEVQLVESGGGVVQPGRSLRLSCAASGFTFSSYAMSWVRQAPGKGLEWVASISSAGTYIYYADSVKGRFTISRDNSKNTLYLQMNGLRAEDTAVYYCARHGNGYDPLWYFDVWGQGTLVTVSS

SEQ ID NO: 175 - protein, 254 aa, synthetic construct
DVQMTQSPILLSASVGDRVTITCRSSQSLVHYNGYTYVHWYQQRTNGSPRLLIYKVSNRFSGVPDRFSGSGSGTDFTLTISSLQPEDIADYFCQQTTHVPLTFGAGTKVEIKGGGGSGGGGSGGGGSGGGGSEVQLVESGGGVVQPGRSLRLSCAASGFTFSSYAMSWVRQAPGKGLEWVASISSAGTYIYYADSVKGRFTISRDNSKNTLYLQMNGLRAEDTAVYYCARHGNGYDPLWYFDVWGQGTLVTVSS

SEQ ID NO: 176 - protein, 254 aa, synthetic construct
DVVMTQSPDSLAVSLGERATINCRSSQSLVHYNGYTYLHWYQQKPGQPPKLLIYKVSNRFSGVPDRFSGSGSGTDFTLTISSLQAEDVAVYFCSQTTHVPLTFGAGTKLELRGGGGSGGGGSGGGGSGGGGSEVQLVESGGGVVQPGRSLRLSCAASGFTFSSYAMSWVRQAPGKGLEWVASISSAGTYIYYADSVKGRFTISRDNSKNTLYLQMNGLRAEDTAVYYCARHGNGYDPLWYFDVWGQGTLVTVSS

SEQ ID NO: 177 - protein, 254 aa, synthetic construct
DVVMTQSPDTLSLSPGERATLSCRSSQSLVHYNGYTYLHWYQQQPGQAPRLLIYKVSNRFSGIPDRFSGSGSGTDFTLTISRLEPEDFAVYFCSQTTHVPLTFGQGTKLEIKGGGGSGGGGSGGGGSGGGGSEVQLLESGGGLVQPGGSLRLSCAASGFTFSSYAMSWVRQAPGKGLEWVASISSAGTYIYYTDSVKGRFSISRDNSKNTLYLQMNSLRAEDTAVYYCARHGNGYDPLWYFDVWGQGTTVTVSS

SEQ ID NO: 178 - protein, 254 aa, synthetic construct
DVQMTQSPILLSASVGDRVTITCRSSQSLVHYNGYTYVHWYQQRTNGSPRLLIYKVSNRFSGVPDRFSGSGSGTDFTLTISSLQPEDIADYFCQQTTHVPLTFGAGTKVEIKGGGGSGGGGSGGGGSGGGGSEVQLLESGGGLVQPGGSLRLSCAASGFTFSSYAMSWVRQAPGKGLEWVASISSAGTYIYYTDSVKGRFSISRDNSKNTLYLQMNSLRAEDTAVYYCARHGNGYDPLWYFDVWGQGTTVTVSS

SEQ ID NO: 179 - protein, 254 aa, synthetic construct
DVVMTQSPDTLSLSPGERATLSCRSSQSLVHYNGYTYLHWYQQQPGQAPRLLIYKVSNRFSGIPDRFSGSGSGTDFTLTISRLEPEDFAVYFCSQTTHVPLTFGQGTKLEIKGGGGSGGGGSGGGGSGGGGSQVQLVESGGGLVQPGGSLRLSCAASGFTFSSYAMSWVRQAPGKGLVWVASISSAGTYIYYADSVKGRFTISRDNAKNTLYLQMTSLRAEDTAIYYCARHGNGYDPLWYFDVWGQGALVTVSS

SEQ ID NO: 180 - protein, 254 aa, synthetic construct
DVQMTQSPILLSASVGDRVTITCRSSQSLVHYNGYTYVHWYQQRTNGSPRLLIYKVSNRFSGVPDRFSGSGSGTDFTLTISSLQPEDIADYFCQQTTHVPLTFGAGTKVEIKGGGGSGGGGSGGGGSGGGGSQVQLVESGGGLVQPGGSLRLSCAASGFTFSSYAMSWVRQAPGKGLVWVASISSAGTYIYYADSVKGRFTISRDNAKNTLYLQMTSLRAEDTAIYYCARHGNGYDPLWYFDVWGQGALVTVSS

SEQ ID NO: 181 - protein, 254 aa, synthetic construct
EVVMTQSPGTLSLSPGERATLSCRSSQSLVHYNGYTYLHWYQQKPGQAPRLLIYKVSNRFSGIPARFSGSGSGTDFTLTISRLEPEDLAVYFCSQTTHVPLTFGQGTKVEIKGGGGSGGGGSGGGGSGGGGSQVQLVESGGGLVKPGGSLRLSCAASGFTFSSYAMSWVRQAPGKGLEWVASISSAGTYIYYADSVKGRFTISRDNAKNSLYLQMNSLRAEDTAVYYCARHGNGYDPLWYFDVWGAGTTVTVSS

SEQ ID NO: 182 - protein, 254 aa, synthetic construct
DVQMTQSPILLSASVGDRVTITCRSSQSLVHYNGYTYVHWYQQRTNGSPRLLIYKVSNRFSGVPDRFSGSGSGTDFTLTISSLQPEDIADYFCQQTTHVPLTFGAGTKVEIKGGGGSGGGGSGGGGSGGGGSQVQLVESGGGLVKPGGSLRLSCAASGFTFSSYAMSWVRQAPGKGLEWVASISSAGTYIYYADSVKGRFTISRDNAKNSLYLQMNSLRAEDTAVYYCARHGNGYDPLWYFDVWGAGTTVTVSS

SEQ ID NO: 183 - protein, 254 aa, synthetic construct
EVVMTQSPDFQSVTPKEKVTITCRSSQSLVHYNGYTYLHWYQQKPDQSPKLLIYKVSNRFSGVPSRFSGSGSGTDFTLTINSLEAEDAAAYFCSQTTHVPLTFGPGTKVDIKGGGGSGGGGSGGGGSGGGGSEVQLVESGGGLEQPGGSLRLSCAASGFTFSSYAMSWVRQAPGKGLEWVASISSAGTYIYYADSVKGRFTISRDNSKNTLYLQMNSLRAEDTAVYYCARHGNGYDPLWYFDVWGQGTTVTVSS

SEQ ID NO: 184 - protein, 254 aa, synthetic construct
DVQMTQSPILLSASVGDRVTITCRSSQSLVHYNGYTYVHWYQQRTNGSPRLLIYKVSNRFSGVPDRFSGSGSGTDFTLTISSLQPEDIADYFCQQTTHVPLTFGAGTKVEIKGGGGSGGGGSGGGGSGGGGSEVQLVESGGGLEQPGGSLRLSCAASGFTFSSYAMSWVRQAPGKGLEWVASISSAGTYIYYADSVKGRFTISRDNSKNTLYLQMNSLRAEDTAVYYCARHGNGYDPLWYFDVWGQGTTVTVSS

SEQ ID NO: 185 - protein, 254 aa, synthetic construct
DVQMTQSPILLSASVGDRVTITCRSSQSLVHYNGYTYVHWYQQKPGKAPKLLIYKVSNLFSGVPSRFSGSGSGTDFTLTISSLQPEDIADYFCQQTTHVPLTFGAGTKVEIKGGGGSGGGGSGGGGSGGGGSEVQLVESGGGLVQPGGSLRLSCAASGFTFSSYAMSWVRQAPGKGLEWVSAISSAGTYIYYADSVKGRFTISRDNSKNTLYLQMNSLRAEDTAVYYCARHGNGYDPLWYFDVWGQGTTVTVSS

SEQ ID NO: 186 - protein, 122 aa, synthetic construct
EVQLVESGGGLVQPGGSLRLSCAASGFTFSSYAMSWVRQAPGKGLEWVSAISSAGTYIYYADSVKGRFTISRDNSKNTLYLQMNSLRAEDTAVYYCARHGNGYDPLWYFDVWGQGTTVTVSS

SEQ ID NO: 187 - protein, 5 aa, synthetic construct
SYAMS

SEQ ID NO: 188 - protein, 17 aa, synthetic construct
AISSAGTYIYYADSVKG

SEQ ID NO: 189 - protein, 13 aa, synthetic construct
HGNGYDPLWYFDV

SEQ ID NO: 190 - protein, 112 aa, synthetic construct
DIQMTQSPSSLSASVGDRVTITCRASQSLVHYNGYTYLNWYQQKPGKAPKLLIYKVSNRFSGVPDRFSGSGSGTDFTLTISSLQPEDFATYYCQQTTHVPLTFGQGTKVEIK

SEQ ID NO: 191 - protein, 16 aa, synthetic construct
RASQSLVHYNGYTYLN

SEQ ID NO: 192 - protein, 7 aa, synthetic construct
KVSNRFS

SEQ ID NO: 193 - protein, 9 aa, synthetic construct
QQTTHVPLT

SEQ ID NO: 194 - protein, 493 aa, synthetic construct
SSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSLSASVGDRVTITCRASQSLVHYNGYTYLNWYQQKPGKAPKLLIYKVSNRFSGVPDRFSGSGSGTDFTLTISSLQPEDFATYYCQQTTHVPLTFGQGTKVEIKGGGGSGGGGSGGGGSGGGGSEVQLVESGGGLVQPGGSLRLSCAASGFTFSSYAMSWVRQAPGKGLEWVSAISSAGTYIYYADSVKGRFTISRDNSKNTLYLQMNSLRAEDTAVYYCARHGNGYDPLWYFDVWGQGTTVTVSSS

SEQ ID NO: 195 - protein, 732 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSPSQVQLVQSGAEVKKPGASVKVSCKASGYTFTRSTMHWVRQAPGQGLEWIGYINPSSAYTNYAQKFQGRVTLTADKSTSTAYMELSSLRSEDTAVYYCASPQVHYDYNGFPYWGQGTLVTVSSGGGGSGGGGSGGGGSGGGGSDIQMTQSPSSLSASVGDRVTITCRASSSVSYMNWYQQKPGKAPKRWIYDSSKLASGVPSRFSGSGSGTDFTLTISSLQPEDFATYYCQQWSRNPPTFGQGTKVEIKRS

SEQ ID NO: 196 - protein, 477 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 197 - protein, 732 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 198 - protein, 732 aa, synthetic construct; identical to SEQ ID NO: 195

SEQ ID NO: 199 - protein, 731 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 200 - protein, 730 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSEVQLVQSGAEVKKPGASVKVSCKASGYTFTSYWMNWVRQAPGQGLEWMGNIYPSGGSTNYAQKFQGRVTMTVDTSTSTVYMELSSLRSEDTAVYYCASFSDGYYAYAMDYWGQGTLVTVSSGGGGSGGGGSGGGGSGGGGSEIVMTQSPATLSLSPGERATLSCRASQSVSSYLNWYQQKPGQAPRLLIYYASRRHTGIPARFSGSGSGTDFTLTISSLQPEDFAVYYCQQGYNLPYTFGQGTKVEIK

SEQ ID NO: 201 - protein, 739 aa, synthetic construct
EVQLVQSGAEVKKPGASVKVSCKASGYTFTSYWMNWVRQAPGQGLEWMGNIYPSGGSTNYAQKFQGRVTMTVDTSTSTVYMELSSLRSEDTAVYYCASFSDGYYAYAMDYWGQGTLVTVSSGGGGSGGGGSGGGGSGGGGSEIVMTQSPATLSLSPGERATLSCRASQSVSSYLNWYQQKPGQAPRLLIYYASRRHTGIPARFSGSGSGTDFTLTISSLQPEDFAVYYCQQGYNLPYTFGQGTKVEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDAGSYVRWYFDHWGQGTLVTVSS

SEQ ID NO: 202 - protein, 255 aa, synthetic construct
DIQMTQSPSTLSASVGDRVTITCKSSQSLLFSVNQKNYLAWYQQKPGKAPKLLIYWASTRESGVPARFSGSGSGTEFTLTISSLQPDDFATYYCQHYYSYPWTFGQGTKVEVKGGGGSGGGGSGGGGSGGGGSQVQLVQSGAEVKKPGASVKVSCKASGYTFSTSWIEWIKQAPGQGLEWIGEILPGSGSTNYNENFRNKATFTADTSISTAYMELSRLRSDDTAVYYCARGDDGSYVRWYFDVWGQGTLVTVSS

SEQ ID NO: 203 - protein, 255 aa, synthetic construct
DIQMTQSPSSLSASVGDRVTITCKSSQSLLFSVNQKNYLAWYQQKPGKAPKLLIYWASTRESGVPSRFSGSGSGTDFTFTISSLQPEDIATYYCQHYYSYPWTFGGGTKVEIKGGGGSGGGGSGGGGSGGGGSQVQLVQSGAEVKKPGASVKVSCKASGYTFSTSWIEWIKQAPGQGLEWIGEILPGSGSTNYNENFRNKATFTADTSISTAYMELSRLRSDDTAVYYCARGDDGSYVRWYFDVWGQGTLVTVSS

SEQ ID NO: 204 - protein, 255 aa, synthetic construct
QIVMTQSPGTLSLSPGERATMTCKSSQSLLFSVNQKNYLAWYQQKPGQAPRLLIYWASTRESGVPDRFSGSGSGTDFTLTISRLEPEDAATYYCQHYYSYPWTFGAGTKLEIKGGGGSGGGGSGGGGSGGGGSQVKLVQSGAEVKKPGASVKVSCKASGYTFSTSWIEWIRQAPGQGLEWIGEILPGSGSTNYAQKFQGRATFTADTSTSTAYMELSSLRSEDTAVYYCARGDDGSYVRWYFDVWGQGTTVTVSS

SEQ ID NO: 205 - protein, 255 aa, synthetic construct
DIVMTQSPDSLALSLGERATMNCKSSQSLLFSVNQKNYLAWYQQKPGQPPKLLIYWASTRESGVPDRFSGSGSGTDFTLTISSLQAEDVAVYYCQHYYSYPWTFGQGTKLEIKGGGGSGGGGSGGGGSGGGGSQVKLVQSGAEVKKPGASVKVSCKASGYTFSTSWIEWIRQAPGQGLEWIGEILPGSGSTNYAQKFQGRATFTADTSTSTAYMELSSLRSEDTAVYYCARGDDGSYVRWYFDVWGQGTTVTVSS

SEQ ID NO: 206 - protein, 255 aa, synthetic construct
DIVMTQSPDSLALSLGERATMNCKSSQSLLFSVNQKNYLAWYQQKPGQPPKLLIYWASTRESGVPDRFSGSGSGTDFTLTISSLQAEDVAVYYCQHYYSYPWTFGQGTKLEIKGGGGSGGGGSGGGGSGGGGSQVQLVQSGAEVKKPGASVKVSCKASGYTFSTSWIEWVRQAPGQGLEWIGEILPGSGSTNYAQKFQGRATFTADTSTSTAYMELSSLRSEDTAVYYCARGDDGSYVRWYFDVWGQGTTVTVSS

SEQ ID NO: 207 - protein, 255 aa, synthetic construct
DIVMTQSPDSLALSLGERATMNCKSSQSLLFSVNQKNYLAWYQQKPGQPPKLLIYWASTRESGVPDRFSGSGSGTDFTLTISSLQAEDVAVYYCQHYYSYPWTFGQGTKLEIKGGGGSGGGGSGGGGSGGGGSQVQLVQSGAEVKKPGASVKVSCKASGYTFSTSWMEWVRQAPGQGLEWMGEILPGSGSTNYAQKFQGRVTMTRDTSTSTVYMELSSLRSEDTAVYYCARGDDGSYVRWYFDVWGQGTTVTVSS

SEQ ID NO: 208 - protein, 255 aa, synthetic construct
QIVMTQSPGTLSLSPGERATMTCKSSQSLLFSVNQKNYLAWYQQKPGQAPRLLIYWASTRESGVPDRFSGSGSGTDFTLTISRLEPEDAATYYCQHYYSYPWTFGAGTKLEIKGGGGSGGGGSGGGGSGGGGSQVQLVQSGAEVKKPGASVKVSCKASGYTFSTSWMEWVRQAPGQGLEWMGEILPGSGSTNYAQKFQGRVTMTRDTSTSTVYMELSSLRSEDTAVYYCARGDDGSYVRWYFDVWGQGTTVTVSS

SEQ ID NO: 209 - protein, 255 aa, synthetic construct
DIVMTQSPDSLAVSLGERATINCKSSQSLLFSVNQKNYLAWYQQKPGQPPKLLIYWASTRESGVPDRFSGSGSGTDFTLTISSLQAEDVAVYYCQHYYSYPWTFGQGTKVEIKGGGGSGGGGSGGGGSGGGGSQVQLVQSGAEVKKPGASVKVSCKASGYTFSTSWMEWVRQAPGQGLEWMGEILPGSGSTNYAQKFQGRVTMTRDTSTSTVYMELSSLRSEDTAVYYCARGDDGSYVRWYFDVWGQGTTVTVSS

SEQ ID NO: 210 - protein, 255 aa, synthetic construct
QIVMTQSPDSLALSLGERATMNCKSSQSLLFSVNQKNYLAWYQQKPGQAPRLLIYWASTRESGVPDRFSGSGSGTDFTLTISRLEPEDVATYYCQHYYSYPWTFGAGTKLEIKGGGGSGGGGSGGGGSGGGGSQVQLVQSGAEVKKPGASVKVSCKASGYTFSTSWIEWVRQAPGQGLEWIGEILPGSGSTNYAQKFQGRATFTADTSTSTAYMELSSLRSEDTAVYYCARGDDGSYVRWYFDVWGQGTTVTVSS

SEQ ID NO: 211 - protein, 255 aa, synthetic construct
DIVMTQSPDSLALSLGERATMNCKSSQSLLFSVNQKNYLAWYQQKPGQAPRLLIYWASTRESGVPDRFSGSGSGTDFTLTISRLEPEDVATYYCQHYYSYPWTFGAGTKLEIKGGGGSGGGGSGGGGSGGGGSQVQLVQSGAEVKKPGASVKVSCKASGYTFSTSWIEWVRQAPGQGLEWIGEILPGSGSTNYAQKFQGRATFTADTSTSTAYMELSSLRSEDTAVYYCARGDDGSYVRWYFDVWGQGTTVTVSS

SEQ ID NO: 212 - protein, 255 aa, synthetic construct
QIVMTQSPDSLALSLGERATMNCKSSQSLLFSVNQKNYLAWYQQKPGQPPKLLIYWASTRESGVPDRFSGSGSGTDFTLTISRLEPEDVATYYCQHYYSYPWTFGAGTKLEIKGGGGSGGGGSGGGGSGGGGSQVQLVQSGAEVKKPGASVKVSCKASGYTFSTSWIEWVRQAPGQGLEWIGEILPGSGSTNYAQKFQGRATFTADTSTSTAYMELSSLRSEDTAVYYCARGDDGSYVRWYFDVWGQGTTVTVSS

SEQ ID NO: 213 - protein, 255 aa, synthetic construct
QIVMTQSPDSLALSLGERATMNCKSSQSLLFSVNQKNYLAWYQQKPGQAPRLLIYWASTRESGVPDRFSGSGSGTDFTLTISRLEPEDVAVYYCQHYYSYPWTFGQGTKLEIKGGGGSGGGGSGGGGSGGGGSQVQLVQSGAEVKKPGASVKVSCKASGYTFSTSWIEWVRQAPGQGLEWIGEILPGSGSTNYAQKFQGRATFTADTSTSTAYMELSSLRSEDTAVYYCARGDDGSYVRWYFDVWGQGTTVTVSS

SEQ ID NO: 214 - protein, 255 aa, synthetic construct
QIVMTQSPDSLALSLGERATMNCKSSQSLLFSVNQKNYLAWYQQKPGQAPRLLIYWASTRESGVPDRFSGSGSGTDFTLTISSLQAEDVATYYCQHYYSYPWTFGAGTKLEIKGGGGSGGGGSGGGGSGGGGSQVQLVQSGAEVKKPGASVKVSCKASGYTFSTSWIEWVRQAPGQGLEWIGEILPGSGSTNYAQKFQGRATFTADTSTSTAYMELSSLRSEDTAVYYCARGDDGSYVRWYFDVWGQGTTVTVSS

SEQ ID NO: 215 - protein, 255 aa, synthetic construct
QIVMTQSPDSLAVSLGERATINCKSSQSLLFSVNQKNYLAWYQQKPGQAPRLLIYWASTRESGVPDRFSGSGSGTDFTLTISRLEPEDVATYYCQHYYSYPWTFGAGTKVEIKGGGGSGGGGSGGGGSGGGGSQVQLVQSGAEVKKPGASVKVSCKASGYTFSTSWIEWVRQAPGQGLEWIGEILPGSGSTNYAQKFQGRATFTADTSTSTAYMELSSLRSEDTAVYYCARGDDGSYVRWYFDVWGQGTTVTVSS

SEQ ID NO: 216 - protein, 255 aa, synthetic construct
QIVMTQSPDSLALSLGERATMNCKSSQSLLFSVNQKNYLAWYQQKPGQAPRLLIYWASTRESGVPDRFSGSGSGTDFTLTISRLEPEDVATYYCQHYYSYPWTFGAGTKLEIKGGGGSGGGGSGGGGSGGGGSQVQLVQSGAEVKKPGASVKVSCKASGYTFSTSWMEWVRQAPGQGLEWMGEILPGSGSTNYAQKFQGRVTMTRDTSTSTVYMELSSLRSEDTAVYYCARGDDGSYVRWYFDVWGQGTTVTVSS

SEQ ID NO: 217 - protein, 255 aa, synthetic construct
QIVMTQSPDSLALSLGERATMNCKSSQSLLFSVNQKNYLAWYQQKPGQPPKLLIYWASTRESGVPDRFSGSGSGTDFTLTISRLEPEDVATYYCQHYYSYPWTFGAGTKLEIKGGGGSGGGGSGGGGSGGGGSQVQLVQSGAEVKKPGASVKVSCKASGYTFSTSWMEWVRQAPGQGLEWMGEILPGSGSTNYAQKFQGRVTMTRDTSTSTVYMELSSLRSEDTAVYYCARGDDGSYVRWYFDVWGQGTTVTVSS

SEQ ID NO: 218 - protein, 255 aa, synthetic construct
QIVMTQSPDSLALSLGERATMNCKSSQSLLFSVNQKNYLAWYQQKPGQAPRLLIYWASTRESGVPDRFSGSGSGTDFTLTISRLEPEDVAVYYCQHYYSYPWTFGQGTKLEIKGGGGSGGGGSGGGGSGGGGSQVQLVQSGAEVKKPGASVKVSCKASGYTFSTSWMEWVRQAPGQGLEWMGEILPGSGSTNYAQKFQGRVTMTRDTSTSTVYMELSSLRSEDTAVYYCARGDDGSYVRWYFDVWGQGTTVTVSS

SEQ ID NO: 219 - protein, 255 aa, synthetic construct
QIVMTQSPDSLALSLGERATMNCKSSQSLLFSVNQKNYLAWYQQKPGQAPRLLIYWASTRESGVPDRFSGSGSGTDFTLTISSLQAEDVATYYCQHYYSYPWTFGAGTKLEIKGGGGSGGGGSGGGGSGGGGSQVQLVQSGAEVKKPGASVKVSCKASGYTFSTSWMEWVRQAPGQGLEWMGEILPGSGSTNYAQKFQGRVTMTRDTSTSTVYMELSSLRSEDTAVYYCARGDDGSYVRWYFDVWGQGTTVTVSS

SEQ ID NO: 220 - protein, 255 aa, synthetic construct
QIVMTQSPDSLAVSLGERATINCKSSQSLLFSVNQKNYLAWYQQKPGQAPRLLIYWASTRESGVPDRFSGSGSGTDFTLTISRLEPEDVATYYCQHYYSYPWTFGAGTKVEIKGGGGSGGGGSGGGGSGGGGSQVQLVQSGAEVKKPGASVKVSCKASGYTFSTSWMEWVRQAPGQGLEWMGEILPGSGSTNYAQKFQGRVTMTRDTSTSTVYMELSSLRSEDTAVYYCARGDDGSYVRWYFDVWGQGTTVTVSS

SEQ ID NO: 221 - protein, 255 aa, synthetic construct; identical to SEQ ID NO: 204

SEQ ID NO: 222 - protein, 255 aa, synthetic construct
DIQMTQSPSTLSASVGDRVTITCKSSQSLLFSVNQKNYLAWYQQKPGKAPKLLIYWASTRESGVPSRFSGSGSGTDFTLTISSLQPDDFATYYCQHYYSYPWTFGQGTKVEVKGGGGSGGGGSGGGGSGGGGSQVQLVQSGAELKKPGSSVKVSCKASGYTFSTSWIEWIKQAPGQGLEWIGRILPGSGSVHYNQDFKDKATFTADTSTNTAYMELSSLRSEDTAVYYCARGDDGSYVRWYFDVWGQGTLVTVSS

SEQ ID NO: 223 - protein, 255 aa, synthetic construct
DIVMTQSPDSLAVSLGERATINCKSSQSLLFSVNQKNYLAWYQQKPGQPPKLLIYWASYRESGVPDRFSGSGSGTDFTLTISSLQAEDVAVYYCQHYYSYPWTFGGGTKVEIKGGGGSGGGGSGGGGSGGGGSQVQLVQSGAEVKKPGASVKVSCKASGYTFSTSWIEWIRQAPGQGLEWIGNILPGSGSTNYNEKFKNRATFTADTSTSTAYMELSSLRSEDTAVYYCARGDDGSYVRWYFDVWGQGTLVTVSS

SEQ ID NO: 224 - protein, 255 aa, synthetic construct
EIVMTQSPATLSLSPGERATLSCRSSQSLLFSVNQKNYIAWYQQKPGQAPRLLIYWASTRESGVPDRFSGSGSGTDFTLTISSLEPEDFAVYYCQHYYSYPWTFGQGTKLEIKGGGGSGGGGSGGGGSGGGGSQVQLVQSGAEVKKPGASVKVSCKATGYTFSTSWIEWIRQAPGQRLEWIGRILPGSGSTNYNEKFKGKATFTADTSASTAYMELSSLRSEDTAVYYCARGDDGSYVRWYFDFWGQGTLVTVSS

SEQ ID NO: 225 - protein, 255 aa, synthetic construct
DIQMTQSPSSLSASVGDRVTITCQSSQSLLFSVNQKNYLAWYQQKPGKAPKLLIYWASTRESGVPDRFSGSGSGTDFTFTISSLQPEDIATYYCQHYYSYPWTFGGGTKVEIKGGGGSGGGGSGGGGSGGGGSQVKLVQSGAEVKKPGASVKVSCKATGYTFSTSWIEWIRQAPGQGLEWIGEILPGSGSTNYNEKFKGKATFTADTSTSTAYMELSSLRSEDTAVYYCARGDDGSYVRWYFDVWGQGTTVTVSS

SEQ ID NO: 226 - protein, 255 aa, synthetic construct
DIQMTQSPSSLSASVGDRVTITCRSSQSLLFSVNQKNYVAWYQQKPGKAPKLLIYWASTRESGVPDRFSGSGSGTDFTLTISSLQPEDFATYYCQHYYSYPWTFEQGTKVEIKGGGGSGGGGSGGGGSGGGGSEVQLVESGGGLVQPGGSLRLSCAATGYTFSTSWIEWIRQAPGKGLEWIGLILPGSGSTNYNEKFKGKATFSANTSKNTAYLQMNSLRAEDTAVYYCARGDDGSYVRWYFDYWGQGTLVTVSS

SEQ ID NO: 227 - protein, 255 aa, synthetic construct
DIQMTQSPSSLSASVGDRVTITCRSSQSLLFSVNQKNYVAWYQQKPGKAPKLLIYWASFLYSGVPSRFSGSGSGTDFTLTISSLQPEDFATYYCQHYYSYPWTFGQGTKVEIKGGGGSGGGGSGGGGSGGGGSEVQLVESGGGLVQPGGSLRLSCAASGYTFSTSWIEWIRQAPGKGLEWIGRILPGSGSTNYADSVKGRATFSADTSKNTAYLQMNSLRAEDTAVYYCARGDDGSYVRWYFDVWGQGTLVTVSS

SEQ ID NO: 228 - protein, 255 aa, synthetic construct
EIVMTQSPDFQSVTPKEKVTITCRSSQSLLFSVNQKNYLAWYQQKPDQSPKLLIYWASQSFSGVPSRFSGSGSGTDFTLTINSLEAEDAAAYYCQHYYSYPWTFGPGTKVDIKGGGGSGGGGSGGGGSGGGGSQVQLVESGGGVVQPGRSLRLSCAASGYTFSTSWIEWIRQAPAKGLEWIGIILPGSGSTYYADSVKGRATFSADTSKNTAYLQMNGLRAEDTAVYYCARGDDGSYVRWYFDVWGQGTLVTVSS

SEQ ID NO: 229 - protein, 255 aa, synthetic construct
DIQMTQSPSSLSASVGDRVTITCKSSQSLLFSVNQKNYVAWYQQKPGKAPKLLIYWASYRYTGVPSRFSGSGSGTDFTLTISSLQPEDFATYYCQHYYSYPWTFGQGTKVEIKGGGGSGGGGSGGGGSGGGGSEVQLVESGGGLVQPGGSLRLSCAASGYTFSTSWIEWIRQAPGKGLEWIGDILPGSGSTIYNQRFKGRATFSADTSKNTAYLQMNSLRAEDTAVYYCARGDDGSYVRWYFDVWGQGTLVTVSS

SEQ ID NO: 230 - protein, 255 aa, synthetic construct
DIVMTQSPDSLAVSLGERATINCKSSQSLLFSVNQKNYLAWYQQKPGQPPKLLIYWASTRESGVPDRFSGSGSGTDFTLTISSLQAEDVAVYYCQHYYSYPWTFGGGTKLEIKGGGGSGGGGSGGGGSGGGGSQVQLVQSGAEVKKPGSSVKVSCKASGYTFSTSWIEWIRQAPGQGLEWIGGILPGSGSTNYAQKFQGRATFTADTSTSTAYMELSSLRSEDTAVYYCARGDDGSYVRWYFDVWGAGTTVTVSS

SEQ ID NO: 231 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKATGYTFSTSWIEWIRQAPGQGLEWIGSILPGSGSTNYNEKFKGKATFTADTSTSTAYMELSSLRSEDTAVYYCARGDDGSYVRWYFDHWGQGTLVTVSS

SEQ ID NO: 232 - protein, 255 aa, synthetic construct
DIQMTQSPSSVSASVGDRVTITCRASQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKATGYTFSTSWIEWIRQAPGQGLEWIGSILPGSGSTNYNEKFKGKATFTADTSTSTAYMELSSLRSEDTAVYYCARGDDGSYVRWYFDHWGQGTLVTVSS

SEQ ID NO: 233 - protein, 255 aa, synthetic construct
DIQMTQSPSSVSASVGDRVTITCRSSQSLLFSVNQKNYLAWYQQKPGKAPKLLIYWASSRQSGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKATGYTFSTSWIEWIRQAPGQGLEWIGSILPGSGSTNYNEKFKGKATFTADTSTSTAYMELSSLRSEDTAVYYCARGDDGSYVRWYFDHWGQGTLVTVSS

SEQ ID NO: 234 - protein, 255 aa, synthetic construct
DIQMTQSPSSVSASVGDRVTITCRSSQSLLFSVNQKNYLAWYQQKPGKAPKLLIYWASTLESGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKATGYTFSTSWIEWIRQAPGQGLEWIGSILPGSGSTNYNEKFKGKATFTADTSTSTAYMELSSLRSEDTAVYYCARGDDGSYVRWYFDHWGQGTLVTVSS

SEQ ID NO: 235 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWIGSILPGSGSTNYNEKFKGKATFTADTSTSTAYMELSSLRSEDTAVYYCARGDDGSYVRWYFDHWGQGTLVTVSS

SEQ ID NO: 236 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWISWVRQAPGQGLEWIGGILPGSGSTNYNEKFKGKATFTADTSTSTAYMELSSLRSEDTAVYYCARGDDGSYVRWYFDHWGQGTLVTVSS

SEQ ID NO: 237 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYNEKFKGKVTITADTSTSTAYMELSSLRSEDTAVYYCARGDDGSYVRWYFDHWGQGTLVTVSS

SEQ ID NO: 238 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWIGSILPGSGSTNYAEKFKGKATFTADTSTSTAYMELSSLRSEDTAVYYCARGDDGSYVRWYFDHWGQGTLVTVSS

SEQ ID NO: 239 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWIGSILPGSGSTNYNQKFQGRATFTADTSTSTAYMELSSLRSEDTAVYYCARGDDGSYVRWYFDHWGQGTLVTVSS

SEQ ID NO: 240 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWIGSILPGSGSTNYNEKFKGKATFTADESTSTAYMELSSLRSEDTAVYYCARGDDGSYVRWYFDHWGQGTLVTVSS

SEQ ID NO: 241 - protein, 255 aa, synthetic construct
DIQMTQSPSSVSASVGDRVTITCRASQSLLFSVNQKNYLAWYQQKPGKAPKLLIYWASSRQSGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKATGYTFSTSWIEWIRQAPGQGLEWIGSILPGSGSTNYNEKFKGKATFTADTSTSTAYMELSSLRSEDTAVYYCARGDDGSYVRWYFDHWGQGTLVTVSS

SEQ ID NO: 242 - protein, 255 aa, synthetic construct
DIQMTQSPSSVSASVGDRVTITCRSSQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKATGYTFSTSWIEWIRQAPGQGLEWIGSILPGSGSTNYNEKFKGKATFTADTSTSTAYMELSSLRSEDTAVYYCARGDDGSYVRWYFDHWGQGTLVTVSS

SEQ ID NO: 243 - protein, 255 aa, synthetic construct
DIQMTQSPSSVSASVGDRVTITCRASQSLLFSVNQKNYLAWYQQKPGKAPKLLIYWASTLESGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKATGYTFSTSWIEWIRQAPGQGLEWIGSILPGSGSTNYNEKFKGKATFTADTSTSTAYMELSSLRSEDTAVYYCARGDDGSYVRWYFDHWGQGTLVTVSS

SEQ ID NO: 244 - protein, 255 aa, synthetic construct
DIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKATGYTFSTSWIEWIRQAPGQGLEWIGSILPGSGSTNYNEKFKGKATFTADTSTSTAYMELSSLRSEDTAVYYCARGDDGSYVRWYFDHWGQGTLVTVSS

SEQ ID NO: 245 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWISWVRQAPGQGLEWMGGILPGSGSTNYNEKFKGKVTITADTSTSTAYMELSSLRSEDTAVYYCARGDDGSYVRWYFDHWGQGTLVTVSS

SEQ ID NO: 246 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWISWVRQAPGQGLEWIGGILPGSGSTNYAEKFKGKATFTADTSTSTAYMELSSLRSEDTAVYYCARGDDGSYVRWYFDHWGQGTLVTVSS

SEQ ID NO: 247 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWISWVRQAPGQGLEWIGGILPGSGSTNYNQKFQGRATFTADTSTSTAYMELSSLRSEDTAVYYCARGDDGSYVRWYFDHWGQGTLVTVSS

SEQ ID NO: 248 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWISWVRQAPGQGLEWIGGILPGSGSTNYNEKFKGKATFTADESTSTAYMELSSLRSEDTAVYYCARGDDGSYVRWYFDHWGQGTLVTVSS

SEQ ID NO: 249 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAEKFKGKVTITADTSTSTAYMELSSLRSEDTAVYYCARGDDGSYVRWYFDHWGQGTLVTVSS

SEQ ID NO: 250 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYNQKFQGRVTITADTSTSTAYMELSSLRSEDTAVYYCARGDDGSYVRWYFDHWGQGTLVTVSS

SEQ ID NO: 251 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWIGSILPGSGSTNYAQKFQGRATFTADTSTSTAYMELSSLRSEDTAVYYCARGDDGSYVRWYFDHWGQGTLVTVSS

SEQ ID NO: 252 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWIGSILPGSGSTNYAEKFKGKATFTADESTSTAYMELSSLRSEDTAVYYCARGDDGSYVRWYFDHWGQGTLVTVSS

SEQ ID NO: 253 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWIGSILPGSGSTNYNQKFQGRATFTADESTSTAYMELSSLRSEDTAVYYCARGDDGSYVRWYFDHWGQGTLVTVSS

SEQ ID NO: 254 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWISWVRQAPGQGLEWMGGILPGSGSTNYAEKFKGKVTITADTSTSTAYMELSSLRSEDTAVYYCARGDDGSYVRWYFDHWGQGTLVTVSS

SEQ ID NO: 255 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWISWVRQAPGQGLEWMGGILPGSGSTNYNQKFQGRVTITADTSTSTAYMELSSLRSEDTAVYYCARGDDGSYVRWYFDHWGQGTLVTVSS

SEQ ID NO: 256 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWISWVRQAPGQGLEWMGGILPGSGSTNYNEKFKGKVTITADESTSTAYMELSSLRSEDTAVYYCARGDDGSYVRWYFDHWGQGTLVTVSS

SEQ ID NO: 257 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYVRWYFDHWGQGTLVTVSS

SEQ ID NO: 258 - protein, 255 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAEKFKGKVTITADESTSTAYMELSSLRSEDTAVYYCARGDDGSYVRWYFDHWGQGTLVTVSS

SEQ ID NO: 259 - protein, 366 aa, synthetic construct
CAGGTTCAACTGGTGCAAAGCGGGGCTGAGGTAAAAAAGCCCGGTAGCTCTGTGAAGGTGTCTTGTAAAGCCAGCGGATATACCTTTTCAACTTCCTGGATTGAGTGGATAAGGCAAGCCCCAGGGCAGGGGCTGGAGTGGATGGGCTCTATTCTGCCTGGGAGCGGATCAACTAATTATGCCCAGAAATTTCAAGGTCGGGTTACTATAACAGCAGACACCTCTACAAGTACCGCCTACATGGAGCTGAGTTCCTTGCGATCTGAGGATACAGCCGTATATTATTGTGCAAGGGGTGACGACGGAAGCTACGTCCGATGGTACTTCGATCATTGGGGACAGGGAACTCTCGTAACCGTATCATCT

SEQ ID NO: 260 - protein, 339 aa, synthetic construct
GATATACAGATGACCCAATCCCCTAGTTCCGTCTCAGCTTCAGTTGGCGACAGAGTAACTATTACATGCCGTGCTAGCCAATCCCTTCTTTTCTCAGTGAATCAGAAAAACTATCTGGCATGGTATCAACAAAAGCCCGGCAAGGCCCCCAAGCTCCTCATTTACTGGGCTAGCTCATTGCAGTCAGGAGTTCCAAGCCGTTTTTCCGGGTCAGGGTCTGGCACCGATTTTACTTTGACCATCAGTTCTTTGCAACCCGAAGACTTTGCTACATATTACTGTCAACATTACTATAGTTATCCCTGGACATTTGGACAAGGAACCAAAGTCGAGATTAAA

SEQ ID NO: 261 - protein, 248 aa, synthetic construct
DIQMTQSPASLSASVGETVTITCRASENIYSFLAWYQQKQGKSPQLLVYKAKTLTEGVPSRFSGSGSGTQFSLKINSLQPEDFGSYYCQHHYGSPPETFGGGTKVEIKGGGGSGGGGSGGGGSGGGGSDVKLVESGGGLVKPGGSLKLSCAASGFTFSSYTMSWVRQTPEKRLEWVATISSGGNYTYYPDSVKGRFTISRDNAKNTLYLQLSSLKSEDTAMYYCTRDQAYYGNLFTYWGQGTLVTVSA

SEQ ID NO: 262 - protein, 243 aa, synthetic construct
DIQMTQTTSSLSASLGDRVTISCSASQGIRNYLNWYQQKPDGTVKLLIYYTSTLHSGVPSRFSGSGSGTDYSLTISNLEPEDIATYYCQQYSKLPWTFGGGTRLEIKGGGGSGGGGSGGGGSGGGGSQIQLQQSGPELVKPGASVKISCKASGYTFTDYYINWVKQKPEQGLEWIGWTFPGSINTKYNEKFKGKATLTVDTSSSTAYMQLNSLTSEDTAVYFCARSPYEFDYWGQGTTLTVSS

SEQ ID NO: 263 - protein, 246 aa, synthetic construct
DIQMTQTTSSLSASLGDRVTISCSASQGIRNYLNWYQQKPDGTVKLLIYYTSTLHSGVPSRFSGSGSGTDYSLTISNLEPEDIATYYCQQYSKLPWTFGGGTRLEIKGGGGSGGGGSGGGGSGGGGSEVQLQESGPSLVKPSQTLSLTCSVTGDSITSGYWNWIRKFPGKKLEYMGYISYTGSTYYNPSLKSRISIARDTSKNQYFLQLNSVTTEDTATYYCTRGENWDGGYFDYWGQGTTLTVSS

SEQ ID NO: 264 - protein, 246 aa, synthetic construct
DIVMTQSHKFMSTSVGDRVTITCKASQDVGTAVAWYQKKPGQSPKLLISWASTRHTGVPDRFTGSGSGTDFTLTISNVQSEDLADYFCQQYSSYPYTFGGGTNLEIKGGGGSGGGGSGGGGSGGGGSQGQMQQSGAELVKPGASVKLSCKTSGFTFSSSYITWLKQKPGQSLEWIAWIYVGTGGISYNQKFTGKAQLTVDTSSSTAYMQFSSLTTEDSAIYYCARHGRYHWYFDVWGAGTTVTVSS

SEQ ID NO: 265 - protein, 248 aa, synthetic construct
DVQITQSPSYLAASPGETITINCRASKSISKYLAWYQEKPGKTNKLLIYSGSTLQSGIPSRFSGSGSGTDFTLTISSLEPEDFAMYYCQQHNEYPFTFGAGTKLEIKGGGGSGGGGSGGGGSGGGGSEVQLQESGPSLVKPSQTLSLTCSVTGDSITSGYWNWIRKFPGNRLEYMGFRSYSGTTYYNPSLKSRISITRDTSKNQYYLQLNSVTTEDTATYYCARFSDFGSSENTMDYWGQGTSVTVSS

SEQ ID NO: 266 - protein, 250 aa, synthetic construct
DIVLTQSPASLAVSLGQRATISCRASKSVSKSGYSYLHWYQQKPGQTPKLLIYLASNLESGVPARFSGSGSGTDFTLNIHPVEEEDAATYYCQHSWELPYTFGGGTKLEIKGGGGSGGGGSGGGGSGGGGSEVQLQESGPSLVKPSQTLSLTCSVTGDSITSGYWNWIRKFPGKKLEYMGYISYTGSTYYNPSLKSRISIARDTSKNQYFLQLNSVTTEDTATYYCTRGENWDGGYFDYWGQGTTLTVSS

SEQ ID NO: 267 - protein, 245 aa, synthetic construct
DIQMTQTTSSLSASLGDRVTISCRASQDIRNYLNWYQQKPDGTVKLLIYYTSRLQSGVPSRFSGSGSGTDYSLTITTLEQEDFATYFCQQGNMFPYTFGGGTKLEIKGGGGSGGGGSGGGGSGGGGSKVQLQQSGAEVVKPGASVKLSCKASGYNLTEYIIHWVKQRSGQGLEWIGWFYPGSGSIKYNEKFKDKATLTADKSSTTVYMDLSRLTSEDSAVYFCARHGLYYGFPYWGQGTSVTVSS

SEQ ID NO: 268 - protein, 213 aa, synthetic construct
DIQMTQSPSSLSASLGGKVTITCKASQDINKYIAWYQHKPGKGPRLLIHYTSILQPGIPSRFSGSGSGRDYSFSISNLEPEDIATYYCLQYDNLRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 452 aa, synthetic construct
EVKFEESGGGLVQPGGSMKLSCFASGFTFSYYWMNWVRQSPEKGLEWIAEIRLKSNNYATHYAESVKGRFTISRDDSKSSVYLEMNNLRAEDTGIYHCTRPPIYYGNYEAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 270 - protein, 214 aa, synthetic construct
DIVMTQSHKFMSTSVGDRVTITCKASQDVGTAVAWYQKKPGQSPKLLISWASTRHTGVPDRFTGSGSGTDFTLTISNVQSEDLADYFCQQYSSYPYTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 451 aa, synthetic construct
EVQLQESGPSLVKPSQTLSLTCSVTGDSITSGYWNWIRKFPGNRLEYMGFRSYSGTTYYNPSLKSRISITRDTSKNQYYLQLNSVTTEDTATYYCARFSDFGSSENTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 272 - protein, 741 aa, synthetic construct
DIQMTQSPSSLSASVGDRVTINCQASQSIDSNLAWFQQKPGQPPKLLIYRASNLASGVPDRFSGSGSGTDFTLTISSLEAEDVATYYCLGGVGAVSYRTSFGGGTKVEIKGGGGSGGGGSGGGGSGGGGSEVQLVESGGGLVQPGRSLRLSCTASGSDINDYPITWVRQAPGQGLEWIGFINSGGSTWYASWVKGRFTISRDDSKSIAYLQMNSLKTEDTAVYYCARGYSTYYRDFNIWGQGTLVTVSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDAGSYVRWYFDHWGQGTLVTVSS

SEQ ID NO: 273 - protein, 733 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDAGSYVRWYFDHWGQGTLVTVSS

SEQ ID NO: 274 - protein, 741 aa, synthetic construct
DIQMTQSPSSLSASVGDRVTINCQASQSIDSNLAWFQQKPGQPPKLLIYRASNLASGVPDRFSGSGSGTDFTLTISSLEAEDVATYYCLGGVGAVSYRTSFGGGTKVEIKGGGGSGGGGSGGGGSGGGGSEVQLVESGGGLVQPGRSLRLSCTASGSDINDYPITWVRQAPGQGLEWIGFINSGGSTWYASWVKGRFTISRDDSKSIAYLQMNSLKTEDTAVYYCARGYSTYYRDFNIWGQGTLVTVSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYVRWYFDHWGQGTLVTVSS

SEQ ID NO: 275 - protein, 733 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYVRWYFDHWGQGTLVTVSS

SEQ ID NO: 276 - protein, 106 aa, synthetic construct
DIQMTQSPSSLSTSLGGKVTITCKASQDINKFISWYQHKPGKGPRLLIHYTSSLQPGIPSRFSGSGSGRDYSFSISNLEPEDIATYYCLQYDNLYTFGGGTKLEIK

SEQ ID NO: 277 - protein, 120 aa, synthetic construct
EVKLVESGGGLVQPGGSRKLSCAASGFTFSDYGMAWVRQAPGKGPEWVAFITSLAYIIFYSDTVTGRFTISRENAKNTLYLEMSSLRSEDTAVYYCARNYGSSPYYFDYWGQGTTLTVSS

SEQ ID NO: 278 - protein, 113 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARYFDYWGQGTLVTVSS

SEQ ID NO: 279 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 280 - protein, 737 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYNRWYFDHWGQGTLVTVSS

SEQ ID NO: 281 - protein, 114 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDAFDVWGQGTMVTVSS

SEQ ID NO: 282 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKLEIK

SEQ ID NO: 283 - protein, 115 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARAEYFQHWGQGTLVTVSS

SEQ ID NO: 284 - protein, 107 aa, synthetic construct
DIQMTQSPSSLSASVGDRVTITCRASQGISNYLAWFQQKPGKAPKSLIYAASSLQSGVPSRFSGSGSGTDFTLTISSLQPEDFATYYCQQYNSYPWTFGQGTKVEIK

SEQ ID NO: 285 - protein, 118 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYYYYYGMDVWGQGTTVTVSS

SEQ ID NO: 286 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKVEIK

SEQ ID NO: 287 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLR

SEQ ID NO: 288 - protein, 120 aa, synthetic construct; identical to SEQ ID NO: 277

SEQ ID NO: 289 - protein, 120 aa, synthetic construct
EVQLVESGGGLVQPGGSLRLSCAASGFTFSDYGMNWVRQAPGKGPEWVASITSLAYIIYYADSVKGRFTISRDNAKNSLYLQMNSLRAEDTAVYYCARNYGSSPYYFDYWGQGTLVTVSS

SEQ ID NO: 290 - protein, 120 aa, synthetic construct
EVQLLESGGGLVQPGGSLRLSCAASGFTFSDYGMSWVRQAPGKGPEWVAAITSLAYIIYYADSVKGRFTISRDNSKNTLYLQMNSLRAEDTAVYYCARNYGSSPYYFDYWGQGTLVTVSS

SEQ ID NO: 291 - protein, 120 aa, synthetic construct
EVQLVESGGGLVKPGGSLRLSCAASGFTFSDYGMAWVRQAPGKGLEWVAFITSLAYIIYYADSVKGRFTISRDNAKNSLYLQMNSLRAEDTAVYYCARNYGSSPYYFDYWGQGTLVTVSS

SEQ ID NO: 292 - protein, 120 aa, synthetic construct; identical to SEQ ID NO: 4

SEQ ID NO: 293 - protein, 107 aa, synthetic construct
DIQMTQSPSSLSTSLGGKVTITCKASQDINKFISWYQHKPGKGPRLLIHYTSSLQPGIPSRFSGSGSGRDYSFSISNLEPEDIATYYCLQSYDNLYTFGGGTKLEIK

SEQ ID NO: 294 - protein, 107 aa, synthetic construct
DIQMTQSPSSLSASVGDRVTITCRASQDINKFLSWYQHKPGKAPKLLIHYTSSLQPGVPSRFSGSGSGRDYTLTISSLQPEDFATYYCLQSYDNLYTFGQGTKLEIK

SEQ ID NO: 295 - protein, 107 aa, synthetic construct; identical to SEQ ID NO: 294

SEQ ID NO: 296 - protein, 107 aa, synthetic construct
DIQMTQSPSSLSASVGDRVTITCRASQDINKFLSWYQQKPGKAPKLLIHYTSSLQPGVPSRFSGSGSGTDFTLTISSLQPEDFATYYCLQSYDNLYTFGQGTKLEIK

SEQ ID NO: 297 - protein, 107 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQSYDNLYTFGQGTKLEIK

SEQ ID NO: 298 - protein, 122 aa, synthetic construct
QVQLQQSGAELMNPGASVRISCKATGYTFSTSWIEWIKQRPGHGLEWIGEILPGSGSTNYNEKFKGKATFTADTSSNTAYMQLSSLTFEDSAVYYCARGDDGSYVRWYFDVWGAGTTVTVSS

SEQ ID NO: 299 - protein, 122 aa, synthetic construct
QVQLVQSGAEVKKPGASVKVSCKASGYTFSTSWIEWIKQAPGQGLEWIGEILPGSGSTNYNENFRNKATFTADTSISTAYMELSRLRSDDTAVYYCARGDDGSYVRWYFDVWGQGTLVTVSS

SEQ ID NO: 300 - protein, 122 aa, synthetic construct; identical to SEQ ID NO: 299

SEQ ID NO: 301 - protein, 122 aa, synthetic construct
QVKLVQSGAEVKKPGASVKVSCKASGYTFSTSWIEWIRQAPGQGLEWIGEILPGSGSTNYAQKFQGRATFTADTSTSTAYMELSSLRSEDTAVYYCARGDDGSYVRWYFDVWGQGTTVTVSS

SEQ ID NO: 302 - protein, 122 aa, synthetic construct; identical to SEQ ID NO: 301

SEQ ID NO: 303 - protein, 122 aa, synthetic construct
QVQLVQSGAEVKKPGASVKVSCKASGYTFSTSWIEWVRQAPGQGLEWIGEILPGSGSTNYAQKFQGRATFTADTSTSTAYMELSSLRSEDTAVYYCARGDDGSYVRWYFDVWGQGTTVTVSS

SEQ ID NO: 304 - protein, 122 aa, synthetic construct
QVQLVQSGAEVKKPGASVKVSCKASGYTFSTSWMEWVRQAPGQGLEWMGEILPGSGSTNYAQKFQGRVTMTRDTSTSTVYMELSSLRSEDTAVYYCARGDDGSYVRWYFDVWGQGTTVTVSS

SEQ ID NO: 305 - protein, 122 aa, synthetic construct; identical to SEQ ID NO: 304

SEQ ID NO: 306 - protein, 122 aa, synthetic construct; identical to SEQ ID NO: 304

SEQ ID NO: 307 - protein, 122 aa, synthetic construct; identical to SEQ ID NO: 303

SEQ ID NO: 308 - protein, 122 aa, synthetic construct; identical to SEQ ID NO: 303

SEQ ID NO: 309 - protein, 122 aa, synthetic construct; identical to SEQ ID NO: 303

SEQ ID NO: 310 - protein, 122 aa, synthetic construct; identical to SEQ ID NO: 303

SEQ ID NO: 311 - protein, 122 aa, synthetic construct; identical to SEQ ID NO: 303

SEQ ID NO: 312 - protein, 122 aa, synthetic construct; identical to SEQ ID NO: 303

SEQ ID NO: 313 - protein, 122 aa, synthetic construct; identical to SEQ ID NO: 304

SEQ ID NO: 314 - protein, 122 aa, synthetic construct; identical to SEQ ID NO: 304

SEQ ID NO: 315 - protein, 122 aa, synthetic construct; identical to SEQ ID NO: 304

SEQ ID NO: 316 - protein, 122 aa, synthetic construct; identical to SEQ ID NO: 304

SEQ ID NO: 317 - protein, 122 aa, synthetic construct; identical to SEQ ID NO: 304

SEQ ID NO: 318 - protein, 122 aa, synthetic construct; identical to SEQ ID NO: 301

SEQ ID NO: 319 - protein, 113 aa, synthetic construct
DIVMSQSPSSLAVSVGEKVTMSCKSSQSLLFSVNQKNYLAWYQQKPGQSPKLLIYWASTRESGVPDRFTGSGSGTDFTLTIGSVKAEDLAVYYCQHYYSYPWTFGGGTKLEIK

SEQ ID NO: 320 - protein, 113 aa, synthetic construct
DIQMTQSPSTLSASVGDRVTITCKSSQSLLFSVNQKNYLAWYQQKPGKAPKLLIYWASTRESGVPARFSGSGSGTEFTLTISSLQPDDFATYYCQHYYSYPWTFGQGTKVEVK

SEQ ID NO: 321 - protein, 113 aa, synthetic construct
DIQMTQSPSSLSASVGDRVTITCKSSQSLLFSVNQKNYLAWYQQKPGKAPKLLIYWASTRESGVPSRFSGSGSGTDFTFTISSLQPEDIATYYCQHYYSYPWTFGGGTKVEIK

SEQ ID NO: 322 - protein, 113 aa, synthetic construct
QIVMTQSPGTLSLSPGERATMTCKSSQSLLFSVNQKNYLAWYQQKPGQAPRLLIYWASTRESGVPDRFSGSGSGTDFTLTISRLEPEDAATYYCQHYYSYPWTFGAGTKLEIK

SEQ ID NO: 323 - protein, 113 aa, synthetic construct
DIVMTQSPDSLALSLGERATMNCKSSQSLLFSVNQKNYLAWYQQKPGQPPKLLIYWASTRESGVPDRFSGSGSGTDFTLTISSLQAEDVAVYYCQHYYSYPWTFGQGTKLEIK

SEQ ID NO: 324 - protein, 113 aa, synthetic construct; identical to SEQ ID NO: 323

SEQ ID NO: 325 - protein, 113 aa, synthetic construct; identical to SEQ ID NO: 323

SEQ ID NO: 326 - protein, 113 aa, synthetic construct; identical to SEQ ID NO: 322

SEQ ID NO: 327 - protein, 113 aa, synthetic construct
DIVMTQSPDSLAVSLGERATINCKSSQSLLFSVNQKNYLAWYQQKPGQPPKLLIYWASTRESGVPDRFSGSGSGTDFTLTISSLQAEDVAVYYCQHYYSYPWTFGQGTKVEIK

SEQ ID NO: 328 - protein, 113 aa, synthetic construct
QIVMTQSPDSLALSLGERATMNCKSSQSLLFSVNQKNYLAWYQQKPGQAPRLLIYWASTRESGVPDRFSGSGSGTDFTLTISRLEPEDVATYYCQHYYSYPWTFGAGTKLEIK

SEQ ID NO: 329 - protein, 113 aa, synthetic construct
DIVMTQSPDSLALSLGERATMNCKSSQSLLFSVNQKNYLAWYQQKPGQAPRLLIYWASTRESGVPDRFSGSGSGTDFTLTISRLEPEDVATYYCQHYYSYPWTFGAGTKLEIK

SEQ ID NO: 330 - protein, 113 aa, synthetic construct
QIVMTQSPDSLALSLGERATMNCKSSQSLLFSVNQKNYLAWYQQKPGQPPKLLIYWASTRESGVPDRFSGSGSGTDFTLTISRLEPEDVATYYCQHYYSYPWTFGAGTKLEIK

SEQ ID NO: 331 - protein, 113 aa, synthetic construct
QIVMTQSPDSLALSLGERATMNCKSSQSLLFSVNQKNYLAWYQQKPGQAPRLLIYWASTRESGVPDRFSGSGSGTDFTLTISRLEPEDVAVYYCQHYYSYPWTFGQGTKLEIK

SEQ ID NO: 332 - protein, 113 aa, synthetic construct
QIVMTQSPDSLALSLGERATMNCKSSQSLLFSVNQKNYLAWYQQKPGQAPRLLIYWASTRESGVPDRFSGSGSGTDFTLTISSLQAEDVATYYCQHYYSYPWTFGAGTKLEIK

SEQ ID NO: 333 - protein, 113 aa, synthetic construct
QIVMTQSPDSLAVSLGERATINCKSSQSLLFSVNQKNYLAWYQQKPGQAPRLLIYWASTRESGVPDRFSGSGSGTDFTLTISRLEPEDVATYYCQHYYSYPWTFGAGTKVEIK

SEQ ID NO: 334 - protein, 113 aa, synthetic construct; identical to SEQ ID NO: 328

SEQ ID NO: 335 - protein, 113 aa, synthetic construct; identical to SEQ ID NO: 330

SEQ ID NO: 336 - protein, 113 aa, synthetic construct; identical to SEQ ID NO: 331

SEQ ID NO: 337 - protein, 113 aa, synthetic construct; identical to SEQ ID NO: 332

SEQ ID NO: 338 - protein, 113 aa, synthetic construct; identical to SEQ ID NO: 333

SEQ ID NO: 339 - protein, 113 aa, synthetic construct; identical to SEQ ID NO: 322

SEQ ID NO: 340 - protein, 122 aa, synthetic construct; identical to SEQ ID NO: 298

SEQ ID NO: 341 - protein, 122 aa, synthetic construct
QVQLVQSGAELKKPGSSVKVSCKASGYTFSTSWIEWIKQAPGQGLEWIGRILPGSGSVHYNQDFKDKATFTADTSTNTAYMELSSLRSEDTAVYYCARGDDGSYVRWYFDVWGQGTLVTVSS

SEQ ID NO: 342 - protein, 122 aa, synthetic construct
QVQLVQSGAEVKKPGASVKVSCKASGYTFSTSWIEWIRQAPGQGLEWIGNILPGSGSTNYNEKFKNRATFTADTSTSTAYMELSSLRSEDTAVYYCARGDDGSYVRWYFDVWGQGTLVTVSS

SEQ ID NO: 343 - protein, 122 aa, synthetic construct
QVQLVQSGAEVKKPGASVKVSCKATGYTFSTSWIEWIRQAPGQRLEWIGRILPGSGSTNYNEKFKGKATFTADTSASTAYMELSSLRSEDTAVYYCARGDDGSYVRWYFDFWGQGTLVTVSS

SEQ ID NO: 344 - protein, 122 aa, synthetic construct
QVKLVQSGAEVKKPGASVKVSCKATGYTFSTSWIEWIRQAPGQGLEWIGEILPGSGSTNYNEKFKGKATFTADTSTSTAYMELSSLRSEDTAVYYCARGDDGSYVRWYFDVWGQGTTVTVSS

SEQ ID NO: 345 - protein, 122 aa, synthetic construct
EVQLVESGGGLVQPGGSLRLSCAATGYTFSTSWIEWIRQAPGKGLEWIGLILPGSGSTNYNEKFKGKATFSANTSKNTAYLQMNSLRAEDTAVYYCARGDDGSYVRWYFDYWGQGTLVTVSS

SEQ ID NO: 346 - protein, 122 aa, synthetic construct
EVQLVESGGGLVQPGGSLRLSCAASGYTFSTSWIEWIRQAPGKGLEWIGRILPGSGSTNYADSVKGRATFSADTSKNTAYLQMNSLRAEDTAVYYCARGDDGSYVRWYFDVWGQGTLVTVSS

SEQ ID NO: 347 - protein, 122 aa, synthetic construct
QVQLVESGGGVVQPGRSLRLSCAASGYTFSTSWIEWIRQAPAKGLEWIGIILPGSGSTYYADSVKGRATFSADTSKNTAYLQMNGLRAEDTAVYYCARGDDGSYVRWYFDVWGQGTLVTVSS

SEQ ID NO: 348 - protein, 122 aa, synthetic construct
EVQLVESGGGLVQPGGSLRLSCAASGYTFSTSWIEWIRQAPGKGLEWIGDILPGSGSTIYNQRFKGRATFSADTSKNTAYLQMNSLRAEDTAVYYCARGDDGSYVRWYFDVWGQGTLVTVSS

SEQ ID NO: 349 - protein, 122 aa, synthetic construct
QVQLVQSGAEVKKPGSSVKVSCKASGYTFSTSWIEWIRQAPGQGLEWIGGILPGSGSTNYAQKFQGRATFTADTSTSTAYMELSSLRSEDTAVYYCARGDDGSYVRWYFDVWGAGTTVTVSS

SEQ ID NO: 350 - protein, 122 aa, synthetic construct
QVQLVQSGAEVKKPGSSVKVSCKATGYTFSTSWIEWIRQAPGQGLEWIGSILPGSGSTNYNEKFKGKATFTADTSTSTAYMELSSLRSEDTAVYYCARGDDGSYVRWYFDHWGQGTLVTVSS

SEQ ID NO: 351 - protein, 122 aa, synthetic construct; identical to SEQ ID NO: 350

SEQ ID NO: 352 - protein, 122 aa, synthetic construct; identical to SEQ ID NO: 350

SEQ ID NO: 353 - protein, 122 aa, synthetic construct; identical to SEQ ID NO: 350

SEQ ID NO: 354 - protein, 122 aa, synthetic construct
QVQLVQSGAEVKKPGSSVKVSCKASGYTFSTSWIEWIRQAPGQGLEWIGSILPGSGSTNYNEKFKGKATFTADTSTSTAYMELSSLRSEDTAVYYCARGDDGSYVRWYFDHWGQGTLVTVSS

SEQ ID NO: 355 - protein, 122 aa, synthetic construct
QVQLVQSGAEVKKPGSSVKVSCKASGYTFSTSWISWVRQAPGQGLEWIGGILPGSGSTNYNEKFKGKATFTADTSTSTAYMELSSLRSEDTAVYYCARGDDGSYVRWYFDHWGQGTLVTVSS

SEQ ID NO: 356 - protein, 122 aa, synthetic construct
QVQLVQSGAEVKKPGSSVKVSCKASGYTFSTSWIEWIRQAPGQGLEWMGSILPGSGSTNYNEKFKGKVTITADTSTSTAYMELSSLRSEDTAVYYCARGDDGSYVRWYFDHWGQGTLVTVSS

SEQ ID NO: 357 - protein, 122 aa, synthetic construct
QVQLVQSGAEVKKPGSSVKVSCKASGYTFSTSWIEWIRQAPGQGLEWIGSILPGSGSTNYAEKFKGKATFTADTSTSTAYMELSSLRSEDTAVYYCARGDDGSYVRWYFDHWGQGTLVTVSS

SEQ ID NO: 358 - protein, 122 aa, synthetic construct
QVQLVQSGAEVKKPGSSVKVSCKASGYTFSTSWIEWIRQAPGQGLEWIGSILPGSGSTNYNQKFQGRATFTADTSTSTAYMELSSLRSEDTAVYYCARGDDGSYVRWYFDHWGQGTLVTVSS

SEQ ID NO: 359 - protein, 122 aa, synthetic construct
QVQLVQSGAEVKKPGSSVKVSCKASGYTFSTSWIEWIRQAPGQGLEWIGSILPGSGSTNYNEKFKGKATFTADESTSTAYMELSSLRSEDTAVYYCARGDDGSYVRWYFDHWGQGTLVTVSS

SEQ ID NO: 360 - protein, 122 aa, synthetic construct; identical to SEQ ID NO: 350

SEQ ID NO: 361 - protein, 122 aa, synthetic construct; identical to SEQ ID NO: 350

SEQ ID NO: 362 - protein, 122 aa, synthetic construct; identical to SEQ ID NO: 350

SEQ ID NO: 363 - protein, 122 aa, synthetic construct; identical to SEQ ID NO: 350

SEQ ID NO: 364 - protein, 122 aa, synthetic construct
QVQLVQSGAEVKKPGSSVKVSCKASGYTFSTSWISWVRQAPGQGLEWMGGILPGSGSTNYNEKFKGKVTITADTSTSTAYMELSSLRSEDTAVYYCARGDDGSYVRWYFDHWGQGTLVTVSS

SEQ ID NO: 365 - protein, 122 aa, synthetic construct
QVQLVQSGAEVKKPGSSVKVSCKASGYTFSTSWISWVRQAPGQGLEWIGGILPGSGSTNYAEKFKGKATFTADTSTSTAYMELSSLRSEDTAVYYCARGDDGSYVRWYFDHWGQGTLVTVSS

SEQ ID NO: 366 - protein, 122 aa, synthetic construct
QVQLVQSGAEVKKPGSSVKVSCKASGYTFSTSWISWVRQAPGQGLEWIGGILPGSGSTNYNQKFQGRATFTADTSTSTAYMELSSLRSEDTAVYYCARGDDGSYVRWYFDHWGQGTLVTVSS

SEQ ID NO: 367 - protein, 122 aa, synthetic construct
QVQLVQSGAEVKKPGSSVKVSCKASGYTFSTSWISWVRQAPGQGLEWIGGILPGSGSTNYNEKFKGKATFTADESTSTAYMELSSLRSEDTAVYYCARGDDGSYVRWYFDHWGQGTLVTVSS

SEQ ID NO: 368 - protein, 122 aa, synthetic construct
QVQLVQSGAEVKKPGSSVKVSCKASGYTFSTSWIEWIRQAPGQGLEWMGSILPGSGSTNYAEKFKGKVTITADTSTSTAYMELSSLRSEDTAVYYCARGDDGSYVRWYFDHWGQGTLVTVSS

SEQ ID NO: 369 - protein, 122 aa, synthetic construct; identical to SEQ ID NO: 24

SEQ ID NO: 370 - protein, 122 aa, synthetic construct
QVQLVQSGAEVKKPGSSVKVSCKASGYTFSTSWIEWIRQAPGQGLEWIGSILPGSGSTNYAQKFQGRATFTADTSTSTAYMELSSLRSEDTAVYYCARGDDGSYVRWYFDHWGQGTLVTVSS

SEQ ID NO: 371 - protein, 122 aa, synthetic construct
QVQLVQSGAEVKKPGSSVKVSCKASGYTFSTSWIEWIRQAPGQGLEWIGSILPGSGSTNYAEKFKGKATFTADESTSTAYMELSSLRSEDTAVYYCARGDDGSYVRWYFDHWGQGTLVTVSS

SEQ ID NO: 372 - protein, 122 aa, synthetic construct
QVQLVQSGAEVKKPGSSVKVSCKASGYTFSTSWIEWIRQAPGQGLEWIGSILPGSGSTNYNQKFQGRATFTADESTSTAYMELSSLRSEDTAVYYCARGDDGSYVRWYFDHWGQGTLVTVSS

SEQ ID NO: 373 - protein, 122 aa, synthetic construct
QVQLVQSGAEVKKPGSSVKVSCKASGYTFSTSWISWVRQAPGQGLEWMGGILPGSGSTNYAEKFKGKVTITADTSTSTAYMELSSLRSEDTAVYYCARGDDGSYVRWYFDHWGQGTLVTVSS

SEQ ID NO: 374 - protein, 122 aa, synthetic construct
QVQLVQSGAEVKKPGSSVKVSCKASGYTFSTSWISWVRQAPGQGLEWMGGILPGSGSTNYNQKFQGRVTITADTSTSTAYMELSSLRSEDTAVYYCARGDDGSYVRWYFDHWGQGTLVTVSS

SEQ ID NO: 375 - protein, 122 aa, synthetic construct
QVQLVQSGAEVKKPGSSVKVSCKASGYTFSTSWISWVRQAPGQGLEWMGGILPGSGSTNYNEKFKGKVTITADESTSTAYMELSSLRSEDTAVYYCARGDDGSYVRWYFDHWGQGTLVTVSS

SEQ ID NO: 376 - protein, 122 aa, synthetic construct; identical to SEQ ID NO: 13

SEQ ID NO: 377 - protein, 122 aa, synthetic construct
QVQLVQSGAEVKKPGSSVKVSCKASGYTFSTSWIEWIRQAPGQGLEWMGSILPGSGSTNYAEKFKGKVTITADESTSTAYMELSSLRSEDTAVYYCARGDDGSYVRWYFDHWGQGTLVTVSS

SEQ ID NO: 378 - protein, 122 aa, synthetic construct; identical to SEQ ID NO: 13

SEQ ID NO: 379 - protein, 113 aa, synthetic construct; identical to SEQ ID NO: 319

SEQ ID NO: 380 - protein, 113 aa, synthetic construct
DIQMTQSPSTLSASVGDRVTITCKSSQSLLFSVNQKNYLAWYQQKPGKAPKLLIYWASTRESGVPSRFSGSGSGTDFTLTISSLQPDDFATYYCQHYYSYPWTFGQGTKVEVK

SEQ ID NO: 381 - protein, 113 aa, synthetic construct
DIVMTQSPDSLAVSLGERATINCKSSQSLLFSVNQKNYLAWYQQKPGQPPKLLIYWASYRESGVPDRFSGSGSGTDFTLTISSLQAEDVAVYYCQHYYSYPWTFGGGTKVEIK

SEQ ID NO: 382 - protein, 113 aa, synthetic construct
EIVMTQSPATLSLSPGERATLSCRSSQSLLFSVNQKNYIAWYQQKPGQAPRLLIYWASTRESGVPDRFSGSGSGTDFTLTISSLEPEDFAVYYCQHYYSYPWTFGQGTKLEIK

SEQ ID NO: 383 - protein, 113 aa, synthetic construct
DIQMTQSPSSLSASVGDRVTITCQSSQSLLFSVNQKNYLAWYQQKPGKAPKLLIYWASTRESGVPDRFSGSGSGTDFTFTISSLQPEDIATYYCQHYYSYPWTFGGGTKVEIK

SEQ ID NO: 384 - protein, 113 aa, synthetic construct
DIQMTQSPSSLSASVGDRVTITCRSSQSLLFSVNQKNYVAWYQQKPGKAPKLLIYWASTRESGVPDRFSGSGSGTDFTLTISSLQPEDFATYYCQHYYSYPWTFEQGTKVEIK

SEQ ID NO: 385 - protein, 113 aa, synthetic construct
DIQMTQSPSSLSASVGDRVTITCRSSQSLLFSVNQKNYVAWYQQKPGKAPKLLIYWASFLYSGVPSRFSGSGSGTDFTLTISSLQPEDFATYYCQHYYSYPWTFGQGTKVEIK

SEQ ID NO: 386 - protein, 113 aa, synthetic construct
EIVMTQSPDFQSVTPKEKVTITCRSSQSLLFSVNQKNYLAWYQQKPDQSPKLLIYWASQSFSGVPSRFSGSGSGTDFTLTINSLEAEDAAAYYCQHYYSYPWTFGPGTKVDIK

SEQ ID NO: 387 - protein, 113 aa, synthetic construct
DIQMTQSPSSLSASVGDRVTITCKSSQSLLFSVNQKNYVAWYQQKPGKAPKLLIYWASYRYTGVPSRFSGSGSGTDFTLTISSLQPEDFATYYCQHYYSYPWTFGQGTKVEIK

SEQ ID NO: 388 - protein, 113 aa, synthetic construct
DIVMTQSPDSLAVSLGERATINCKSSQSLLFSVNQKNYLAWYQQKPGQPPKLLIYWASTRESGVPDRFSGSGSGTDFTLTISSLQAEDVAVYYCQHYYSYPWTFGGGTKLEIK

SEQ ID NO: 389 - protein, 113 aa, synthetic construct
DIQMTQSPSSVSASVGDRVTITCRSSQSLLFSVNQKNYLAWYQQKPGKAPKLLIYWASTRESGVPDRFSGSGSGTDFTLTISSLQPEDFATYYCQHYYSYPWTFGQGTKVEIK

SEQ ID NO: 390 - protein, 113 aa, synthetic construct
DIQMTQSPSSVSASVGDRVTITCRASQSLLFSVNQKNYLAWYQQKPGKAPKLLIYWASTRESGVPDRFSGSGSGTDFTLTISSLQPEDFATYYCQHYYSYPWTFGQGTKVEIK

SEQ ID NO: 391 - protein, 113 aa, synthetic construct
DIQMTQSPSSVSASVGDRVTITCRSSQSLLFSVNQKNYLAWYQQKPGKAPKLLIYWASSRQSGVPDRFSGSGSGTDFTLTISSLQPEDFATYYCQHYYSYPWTFGQGTKVEIK

SEQ ID NO: 392 - protein, 113 aa, synthetic construct
DIQMTQSPSSVSASVGDRVTITCRSSQSLLFSVNQKNYLAWYQQKPGKAPKLLIYWASTLESGVPSRFSGSGSGTDFTLTISSLQPEDFATYYCQHYYSYPWTFGQGTKVEIK

SEQ ID NO: 393 - protein, 113 aa, synthetic construct; identical to SEQ ID NO: 389

SEQ ID NO: 394 - protein, 113 aa, synthetic construct; identical to SEQ ID NO: 389

SEQ ID NO: 395 - protein, 113 aa, synthetic construct; identical to SEQ ID NO: 389

SEQ ID NO: 396 - protein, 113 aa, synthetic construct; identical to SEQ ID NO: 389

SEQ ID NO: 397 - protein, 113 aa, synthetic construct; identical to SEQ ID NO: 389

SEQ ID NO: 398 - protein, 113 aa, synthetic construct; identical to SEQ ID NO: 389

SEQ ID NO: 399 - protein, 113 aa, synthetic construct
DIQMTQSPSSVSASVGDRVTITCRASQSLLFSVNQKNYLAWYQQKPGKAPKLLIYWASSRQSGVPDRFSGSGSGTDFTLTISSLQPEDFATYYCQHYYSYPWTFGQGTKVEIK

SEQ ID NO: 400 - protein, 113 aa, synthetic construct
DIQMTQSPSSVSASVGDRVTITCRSSQSLLFSVNQKNYLAWYQQKPGKAPKLLIYWASSLQSGVPSRFSGSGSGTDFTLTISSLQPEDFATYYCQHYYSYPWTFGQGTKVEIK

SEQ ID NO: 401 - protein, 113 aa, synthetic construct
DIQMTQSPSSVSASVGDRVTITCRASQSLLFSVNQKNYLAWYQQKPGKAPKLLIYWASTLESGVPSRFSGSGSGTDFTLTISSLQPEDFATYYCQHYYSYPWTFGQGTKVEIK

SEQ ID NO: 402 - protein, 113 aa, synthetic construct; identical to SEQ ID NO: 17

SEQ ID NO: 403 - protein, 113 aa, synthetic construct; identical to SEQ ID NO: 389

SEQ ID NO: 404 - protein, 113 aa, synthetic construct; identical to SEQ ID NO: 389

SEQ ID NO: 405 - protein, 113 aa, synthetic construct; identical to SEQ ID NO: 389

SEQ ID NO: 406 - protein, 113 aa, synthetic construct; identical to SEQ ID NO: 389

SEQ ID NO: 407 - protein, 113 aa, synthetic construct; identical to SEQ ID NO: 389

SEQ ID NO: 408 - protein, 113 aa, synthetic construct; identical to SEQ ID NO: 389

SEQ ID NO: 409 - protein, 113 aa, synthetic construct; identical to SEQ ID NO: 389

SEQ ID NO: 410 - protein, 113 aa, synthetic construct; identical to SEQ ID NO: 389

SEQ ID NO: 411 - protein, 113 aa, synthetic construct; identical to SEQ ID NO: 389

SEQ ID NO: 412 - protein, 113 aa, synthetic construct; identical to SEQ ID NO: 389

SEQ ID NO: 413 - protein, 113 aa, synthetic construct; identical to SEQ ID NO: 389

SEQ ID NO: 414 - protein, 113 aa, synthetic construct; identical to SEQ ID NO: 389

SEQ ID NO: 415 - protein, 113 aa, synthetic construct; identical to SEQ ID NO: 389

SEQ ID NO: 416 - protein, 113 aa, synthetic construct; identical to SEQ ID NO: 389

SEQ ID NO: 417 - protein, 113 aa, synthetic construct; identical to SEQ ID NO: 17

SEQ ID NO: 418 - protein, 122 aa, synthetic construct
EVMLVESGGGLVKPGGSLKLSCAASGFTFSSYAMSWVRQTPERRLEWVASISSAGTYIYYPDSVKGRFTISRDNAKNILYLQMNSLRSEDTAMYYCARHGNGYDPLWYFDVWGAGTTVTVSS

SEQ ID NO: 419 - protein, 122 aa, synthetic construct
EVQLVESGGGLVQPGRSLRLSCAASGFTFSSYAMSWVRQAPGKGLEWVASISSAGTYIYYADSVQGRFTISRDNGKNSLYLQMNSLRAEDTALYYCARHGNGYDPLWYFDVWGQGTMVTVSS

SEQ ID NO: 420 - protein, 122 aa, synthetic construct; identical to SEQ ID NO: 419

SEQ ID NO: 421 - protein, 122 aa, synthetic construct; identical to SEQ ID NO: 419

SEQ ID NO: 422 - protein, 122 aa, synthetic construct; identical to SEQ ID NO: 419

SEQ ID NO: 423 - protein, 122 aa, synthetic construct
EVQLVESGGGVVQPGRSLRLSCAASGFTFSSYAMSWVRQAPGKGLEWVASISSAGTYIYYADSVKGRFTISRDNSKNTLYLQMNGLRAEDTAVYYCARHGNGYDPLWYFDVWGQGTLVTVSS

SEQ ID NO: 424 - protein, 122 aa, synthetic construct; identical to SEQ ID NO: 423

SEQ ID NO: 425 - protein, 122 aa, synthetic construct; identical to SEQ ID NO: 423

SEQ ID NO: 426 - protein, 122 aa, synthetic construct
EVQLLESGGGLVQPGGSLRLSCAASGFTFSSYAMSWVRQAPGKGLEWVASISSAGTYIYYTDSVKGRFSISRDNSKNTLYLQMNSLRAEDTAVYYCARHGNGYDPLWYFDVWGQGTTVTVSS

SEQ ID NO: 427 - protein, 122 aa, synthetic construct; identical to SEQ ID NO: 426

SEQ ID NO: 428 - protein, 122 aa, synthetic construct
QVQLVESGGGLVQPGGSLRLSCAASGFTFSSYAMSWVRQAPGKGLVWVASISSAGTYIYYADSVKGRFTISRDNAKNTLYLQMTSLRAEDTAIYYCARHGNGYDPLWYFDVWGQGALVTVSS

SEQ ID NO: 429 - protein, 122 aa, synthetic construct; identical to SEQ ID NO: 428

SEQ ID NO: 430 - protein, 122 aa, synthetic construct
QVQLVESGGGLVKPGGSLRLSCAASGFTFSSYAMSWVRQAPGKGLEWVASISSAGTYIYYADSVKGRFTISRDNAKNSLYLQMNSLRAEDTAVYYCARHGNGYDPLWYFDVWGAGTTVTVSS

SEQ ID NO: 431 - protein, 122 aa, synthetic construct; identical to SEQ ID NO: 430

SEQ ID NO: 432 - protein, 122 aa, synthetic construct
EVQLVESGGGLEQPGGSLRLSCAASGFTFSSYAMSWVRQAPGKGLEWVASISSAGTYIYYADSVKGRFTISRDNSKNTLYLQMNSLRAEDTAVYYCARHGNGYDPLWYFDVWGQGTTVTVSS

SEQ ID NO: 433 - protein, 122 aa, synthetic construct; identical to SEQ ID NO: 432

SEQ ID NO: 434 - protein, 122 aa, synthetic construct; identical to SEQ ID NO: 186

SEQ ID NO: 435 - protein, 122 aa, synthetic construct; identical to SEQ ID NO: 186

SEQ ID NO: 436 - protein, 112 aa, synthetic construct
DVVMTQTPLSLPVSLGDQASISCRSSQSLVHYNGYTYLHWYLQKPGQSPKLLIYKVSNRFSGVPDRFSGSGSGTDFTLKISRVEAEDLGVYFCSQTTHVPLTFGAGTKLELR

SEQ ID NO: 437 - protein, 112 aa, synthetic construct
EVVMTQSPGTLSLSPGERATLSCRSSQSLVHYNGYTYLHWYQQKPGQAPRLLIYKVSNRFSGIPARFSGSGSGTDFTLTISRLEPEDLAVYFCSQTTHVPLTFGQGTKVEIK

SEQ ID NO: 438 - protein, 112 aa, synthetic construct
EVVMTQSPDTLSVSPGERATLSCRSSQSLVHYNGYTYLHWYQQKPGQVPRLLIYKVSNRFSGVPARFTGSGSGTEFTLTISSLQSEDFAVYFCSQTTHVPLTFGQGTKLEIK

SEQ ID NO: 439 - protein, 112 aa, synthetic construct
EVVMTQSPDFQSVTPKEKVTITCRSSQSLVHYNGYTYLHWYQQKPDQSPKLLIYKVSNRFSGVPSRFSGSGSGTDFTLTINSLEAEDAAAYFCSQTTHVPLTFGPGTKVDIK

SEQ ID NO: 440 - protein, 112 aa, synthetic construct
DVQMTQSPILLSASVGDRVTITCRSSQSLVHYNGYTYVHWYQQRTNGSPRLLIYKVSNRFSGVPDRFSGSGSGTDFTLTISSLQPEDIADYFCQQTTHVPLTFGAGTKVEIK

SEQ ID NO: 441 - protein, 112 aa, synthetic construct; identical to SEQ ID NO: 439

SEQ ID NO: 442 - protein, 112 aa, synthetic construct; identical to SEQ ID NO: 440

SEQ ID NO: 443 - protein, 112 aa, synthetic construct
DVVMTQSPDSLAVSLGERATINCRSSQSLVHYNGYTYLHWYQQKPGQPPKLLIYKVSNRFSGVPDRFSGSGSGTDFTLTISSLQAEDVAVYFCSQTTHVPLTFGAGTKLELR

SEQ ID NO: 444 - protein, 112 aa, synthetic construct
DVVMTQSPDTLSLSPGERATLSCRSSQSLVHYNGYTYLHWYQQQPGQAPRLLIYKVSNRFSGIPDRFSGSGSGTDFTLTISRLEPEDFAVYFCSQTTHVPLTFGQGTKLEIK

SEQ ID NO: 445 - protein, 112 aa, synthetic construct; identical to SEQ ID NO: 440

SEQ ID NO: 446 - protein, 112 aa, synthetic construct; identical to SEQ ID NO: 444

SEQ ID NO: 447 - protein, 112 aa, synthetic construct; identical to SEQ ID NO: 440

SEQ ID NO: 448 - protein, 112 aa, synthetic construct; identical to SEQ ID NO: 437

SEQ ID NO: 449 - protein, 112 aa, synthetic construct; identical to SEQ ID NO: 440

SEQ ID NO: 450 - protein, 112 aa, synthetic construct; identical to SEQ ID NO: 439

SEQ ID NO: 451 - protein, 112 aa, synthetic construct; identical to SEQ ID NO: 440

SEQ ID NO: 452 - protein, 111 aa, synthetic construct
DVQMTQSPILLSASVGDRVTITCRSSQSLVHYNGYTYVHWYQQKPGKAPKLLIYKVSNFSGVPSRFSGSGSGTDFTLTISSLQPEDIADYFCQQTTHVPLTFGAGTKVEIK

SEQ ID NO: 453 - protein, 112 aa, synthetic construct; identical to SEQ ID NO: 190

SEQ ID NO: 454 - protein, 173 aa, synthetic construct
EPPTACSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMR

SEQ ID NO: 455 - protein, 173 aa, synthetic construct
EPPTACSDKQYLHDGQCCDLCQPGSRLTSHCTALEKTQCHPCDSGEFSAQWNREIRCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLR

SEQ ID NO: 456 - protein, 173 aa, synthetic construct
EPPTACREKQYLINSQCCSLCQPGQKLVSDCTEFTETECLPCGESEFLDTWNRETHCHQHRHCEPNQGLRVKKEGTAESDTVCTCKEGQHCTSKDCEACAQHRSCSPGFGVKQIATGVSDTICEPCPVGFFSNVSSAFEKCHPWTSCETKDLVVQQAGTNKTDVVCGPQDRLR

SEQ ID NO: 457 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHTPCIPGFGVMEMATETTDTVCHPCPVGFFSNQSSLFEKCYPWTSCEDKNLEVLQKGTSQTNVICGLKSRMR

SEQ ID NO: 458 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCKEGQHCTSKDCEACAQHRSCSPGFGVKQIATETTDTICEPCPVGFFSNVSSAFEKCHPWTSCETKDLVVQQAGTNKTDVVCGPQDRLR

SEQ ID NO: 459 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHTPCIPGFGVKQIATGVSDTICEPCPVGFFSNQSSLFEKCHPWTSCETKDLVVQQAGTSQTDVVCGPQDRLR

SEQ ID NO: 460 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVMEMATETTDTVCHPCPVGFFSNVSSAFEKCYPWTSCETKDLVVQQAGTNKTDVVCGPQDRLR

SEQ ID NO: 461 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCYPWTSCEDKNLEVLQKGTSQTNVICGLKSRMR

SEQ ID NO: 462 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVMQIATGVSDTICEPCPVGFFSNVSSAFEKCHPWTSCETKDLVVQQAGTNKTDVVCGPQDRLR

SEQ ID NO: 463 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKEIATGVSDTICEPCPVGFFSNVSSAFEKCHPWTSCETKDLVVQQAGTNKTDVVCGPQDRLR

SEQ ID NO: 464 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQMATGVSDTICEPCPVGFFSNVSSAFEKCHPWTSCETKDLVVQQAGTNKTDVVCGPQDRLR

SEQ ID NO: 465 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATEVSDTICEPCPVGFFSNVSSAFEKCHPWTSCETKDLVVQQAGTNKTDVVCGPQDRLR

SEQ ID NO: 466 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGTSDTICEPCPVGFFSNVSSAFEKCHPWTSCETKDLVVQQAGTNKTDVVCGPQDRLR

SEQ ID NO: 467 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVTDTICEPCPVGFFSNVSSAFEKCHPWTSCETKDLVVQQAGTNKTDVVCGPQDRLR

SEQ ID NO: 468 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTVCEPCPVGFFSNVSSAFEKCHPWTSCETKDLVVQQAGTNKTDVVCGPQDRLR

SEQ ID NO: 469 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICHPCPVGFFSNVSSAFEKCHPWTSCETKDLVVQQAGTNKTDVVCGPQDRLR

SEQ ID NO: 470 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCYPWTSCETKDLVVQQAGTNKTDVVCGPQDRLR

SEQ ID NO: 471 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCAPGFGVKQIATGVSDTICEPCPVGFFSNVSSAFEKCHPWTSCETKDLVVQQAGTNKTDVVCGPQDRLR

SEQ ID NO: 472 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGAGVKQIATGVSDTICEPCPVGFFSNVSSAFEKCHPWTSCETKDLVVQQAGTNKTDVVCGPQDRLR

SEQ ID NO: 473 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVAQIATGVSDTICEPCPVGFFSNVSSAFEKCHPWTSCETKDLVVQQAGTNKTDVVCGPQDRLR

SEQ ID NO: 474 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKAIATGVSDTICEPCPVGFFSNVSSAFEKCHPWTSCETKDLVVQQAGTNKTDVVCGPQDRLR

SEQ ID NO: 475 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGASDTICEPCPVGFFSNVSSAFEKCHPWTSCETKDLVVQQAGTNKTDVVCGPQDRLR

SEQ ID NO: 476 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVADTICEPCPVGFFSNVSSAFEKCHPWTSCETKDLVVQQAGTNKTDVVCGPQDRLR

SEQ ID NO: 477 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTACEPCPVGFFSNVSSAFEKCHPWTSCETKDLVVQQAGTNKTDVVCGPQDRLR

SEQ ID NO: 478 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICAPCPVGFFSNVSSAFEKCHPWTSCETKDLVVQQAGTNKTDVVCGPQDRLR

SEQ ID NO: 479 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEACPVGFFSNVSSAFEKCHPWTSCETKDLVVQQAGTNKTDVVCGPQDRLR

SEQ ID NO: 480 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCAVGFFSNVSSAFEKCHPWTSCETKDLVVQQAGTNKTDVVCGPQDRLR

SEQ ID NO: 481 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPAGFFSNVSSAFEKCHPWTSCETKDLVVQQAGTNKTDVVCGPQDRLR

SEQ ID NO: 482 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGAFSNVSSAFEKCHPWTSCETKDLVVQQAGTNKTDVVCGPQDRLR

SEQ ID NO: 483 - protein, 173 aa, synthetic construct
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEACHPWTSCETKDLVVQQAGTNKTDVVCGPQDRLR

SEQ ID NO: 484 - protein, 122 aa, synthetic construct
QVQLVQSGAEVKKPGSSVKVSCKASGYTFSTSWIEWIRQAPGQCLEWMGSILPGSGSTNYNQKFQGRVTITADTSTSTAYMELSSLRSEDTAVYYCARGDDGSYNRWYFDHWGQGTLVTVSS

SEQ ID NO: 485 - protein, 113 aa, synthetic construct
DIQMTQSPSSVSASVGDRVTITCRASQSLTFSVNQKNYLAWYQQKPGKAPKLLIYWASSLQSGVPSRFSGSGSGTDFTLTISSLQPEDFATYYCQHYYSYPWTFGCGTKVEIK

SEQ ID NO: 486 - protein, 255 aa, synthetic construct
DIQMTQSPSSVSASVGDRVTITCRASQSLTFSVNQKNYLAWYQQKPGKAPKLLIYWASSLQSGVPSRFSGSGSGTDFTLTISSLQPEDFATYYCQHYYSYPWTFGCGTKVEIKGGGGSGGGGSGGGGSGGGGSQVQLVQSGAEVKKPGSSVKVSCKASGYTFSTSWIEWIRQAPGQCLEWMGSILPGSGSTNYNQKFQGRVTITADTSTSTAYMELSSLRSEDTAVYYCARGDDGSYNRWYFDHWGQGTLVTVSS

SEQ ID NO: 487 - protein, 737 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLTFSVNQKNYLAWYQQKPGKAPKLLIYWASSLQSGVPSRFSGSGSGTDFTLTISSLQPEDFATYYCQHYYSYPWTFGCGTKVEIKGGGGSGGGGSGGGGSGGGGSQVQLVQSGAEVKKPGSSVKVSCKASGYTFSTSWIEWIRQAPGQCLEWMGSILPGSGSTNYNQKFQGRVTITADTSTSTAYMELSSLRSEDTAVYYCARGDDGSYNRWYFDHWGQGTLVTVSS

SEQ ID NO: 488 - protein, 255 aa, synthetic construct
DIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGCGTKVEIKGGGGSGGGGSGGGGSGGGGSQVQLVQSGAEVKKPGSSVKVSCKASGYTFSTSWIEWIRQAPGQCLEWMGSILPGSGSTNYNQKFQGRVTITADTSTSTAYMELSSLRSEDTAVYYCARGDDGSYNRWYFDHWGQGTLVTVSS

SEQ ID NO: 489 - protein, 737 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGCGTKVEIKGGGGSGGGGSGGGGSGGGGSQVQLVQSGAEVKKPGSSVKVSCKASGYTFSTSWIEWIRQAPGQCLEWMGSILPGSGSTNYNQKFQGRVTITADTSTSTAYMELSSLRSEDTAVYYCARGDDGSYNRWYFDHWGQGTLVTVSS

SEQ ID NO: 490 - protein, 476 aa, synthetic construct
DIQMTQSPSSLSASVGDRVTITCRASQDINKFLSWYQQKPGKAPKLLIHYTSSLQSGVPSRFSGSGSGTDFTLTISSLQPEDFATYYCLQYDNLYTFGQGTKLEIKGGGGSGGGGSGGGGSGGGGSEVQLVESGGGLVQPGGSLRLSCAASGFTFSDYGMAWVRQAPGKGLEWVSFITSLAYIIYYADSVKGRFTISRDNAKNSLYLQMNSLRAEDTAVYYCARNYGSSPYYFDYWGQGTLVTVSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 491 - protein, 492 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYVRWYFDHWGQGTLVTVSS

SEQ ID NO: 492 - protein, 491 aa, synthetic construct
EPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSVSASVGDRVTITCRASQSLLFSVNQKNYLAWYQQKPGKAPKLLIYWASSLQSGVPSRFSGSGSGTDFTLTISSLQPEDFATYYCQHYYSYPWTFGQGTKVEIKGGGGSGGGGSGGGGSGGGGSQVQLVQSGAEVKKPGSSVKVSCKASGYTFSTSWIEWIRQAPGQGLEWMGSILPGSGSTNYAQKFQGRVTITADTSTSTAYMELSSLRSEDTAVYYCARGDDGSYVRWYFDHWGQGTLVTVSS

SEQ ID NO: 493 - protein, 214 aa, synthetic construct
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 494 - protein, 452 aa, synthetic construct
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

(19) United States
(12) Patent Application Publication
(10) Pub. No.: US 2026/0217833 A1
(43) Pub. Date: Jul. 30, 2026

(54) BISPECIFIC PD-L1 AND CD40 BINDING MOLECULES AND USES THEREOF

(71) Applicant: Aptevo Research and Development LLC, Seattle, WA (US)

(72) Inventors: Peter PAVLIK, Kingston, WA (US); Michelle H. NELSON, Kent, WA (US); Matthew J. BAILEY, Seattle, WA (US); Lynda MISHER, Elkins Park, PA (US); Elizabeth R. HAGLIN, Bellevue, WA (US); Secil FRANKE-WELCH, Seattle, WA (US); Hilario RAMOS, Austin, TX (US); David BIENVENUE, Lake Forest Park, WA (US)

(21) Appl. No.: 19/145,727

(22) PCT Filed: Jan. 5, 2024
(86) PCT No.: PCT/US2024/010583

§ 371 Date: Jul. 3, 2025

Related U.S. Application Data
(60) Provisional application No. 63/478,799, filed on Jan. 6, 2023.

(51) Int. Cl. C07K 16/28

(52) U.S. Cl. CPC C07K 16/2827; C07K 16/2803; C07K 16/2863; C07K 16/2875; C07K 2317/31

(57) ABSTRACT

Provided herein are bispecific antigen-binding molecules that bind to PD-L1 and/or CD40. Further provided herein are therapeutic uses of and methods of producing such molecules and related pharmaceutical compositions, nucleic acid molecules, expression vectors and host cells.

### CROSS REFERENCE TO RELATED APPLICATION

This application claims priority from U.S. Provisional Application Ser. No. 63/478,799, filed Jan. 6, 2023, the disclosure of which is incorporated by reference herein in its entirety.

### REFERENCE TO AN ELECTRONIC SEQUENCE LISTING

The contents of the electronic sequence listing (APVO_070_01WO_SeqList_ST26.xml; Size: 536,960 bytes; and Date of Creation: Jan. 3, 2024) are herein incorporated by reference in their entirety.

### TECHNICAL FIELD

The disclosure relates to bispecific antigen-binding molecules and therapeutic uses of such molecules.

### BACKGROUND

Despite major advances in developing cancer therapies, cancer continues to be a global health burden. In 2023, there were nearly two million new cancer cases and 609, 820 cancer deaths were projected in the United States (Siegal et al., CA Cancer J Clin. 73:17-48 (2023)). Therefore, there is a need for new and effective cancer therapies, especially those that are less toxic and more targeted than traditional chemotherapy and irradiation. Novel therapies that have utilized the patients' own immune system have been attractive as it has been shown that tumor-specific T cell can both eliminate tumor and mount a memory response to prevent recurrence.

On the surface of immune cells are a series of proteins that are meant to enhance (co-stimulatory) or dampen (co-inhibitory) the immune response when triggered. Enhancing the immune response is required to fully combat foreign pathogens through cell proliferation, differentiation, and memory cell generation. During cell activation, co-stimulatory molecule interaction between immune cell types plays a major role in the nature of immune response that is generated.

Dampening of the immune response is beneficial to prevent over activation of the immune system that could lead to adverse effects such as allergy and asthma. Therefore, these proteins are referred to as a brake or a checkpoint. Checkpoint proteins are a way that the immune system communicates between many immune and non-immune cells. Cancer cells have hijacked this pathway to evade detection and killing by immune cells.

Checkpoint inhibitors have now been developed to target several inhibitory pathways. The most therapeutically successful target the PD-1/PD-L1 pathway. PD-L1 is expressed on resting T cells, B cells, DCs and macrophages and enhanced upon activation. Its normal function is to regulate the balance between activation and tolerance of T cells through the interaction with its receptors PD-1 and CD80. Additionally, PD-L1 is expressed on a wide array of cancers and is associated with reduced survival and unfavorable prognosis of patients. Antibodies that block the interaction between PD-1 and PD-L1 can remove the immunosuppressive break leading to enhanced tumor cytotoxicity.

Stimulatory receptors are utilized to enhance an immune response and generate mature cells. CD40 is one such molecule that is expressed on antigen presenting cells (B cells, macrophages and DCs). The interaction with its ligand (CD40L) on activated CD4 T cells activates APC leading to mature cells that present antigen, upregulation of costimulatory molecules and secrete inflammatory cytokines resulting in the priming of CD8 T cells.

### SUMMARY

Provided herein is a PD-L1 binding polypeptide that binds specifically to human PD-L1; wherein the PD-L1 binding polypeptide comprises, in order from amino terminus to carboxyl terminus or from carboxyl terminus to amino terminus, (a) a first binding domain; (b) a hinge region; (c) an immunoglobulin constant region; and (d) a second binding domain; wherein the first binding domain is a PD-L1 binding domain and the second binding domain binds an immunostimulatory protein; or wherein the first binding domain binds an immunostimulatory protein and the second binding domain is a PD-L1 binding domain. In some embodiments, the immunostimulatory protein is CD40, 4-1BB, CD3 or OX40.

In some embodiments of the PD-L1 binding domain, the PD-L1 binding domain comprises (i) an immunoglobulin heavy chain variable region (VH) comprising HCDR1, HCDR2, and HCDR3; and (ii) an immunoglobulin light chain variable region (VL) comprising LCDR1, LCDR2, and LCDR3. In some embodiments of the PD-L1 binding domain, the HCDR1 comprises SEQ ID NO: 1, the HCDR2 comprises SEQ ID NO: 2, and the HCDR3 comprises SEQ ID NO: 3, the LCDR1 comprises SEQ ID NO:5, the LCDR2 comprises SEQ ID NO: 6, and the LCDR3 comprises SEQ ID NO: 7. In some embodiments, the VH comprises SEQ ID NO: 4; and the VL comprises SEQ ID NO: 8. In some embodiments, the PD-L1 binding domain comprises SEQ ID NO: 9.

Further provided herein is a CD40 binding polypeptide that binds specifically to human CD40; wherein the CD40 binding polypeptide comprises, in order from amino terminus to carboxyl terminus or from carboxyl terminus to amino terminus, (a) a first binding domain; (b) a hinge region; (c) an immunoglobulin constant region; and (d) a second binding domain; wherein the first binding domain is a CD40 binding domain, and the second binding domain binds a tumor-associated antigen; or wherein the first binding domain binds a tumor-associated antigen, and the second binding domain is a CD40 binding domain. In some embodiments, the tumor-associated antigen is PD-L1, ROR1, or EGFR.

In some embodiments, the CD40 binding domain comprises (i) an immunoglobulin heavy chain variable region (VH) comprising HCDR1, HCDR2, and HCDR3; and (ii) an immunoglobulin light chain variable region (VL) comprising LCDR1, LCDR2, and LCDR3.

In some embodiments of the CD40 binding domain, (a) the HCDR1 comprises SEQ ID NO: 10, the HCDR2 comprises SEQ ID NO: 11, and the HCDR3 comprises SEQ ID NO: 12, the LCDR1 comprises SEQ ID NO: 14, the LCDR2 comprises SEQ ID NO: 15, and the LCDR3 comprises SEQ ID NO: 16; (b) the HCDR1 comprises SEQ ID NO: 10, the HCDR2 comprises SEQ ID NO: 23, and the HCDR3 comprises SEQ ID NO: 12, the LCDR1 comprises SEQ ID NO: 14, the LCDR2 comprises SEQ ID NO: 15, and the LCDR3 comprises SEQ ID NO: 16; (c) the HCDR1 comprises SEQ ID NO: 10, the HCDR2 comprises SEQ ID NO: 11, and the HCDR3 comprises SEQ ID NO: 31, the LCDR1 comprises SEQ ID NO: 14, the LCDR2 comprises SEQ ID NO: 15, and the LCDR3 comprises SEQ ID NO: 16; (d) the HCDR1 comprises SEQ ID NO: 10, the HCDR2 comprises SEQ ID NO: 23, and the HCDR3 comprises SEQ ID NO: 31, the LCDR1 comprises SEQ ID NO: 14, the LCDR2 comprises SEQ ID NO: 15, and the LCDR3 comprises SEQ ID NO: 16. (e) the HCDR1 comprises SEQ ID NO: 10, the HCDR2 comprises SEQ ID NO: 11, and the HCDR3 comprises SEQ ID NO: 12, the LCDR1 comprises SEQ ID NO: 37, the LCDR2 comprises SEQ ID NO: 15, and the LCDR3 comprises SEQ ID NO:16; (f) the HCDR1 comprises SEQ ID NO: 10, the HCDR2 comprises SEQ ID NO: 11, and the HCDR3 comprises SEQ ID NO: 12, the LCDR1 comprises SEQ ID NO: 41, the LCDR2 comprises SEQ ID NO: 15, and the LCDR3 comprises SEQ ID NO: 16; (g) the HCDR1 comprises SEQ ID NO: 10, the HCDR2 comprises SEQ ID NO: 45, and the HCDR3 comprises SEQ ID NO: 12, the LCDR1 comprises SEQ ID NO: 14, the LCDR2 comprises SEQ ID NO: 15, and the LCDR3 comprises SEQ ID NO: 16; (h) the HCDR1 comprises SEQ ID NO: 10 the HCDR2 comprises SEQ ID NO: 49, and the HCDR3 comprises SEQ ID NO: 12, the LCDR1 comprises SEQ ID NO: 14, the LCDR2 comprises SEQ ID NO: 15, and the LCDR3 comprises SEQ ID NO: 16; or (i) the HCDR1 comprises SEQ ID NO: 10, the HCDR2 comprises SEQ ID NO: 23, and the HCDR3 comprises SEQ ID NO: 31, the LCDR1 comprises SEQ ID NO: 37, the LCDR2 comprises SEQ ID NO: 15, and the LCDR3 comprises SEQ ID NO: 16.

In some embodiments of the CD40 binding domain, (a) the VH comprises SEQ ID NO: 13; and the VL comprises SEQ ID NO: 17; (b) the VH comprises SEQ ID NO: 24; and the VL comprises SEQ ID NO: 17; (c) the VH comprises SEQ ID NO: 27; and the VL comprises SEQ ID NO: 28; (d) the VH comprises SEQ ID NO: 32; and the VL comprises SEQ ID NO: 17; (e) the VH comprises SEQ ID NO:34; and the VL comprises SEQ ID NO: 17; (f) the VH comprises SEQ ID NO: 13; and the VL comprises SEQ ID NO: 38; (g) the VH comprises SEQ ID NO: 13, and the VL comprises SEQ ID NO: 42; (h) the VH comprises SEQ ID NO: 46; and the VL comprises SEQ ID NO: 17; (i) the VH comprises SEQ ID NO: 50, and the VL comprises SEQ ID NO: 17; (j) the VH comprises SEQ ID NO: 484; and the VL comprises SEQ ID NO: 485; or (k) the VH comprises SEQ ID NO: 484; and the VL comprises SEQ ID NO: 28.

In some embodiments, the CD40 binding domain comprises SEQ ID NO: 18, SEQ ID NO: 25, SEQ ID NO: 29, SEQ ID NO: 33, SEQ ID NO: 35, SEQ ID NO: 39, SEQ ID NO: 43, SEQ ID NO: 47, SEQ ID NO: 51, SEQ ID NO: 486, or SEQ ID NO: 488.

Provided herein is a binding polypeptide that binds specifically to human PD-L 1 and human CD40; wherein the binding polypeptide comprises, in order from amino terminus to carboxyl terminus or from carboxyl terminus to amino terminus, (a) a first binding domain; (b) a hinge region; (c) an immunoglobulin constant region; and (d) a second binding domain; wherein the first binding domain is a PD-L1 binding domain and the second binding domain is a CD40 binding domain; or wherein the first binding domain is a CD40 binding domain and the second binding domain is a PD-L1 binding domain.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO: 10, a HCDR2 comprising SEQ ID NO: 11, and a HCDR3 comprising SEQ ID NO: 12; and a VL comprising a LCDR1 comprising SEQ ID NO:14, a LCDR2 comprising SEQ ID NO: 15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO:10, a HCDR2 comprising SEQ ID NO:23, and a HCDR3 comprising SEQ ID NO: 12; and a VL comprising a LCDR1 comprising SEQ ID NO:14, a LCDR2 comprising SEQ ID NO: 15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO: 10, a HCDR2 comprising SEQ ID NO: 11, and a HCDR3 comprising SEQ ID NO: 31; and a VL comprising a LCDR1 comprising SEQ ID NO:14, a LCDR2 comprising SEQ ID NO: 15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO:10, a HCDR2 comprising SEQ ID NO:23, and a HCDR3 comprising SEQ ID NO: 31; and a VL comprising a LCDR1 comprising SEQ ID NO:14, a LCDR2 comprising SEQ ID NO: 15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO; 10, a HCDR2 comprising SEQ ID NO: 11, and a HCDR3 comprising SEQ ID NO:12; and a VL comprising a LCDR1 comprising SEQ ID NO:37, a LCDR2 comprising SEQ ID NO: 15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO:10, a HCDR2 comprising SEQ ID NO: 11, and a HCDR3 comprising SEQ ID NO: 12; and a VL comprising a LCDR1 comprising SEQ ID NO:41, a LCDR2 comprising SEQ ID NO:15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO:10, a HCDR2 comprising SEQ ID NO: 45, and a HCDR3 comprising SEQ ID NO: 12; and a VL comprising a LCDR1 comprising SEQ ID NO:14, a LCDR2 comprising SEQ ID NO: 15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO:10, a HCDR2 comprising SEQ ID NO: 49, and a HCDR3 comprising SEQ ID NO: 12; and a VL comprising a LCDR1 comprising SEQ ID NO:14, a LCDR2 comprising SEQ ID NO: 15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO; 10, a HCDR2 comprising SEQ ID NO: 23, and a HCDR3 comprising SEQ ID NO: 31; and a VL comprising a LCDR1 comprising SEQ ID NO:37, a LCDR2 comprising SEQ ID NO: 15, and a LCDR3 comprising SEQ ID NO: 16.

Provided herein is a binding polypeptide, wherein (a) the PD-L1 binding domain comprises a VH comprising SEQ ID NO: 4 and a VL comprising SEQ ID NO: 8; and the CD40 binding domain comprises a VH comprising SEQ ID NO: 13 and a VL comprising SEQ ID NO: 17; (b) the PD-L1 binding domain comprises a VH comprising SEQ ID NO: 4 and a VL comprising SEQ ID NO: 8; and the CD40 binding domain comprises a VH comprising SEQ ID NO: 24 and a VL comprising SEQ ID NO: 17; (c) the PD-L1 binding domain comprises a VH comprising SEQ ID NO: 4 and a VL comprising SEQ ID NO: 8; and the CD40 binding domain comprises a VH comprising SEQ ID NO: 27 and a VL comprising SEQ ID NO: 28; (d) the PD-L1 binding domain comprises a VH comprising SEQ ID NO: 4 and a VL comprising SEQ ID NO: 8; and the CD40 binding domain comprises a VH comprising SEQ ID NO: 32 and a VL comprising SEQ ID NO: 17; (e) the PD-L1 binding domain comprises a VH comprising SEQ ID NO: 4 and a VL comprising SEQ ID NO: 8; and the CD40 binding domain comprises a VH comprising SEQ ID NO: 34 and a VL comprising SEQ ID NO: 17; (f) the PD-L1 binding domain comprises a VH comprising SEQ ID NO: 4 and a VL comprising SEQ ID NO: 8; and the CD40 binding domain comprises a VH comprising SEQ ID NO: 13 and a VL comprising SEQ ID NO: 38; (g) the PD-L1 binding domain comprises a VH comprising SEQ ID NO: 4 and a VL comprising SEQ ID NO: 8; and the CD40 binding domain comprises a VH comprising SEQ ID NO: 13 and a VL comprising SEQ ID NO: 42; (h) the PD-L1 binding domain comprises a VH comprising SEQ ID NO: 4 and a VL comprising SEQ ID NO: 8; and the CD40 binding domain comprises a VH comprising SEQ ID NO: 46 and a VL comprising SEQ ID NO: 17; (i) the PD-L1 binding domain comprises a VH comprising SEQ ID NO: 4 and a VL comprising SEQ ID NO: 8; and the CD40 binding domain comprises a VH comprising SEQ ID NO: 50 and a VL comprising SEQ ID NO: 17; (j) the PD-L1 binding domain comprises a VH comprising SEQ ID NO: 4 and a VL comprising SEQ ID NO: 8; and the CD40 binding domain comprises a VH comprising SEQ ID NO: 484 and a VL comprising SEQ ID NO: 485; or (k) the PD-L1 binding domain comprises a VH comprising SEQ ID NO: 4 and a VL comprising SEQ ID NO: 8; and the CD40 binding domain comprises a VH comprising SEQ ID NO: 484 and a VL comprising SEQ ID NO: 28.

Provided herein is a binding polypeptide, wherein (a) the PD-L1 binding domain comprises SEQ ID NO: 9; and the CD40 binding domain comprises SEQ ID NO: 18; (b) the PD-L1 binding domain comprises SEQ ID NO: 9; and the CD40 binding domain comprises SEQ ID NO: 25; (c) the PD-L1 binding domain comprises SEQ ID NO: 9; and the CD40 binding domain comprises SEQ ID NO: 29; (d) the PD-L1 binding domain comprises SEQ ID NO: 9; and the CD40 binding domain comprises SEQ ID NO: 33; (e) the PD-L1 binding domain comprises SEQ ID NO: 9; and the CD40 binding domain comprises SEQ ID NO: 35; (f) the PD-L1 binding domain comprises SEQ ID NO: 9; and the CD40 binding domain comprises SEQ ID NO: 39; (g) the PD-L1 binding domain comprises SEQ ID NO: 9; and the CD40 binding domain comprises SEQ ID NO: 43; (h) the PD-L1 binding domain comprises SEQ ID NO: 9; and the CD40 binding domain comprises SEQ ID NO: 47; (i) the PD-L1 binding domain comprises SEQ ID NO: 9; and the CD40 binding domain comprises SEQ ID NO: 51; (j) the PD-L1 binding domain comprises SEQ ID NO: 9; and the CD40 binding domain comprises SEQ ID NO: 486; or (k) the PD-L1 binding domain comprises SEQ ID NO: 9; and the CD40 binding domain comprises SEQ ID NO: 488.

Provided herein is a binding polypeptide comprising SEQ ID NO: 19, SEQ ID NO: 26, SEQ ID NO: 30, SEQ ID NO: 280, SEQ ID NO: 36, SEQ ID NO: 40, SEQ ID NO: 44, SEQ ID NO: 48, SEQ ID NO: 52, SEQ ID NO: 487, or SEQ ID NO: 489.

In some embodiments, the hinge is an immunoglobulin hinge.

In some embodiments, the immunoglobulin constant region comprises immunoglobulin CH2 and CH3 domains of IgG1, IgG2, IgG3, IgG4, IgA1, IgA2 or IgD.

In some embodiments, the immunoglobulin constant region comprises a human IgG1 CH2 domain comprising an amino acid substitution at one or more of the following residues, according to the EU numbering system: E233, L234, L235, G236, G237, E318, K320, K322. In some embodiments, the amino acid substitution at residue E233 is E233P. In some embodiments, the amino acid substitution at residue L234 is selected from the group consisting of L234A and L234V. In some embodiments, the amino acid substitution at residue L235 is L235A. In some embodiments, the amino acid substitution at residue G237 is G237A. In some embodiments, the amino acid substitution at E318 is E318A. In some embodiments, the amino acid substitution at K320 is K320A. In some embodiments, the amino acid substitution at K322 is K322A. In some embodiments, residue G236 according to the EU numbering system) is deleted.

Provided herein is a dimeric protein comprising a binding polypeptide disclosed herein. In some embodiments, the dimeric protein is a homodimer.

Provided herein is a composition comprising a binding polypeptide disclosed herein, and a pharmaceutically acceptable carrier, diluent or excipient. Provided herein is a composition comprising a dimeric protein of disclosed herein, and a pharmaceutically acceptable carrier, diluent or excipient.

Provided herein is a nucleic acid molecule comprising a nucleotide sequence encoding a binding polypeptide disclosed herein. In some embodiments, the nucleotide sequence comprises SEQ ID NO: 78, SEQ ID NO: 79, SEQ ID NO: 259, or SEQ ID NO: 260. Provided herein is an expression vector comprising a nucleic acid molecule disclosed herein. Provided herein is a recombinant host cell comprising a nucleic acid molecule disclosed herein or an expression vector disclosed herein.

Provided herein is a method of producing a binding polypeptide, the method comprising: culturing a recombinant host cell disclosed herein under conditions whereby the nucleic acid molecule is expressed, thereby producing the binding polypeptide; and isolating the binding polypeptide from the host cell or culture.

Provided herein is a method for treating cancer in a subject in need thereof, the method comprising administering to the subject a therapeutically effective amount of a binding polypeptide, a dimeric protein, or a composition disclosed herein.

Provided herein is a method for treating a symptom of cancer in a subject in need thereof, the method comprising administering to the subject a therapeutically effective amount of a binding polypeptide, a dimeric protein, or a composition disclosed herein.

In any of the embodiments of the methods for treatment provided herein, the cancer may be a solid tumor. In any of the embodiments of the methods for treatment provided herein, the cancer may express PD-L1.

In any of the embodiments of the methods for treatment provided herein, the cancer may be head and neck cancer, melanoma, lung cancer, brain cancer, thymus cancer, breast cancer, liver cancer, pancreatic cancer, kidney cancer, ovarian cancer, bladder cancer, gastrointestinal tract cancer or colon cancer.

Provided herein is a binding polypeptide, a dimeric protein, or a composition disclosed herein for use as a medicament.

### BRIEF DESCRIPTION OF THE DRAWINGS

FIG. 1 depicts an amino acid alignment illustrating progress of humanization from mouse anti-PD-L1 clone 5F9 (PDL01034) to fully humanized antibody sequence of the PDL01152 molecule. Alignments to the closest human immunoglobulin germline V and J segments for VH and VL chains are shown. CDRs (Kabat definition) are indicated in bold underlined font. In the VH alignments, the sequences from top to bottom are SEQ ID NOs: 288-292 and IGHV3-48+IGHJ4 (SEQ ID NO: 278). In the VL alignments, the sequences from top to bottom are SEQ ID NOs: 293-297 and IGKV1-39+IGKJ2 (SEQ ID NO: 279).

FIG. 2A-FIG. 2F show binding of scFv anti-PD-L1 constructs to human (FIG. 2A and FIG. 2D), cynomolgus (FIG. 2B and FIG. 2E) and parental PD-L1-expressing CHOK1SV (FIG. 2C and FIG. 2F) cells. Serial dilutions of N-terminal anti-PD-L1 constructs were incubated with transfected target cells and subsequently labelled with SULFO TAG-labeled goat anti-human IgG secondary antibody. Binding was quantified by MSD (Meso Scale Discovery instrument). The y-axis displays the signal in electrochemiluminescence units over background signal. (See Example 7.)

FIG. 3A-FIG. 3B show binding of anti-PD-L1 leads PDL01034 (FIG. 3A) or PDL01036 (FIG. 3B) to various cell lines that expressed PD-L1 or were negative controls to assess non-specific binding. Serial dilutions of N-terminal anti-PD-L1 constructs were incubated with transfected target cells or tumor cell lines and subsequently labelled with SULFO TAG-labeled goat anti-human IgG secondary antibody. Binding was quantified by MSD (Meso Scale Discovery instrument). The y-axis displays the signal in electrochemiluminescence units over background signal. (See Example 8.)

FIG. 4A-FIG. 4B show the ability of hybridoma antibody clones 1D5, 2H7 and 5B9 to block CD40 receptor-CD40 ligand binding on cells, two sets of experiments were carried out. In the first assay (FIG. 4A), CD40 expressing cells were saturated by His-tagged CD40L (BPS Bioscience) followed by binding of ADAPTIRs. Cells were incubated with PE-labelled goat-anti-hulgG F(ab′)2 (Jackson). In the second assay (FIG. 4B), CD40 expressing cells were saturated by antibodies followed by CD40L and detected using PE-labelled anti-His (Biolegend). All cells were washed and analyzed by flow cytometry. (See Example 12.)

FIG. 5A-FIG. 5B show the binding of anti-PD-L1 and anti-CD40 bispecific proteins with various structures and binding valency. Serial dilutions of constructs were incubated with CHOK1SV cells transfected with CD40 (FIG. 5A) or PD-L1 (FIG. 5B) and subsequently labelled with a fluorescently-conjugated goat-α-human Fc secondary antibody. The y-axis displays the mean fluorescence intensity units (MFI). (See Example 13.)

FIG. 6 shows the PD-L1/PD-1 blocking ability of anti-PD-L1 and anti-CD40 bispecific proteins with various structures and binding valency. Serial dilutions of constructs were incubated with the human PD-1 NFAT luciferase reporter Jurkat line (BPS) and TCR Activator PD-L1-expressing CHO target cells (BPS). After 6 hours, Bio-Glo luciferase reagent (Promega) was added and luminescence was read on a MicroBeta 2450 Microplate Counter (Perkin Elmer). The y-axis displays the relative light units over background signal. (See Example 13.)

FIG. 7A-FIG. 7B show the CD40 signaling ability of anti-PD-L1 and anti-CD40 bispecific proteins with various structures and binding valency. Serial dilutions of constructs were incubated with the human CD40/NFκB HEK293 luciferase reporter cells (BPS) and PD-L1-expressing (FIG. 7A) or parental (FIG. 7B) CHOK1SV target cells. After 6 hours, Bio-Glo luciferase reagent (Promega) was added luminescence was read on a MicroBeta 2450 Microplate Counter (Perkin Elmer). The y-axis displays the relative light units over background signal. (See Example 13.)

FIG. 8A depicts an amino acid alignment illustrating progress of humanization from mouse anti-CD40 clone 1D5 (CD401016) to fully humanized antibody sequence CD401164. Alignments to the closest human immunoglobulin germline V and J segments for VH chains are shown. CDRs (Kabat definition) are indicated in bold underlined font. Sequences in order from top to bottom are SEQ ID NOs: 298-318 and 281.

FIG. 8B depicts an amino acid alignment illustrating progress of humanization from mouse anti-CD40 clone 1D5 (CD401016) to fully humanized antibody sequence CD401164. Alignments to the closest human immunoglobulin germline V and J segments for VL chains are shown. CDRs (Kabat definition) are indicated in bold underlined font. Sequences in order from top to bottom are SEQ ID NOs: 319-339 and 282.

FIG. 8C depicts an amino acid alignment illustrating progress of humanization from mouse anti-CD40 clone 1D5 (CD401016) to fully humanized antibody sequence CD401242. Alignments to the closest human immunoglobulin germline V and J segments for VH chains are shown. CDRs (Kabat definition) are indicated in bold underlined characters. Sequences in order from top to bottom are SEQ ID NOs: 340-378 and 283.

FIG. 8D depicts an amino acid alignment illustrating progress of humanization from mouse anti-CD40 clone 1D5 (CD401016) to fully humanized antibody sequence CD401242. Alignments to the closest human immunoglobulin germline V and J segments for VL chains are shown. CDRs (Kabat definition) are indicated in bold underlined font. Sequences in order from top to bottom are SEQ ID NOs: 379-417 and 284.

FIG. 9A depicts an amino acid alignment illustrating progress of humanization from mouse anti-CD40 clone 5B9 (CD401018) clone to fully humanized antibody sequence CD401133. Alignments to the closest human immunoglobulin germline V and J segments for VH chains are shown. CDRs (Kabat definition) are indicated in bold underlined font. Sequences in order from top to bottom are SEQ ID NOs: 418-435 and 285.

FIG. 9B depicts an amino acid alignment illustrating progress of humanization from mouse anti-CD40 clone 5B9 (CD401018) clone to fully humanized antibody sequence CD401133. Amino acid alignment illustrating progress of humanization from mouse clone to fully humanized antibody sequence. Alignments to the closest human immunoglobulin germline V and J segments for VL chains are shown. CDRs (Kabat definition) are indicated in bold underlined font. Sequences in order from top to bottom are SEQ ID NOs: 436-453 and 286.

FIG. 10A-FIG. 10E show the binding of multiple rounds of humanized anti-CD40 scFv antibodies. Serial dilutions of N-terminus scFv constructs were incubated with CHOK1SV cells transfected with human CD40 and subsequently labelled with a fluorescently-conjugated goat-α-human Fc secondary antibody. The y-axis displays the mean fluorescence intensity units (MFI). (See Example 21.)

FIG. 11A-FIG. 11B show the binding of anti-PD-L1 and anti-CD40 ADAPTIR antibodies to tumor cell lines. Serial dilutions of ADAPTIR constructs were incubated with Daudi or MDA-MB-231 tumor cells and subsequently labelled with a fluorescently-conjugated goat-α-human Fc secondary antibody. The y-axis displays the mean fluorescence intensity units (MFI). (See Example 22.)

FIG. 12A-FIG. 12E show the binding of optimized anti-PD-L1 and anti-CD40 ADAPTIR antibodies to multiple cell lines. Serial dilutions of ADAPTIR constructs were incubated with human (FIG. 12A), cynomolgus (FIG. 12B) CHOK1SV/CD40, Daudi (FIG. 12C), human CHOK1SV/PD-L1 (FIG. 12D) or parental CHOK1SV (FIG. 12E) cells. Following binding, cells are subsequently labelled with a fluorescently-conjugated goat-α-human Fc secondary antibody. The y-axis displays the mean fluorescence intensity units (MFI). (See Example 24.)

FIG. 13A-FIG. 13C show the functionality of anti-PD-L1 and anti-CD40 ADAPTIR antibodies. (FIG. 13A) The PD-L1/PD-1 blockade assay combined serial dilutions of ADAPT1Rs and incubated them with the human PD-1 NFAT luciferase reporter Jurkat line (BPS) and TCR Activator PD-L1-expressing CHO target cells (BPS). (FIG. 13B and FIG. 13C) The CD40 reporter assay combined serially diluted ADAPTIRs with human CD40/NFκB HEK293 luciferase reporter cells (BPS) and PD-L1-expressing (FIG. 13B) or parental CHOK1SV (FIG. 13C) target cells. After 6 hours, Bio-Glo luciferase reagent (Promega) was added luminescence was read on a MicroBeta 2450 Microplate Counter (Perkin Elmer). The y-axis displays the relative light units over background signal. (See Example 25.)

FIG. 14A-FIG. 14B show the activity of optimized anti-PD-L1×anti-CD40 ADAPTIR bispecifics to target and kill tumors in vitro. SCC152 (ATCC) was serially transduced with lentivirus to express Nuclight Orange (NLO, Sartorius), human PD-L1 and EBV protein (Vectorbuilder). In FIG. 14A, target cells that were antibiotically selected to be triple positive for NLO, human PD-L1 and EBV protein are shown using flowcytometry on a BD FACSymphony. For cytotoxicity assay, M2 macrophages, immature DC and EBV-specific T cells ere separately prepared prior to the assay. Adherant target SCC152 cells expressing NLO, PD-L1 and EBV protein were plated one day prior to assay setup. Titrated ADAPTIRS or controls, macrophages, DC and T cells are then added to the SCC152 target cells on day 0 of the assay. The assay plate was imaged every 8 hours in an Incucyte reader (Sartorius) over the course of 6 days. In FIG. 14B, the graphical representation of the number of live SCC152 tumor cells when 0.25 nM of test or control molecules are added. (See Example 26.)

FIG. 15 depicts a protein sequence alignment of CD40 extra-cellular domains used for epitope mapping of ID5 antibody. All proteins were constructed with 2×FLAG tag on N-terminus and human CD40 transmembrane and intra-cellular domain and expressed in CHO cells. Binding was examined by flow cytometry with anti-FLAG antibody and 1D5 antibody. Binding signal to all constructs was normalized to FLAG signal and to human CD40 signal and is shown on right as % of binding to human CD40. Sequences in order from top to bottom are SEQ ID NOs: 287 and 454-483.

FIG. 16A-FIG. 16B show the functionality of anti-CD40 ADAPTIR bispecifics to target either ROR1 or EGFR tumor-associated antigens. The CD40 reporter assay combined serially diluted ADAPTIRs with human CD40/NFκB HEK293 luciferase reporter cells (BPS) and tumor target cells. In FIG. 16A, the bispecifics were made with an anti-ROR1 binding domain. The targeting cell line used in the assay was Kasumi-2. In FIG. 16B, the bispecifics were made with an anti-EGFR binding domain. The targeting cell line used in the assay was PC-3. After 6 hours of incubation. Bio-Glo luciferase reagent (Promega) was added luminescence was read on a MicroBeta 2450 Microplate Counter (Perkin Elmer). The y-axis displays the relative light units over background signal. (See Example 30.)

FIG. 17A-FIG. 17C show the functionality of anti-PD-L1 ADAPTIR bispecifics to induce NFκB signaling of CD3, OX40 or 4-1BB. (FIG. 17A) Jurkat cells carrying a luciferase reporter gene under the control of an NFκB promoter were co-cultured with the CD3-binding bispecific construct PC401020 to induce target-dependent activation of CD3. (FIG. 17B) For the OX40 reporter assay, Jurkat cells transfected to expressed human OX40 and the reporter gene were co-cultured with the OX40-binding bispecific construct PC401022 to induce target-dependent activation of OX40. (FIG. 17C) For the 4-4BB assay, Jurkat cells transfected to expressed human 4-4BB and the reporter gene were co-cultured with the 4-1BB-binding bispecific construct PC401021 to induce target-dependent activation of 4-1BB. Serially diluted constructs were incubated with target cells and an NFκB reporter cell line for 5 hours, followed with the addition of Bio-Glo. The y-axis displays the RLU (See Example 31.)

FIG. 18A-FIG. 18D show the binding of optimized anti-PD-L1 and anti-CD40 ADAPTIR antibodies with stabilized binding domains to multiple cell lines. Serial dilutions of ADAPTIR constructs were incubated with Daudi tumor (FIG. 18A), cynomolgus CHOK1SV/CD40 (FIG. 18B), human CHOK1SV/PD-L1 (FIG. 18C) or parental (FIG. 18D) CHOK1SV cells. Following binding, cells were subsequently labelled with a fluorescently-conjugated goat-α-human Fc secondary antibody. The y-axis displays the mean fluorescence intensity units (MFI). (See Example 32.)

FIG. 19A-FIG. 19B show the functionality of anti-PD-L1 and anti-CD40 ADAPTIR antibodies with stabilized binding domains. The CD40 reporter assay combined serially diluted ADAPTIRs with human CD40/NFκB HEK293 luciferase reporter cells (BPS) and PD-L1-expressing CHOK1SV target cells (FIG. 19A). The PD-L1/PD-1 blockade assay combined serial dilutions of ADAPTIRs and incubated them with the human PD-1 NFAT luciferase reporter Jurkat line (BPS) and TCR Activator PD-L1-expressing CHO target cells (BPS) (FIG. 19B). After 6 hours, Bio-Glo luciferase reagent (Promega) was added and luminescence was read on a MicroBeta 2450 Microplate Counter (Perkin Elmer). The y-axis displays the relative light units over background signal. (See Example 33.)

FIG. 20A-FIG. 20B show the functionality of anti-PD-L1 and anti-CD40 ADAPTIR antibodies with stabilized binding domains and the requirement for crosslinking. The CD40 reporter assay combined serially diluted ADAPTIRs containing a wildtype Fc or FcγR null Fc with human CD40/NFκB HEK293 luciferase reporter cells (BPS) and CD64-expressing (FIG. 20A) or parental (FIG. 20B) CHOK1SV target cells. After 6 hours, Bio-Glo luciferase reagent (Promega) was added luminescence was read on a MicroBeta 2450 Microplate Counter (Perkin Elmer). The y-axis displays the relative light units over background signal. (See Example 34.)

FIG. 21 shows the in vivo functionality of anti-PD-L1 and anti-CD40 ADAPTIR antibodies with stabilized binding domains to target and kill tumor. Five hundred thousand human PD-L1-expressing MC38 tumor cells were injected SC into the right flank of female huCD40/PD-1/PD-L1 triple knock-in mice (n=5/group). Treatments were administered by IP injection on days 0, 2, 7, 10, 14 and 17 at 3 mg/kg. Mean tumor volume for each group was plotted ±SEM. Mice that reached a tumor endpoint of equal to or greater than 1500 mm³ had the last recorded tumor volume used at future time points. (See Example 35.)

FIG. 22A-FIG. 22B show the activity of optimized anti-PD-L1×anti-CD40 ADAPTIR bispecifics with stabilized binding domains able to stimulate DCs in vitro. Immature DCs were cultured alone (FIG. 22A) or with SCC152 (ATCC) target cells transduced with lentivirus to express human PD-L1 protein (Vectorbuilder) expressing PD-L1 (FIG. 22B) and titrated ADAPTIRS or controls. Cells were harvested after 48 hours and fluorescently-labeled for CD86. (See Example 36.)

FIG. 23A-FIG. 23C show the activity of optimized anti-PD-L1×anti-CD40 ADAPTIR bispecifics with stabilized binding domains to target and kill tumors in vitro. SCC152 transduced with human PD-L1 and EBV protein (Vectorbuilder) were cocultured with immature DC and EBV-specific T cells. At 48 hours, supernatant was collected and analyzed for IL-12 (FIG. 23A), IFN-γ (FIG. 23B) and TNF-α (FIG. 23C) using a multiplex magnetic bead cytokine assay (Milliplex). (See Example 37.)

FIG. 24A-FIG. 24B show the activity of optimized anti-PD-L1×anti-CD40 ADAPTIR bispecifics with stabilized binding domains to target and kill tumors in vitro. SCC152 (ATCC) was serially transduced with lentivirus to express Nuclight Orange (NLO, Sartorius), human PD-L1 and EBV protein (Vectorbuilder). For cytotoxicity assay, M2 macrophages, immature DC and EBV-specific T cells are separately prepared prior to the assay. Adherent target SCC152 cells expressing NLO, PD-L1 and EBV protein were plated one day prior to assay setup. Titrated ADAPTIRS or controls, macrophages, DC and T cells are then added to the SCC152 target cells on day 0 of the assay. The assay plate was imaged every 8 hours in an Incucyte reader (Sartorius) over the course of 6 days. In FIG. 24A, the graphical representation of the number of live SCC152 tumor cells when 0.22 nM of test or control molecules are added over time. At 160 hours, the tumor cell count was evaluated and plotted to demonstrate the ability of ADAPTIRs to enhance tumor cell killing by T cells (FIG. 24B) (See Example 38.)

### DETAILED DESCRIPTION

Provided herein are bispecific polypeptides that bind specifically to PD-L1 and/or CD40. Bispecific molecules that target PD-L1 and/or CD40 are useful for priming anti-tumor immune responses against cancer cells expressing a tumor-associated antigen. Molecules provided herein combine a tumor targeting arm (e.g., a PD-L1 targeting arm) with immune system agonism (e.g., via a CD40 targeting arm) to cause tumor dependent crosslinking and agonism of CD40. Bispecific molecules (and PD-L1 binding domains and CD40 binding domains) provided herein were engineered to combine specific binding with advantageous manufacturability properties, such as thermal stability, solubility and resistance to sheer stress.

### Definitions

The term “about” when immediately preceding a numerical value means±up to 10% of the numerical value. For example, “about 40” means±up to 10% of 40 (i.e., from 36 to 44), ±up to 10%, ±up to 9%, ±up to 8%, ±up to 7%, ±up to 6%, ±up to 5%, ±up to 4%, ±up to 3%, ±up to 2%, ±up to 1%, ±up to less than 1%, or any other value or range of values therein.

As used herein, the term “binding domain” or “binding region” refers to the domain, region, portion, or site of a protein, polypeptide, oligopeptide, peptide, antibody, or binding domain derived from an antibody that possesses the ability to specifically recognize and bind to a target molecule, such as an antigen, ligand, receptor, substrate, or inhibitor. Exemplary binding domains include antibodies and antibody-like proteins or domains, antibody heavy and light chain variable regions, and single-chain antibody variable regions (e.g., domain antibodies, sFv, scFv, scFab). In certain embodiments, the binding domain comprises or consists of an antigen binding site (e.g., comprising a variable heavy chain sequence and variable light chain sequence or three light chain complementary determining regions (CDRs) and three heavy chain CDRs from an antibody placed into alternative framework regions (FRs) (e.g., human FRs optionally comprising one or more amino acid substitutions). A variety of assays are known for identifying binding domains of the present disclosure that specifically bind a particular target, including Western blot, ELISA, phage display library screening, and BIACORE® interaction analysis. In some embodiments, the polypeptides of the instant disclosure comprise a binding domain that specifically binds to a target antigen expressed by a target cell. In some embodiments, the polypeptides of the present invention comprise a binding domain that specifically binds to a target antigen expressed by an effector cell. In some embodiments, the polypeptides of the present invention are multispecific polypeptides and comprise two or more binding domains.

A binding domain or protein comprising a binding domain “specifically binds” a target if it binds the target with an affinity or Kₐ (i.e., an equilibrium association constant of a particular binding interaction with units of 1/M) equal to or greater than 10⁵ M⁻¹, while not significantly binding other components present in a test sample. Binding domains can be classified as “high affinity” binding domains and “low affinity” binding domains. “High affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding domains refer to those binding domains with a Kₐ of up to 10⁷ M⁻¹, up to 10⁶ M⁻¹, up to 10¹ M⁻¹. Alternatively, affinity can be defined as an equilibrium dissociation constant (K_{d}) of a particular binding interaction with units of M (e.g., 10⁻⁵ M to 10⁻¹³, or about 500 nM, about 300 nM, about 250 nM, about 200 nM, about 150 nM, about 100 nM, about 50 nM, about 25 nM, about 10 nM, or about 5 nM). Affinities of binding domain polypeptides and single chain polypeptides according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

As used herein, a “conservative substitution” is recognized in the art as a substitution of one amino acid for another amino acid that has similar properties. Exemplary conservative substitutions are well-known in the art (see, e.g., PCT Application Publication No. WO 97/09433, page 10, published Mar. 13, 1997: Lehninger, Biochemistry, Second Edition; Worth Publishers, Inc. NY:NY (1975). pp. 71-77; Lewin, Genes IV, Oxford University Press, NY and Cell Press, Cambridge, MA (1990), p. 8).

As used herein, the term “derivative” refers to a modification of one or more amino acid residues of a peptide by chemical or biological means, either with or without an enzyme, e.g., by glycosylation, alkylation, acylation, ester formation, or amide formation.

As used herein, a polypeptide or amino acid sequence “derived from” a designated polypeptide or protein refers to the origin of the polypeptide. In certain embodiments, the polypeptide or amino acid sequence which is derived from a particular sequence (sometimes referred to as the “parent” or “parental” sequence) and has an amino acid sequence that is essentially identical to the parent sequence or a portion thereof, wherein the portion consists of at least about 10-20 amino acids, at least about 20-30 amino acids, or at least about 30-50 amino acids, or at least about 50-150 amino acids, or which is otherwise identifiable to one of ordinary skill in the art as having its origin in the parent sequence. For example, a binding domain (e.g, a Fab, F(ab′)2, Fab′, scFv, single domain antibody (sdAb), etc.) can be derived from an antibody. In some embodiments, a binding domain sequence is derived from an antibody or protein by means of a computer algorithm or in silico.

Polypeptides derived from another polypeptide can have one or more mutations or alterations relative to the parent polypeptide, e.g., one or more amino acid residues which have been substituted with another amino acid residue or which has one or more amino acid insertions or deletions. In such embodiments, polypeptides derived from a parent polypeptide and comprising one or more mutations or alteration are referred to as “variants.” As used herein, the term “variant” or “variants” refers to a polynucleotide or polypeptide with a sequence differing from that of a reference polynucleotide or polypeptide, but retaining essential properties thereof. Generally, variant polynucleotide or polypeptide sequences are overall closely similar, and, in many regions, identical to the reference polynucleotide or polypeptide. For instance, a variant polynucleotide or polypeptide may exhibit at least about 70%, at least about 80%, at least about 90%, at least about 91%, at least about 92%, at least about 93%, at least about 94%, at least about 95%, at least about 96%, at least about 97%, at least about 98% or at least about 99% sequence identity compared to the active portion or full-length reference polynucleotide or polypeptide. The polypeptide can comprise an amino acid sequence which is not naturally occurring. Such variations necessarily have less than 100% sequence identity or similarity with the parent polypeptide. In one embodiment, the variant will have an amino acid sequence from about 60% to less than 100% amino acid sequence identity or similarity with the amino acid sequence of the parent polypeptide. In another embodiment, the variant will have an amino acid sequence from about 75% to less than 100%, from about 80% to less than 100%, from about 85% to less than 100%, from about 90% to less than 100%, from about 95% to less than 100% amino acid sequence identity or similarity with the amino acid sequence of the parent polypeptide.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage sequence identity is calculated by determining the number of positions at which the identical nucleic acid base or amino acid residue occurs in both sequences to yield the number of identical positions. The number of identical positions is then divided by the total number of positions in the comparison window and multiplied by 100 to yield the percentage of sequence identity. Percentage of sequence identity is determined by comparing two optimally aligned sequences over a comparison window. The comparison window for polynucleotide sequences can be, for instance, at least about 20, 30, 40, 50, 60, 70, 80, 90, 100, 110, 120, 130, 140, 150, 160, 170, 180, 190, 200, 300, 400, 500, 600, 700, 800, 900 or 1000 or more nucleic acids in length. The comparison window for polypeptide sequences can be, for instance, at least about 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option. Two nucleotide or amino acid sequences are considered to have “substantially similar sequence identity” or “substantial sequence identity” if the two sequences have at least about 80%, at least about 85%, at least about 90%, at least about 95%, at least about 96%, at least about 97%, at least about 98%, or at least about 99% sequence identity relative to each other.

As used herein, unless otherwise provided, a position of an amino acid residue in a variable region of an immunoglobulin molecule is numbered according to either the IMGT criteria (Brochet et al, Nucl. Acids Res. (2008) 36, W503-508) or the Kabat numbering convention, or according to EU nomenclature (Ward et al., 1995 Therap. Immunol. 2:77-94), and a position of an amino acid residue in a constant region of an immunoglobulin molecule is numbered according to EU nomenclature (Ward et al., 1995 Therap. Immunol. 2:77-94). The Kabat numbering convention (Kabat, Sequences of Proteins of Immunological Interest, 5′ ed. Bethesda, MD: Public Health Service, National Institutes of Health (1991)) is an alternative system used to refer to a position of an amino acid residue in a variable region of an immunoglobulin molecule and is sometimes used to refer to a position of an amino acid residue in a variable region of an immunoglobulin molecule herein.

As used herein, the term “dimer” refers to a biological entity that consists of two subunits associated with each other via one or more forms of intramolecular forces, including covalent bonds (e.g., disulfide bonds) and other interactions (e.g., electrostatic interactions, salt bridges, hydrogen bonding, and hydrophobic interactions), and is stable under appropriate conditions (e.g., under physiological conditions, in an aqueous solution suitable for expressing, purifying, and/or storing recombinant proteins, or under conditions for non-denaturing and/or non-reducing electrophoresis). The terms “heterodimer” or “heterodimeric protein,” as used herein, refers to a dimer formed from two different polypeptides. A heterodimer does not include an antibody formed from four polypeptides (i.e., two light chains and two heavy chains). The terms “homodimer” or “homodimeric protein,” as used herein, refers to a dimer formed from two identical polypeptides.

“Fc region” or “Fc domain” refers to a polypeptide sequence corresponding to or derived from the portion of a source antibody that is capable of binding to Fc receptors on cells and/or the C1q component of complement, thereby mediating the effector function of an antibody. Fc stands for “fragment crystalline.” the fragment of an antibody that will readily form a protein crystal. Distinct protein fragments, which were originally described by proteolytic digestion, can define the overall general structure of an immunoglobulin protein. As originally defined in the literature, the Fc region is a homodimeric protein comprising two polypeptides that are associated by disulfide bonds, and each comprising a hinge region, a CH2 domain, and a CH3 domain. However, more recently the term has been applied to the single chain monomer component consisting of CH3, CH2, and at least a portion of the hinge sufficient to form a disulfide-linked dimer with a second such chain. As such, and depending on the context, use of the terms “Fc region” or “Fc domain” will refer herein to either the dimeric form or the individual monomers that associate to form the dimeric protein. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc includes variants of naturally occurring sequences.

An “immunoglobulin constant region” or “constant region” or “constant domain” is a term defined herein to refer to a peptide or polypeptide sequence that corresponds to or is derived from part or all of one or more constant domains of an immunoglobulin. In certain embodiments, the constant region comprises IgG CH2 and CH3 domains, e.g., IgG1 CH2 and CH3 domains. In certain embodiments, the constant region does not comprise a CH1 domain. In certain embodiments, the constant domains making up the constant region are human. In some embodiments, the constant region of a fusion protein of this disclosure lacks or has minimal effector functions while retaining the ability to bind some Fc receptors such as the neonatal Fc receptor (FcRn) and retaining a relatively long half-life in vivo. For example, the constant region of a fusion protein of this disclosure do not result in, or substantially reduce the induction of antibody-dependent cell-mediated cytotoxicity (ADCC), antibody-dependent cell-mediated phagocytosis (ADCP), complement activation, and/or complement-dependent cytotoxicity (CDC). In other variations, a fusion protein of this disclosure comprises constant domains that retain one or more effector functions, such as of one or both of ADCC and CDC. In certain embodiments, a binding domain of this disclosure is fused to a human IgG1 constant region, wherein the IgG1 constant region has one or more of the following amino acids mutated: E233, L234, L235, G236, G237, E318, K320, K322, or any combination thereof (numbering according to EU). For example, any one or more of these amino acids can be changed to alanine, valine, or proline. In some embodiments, the IgG1 constant region has one or more of the following amino acids mutations: E233P, L234A, L234V, L235A, G237A, E318A, K320A, K322A. In some embodiments, an IgG1 Fc domain has each of E233, L234, L235. G236, G237, E318, K320, K322 (according to EU numbering) mutated. In some embodiments, the IgG1 constant region has one or more of the following amino acids deleted: E233, L234, L235, G236, G237, E318, K320, K322 (numbering according to EU). In some embodiments, the IgG1 constant region has each of E233, L234, L235, G236, G237, E318, K320, K322 deleted (numbering according to EU).

The terms “light chain variable region” (also referred to as “light chain variable domain” or “Vt.”) and “heavy chain variable region” (also referred to as “heavy chain variable domain” or “VH”) refer to the variable binding region from an antibody light and heavy chain, respectively. The variable binding regions are made up of discrete, well-defined sub-regions known as “complementarity determining regions” (CDRs) and “framework regions” (FRs). In some embodiments, the FRs are humanized. The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). A “Fab” (fragment antigen binding) is the part of an antibody that binds to antigens and includes the variable region and CH1 domain of the heavy chain linked to the light chain via an inter-chain disulfide bond.

As used herein, the term “linker” generally refers to a short polypeptide sequence connecting two sub-domains of a polypeptide. Non-limiting examples of linkers include flexible linkers comprising glycine-serine repeats, and linkers derived from (a) an interdomain region of a transmembrane protein (e.g., a type I transmembrane protein); (b) a stalk region of a type II C-lectin; or (c) an immunoglobulin hinge. In some embodiments, a linker provides a spacer function compatible with interaction of the two sub-binding domains so that the resulting polypeptide retains a specific binding affinity to the same target molecule as an antibody that comprises the same light and heavy chain variable regions. In certain embodiments, a linker is comprised of five to about 35 amino acids, for instance, about 15 to about 25 amino acids. As used herein, the phrase a “linker between CH3 and CH1 or CL” refers to one or more amino acid residues (e.g., about 2-12, about 2-10, about 4-10, about 5-10, about 6-10, about 7-10, about 8-10, about 9-10, about 8-12, about 9-12, or about 10-12) between the C-terminus of a CH3 domain (e.g., a wild type CH3 or a mutated CH3) and the N-terminus of a CH1 domain or CL domain (e.g., Cc).

In some embodiments, depending on context, a linker may refer to (1) a polypeptide region between V_{H} and V_{L} regions in a single-chain F_{V} (scFv) or (2) a polypeptide region between a first binding domain and a second binding domain in a multispecific polypeptide comprising two binding domains. In the later example, wherein a linker connects two or more binding domains, such a linker is referred to herein as a “Fc-binding domain linker.” In some embodiments, a Fc-binding domain linker may directly link or connect two or more binding domains, resulting in a construct comprising the following structure: binding domain-Fc-binding domain linker-binding domain. In some embodiments, the multispecific polypeptides described herein comprise, in order from amino-terminus to carboxyl-terminus (i) a first binding domain, (ii) a Fc-binding domain linker, and (iii) a second binding domain. In some embodiments, a multispecific polypeptide comprises, in order from amino-terminus to carboxyl-terminus (i) a second binding domain, (ii) a Fc-binding domain linker, and (iii) a first binding domain. In some embodiments, a Fc-binding domain linker may link or connect two or more binding domains by linking at least one binding domain to a non-binding domain polypeptide, such as an immunoglobulin Fc domain (i.e., a polypeptide comprising the structure: Ig hinge-Ig constant region). In such embodiments, the resulting constructs may comprise the following structure: binding domain-Fc domain-Fc-binding domain linker-binding domain. In some embodiments, the multispecific polypeptides described herein comprise, in order from amino-terminus to carboxyl-terminus: (i) a first binding domain, (ii) a hinge region, (iii) an immunoglobulin constant region, (iv) a Fc-binding domain linker, and (v) a second binding domain. In some embodiments, a multispecific polypeptide comprises, in order from amino-terminus to carboxyl-terminus (i) a second binding domain, (ii) a Fc-binding domain linker, (iii) an immunoglobulin constant region, (iv) a hinge region, and (v) a first binding domain. A polypeptide region between an immunoglobulin constant region and a second binding domain in a multispecific polypeptide comprising two binding domains (e.g., a Fc-binding domain linker) may also be referred to as a “carboxyl-terminus linker” or an “amino-terminus linker” depending on the orientation of the domains within the multispecific polypeptide.

In some embodiments, a “hinge” or a “hinge region” refers to a polypeptide derived from an immunoglobulin hinge region and located between a binding domain and an immunoglobulin constant region in a polypeptide described herein. A “wild-type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody. In certain embodiments, a wild type immunoglobulin hinge region sequence is human, and can comprise a human IgG hinge region (e.g., and IgG1, IgG2, IgG3, or IgG4 hinge region).

An “altered immunoglobulin hinge region” or “variant immunoglobulin hinge region” refers to a hinge region polypeptide with one or more mutations, substitutions, insertions, or deletions compared to a corresponding parental wild-type immunoglobulin hinge region. In certain embodiments, an altered immunoglobulin hinge region is at least about 70% identical to a wild-type immunoglobulin hinge region (e.g., at least about 70%, at least about 75%, at least about 80%, at least about 85%, at least about 90%, at least about 95%, at least about 97%, at least about 98%, or at least about 99% identical). In certain embodiments, an altered immunoglobulin hinge region is a fragment of a wild type immunoglobulin hinge region that has a length of about 5 amino acids (e.g., about 5, 6, 7, 8, 9, 10, 11, 12, 13, 14, 15, 16, 17, 18, 19, 20, or more amino acids) up to about 120 amino acids (for instance, having a length of about 10 to about 40 amino acids or about 15 to about 30 amino acids or about 15 to about 20 amino acids or about 20 to about 25 amino acids). Typically, an altered immunoglobulin hinge region that is a fragment of a wild type immunoglobulin hinge region comprises an IgG core hinge region (e.g., a polypeptide comprising the sequence C-X-X-C, wherein X is any amino acid) as disclosed in U.S. Patent Application Publication Nos. 2013/0129723 and 2013/0095097.

As used herein, the term “humanized” refers to a process of making an antibody or immunoglobulin binding proteins and polypeptides derived from a non-human species (e.g., mouse or rat) less immunogenic to humans, while still retaining antigen-binding properties of the original antibody, using genetic engineering techniques. In some embodiments, the binding domain(s) of an antibody or immunoglobulin binding proteins and polypeptides (e.g., light and heavy chain variable regions, Fab, scFv) are humanized. Non-human binding domains can be humanized using techniques known as CDR grafting (Jones et al., Nature 321:522 (1986)) and variants thereof, including “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Nat Acad Sci USA 86:10029-10033; Co, et al., 1991 Proc Natl Acad Sci USA 88:2869-2873; Co, et al., 1992 J Immunol 148:1149-1154), and “veneering” (Mark, er al., “Derivation of therapeutically active humanized and veneered anti-CD18 antibodies.” In: Metcalf B W, Dalton B J, eds. Cellular adhesion: molecular definition to therapeutic potential. New York: Plenum Press, 1994: 291-312). If derived from a non-human source, other regions of the antibody or immunoglobulin binding proteins and polypeptides, such as the hinge region and constant region domains, can also be humanized. Knowledge about humanized antibodies in the art is applicable to the polypeptides according to the disclosure, even if these polypeptides are not antibodies.

As used herein, the term “patient in need” or “subject in need” refers to a patient or subject at risk of, or suffering from, a disease, disorder or condition that is amenable to treatment or amelioration with a binding protein or multispecific polypeptide or a composition thereof provided herein. “Patient” and “subject” are used interchangeably herein.

As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

As used herein, the term “promoter” refers to a region of DNA involved in binding RNA polymerase to initiate transcription.

As used herein, the terms “nucleic acid,” “nucleic acid molecule,” or “polynucleotide” refer to deoxyribonucleotides or ribonucleotides and polymers thereof in either single- or double-stranded form. Unless specifically limited, the terms encompass nucleic acids containing analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985). J Biol. Chem. 260:2605-2608; Cassol et al. (1992), Rossolini et al. (1994) Mol. Cell. Probes 8:91-98). The term nucleic acid is used interchangeably with gene, cDNA, and mRNA encoded by a gene. As used herein, the terms “nucleic acid,” “nucleic acid molecule,” or “polynucleotide” are intended to include DNA molecules (e.g., eDNA or genomic DNA). RNA molecules (e.g., mRNA), analogs of the DNA or RNA generated using nucleotide analogs, and derivatives, fragments and homologs thereof.

The term “expression” refers to the biosynthesis of a product encoded by a nucleic acid. For example, in the case of nucleic acid segment encoding a polypeptide of interest, expression involves transcription of the nucleic acid segment into mRNA and the translation of mRNA into one or more polypeptides.

The terms “expression unit” and “expression cassette” are used interchangeably herein and denote a nucleic acid segment encoding a polypeptide of interest and capable of providing expression of the nucleic acid segment in a host cell. An expression unit typically comprises a transcription promoter, an open reading frame encoding the polypeptide of interest, and a transcription terminator, all in operable configuration. In addition to a transcriptional promoter and terminator, an expression unit can further include other nucleic acid segments such as, e.g., an enhancer or a polyadenylation signal.

The term “expression vector,” as used herein, refers to a nucleic acid molecule, linear or circular, comprising one or more expression units. In addition to one or more expression units, an expression vector can also include additional nucleic acid segments such as, for example, one or more origins of replication or one or more selectable markers. Expression vectors are generally derived from plasmid or viral DNA, or can contain elements of both.

As used herein, a “polypeptide,” “polypeptide chain.” or “protein” refers to a single, linear and contiguous arrangement of covalently linked amino acids. Polypeptides can form one or more intrachain disulfide bonds. With regard to polypeptides as described herein, reference to modifications or alterations of amino acid residues corresponding to those specified by SEQ ID NO includes post-translational modifications of such residues. The terms polypeptide and protein also encompass embodiments where two polypeptide chains link together in a non-linear fashion, such as via an interchain disulfide bond. For example, a native immunoglobulin molecule is comprised of two heavy chain polypeptides and two light chain polypeptides. Each of the heavy chain polypeptides associate with a light chain polypeptide by virtue of interchain disulfide bonds between the heavy and light chain polypeptides to form two heterodimeric proteins or polypeptides (i.e., a protein comprised of two heterologous polypeptide chains). The two heterodimeric proteins then associate by virtue of additional interchain disulfide bonds between the heavy chain polypeptides to form an immunoglobulin protein or polypeptide. Herein, a protein or polypeptide may be an antibody or an antigen-binding fragment of an antibody.

As used herein, “PD-L1-binding protein” may be used interchangeably with “PD-L1-binding polypeptide.” Such molecules specifically bind to programmed death-ligand 1 protein (PD-L1) (e.g., human PD-L1).

As used herein, “CD40-binding protein” may be used interchangeably with “CD40-binding polypeptide.” Such molecules specifically bind to cluster of differentiation 40 protein (CD40) (e.g., human CD40).

As will be appreciated by one of skill in the art, proteins and polypeptides are defined herein in terms of the amino acid sequences of the individual polypeptide chains, which are indicated by the SEQ ID NOs referenced throughout this disclosure.

Polypeptides and proteins can also comprise non-peptidic components, such as carbohydrate groups. Carbohydrates and other non-peptidic substituents can be added to a protein or polypeptide by the cell in which the protein is produced, and will vary with the type of cell. Proteins and polypeptides are defined herein in terms of their amino acid backbone structures; substituents such as carbohydrate groups are generally not specified, but may be present nonetheless.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl-terminus of the reference sequence, but is not necessarily at the carboxyl-terminus of the complete polypeptide.

As used herein, the term “transformation,” “transfection,” and “transduction” refer to the transfer of nucleic acid (i.e., a nucleotide polymer) into a cell. As used herein, the term “genetic transformation” refers to the transfer and incorporation of DNA, especially recombinant DNA, into a cell. The transferred nucleic acid can be introduced into a cell via an expression vector.

“Antibody-dependent cell-mediated cytotoxicity” and “ADCC,” as used herein, refer to a cell-mediated process in which nonspecific cytotoxic cells that express FcγRs (e.g., monocytic cells such as natural killer (NK) cells and macrophages) recognize bound antibody (or other protein capable of binding FcγRs) on a target cell and subsequently cause lysis of the target cell. In principle, any effector cell with an activating FcγR can be triggered to mediate ADCC. The primary cells for mediating ADCC are NK cells, which express only FcγRIII, whereas monocytes, depending on their state of activation, localization, or differentiation, can express FcγRI, FcγRII, and FcγRIII. For a review of FcγR expression on hematopoietic cells, see, e.g., Ravetch et al., 1991, Annu. Rev. Immunol., 9:457-92.

The term “having ADCC activity,” as used herein in reference to a polypeptide or protein, means that the polypeptide or protein, for example, one comprising an Fc domain (e.g., an immunoglobulin hinge region and an immunoglobulin constant region having CH2 and CH3 domains) such as derived from IgG (e.g., IgG1), is capable of mediating antibody-dependent cell-mediated cytotoxicity (ADCC) through binding of a cytolytic Fc receptor (e.g., FcγRIII) on a cytolytic immune effector cell expressing the Fc receptor (e.g., an NK cell). In some embodiments, a multispecific polypeptide or protein comprising an Fc domain may lack effector function (e.g., null ADCC activity) as the result of mutations in the CH2 and/or CH3 domain.

“Complement-dependent cytotoxicity” and “CDC,” as used herein, refer to a process in which components in normal serum (“complement”), together with an antibody or other C1q-complement-binding protein bound to a target antigen, exhibit lysis of a target cell expressing the target antigen. Complement consists of a group of serum proteins that act in concert and in an orderly sequence to exert their effect.

The terms “classical complement pathway” and “classical complement system,” as used herein, are synonymous and refer to a particular pathway for the activation of complement. The classical pathway requires antigen-antibody complexes for initiation and involves the activation, in an orderly fashion, of nine major protein components designated C1 through C9. For several steps in the activation process, the product is an enzyme that catalyzes the subsequent step. This cascade provides amplification and activation of large amounts of complement by a relatively small initial signal.

The term “having CDC activity,” as used herein in reference to a polypeptide or protein, means that the polypeptide or protein, for example, one comprising an Fc domain (e.g., an immunoglobulin hinge region and an immunoglobulin constant region having CH2 and CH3 domains) such as derived from IgG (e.g., IgG1) is capable of mediating complement-dependent cytotoxicity (CDC) through binding of C1q complement protein and activation of the classical complement system. In some embodiments, a multispecific polypeptide or protein may lack effector function (e.g., null CDC activity) as the result of one or more mutations in the CH2 and/or CH3 domains.

As used herein, the term “effector cell” refers to a cell of the immune system that is capable of lysing or killing a target cell, such as a tumor cell. Herein, an effector cell may refer to a lymphocyte, such as a T cell, a natural killer (NK) cell, or an NKT cell, a monocyte, a macrophage, a dendritic cell, or a granulocyte. In particular embodiments, the term effector cell refers to a T cell, an NK cell, or an NKT cell.

As used herein, the terms “treatment,” “treating,” or “ameliorating” refers to a therapeutic treatment. A treatment is therapeutic if at least one symptom of disease in an individual receiving treatment improves or a treatment can delay worsening of a progressive disease in an individual, or prevent onset of additional associated diseases or symptoms.

As used herein, the term “therapeutically effective amount (or dose)” or “effective amount (or dose)” of a polypeptide or protein described herein or a composition thereof refers to that amount of the compound sufficient to result in amelioration of one or more symptoms of the disease being treated in a statistically significant manner or a statistically significant improvement in organ function. When referring to an individual active ingredient, administered alone, a therapeutically effective dose refers to that ingredient alone. When referring to a combination, a therapeutically effective dose refers to combined amounts of the active ingredients that result in the therapeutic effect, whether administered serially or simultaneously (in the same formulation or concurrently in separate formulations).

As used herein, a “multispecific polypeptide” refers to a polypeptide comprising two or more binding domains each capable of specifically binding to a target antigen. For example, the polypeptides described herein may comprise 2, 3, 4, or more binding domains and may be able to bind 2, 3, 4, or more target antigens. In some embodiments, a multispecific polypeptide is a bispecific polypeptide. Herein, a “bispecific polypeptide” comprises two binding domains and capable of binding to two distinct target antigens. In some embodiments, the bispecific polypeptides described herein comprise a first binding domain that specifically binds to a cell surface antigen expressed on a target cell. In some embodiments, the bispecific polypeptides described herein comprise a binding domain that specifically binds to a cell surface antigen expressed on an effector cell. In particular embodiments, the multispecific polypeptide is an ADAPTIR homodimer bispecific polypeptide in the format scFv-Fc-scFv.

Multispecific polypeptides using scaffolds are disclosed, for instance, in PCT Publication Nos. WO 2007/146968: WO 2010/040105: WO 2010/003108; WO 2016/094873; WO 2017/053469; U.S. Patent Application Publication No. 2006/0051844; and U.S. Pat. Nos. 7,166,707; and 8,409,577, which are each incorporated herein by reference in their entirety. In certain embodiments, the multispecific polypeptides described herein are bispecific polypeptides and may comprise an scFv-Fc-scFv structure, also referred to herein as an ADAPTIR polypeptide. The structure of a polypeptide comprising such a structure comprises, from N-terminus to C-terminus: a first scFv binding domain-a hinge region-an immunoglobulin constant region—a second scFv binding domain. In some embodiments, the structure of the peptide comprises, from N-terminus to C-terminus: a first scFv binding domain, a hinge region, an immunoglobulin constant region, a Fc-binding domain linker; and a second scFv binding domain.

### Bispecific Binding Polypeptides

Provided herein are bispecific binding polypeptides having a first binding domain and a second binding domain, wherein the first binding domain binds to a tumor-associated antigen and the second binding domain binds to an immune system agonist. The bispecific binding polypeptides may have an scFv-Fc-scFv structure. In some embodiments, the tumor-associated antigen is PD-L1. In some embodiments, the immune system agonist is CD40. In some embodiments, a bispecific binding polypeptide binds to PD-L1 and CD40. In some embodiments, a bispecific bind molecule binds to PD-L1 and a protein selected from 4-1BB, CD3 and OX40. In some embodiments, a bispecific binding molecule binds to CD40 and a tumor-associated antigen selected from ROR1 and EGFR.

Provided herein are bispecific binding polypeptides that bind to PD-L1 and CD40 and comprise the sequences of molecules PC401003, PC401119, PC401120, PC401122, PC401124, PC401128, PC401129, PC401132, PC401133, PC401144, or PC401145, as shown in Table 1. Further provided herein are bispecific binding polypeptides that bind to PD-L1 and CD4 and comprise the sequences of the set of twelve CDR sequences of any one of the molecules PC401003, PC401119, PC401120, PC401122 PC401124d PC401128d PC401129, PC40132 PC401133 PC401144, and PC401145. Further provided herein are bispecific binding polypeptides that bind to PD-L1 and CD40 and comprise the sequences of the set of four variable region sequences of any one of the molecules PC401003, PC401119, PC401120, PC401122, PC401124, PC401128, PC401129, PC401132, PC401133 PC401144 and PC401145.

In Table 1, CDR sequences (Kabat definition) are single underlined when shown as part of longer sequences. In the scFv sequences, the scFv linker sequence is in italics, the VL sequence is in bold, and the VHI sequence is in plain font. In the bispecific molecule sequences, the PD-L1 binding domain sequence is in bold, the CD40 binding domain sequence is in italics, the Fe sequence is in plain font, and the H125 linker is double underlined. In the PC401119, PC401120, PC401122, PC401124, PC401128, PC401129, PC401132, PC401133, PC401144, and PC401145 molecules, amino acid substitutions are compared to the corresponding CD4001242 sequence. Humanized 1D5 anti-CD40 sequence are shown in bold.

TABLE 1
Exemplary amino acid sequences of bispecific molecules and their
components

| SEQ ID | | |
|---|---|---|
| NO | Description | Sequence |
| 1 | Humanized 5F9 | DYGMA |
| | anti-PD-L1 | |
| | HCDR1 | |
| 2 | Humanized 5F9 | FITSLAYIIYYADSVKG |
| | anti-PD-L1 | |
| | HCDR2 | |
| 3 | Humanized SF9 | NYGSSPYYEDY |
| | anti-PD-LI | |
| | HCDR3 | |
| 4 | PDL01152 | EVQLVESGGGLVQPGGSLRLSCAASGFTFSDYGMAWVRQAPGKGLEWVS |
| | Humanized 5F9 | FITSLAYIIYYADSVKGRFTISRDNAKNSLYLQMNSLRAEDTAVYYCAR |
| | anti-PD-L1 VH | NYGSSPYYFDYWGQGTLVTVSS |
| 5 | Humanized 5F9 | RASQDINKELS |
| | anti-PD-LI | |
| | LCDRI | |
| 6 | Humanized 5F9 | YTSSLQS |
| | anti-PD-L1 | |
| | LCDR2 | |
| 7 | Humanized 5F9 | LQYDNLYT |
| | anti-PD-L1 | |
| | LCDR3 | |
| 8 | PDL01152 | DIQMTQSPSSLSASVGDRVTITCRASQDINKELSWYQQKPGKAPKLLIH |
| | Humanized 5F9 | YTSSLQSGVPSRESGSGSGTDFTLTISSLQPEDFATYYCLQYDNLYTFG |
| | anti-PD-LI VL | QGTKLEIK |
| 9 | PDL01152 | DIQMTQSPSSLSASVGDRVTITCRASQDINKELSWYQQKPGKAPKLLIH |
| | Humanized 5F9 | YTSSLQSGVPSRFSGSGSGTDFILTISSLOPEDFATYYCLQYDNLYTFG |
| | anti-PD-LI | QGIKLEIKGGGGSGGGGSGGGGSGGGGSEVOLVESGGGLVQPGGSLRLS |
| | scFv | CAASGFTFSDYGMAWVRQAPGKGLEWVSFITSLAYIIYYADSVKGRFTI |
| | | SRDNAKNSLYLQMNSLRAEDTAVYYCARNYGSSPYYFDYWGQGTLVTVS |
| | | S |
| 10 | Humanized 1D5 | TSWIE |
| | anti-CD40 | |
| | HCDR1 | |
| 11 | Humanized 1D5 | SILPGSGSTNYAQKFQG |
| | anti-CD40 | |
| | HCDR2 | |
| 12 | Humanized 1D5 | GDDGSYVRWYFDH |
| | anti-CD40 | |
| | HCDR3 | |
| 13 | CD4001242 | QVQLVQSGAEVKKPGSSVKVSCKASGYTESTSWIEWIRQAPGOGLEWMG |
| | Humanized 1D5 | SILPGSGSTNYAQKFQGRVTITADTSTSTAYMELSSLRSEDTAVYYCAR |
| | anti-CD40 VH | GDDGSYVRWYFDHWGQGTLVTVSS |
| 14 | Humanized 1D5 | RASQSLLFSVNOKNYLA |
| | anti-CD40 | |
| | LCDRI | |
| 15 | Humanized 1D5 | WASSLQS |
| | anti-CD40 | |
| | LCDR2 | |
| 16 | Humanized 1D5 | QHYYSYPWT |
| | anti-CD40 | |
| | LCDR3 | |
| 17 | CD4001242 | DIQMTQSPSSVSASVGDRVTITCRASQSLLFSVNQKNYLAWYQQKPGKA |
| | Humanized 1D5 | PKLLIYWASSLQSGVPSRESGSGSGIDFTLTISSLOPEDFATYYCQHYY |
| | anti-CD40 VL | SYPWTFGQGTKVEIK |
| 18 | CD4001242 | DIQMTQSPSSVSASVGDRVTITCRASQSLLESVNQKNYLAWYQQKPGKA |
| | Humanized 1D5 | PKLLIYWASSLQSGVPSRESGSGSGTDFTLTISSLQPEDFATYYCQHYY |
| | anti-CD40 scFv | SYPWTFGQGTKVEIKGGGGSGGGGSGGGGSGGGGSQVQLVQSGAEVKKP |
| | | GSSVKVSCKASGYTESTSWIEWIRQAPGQGLEWMGSILPGSGSTNYAQK |
| | | FQGRVTITADTSTSTAYMELSSLRSEDTAVYYCARGDDGSYVRWYEDHW |
| | | GQGTLVTVSS |
| 19 | PC401003 | DIQMTQSPSSLSASVGDRVTITCRASQDINKELSWYQQKPGKAPKLLIH |
| | | YTSSLQSGVPSRFSGSGSGIDFTLTISSLQPEDFATYYCLQYDNLYTFG |
| | | QGTKLEIKGGGGSGGGGSGGGGSGGGGSEVOLVESGGGLVQPGGSLRLS |
| | | CAASGFTFSDYGMAWVROAPGKGLEWVSFITSLAYIIYYADSVKGRFTI |
| | | SRDNAKNSLYLOMNSLRAEDTAVYYCARNYGSSPYYEDYWGOGTLVTVS |
| | | SEPKSSDKTHTCPPCPAPPAAAPSVELFPPKPKDTLMISRTPEVTCVVV |
| | | DVSHEDPEVKFNWYVDGVEVHNAKIKPREEQYNSTYRVVSVLTVLHODW |
| | | LNGKEYKCAVSNKALPAPIEKTISKAKGQPREPQVYTLPPSRDELTKNQ |
| | | VSLTCLVKGFYPSDIAVEWESNGQPENNYKTTPPVLDSDGSFFLYSKLT |
| | | 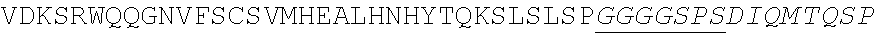 |
| | | SSVSASVGDRVTITCRASQSLLESVNQKNYLAWYQQKPGKAPKLLTYWA |
| | | SSLQSGVPSRESGSGSGIDFTLTISSLQPEDFATYYCQHYYSYPWTFGQ |
| | | GTKVEIKGGGGSGGGGGGGGSGGGGSQVQLVQSGAEVKKPGSSVKVSC |
| | | KASGYTESTSWIEWIRQAPGQGLEWMGSILPGSGSTNYAQKFQGRVTIT |
| | | ADTSTSTAYMELSSLRSEDTAVYYCARGDDGSYVRWYFDHWGQGTLVTV |
| | | SS |
| 20 | scFv linker | GGGGSGGGGSGGGGSGGGGS |
| 21 | H125 linker | GGGGSPS |
| 22 | Fc (hinge-CH2- | EPKSSDKTHTCPPCPAPPAAAPSVELFPPKPKDTLMISRTPEVTCVVVD |
| | CH3) | VSHEDPEVKFNWYVDGVEVHNAKTKPREEQYNSTYRVVSVLTVLHQDWL |
| | | NGKEYKCAVSNKALPAPIEKTISKAKGQPREPQVYTLPPSRDELTKNQV |
| | | SLTCLVKGFYPSDIAVEWESNGQPENNYKTTPPVLDSDGSFFLYSKLTV |
| | | DKSRWQQGNVFSCSVMHEALHNHYTQKSLSLSP |
| 23 | PC401119, | SILPGSGSTNYNQKEQG |
| | PC401124, | |
| | PC401144, and | |
| | PC401145 anti- | |
| | CD40 HCDR2 | |
| 24 | PC401119 anti- | QVQLVQSGAEVKKPGSSVKVSCKASGYTESTSWIEWIRQAPGQGLEWMG |
| | CD40 VH | SILPGSGSTNYNQKFQGRVTITADTSTSTAYMELSSLRSEDTAVYYCAR |
| | | GDDGSYVRWYFDHWGQGTLVTVSS |
| 25 | PC401119 anti- | DIQMTQSPSSVSASVGDRVTITCRASQSLLFSVNQKNYLAWYQQKPGKA |
| | CD40 scFv | PKLLIYWASSLQSGVPSRFSGSGSGTDFTLTISSLQPEDFATYYCQHYY |
| | | SYPWTFGQGTKVEIKGGGGSGGGGSGGGGSGGGGSQVQLVQSGAEVKKP |
| | | GSSVKVSCKASGYTESTSWIEWIRQAPGOGLEWMGSILPGSGSTNYNQK |
| | | FQGRVTITADTSTSTAYMELSSLRSEDTAVYYCARGDDGSYVRWYFDHW |
| | | GQGTLVTVSS |
| 26 | PC401119 | DIQMTQSPSSLSASVGDRVTITCRASQDINKELSWYQQKPGKAPKLLIH |
| | | YTSSLQSGVPSRFSGSGSGTDFTLTISSLQPEDFATYYCLQYDNLYTFG |
| | | QGIKLEIKGGGGSGGGGSGGGGSGGGGSEVQLVESGGGLVQPGGSLRLS |
| | | CAASGETFSDYGMAWVROAPGKGLEWVSFITSLAYIIYYADSVKGRETI |
| | | SRDNAKNSLYLOMNSLRAEDTAVYYCARNYGSSPYYFDYWGQGTLVTVS |
| | | SEPKSSDKTHTCPPCPAPPAAAPSVELFPPKPKDELMISRTPEVTCVVV |
| | | DVSHEDPEVKFNWYVDGVEVHNAKIKPREEQYNSTYRVVSVLTVLHQDW |
| | | LNGKEYKCAVSNKALPAPIEKTISKAKGQPREPQVYTLPPSRDELTKNQ |
| | | VSLTCLVKGFYPSDIAVEWESNGQPENNYKTTPPVLDSDGSFFLYSKLT |
| | | 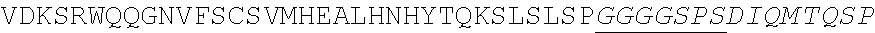 |
| | | SSVSASVGDRVTITCRASQSLLFSVNOKNYLAWYQQKPGKAPKLLIYWA |
| | | SSLQSGVPSRESGSGSGIDFTLTISSLQPEDFATYYCQHYYSYPWTFGQ |
| | | GTKVEIKGGGGGGGGSGGGGSGGGGSQVQLVQSGAEVKKPGSSVKVSC |
| | | KASGYTESTSWIEWIRQAPGOGLEWMGSILPGSGSTNYNQKFQGRVTIT |
| | | ADTSTSTAYMELSSLRSEDTAVYYCARGDDGSYVRWYFDHWGQGTLVTV |
| | | SS |
| 27 | PC401120 anti- | QVQLVQSGAEVKKPGSSVKVSCKASGYTESTSWIEWIRQAPGQCLEWMG |
| | CD40 VH | SILPGSGSTNYAQKFQGRVTITADTSTSTAYMELSSLRSEDTAVYYCAR |
| | | GDDGSYVRWYFDHWGQGTLVTVSS |
| 28 | PC401120 and | DIQMTQSPSSVSASVGDRVTITCRASQSLLFSVNQKNYLAWYQQKPGKA |
| | PC401145 anti- | PKLLIYWASSLQSGVPSRESGSGSGTDFTLTISSLQPEDFATYYCQHYY |
| | CD40 VL | SYPWTFGCGTKVEIK |
| 29 | PC401120 anti- | DIQMTQSPSSVSASVGDRVTITCRASQSLLFSVNQKNYLAWYQQKPGKA |
| | CD40 scFv | PKLLIYWASSLQSGVPSRESGSGSGTDFTLTISSLQPEDFATYYCQHYY |
| | | SYPWTFGCGTKVEIKGGGGSGGGGSGGGGSGGGGSQVQLVQSGAEVKKP |
| | | GSSVKVSCKASGYTESTSWIEWIRQAPGQCLEWMGSILPGSGSTNYAQK |
| | | FQGRVTITADTSTSTAYMELSSLRSEDTAVYYCARGDDGSYVRWYEDHW |
| | | GQGTLVTVSS |
| 30 | PC401120 | DIQMTQSPSSLSASVGDRVTITCRASQDINKELSWYQQKPGKAPKLLIH |
| | | YTSSLQSGVPSRFSGSGSGIDFTLTISSLQPEDFATYYCLQYDNLYTFG |
| | | QGTKLEIKGGGGSGGGGSGGGGSGGGGSEVQLVESGGGLVQPGGSLRLS |
| | | CAASGFTFSDYGMAWVRQAPGKGLEWVSFITSLAYIIYYADSVKGRFTI |
| | | SRDNAKNSLYLOMNSLRAEDTAVYYCARNYGSSPYYEDYWGQGTLVTVS |
| | | SEPKSSDKTHTCPPCPAPPAAAPSVELFPPKPKDTLMISRTPEVTCVVV |
| | | DVSHEDPEVKENWYVDGVEVHNAKTKPREEQYNSTYRVVSVLTVLHQDW |
| | | LNGKEYKCAVSNKALPAPIEKTISKAKGQPREPQVYTLPPSRDELTKNQ |
| | | VSLTCLVKGFYPSDIAVEWESNGQPENNYKTTPPVLDSDGSFFLYSKLT |
| | | 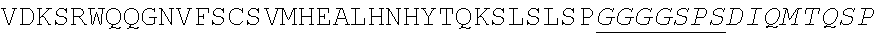 |
| | | SSVSASVGDRVTITCRASQSLLESVNQKNYLAWYQQKPGKAPKLDIYWA |
| | | SSLQSGVPSRESGSGSGTDETITISSLQPEDFATYYCQHYYSYPWTFGC |
| | | GTKVEIKGGGGSGGGGSGGGGGGGGSQVQLVQSGAEVKKPGSSVKVSC |
| | | KASGYTESTSWIEWIRQAPGQCLEWMGSILPGSGSTNYAQKFQGRVTIT |
| | | ADTSTSTAYMELSSLRSEDTAVYYCARGDDGSYVRWYFDHWGQGTLVTV |
| | | SS |
| 31 | PC401122 | GDDGSYNRWYFDH |
| | PC401124, | |
| | PC401144, and | |
| | PC401145 anti- | |
| | CD40 HCDR3 | |
| 32 | PC401122 anti- | QVQLVQSGAEVKKPGSSVKVSCKASGYTESTSWIEWIRQAPGQGLEWMG |
| | CD40 VH | SILPGSGSTNYAQKFQGRVTITADTSTSTAYMELSSLRSEDTAVYYCAR |
| | | GDDGSYNRWYFDHWGQGTLVTVSS |
| 33 | PC401122 anti- | DIQMTQSPSSVSASVGDRVTITCRASQSLLFSVNQKNYLAWYQQKPGKA |
| | CD40 scFv | PKLLIYWASSLQSGVPSRFSGSGSGTDFTLTISSLOPEDFATYYCQHYY |
| | | SYPWTFGQGTKVEIKGGGGSGGGGSGGGGSGGGGSQVQLVQSGAEVKKP |
| | | GSSVKVSCKASGYTESTSWIEWIRQAPGQGLEWMGSILPGSGSTNYAQK |
| | | FQGRVTITADTSTSTAYMELSSLRSEDTAVYYCARGDDGSYNRWYFDHW |
| | | GQGTLVTVSS |
| 280 | PC401122 | DIQMTQSPSSLSASVGDRVTITCRASQDINKELSWYQQKPGKAPKLLIH |
| | | YTSSLQSGVPSRFSGSGSGTDFTLTISSLQPEDFATYYCLQYDNLYTFG |
| | | QGTKLEIKGGGGSGGGGSGGGGGGGGSEVQLVESGGGLVQPGGSLRLS |
| | | CAASGFTFSDYGMAWVRQAPGKGLEWVSFITSLAYIIYYADSVKGRETI |
| | | SRDNAKNSLYLQMNSLRAEDTAVYYCARNYGSSPYYFDYWGQGTLVTVS |
| | | SEPKSSDKTHTCPPCPAPPAAAPSVFLFPPKPKDTLMISRTPEVTCVVV |
| | | DVSHEDPEVKFNWYVDGVEVHNAKTKPREEQYNSTYRVVSVLTVLHQDW |
| | | LNGKEYKCAVSNKALPAPIEKTISKAKGQPREPQVYTLPPSRDELTKNQ |
| | | VSLTCLVKGFYPSDIAVEWESNGQPENNYKTTPPVLDSDGSFFLYSKLT |
| | | 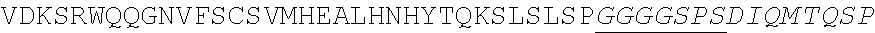 |
| | | SSVSASVGDRVTITCRASQSLLFSVNQKNYLAWYQQKPGKAPKLLIYWA |
| | | SSLQSGVPSRFSGSGSGTDFTLTISSLOPEDFATYYCQHYYSYPWTFGQ |
| | | GTKVEIKGGGGSGGGGSGGGGSGGGGSQVQLVOSGAEVKKPGSSVKVSC |
| | | KASGYTESTSWIEWIRQAPGOGLEWMGSILPGSGSTNYAQKFQGRVTIT |
| | | ADTSTSTAYMELSSLRSEDTAVYYCARGDDGSYNRWYFDHWGQGTLVTV |
| | | SS |
| 34 | PC401124 anti- | QVQLVQSGAEVKKPGSSVKVSCKASGYTESTSWIEWIRQAPGQGLEWMG |
| | CD40 VH | SILPGSGSTNYNQKFQGRVTITADESTSTAYMELSSLRSEDTAVYYCAR |
| | | GDDGSYNRWYFDHWGQGTLVTVSS |
| 35 | PC401124 anti- | DIQMTQSPSSVSASVGDRVTITCRASQSLLFSVNQKNYLAWYQQKPGKA |
| | CD40 scFv | PKLLIYWASSLQSGVPSRESGSGSGTDFILTISSLQPEDFATYYCQHYY |
| | | SYPWTFGQGTKVEIKGGGGGGGGSGGGGSGGGGSQVQLVQSGAEVKKP |
| | | GSSVKVSCKASGYTESTSWIEWIRQAPGOGLEWMGSILPGSGSTNYNQK |
| | | FQGRVTITADTSTSTAYMELSSLRSEDTAVYYCARGDDGSYNRWYFDHW |
| | | GQGTLVTVSS |
| 36 | PC401124 | DIQMTQSPSSLSASVGDRVTITCRASQDINKFLSWYQQKPGKAPKLLIH |
| | | YTSSLQSGVPSRESGSGSGTDFTLTISSLQPEDFATYYCLQYDNLYTFG |
| | | QGTKLEIKGGGGSGGGGSGGGGGGGGSEVQLVESGGGLVQPGGSLRLS |
| | | CAASGFTFSDYGMAWVRQAPGKGLEWVSFITSLAYIIYYADSVKGRFTI |
| | | SRDNAKNSLYLOMNSLRAEDTAVYYCARNYGSSPYYFDYWGQGTLVTVS |
| | | SEPKSSDKTHTCPPCPAPPAAAPSVELFPPKPKDTLMISRTPEVTCVVV |
| | | DVSHEDPEVKENWYVDGVEVHNAKTKPREEQYNSTYRVVSVLTVLHQDW |
| | | LNGKEYKCAVSNKALPAPIEKTISKAKGQPREPQVYTLPPSRDELTKNQ |
| | | VSLTCLVKGFYPSDIAVEWESNGQPENNYKTTPPVLDSDGSFFLYSKLI |
| | | 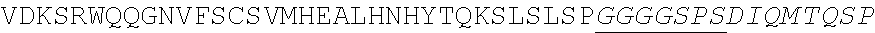 |
| | | SSVSASVGDRVTITCRASQSLLFSVNOKNYLAWYQQKPGKAPKLLIYWA |
| | | SSLQSGVPSRFSGSGSGTDFTLTISSLOPEDFATYYCQHYYSYPWTFGQ |
| | | GTKVEIKGGGGSGGGGSGGGGSGGGGSQVQLVQSGAEVKKPGSSVKVSC |
| | | KASGYTESTSWIEWIRQAPGQGLEWMGSILPGSGSTNYNQKFQGRVTIT |
| | | ADTSTSTAYMELSSLRSEDTAVYYCARGDDGSYNRWYFDHWGQGTLVTV |
| | | SS |
| 37 | PC401128, and | RASQSLTFSVNOKNYLA |
| | PC401144 anti- | |
| | CD40 LCDRI | |
| 38 | PC401128 anti- | DIQMTQSPSSVSASVGDRVTITCRASQSLTFSVNQKNYLAWYQQKPGKA |
| | CD40 VL | PKLLIYWASSLQSGVPSRFSGSGSGTDFTLTISSLOPEDFATYYCQHYY |
| | | SYPWTFGQGTKVEIK |
| 39 | PC401128 anti- | DIQMTQSPSSVSASVGDRVTITCRASQSLTFSVNQKNYLAWYQQKPGKA |
| | CD40 scFv | PKLLIYWASSLQSGVPSRESGSGSGTDFTLTISSLQPEDFATYYCQHYY |
| | | SYPWTFGQGTKVEIKGGGGSGGGGSGGGGSGGGGSQVQLVQSGAEVKKP |
| | | GSSVKVSCKASGYTESTSWIEWIRQAPGOGLEWMGSILPGSGSTNYAQK |
| | | FQGRVTITADTSTSTAYMELSSLRSEDTAVYYCARGDDGSYVRWYEDHW |
| | | GQGTLVTVSS |
| 40 | PC401128 | DIQMTQSPSSLSASVGDRVTITCRASQDINKELSWYQQKPGKAPKLLIH |
| | | YTSSLQSGVPSRFSGSGSGTDFTLTISSLQPEDFATYYCLQYDNLYTFG |
| | | QGTKLEIKGGGGSGGGGGGGGSGGGGSEVQLVESGGGLVQPGGSLRLS |
| | | CAASGETFSDYGMAWVRQAPGKGLEWVSFITSLAYIIYYADSVKGRETI |
| | | SRDNAKNSLYLQMNSLRAEDTAVYYCARNYGSSPYYEDYWGOGTLVTVS |
| | | SEPKSSDKTHTCPPCPAPPAAAPSVELFPPKPKDTLMISRTPEVTCVVV |
| | | DVSHEDPEVKFNWYVDGVEVHNAKIKPREEQYNSTYRVVSVLTVLHQDW |
| | | LNGKEYKCAVSNKALPAPIEKTISKAKGQPREPQVYTLPPSRDELTKNQ |
| | | VSLTCLVKGFYPSDIAVEWESNGQPENNYKTTPPVLDSDGSFFLYSKLT |
| | | 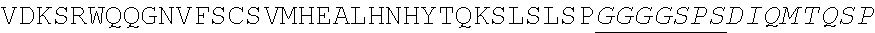 |
| | | SSVSASVGDRVTITCRASQSLTFSVNQKNYLAWYQQKPGKAPKLLIYWA |
| | | SSLQSGVPSRFSGSGSGTDFTLTISSLQPEDFATYYCQHYYSYPWTFGQ |
| | | GTKVEIKGGGGSGGGGSGGGGSGGGGSQVQLVQSGAEVKKPGSSVKVSC |
| | | KASGYTESTSWIEWIRQAPGOGLEWMGSILPGSGSTNYAQKFQGRVTIT |
| | | ADTSTSTAYMELSSLRSEDTAVYYCARGDDGSYVRWYEDHWGQGTLVTV |
| | | SS |
| 41 | PC401129 anti- | RASQSLNFSVNQKNYLA |
| | CD40 LCDRI | |
| 42 | PC401129 anti- | DIQMTQSPSSVSASVGDRVTITCRASQSLNFSVNQKNYLAWYQQKPGKA |
| | CD40 VL | PKLLIYWASSLQSGVPSRFSGSGSGTDFTLTISSLOPEDFATYYCQHYY |
| | | SYPWTFGQGTKVEIK |
| 43 | PC401129 anti- | DIQMTQSPSSVSASVGDRVTITCRASQSLNFSVNQKNYLAWYQQKPGKA |
| | CD40 scFv | PKLLIYWASSLQSGVPSRESGSGSGTDFTLTISSLOPEDFATYYCQHYY |
| | | SYPWTFGQGTKVEIKGGGGSGGGGSGGGGSGGGGSQVOLVOSGAEVKKP |
| | | GSSVKVSCKASGYTESTSWIEWIRQAPGQGLEWMGSILPGSGSTNYAQK |
| | | FQGRVTITADTSTSTAYMELSSLRSEDTAVYYCARGDDGSYVRWYFDHW |
| | | GQGTLVTVSS |
| 44 | PC401129 | DIQMTQSPSSLSASVGDRVTITCRASQDINKELSWYQQKPGKAPKLLIH |
| | | YTSSLQSGVPSRESGSGSGTDFTLTISSLQPEDFATYYCLQYDNLYTFG |
| | | QGTKLEIKGGGGSGGGGSGGGGSGGGGSEVQLVESGGGLVQPGGSIRLS |
| | | CAASGFTFSDYGMAWVRQAPGKGLEWVSFITSLAYIIYYADSVKGRFTI |
| | | SRDNAKNSLYLOMNSLRAEDTAVYYCARNYGSSPYYFDYWGQGTLVTVS |
| | | SEPKSSDKTHTCPPCPAPPAAAPSVFLFPPKPKDTLMISRTPEVTCVVV |
| | | DVSHEDPEVKFNWYVDGVEVHNAKTKPREEQYNSTYRVVSVLTVLHQDW |
| | | LNGKEYKCAVSNKALPAPIEKTISKAKGOPREPQVYTLPPSRDELTKNQ |
| | | VSLTCLVKGFYPSDIAVEWESNGQPENNYKTTPPVLDSDGSFFLYSKLT |
| | | 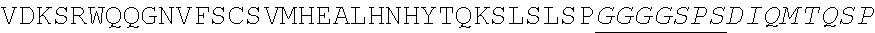 |
| | | SSVSASVGDRVTITCRASQSLNFSVNQKNYLAWYQQKPGKAPKLLIYWA |
| | | SSLQSGVPSRESGSGSGIDFTLTISSLOPEDFATYYCQHYYSYPWTFGQ |
| | | GTKVEIKGGGGSGGGGSGGGGSGGGGSQVQLVQSGAEVKKPGSSVKVSC |
| | | KASGYTESTSWIEWIRQAPGOGLEWMGSILPGSGSTNYAQKFQGRVTIT |
| | | ADTSTSTAYMELSSLRSEDTAVYYCARGDDGSYVRWYFDHWGQGTLVTV |
| | | SS |
| 45 | PC401132 anti- | SINPGSGSTNYAQKFOG |
| | CD40 HCDR2 | |
| 46 | PC401132 anti- | QVQLVQSGAEVKKPGSSVKVSCKASGYTESTSWIEWIRQAPGQGLEWMG |
| | CD40 VH | SINPGSGSTNYAQKFQGRVTITADISTSTAYMELSSLRSEDTAVYYCAR |
| | | GDDGSYVRWYFDHWGQGTLVTVSS |
| 47 | PC401132 anti- | DIQMTQSPSSVSASVGDRVTITCRASQSLLFSVNQKNYLAWYQQKPGKA |
| | CD40 scFv | PKLLIYWASSLQSGVPSRESGSGSGTDFTLTISSLQPEDFATYYCQHYY |
| | | SYPWTFGQGTKVEIKGGGGSGGGGSGGGGSGGGGSQVQLVQSGAEVKKP |
| | | GSSVKVSCKASGYTESTSWIEWIRQAPGOGLEWMGSINPGSGSTNYAQK |
| | | FQGRVTITADTSTSTAYMELSSLRSEDTAVYYCARGDDGSYVRWYFDHW |
| | | GQGTLVTVSS |
| 48 | PC401132 | DIQMTOSPSSLSASVGDRVTITCRASQDINKELSWYQQKPGKAPKLLIH |
| | | YTSSLQSGVPSRESGSGSGTDFTLTISSLQPEDFATYYCLQYDNLYTFG |
| | | QGTKLEIKGGGGSGGGGSGGGGSGGGGSEVQLVESGGGLVQPGGSIRLS |
| | | CAASGFTFSDYGMAWVRQAPGKGLEWVSFITSLAYIIYYADSVKGRFTI |
| | | SRDNAKNSLYLOMNSLRAEDTAVYYCARNYGSSPYYFDYWGQGTLVTVS |
| | | SEPKSSDKTHTCPPCPAPPAAAPSVFLFPPKPKDTLMISRTPEVTCVVV |
| | | DVSHEDPEVKFNWYVDGVEVHNAKTKPREEQYNSTYRVVSVLTVLHQDW |
| | | LNGKEYKCAVSNKALPAPIEKTISKAKGQPREPQVYTLPPSRDELTKNQ |
| | | VSLTCLVKGFYPSDIAVEWESNGQPENNYKTTPPVLDSDGSFFLYSKLT |
| | | 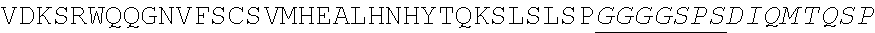 |
| | | SSVSASVGDRVTITCRASQSLLFSVNOKNYLAWYQQKPGKAPKLLIYWA |
| | | SSLQSGVPSRFSGSGSGTDFILTISSLQPEDFATYYCQHYYSYPWTFGQ |
| | | GTKVEIKGGGGSGGGGSGGGGSGGGGSQVQLVQSGAEVKKPGSSVKVSC |
| | | KASGYTESTSWIEWIRQAPGQGLEWMGSINPGSGSTNYAQKFQGRVTIT |
| | | ADTSTSTAYMELSSLRSEDTAVYYCARGDDGSYVRWYFDHWGQGTLVTV |
| | | SS |
| 49 | PC401133 anti- | SIQPGSGSTNYAQKFQG |
| | CD40 HCDR2 | |
| 50 | PC401133 anti- | QVQLVQSGAEVKKPGSSVKVSCKASGYTESTSWIEWIRQAPGQGLEWMG |
| | CD40 VH | SIQPGSGSTNYAQKFQGRVTITADTSTSTAYMELSSLRSEDTAVYYCAR |
| | | GDDGSYVRWYFDHWGQGTLVTVSS |
| 51 | PC401133 anti- | DIQMTQSPSSVSASVGDRVTITCRASQSLLFSVNQKNYLAWYQQKPGKA |
| | CD40 scFv | PKLLIYWASSLQSGVPSRESGSGSGTDFTLTISSLOPEDFATYYCQHYY |
| | | SYPWTFGQGTKVEIKGGGGSGGGGSGGGGSGGGGSQVQLVQSGAEVKKP |
| | | GSSVKVSCKASGYTESTSWIEWIRQAPGOGLEWMGSIQPGSGSTNYAQK |
| | | FQGRVTITADTSTSTAYMELSSLRSEDTAVYYCARGDDGSYVRWYEDHW |
| | | GQGTLVTVSS |
| 52 | PC401133 | DIQMTQSPSSLSASVGDRVTITCRASQDINKELSWYQQKPGKAPKLLIH |
| | | YTSSLQSGVPSRFSGSGSGTDFTLTISSLQPEDFATYYCLQYDNLYTFG |
| | | QGTKLEIKGGGGSGGGGSGGGGSGGGGSEVQLVESGGGLVQPGGSLRLS |
| | | CAASGETFSDYGMAWVROAPGKGLEWVSFITSLAYIIYYADSVKGRETI |
| | | SRDNAKNSLYLQMNSLRAEDTAVYYCARNYGSSPYYEDYWGQGTLVTVS |
| | | SEPKSSDKTHTCPPCPAPPAAAPSVELFPPKPKDTLMISRTPEVTCVVV |
| | | DVSHEDPEVKFNWYVDGVEVHNAKTKPREEQYNSTYRVVSVLTVLHQDW |
| | | LNGKEYKCAVSNKALPAPIEKTISKAKGQPREPQVYTLPPSRDELTKNQ |
| | | VSLTCLVKGFYPSDIAVEWESNGQPENNYKTTPPVLDSDGSFFLYSKLT |
| | | 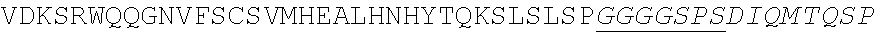 |
| | | SSVSASVGDRVTITCRASQSLLFSVNQKNYLAWYQQKPGKAPKLLIYWA |
| | | SSLQSGVPSRESGSGSGIDFTLTISSLOPEDFATYYCQHYYSYPWTFGQ |
| | | GTKVEIKGGGGSGGGGSGGGGSGGGGSQVQLVQSGAEVKKPGSSVKVSC |
| | | KASGYTESTSWIEWIRQAPGQGLEWMGSIQPGSGSTNYAQKFQGRVTIT |
| | | ADTSTSTAYMELSSLRSEDTAVYYCARGDDGSYVRWYFDHWGQGTLVTV |
| | | SS |
| 484 | PC401144 and | QVQLVQSGAEVKKPGSSVKVSCKASGYTESTSWIEWIRQAPGQCLEWMG |
| | PC401145 anti- | SILPGSGSTNYNQKFQGRVTITADTSTSTAYMELSSLRSEDTAVYYCAR |
| | CD40 VH | GDDGSYNRWYFDHWGQGTLVTVSS |
| 485 | PC401144 anti- | DIQMTQSPSSVSASVGDRVTITCRASQSLTFSVNQKNYLAWYQQKPGKA |
| | CD40 VL | PKLLIYWASSLQSGVPSRESGSGSGTDETLTISSLQPEDFATYYCQHYY |
| | | SYPWTFGCGTKVEIK |
| 486 | PC401144 anti- | DIQMTQSPSSVSASVGDRVTITCRASQSLTFSVNQKNYLAWYQQKPGKA |
| | CD40 scFv | PKLLIYWASSLQSGVPSRFSGSGSGTDFTLTISSLOPEDFATYYCQHYY |
| | | SYPWTFGCGTKVEIKGGGGSGGGGSGGGGSGGGGSQVQLVQSGAEVKKP |
| | | GSSVKVSCKASGYTESTSWIEWIRQAPGQCLEWMGSILPGSGSTNYNQK |
| | | FQGRVTITADTSTSTAYMELSSLRSEDTAVYYCARGDDGSYNRWYFDHW |
| | | GQGTLVTVSS |
| 487 | PC401144 | DIQMTQSPSSLSASVGDRVTIICRASQDINKELSWYQQKPGKAPKLLIH |
| | | YTSSLQSGVPSRFSGSGSGTDFTLTISSLQPEDFATYYCLQYDNLYTFG |
| | | QGTKLEIKGGGGSGGGGSGGGGSGGGGSEVQLVESGGGLVQPGGSLRLS |
| | | CAASGETFSDYGMAWVROAPGKGLEWVSFITSLAYIIYYADSVKGRFTI |
| | | SRDNAKNSLYLQMNSLRAEDTAVYYCARNYGSSPYYEDYWGQGTLVTVS |
| | | SEPKSSDKTHTCPPCPAPPAAAPSVFLFPPKPKDTLMISRTPEVTCVVV |
| | | DVSHEDPEVKENWYVDGVEVHNAKTKPREEQYNSTYRVVSVLTVLHQDW |
| | | LNGKEYKCAVSNKALPAPIEKTISKAKGOPREPQVYTLPPSRDELTKNQ |
| | | VSLTCLVKGFYPSDIAVEWESNGQPENNYKTTPPVLDSDGSFFLYSKLI |
| | | 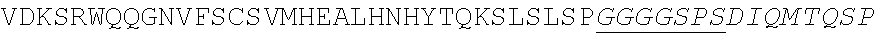 |
| | | SSVSASVGDRVTITCRASQSLTFSVNQKNYLAWYQQKPGKAPKLLIYWA |
| | | SSLQSGVPSRESGSGSGIDFILTISSLQPEDFATYYCQHYYSYPWTFGC |
| | | GTKVEIKGGGGSGGGGSGGGGSGGGGSQVQLVQSGAEVKKPGSSVKVSC |
| | | KASGYTESTSWIEWIRQAPGQCLEWMGSILPGSGSTNYNQKFQGRVTIT |
| | | ADTSTSTAYMELSSERSEDTAVYYCARGDDGSYNRWYEDHWGQGTLVTV |
| | | SS |
| 488 | PC401145 anti- | DIQMTQSPSSVSASVGDRVTITCRASQSLLFSVNOKNYLAWYQQKPGKA |
| | CD40 scFv | PKLLIYWASSLQSGVPSRESGSGSGTDFILTISSLQPEDFATYYCQHYY |
| | | SYPWTFGCGTKVEIKGGGGSGGGGSGGGGSGGGGSQVQLVQSGAEVKKP |
| | | GSSVKVSCKASGYTESTSWIEWIRQAPGQCLEWMGSILPGSGSTNYNQK |
| | | FQGRVTITADTSTSTAYMELSSLRSEDTAVYYCARGDDGSYNRWYFDHW |
| | | GQGTLVTVSS |
| 489 | PC401145 | DIQMTQSPSSLSASVGDRVTITCRASQDINKELSWYQQKPGKAPKLLIH |
| | | YTSSLQSGVPSRESGSGSGTDFTLTISSLQPEDFATYYCLQYDNLYTFG |
| | | QGTKLEIKGGGGSGGGGSGGGGSGGGGSEVQLVESGGGLVQPGGSLRLS |
| | | CAASGETFSDYGMAWVRQAPGKGLEWVSFITSLAYIIYYADSVKGRETI |
| | | SRDNAKNSLYLOMNSLRAEDTAVYYCARNYGSSPYYEDYWGQGTLVTVS |
| | | SEPKSSDKTHTCPPCPAPPAAAPSVFLFPPKPKDTLMISRTPEVTCVVV |
| | | DVSHEDPEVKENWYVDGVEVHNAKTKPREEQYNSTYRVVSVLTVLHQDW |
| | | LNGKEYKCAVSNKALPAPIEKTISKAKGQPREPQVYTLPPSRDELTKNQ |
| | | VSLTCLVKGFYPSDIAVEWESNGQPENNYKTTPPVLDSDGSFFLYSKLT |
| | | 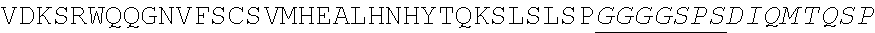 |
| | | SSVSASVGDRVTITCRASQSLLFSVNQKNYLAWYQQKPGKAPKLLIYWA |
| | | SSLQSGVPSRESGSGSGIDFTLTISSLQPEDFATYYCQHYYSYPWTFGC |
| | | GTKVEIKGGGGSGGGGSGGGGSGGGGSQVQLVQSGAEVKKPGSSVKVSC |
| | | KASGYTESTSWIEWIRQAPGQCLEWMGSILPGSGSTNYNQKFQGRVTIT |
| | | ADTSTSTAYMELSSERSEDTAVYYCARGDDGSYNRWYEDHWGQGTLVTV |
| | | SS |

Provided herein is a PD-L1 binding polypeptide that binds specifically to human PD-L1; wherein the PD-L1 binding polypeptide comprises, in order from amino terminus to carboxyl terminus or from carboxyl terminus to amino terminus, (a) a first binding domain; (b) a hinge region; (c) an immunoglobulin constant region; and (d) a second binding domain; wherein the first binding domain is a PD-L1 binding domain and the second binding domain binds an immunostimulatory protein; or wherein the first binding domain binds an immunostimulatory protein and the second binding domain is a PD-L1 binding domain. The first binding domain, the second binding domain, or both binding domains may be an scFv. In some embodiments, the immunostimulatory protein is CD40, 4-1BB, CD3 or OX40.

In some embodiments, the PD-L1 binding domain comprises (i) an immunoglobulin heavy chain variable region (VH) comprising HCDR1, HCDR2, and HCDR3; and (ii) an immunoglobulin light chain variable region (VL) comprising LCDR1, LCDR2, and LCDR3. In some embodiments, the HCDR1 comprises SEQ ID NO: 1, the HCDR2 comprises SEQ ID NO: 2, and the HCDR3 comprises SEQ ID NO: 3, the LCDR1 comprises SEQ ID NO:5, the LCDR2 comprises SEQ ID NO: 6, and the LCDR3 comprises SEQ ID NO: 7. In some embodiments, the VH comprises SEQ ID NO: 4; and the VL comprises SEQ ID NO: 8.

In some embodiments, the PD-L1 binding domain comprises SEQ ID NO: 9. In some embodiments, the PD-L1 binding domain comprises an amino acid sequence having at least 95%, 96%, 97%, 98%, or 99% sequence identity to SEQ ID NO: 9.

In some embodiments, the second binding domain comprises (i) an immunoglobulin heavy chain variable region (VH) comprising HCDR1, HCDR2, and HCDR3; and (ii) an immunoglobulin light chain variable region (VL) comprising LCDR1, LCDR2, and LCDR3.

TABLE 2
Sequences

>PDL01001-SEQ ID NO: 53
FTVTVPKDLYVVEYGSNMTIECKFPVEKQLDLAALIVYWEMEDKNIIQFVHGEEDLKVQHSSYRQ
RARLLKDQLSLGNAALQITDVKLQDAGVYRCMISYGGADYKRITVKVNAPYNKINQRILVVDPVT
SEHELTCQAEGYPKAEVIWTSSDHQVLSGKTTTTNSKREEKLFNVTSTLRINTTTNEIFYCTFRR
LDPEENHTAELVIPELPLAHPPNERSSSLNDIFEAQKIEWHEDYKDDDDKDYKDDDDKDYKDDDD
KHHHHHHHHHH
>PDL01002-SEQ ID NO: 54
FTVTVPKDLYVVEYGSNMTIECKFPVEKQLDLAALIVYWEMEDKNIIQFVHGEEDLKVQHSSYRQ
RARLLKDQLSLGNAALQITDVKLQDAGVYRCMISYGGADYKRITVKVNAPYNKINQRILVVDPVT
SEHELTCQAEGYPKAEVIWTSSDHQVLSGKTTTTNSKREEKLFNVTSTLRINTTTNEIFYCTFRR
LDPEENHTAELVIPELPLAHPPNERSSSEPRGPTIKPCPPCKCPAPNLLGGPSVFIFPPKIKDVL
MISLSPIVTCVVVDVSEDDPDVQISWFVNNVFVHTAQTQTHREDYNSTLRVVSALPIQHQDWMSG
KEFKCKVNNKDLPAPIERTISKPKGSVRAPQVYVLPPPEEEMTKKQVTLTCMVTDFMPEDIYVEW
TNNGKTELNYKNTEPVLDSDGSYFMYSKLRVEKKNWVERNSYSCSVVHEGLHNHHTIKSFSRTPG
K
>PDL01003-SEQ ID NO: 55
FTVTVPKDLYVVEYGSNMTIECKFPVEKQLDLTSLIVYWEMEDKNIIQFVHGEEDLKVQHSNYRQ
RAQLLKDQLSLGNAALRITDVKLQDAGVYRCMISYGGADYKRITVKVNAPYNKINQRILVVDPVT
SEHELTCQAEGYPKAEVIWTSSDHQVLSGKTTTTNSKREEKLLNVTSTLRINTTANEIFYCIFRR
LDPEENHTAELVIPELPLALPPNERSSSLNDIFEAQKIEWHEDYKDDDDKDYKDDDDKDYKDDDD
KHHHHHHHHHH
>PDL01004-SEQ ID NO: 56
FTVTVPKDLYVVEYGSNMTIECKFPVEKQLDLTSLIVYWEMEDKNIIQFVHGEEDLKVQHSNYRQ
RAQLLKDQLSLGNAALRITDVKLQDAGVYRCMISYGGADYKRITVKVNAPYNKINQRILVVDPVT
SEHELTCQAEGYPKAEVIWTSSDHQVLSGKTTTTNSKREEKLLNVTSTLRINTTANEIFYCIFRR
LDPEENHTAELVIPELPLALPPNERSSSEPRGPTIKPCPPCKCPAPNLLGGPSVFIFPPKIKDVL
MISLSPIVTCVVVDVSEDDPDVQISWFVNNVFVHTAQTQTHREDYNSTLRVVSALPIQHQDWMSG
KEFKCKVNNKDLPAPIERTISKPKGSVRAPQVYVLPPPEEEMTKKQVTLTCMVTDFMPEDIYVEW
TNNGKTELNYKNTEPVLDSDGSYFMYSKLRVEKKNWVERNSYSCSVVHEGLHNHHTTKSFSRTPG
K
>PDL01011-SEQ ID NO: 57
FTVTVPKDLYVVEYGSNMTIECKFPVEKQLDLAALIVYWEMEDKNIIQFVHGEEDLKVQHSSYRQ
RARLLKDQLSLGNAALQITDVKLQDAGVYRCMISYGGADYKRITVKVNAPYRKINQRISVDPATS
EHELICQAEGYPEAEVIWTNSDHQPVSGKRSVTTSRTEGMLLNVTSSLRVNATANDVFYCTFWRS
QPGQNHTAELIIPELPATHPPQNRSSSLNDIFEAQKIEWHEDYKDDDDKDYKDDDDKDYKDDDDK
HHHHHHHHHH
>PDL01017-SEQ ID NO: 58
FTVTVPKDLYVVEYGSNMTIECKFPVEKQLDLAALIVYWEMEDKNIIQFVHGEEDLKVQHSSYRQ
RARLLKDQLSLGNAALQITDVKLQDAGVYRCMISYGGADYKRITVKVNAPYRKINQRISVDPATS
EHELICQAEGYPEAEVIWTNSDHQPVSGKRSVTTSRTEGMLLNVTSSLRVNATANDVFYCTFWRS
QPGQNHTAELIIPELPATHPPQNRSSSEPRGPTIKPCPPCKCPAPNLLGGPSVFIFPPKIKDVLM
ISLSPIVTCVVVDVSEDDPDVQISWFVNNVFVHTAQTQTHREDYNSTLRVVSALPIQHQDWMSGK
EFKCKVNNKDLPAPIERTISKPKGSVRAPQVYVLPPPEEEMTKKQVTLTCMVTDFMPEDIYVEWT
NNGKTELNYKNTEPVLDSDGSYFMYSKLRVEKKNWVERNSYSCSVVHEGLHNHHTTKSFSRTPGK
>PDL01021-SEQ ID NO: 59
FTVTVPKDLYVVEYGSNMTIECKFPVEKQLDLAALIVYWEMEDKNIIQFVHGEEDLKVQHSSYRQ
RARLLKDQLSLGNAALQITDVKLQDAGVYRCMISYGGADYKRITVKVNAPYNKINQRILVVDPVT
SEHELTCQAEGYPKAEVIWTSSDHQVLSGKTTTTNSKREEKLFNVTSTLRINTTTNEIFYCTFRR
LDPEENHTAELVIPELPLAHPPNERTHLVILGAILLCLGVALIFIFRLRKGRMMDVKKCGIQDTN
SKKQSDTHLEET
>PDL01022-SEQ ID NO: 60
FTVTVPKDLYVVEYGSNMTIECKFPVEKQLDLTSLIVYWEMEDKNIIQFVHGEEDLKVQHSNYRQ
RAQLLKDQLSLGNAALRITDVKLQDAGVYRCMISYGGADYKRITVKVNAPYNKINQRILVVDPVT
SEHELTCQAEGYPKAEVIWTSSDHQVLSGKTTTTNSKREEKLLNVTSTLRINTTANEIFYCIFRR
LDPEENHTAELVIPELPLALPPNERTHLVILGAIFLLLGVALTFIFYLRKGRMMDMKKCGIRVTN
SKKQRDTQLEET
>PDL01026-SEQ ID NO: 61
FLDSPDRPWNPPTFSPALLVVTEGDNATFTCSFSNTSESFVLNWYRMSPSNQTDKLAAFPEDRSQ
PGQDCRFRVTQLPNGRDFHMSVVRARRNDSGTYLCGAISLAPKAQIKESLRAELRVTERRAEVPT
AHPSPSPRPAGQFQTLVVGVVGGLLGSLVLLVWVLAVICSRAARGTIGARRTGQPLKEDPSAVPV
FSVDYGELDFQWREKTPEPPVPCVPEQTEYATIVFPSGMGTSSPARRGSADGPRSAQPLRPEDGH
CSWPL
>PDL01031_2C11-SEQ ID NO: 62
DVKLVESGGGLVKPGGSLKLSCAASGFTFSSYTMSWVRQTPEKRLEWVATISSGGNYTYYPDSVK
GRFTISRDNAKNTLYLQLSSLKSEDTAMYYCTRDQAYYGNLFTYWGQGTLVTVSAGGGGSGGGGS
GGGGSGGGGSDIQMTQSPASLSASVGETVTITCRASENIYSFLAWYQQKQGKSPQLLVYKAKTLT
EGVPSRFSGSGSGTQFSLKINSLQPEDFGSYYCQHHYGSPPETFGGGTKVEIK
>PDL01032_3G5-SEQ ID NO: 63
QIQLQQSGPELVKPGASVKISCKASGYTFTDYYINWVKQKPEQGLEWIGWTFPGSINTKYNEKFK
GKATLTVDTSSSTAYMQLNSLTSEDTAVYFCARSPYEFDYWGQGTTLTVSSGGGGSGGGGSGGGG
SGGGGSDIQMTQTTSSLSASLGDRVTISCSASQGIRNYLNWYQQKPDGTVKLLIYYTSTLHSGVP
SRFSGSGSGTDYSLTISNLEPEDIATYYCQQYSKLPWTFGGGTRLEIK
>PDL01033_3G11-SEQ ID NO: 64
EVQLQESGPSLVKPSQTLSLTCSVTGDSITSGYWNWIRKFPGKKLEYMGYISYTGSTYYNPSLKS
RISIARDTSKNQYFLQLNSVTTEDTATYYCTRGENWDGGYFDYWGQGTTLTVSSGGGGSGGGGSG
GGGSGGGGSDIQMTQTTSSLSASLGDRVTISCSASQGIRNYLNWYQQKPDGTVKLLIYYTSTLHS
GVPSRFSGSGSGTDYSLTISNLEPEDIATYYCQQYSKLPWTFGGGTRLEIK
>PDL01034_5F9-SEQ ID NO: 65
EVKLVESGGGLVQPGGSRKLSCAASGFTFSDYGMAWVRQAPGKGPEWVAFITSLAYIIFYSDTVT
GRFTISRENAKNTLYLEMSSLRSEDTAVYYCARNYGSSPYYFDYWGQGTTLTVSSGGGGSGGGGS
GGGGSGGGGSDIQMTQSPSSLSTSLGGKVTITCKASQDINKFISWYQHKPGKGPRLLIHYTSSLQ
PGIPSRFSGSGSGRDYSFSISNLEPEDIATYYCLQYDNLYTFGGGTKLEIK
>5F9_HCDR1-DYGMA (SEQ ID NO: 1)
>5F9_HCDR2-FITSLAYIIFYSDTVTG(SEQ ID NO: 66)
>5F9_HCDR3-NYGSSPYYFDY(SEQ ID NO: 3)
>5F9_LCDR1-KASQDINKFIS(SEQ ID NO: 67)
>5F9_LCDR2-YTSSLQP (SEQ ID NO: 68)
>5F9_LCDR3-LQYDNLYT(SEQ ID NO: 7)
>PDL01034_3F9_VH-SEQ ID NO: 276
DIQMTQSPSSLSTSLGGKVTITCKASQDINKFISWYQHKPGKGPRLLIHYTSSLQPGIPSRFSGS
GSGRDYSFSISNLEPEDIATYYCLQYDNLYTFGGGTKLEIK
>PDL01034_5F9_VL-SEQ ID NO: 277
EVKLVESGGGLVQPGGSRKLSCAASGFTFSDYGMAWVRQAPGKGPEWVAFITSLAYIIFYSDTVT
GRFTISRENAKNTLYLEMSSLRSEDTAVYYCARNYGSSPYYFDYWGQGTTLTVSS
>PDL01035_5F11-SEQ ID NO: 69
EVKFEESGGGLVQPGGSMKLSCFASGFTFSYYWMNWVRQSPEKGLEWIAEIRLKSNNYATHYAES
VKGRFTISRDDSKSSVYLEMNNLRAEDTGIYHCTRPPIYYGNYEAYWGQGTLVTVSAGGGGSGGG
GSGGGGSGGGGSDIQMTQSPSSLSASLGGKVTITCKASQDINKYIAWYQHKPGKGPRLLIHYTSI
LQPGIPSRFSGSGSGRDYSFSISNLEPEDIATYYCLQYDNLRTFGGGTKLEIK
>PDL01036_3F6-SEQ ID NO: 70
QGQMQQSGAELVKPGASVKLSCKTSGFTFSSSYITWLKQKPGQSLEWIAWIYVGTGGISYNQKFT
GKAQLTVDTSSSTAYMQFSSLTTEDSAIYYCARHGRYHWYFDVWGAGTTVTVSSGGGGSGGGGSG
GGGSGGGGSDIVMTQSHKFMSTSVGDRVTITCKASQDVGTAVAWYQKKPGQSPKLLISWASTRHT
GVPDRFTGSGSGTDFTLTISNVQSEDLADYFCQQYSSYPYTFGGGTNLEIK
>PDL01037_7A3-1-SEQ ID NO: 71
EVQLQESGPSLVKPSQTLSLTCSVTGDSITSGYWNWIRKFPGNRLEYMGFRSYSGTTYYNPSLKS
RISITRDTSKNQYYLQLNSVTTEDTATYYCARFSDFGSSENTMDYWGQGTSVTVSSGGGGSGGGG
SGGGGSGGGGSDIVMTQSHKFMSTSVGDRVTITCKASQDVGTAVAWYQKKPGQSPKLLISWASTR
HTGVPDRFTGSGSGTDFTLTISNVQSEDLADYFCQQYSSYPYTFGGGTNLEIK
>PDL01038_7A3-2-SEQ ID NO: 72
EVQLQESGPSLVKPSQTLSLTCSVTGDSITSGYWNWIRKFPGNRLEYMGFRSYSGTTYYNPSLKS
RISITRDTSKNQYYLQINSVTTEDTATYYCARFSDFGSSENTMDYWGQGTSVTVSSGGGGSGGGG
SGGGGSGGGGSDVQITQSPSYLAASPGFTITINCRASKSISKYLAWYQEKPGKINKLLIYSGSTL
QSGIPSRFSGSGSGTDFTLTISSLEPEDFAMYYCQQHNEYPFTFGAGTKLEIK
>PDL01039_7H11-SEQ ID NO: 73
EVQLQESGPSLVKPSQTLSLTCSVTGDSITSGYWNWIRKFPGKKLEYMGYISYTGSTYYNPSLKS
RISIARDTSKNQYFLQLNSVTTEDTATYYCTRGENWDGGYFDYWGQGTTLTVSSGGGGSGGGGSG
GGGSGGGGSDIVLTQSPASLAVSLGQRATISCRASKSVSKSGYSYLHWYQQKPGQTPKLLIYLAS
NLESGVPARFSGSGSGTDFTLNIHPVEEEDAATYYCQHSWELPYTEGGGTKLEIK
>PDL01040_10B2-SEQ ID NO: 74
KVQLQQSGAEVVKPGASVKLSCKASGYNLTEYIIHWVKQRSGQGLEWIGWFYPGSGSIKYNEKFK
DKATLTADKSSTTVYMDLSRLTSEDSAVYFCARHGLYYGFPYWGQGTSVTVSSGGGGSGGGGSGG
GGSGGGGSDIQMTQTTSSLSASLGDRVTISCRASQDIRNYLNWYQQKPDGTVKLLIYYTSRLQSG
VPSRFSGSGSGTDYSLTITTLEQEDFATYFCQQGNMFPYTFGGGTKLEIK
>PDL01060-SEQ ID NO: 75
EVQLVESGGGLVQPGGSLRLSCAASGFTFSDYGMNWVRQAPGKGPEWVASITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQHKPGKAPKLLIHYTSSLQ
PGVPSRFSGSGSGRDYTLTISSLQPEDFATYYCLQYDNLYTFGQGTKLEIKSSSEPKSSDKTHTC
PPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGN
VFSCSVMHEALHNHYTQKSLSLSPGK
>PDL01065-SEQ ID NO: 76
EVQLLFSGGGLVQPGGSLRLSCAASGFTFSDYGMSWVRQAPGKGPEWVAAITSLAYIIYYADSVK
GRFTISRDNSKNTLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQHKPGKAPKLLIHYTSSLQ
PGVPSRFSGSGSGRDYTLTISSLQPEDFATYYCLQYDNLYTFGQGTKLEIKSSSEPKSSDKTHTC
PPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGN
VFSCSVMHEALHNHYTQKSLSLSPGK
>PDL01085-SEQ ID NO: 77
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKSSSEPKSSDKTHTC
PPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGN
VFSCSVMHEALHNHYTQKSLSLSPGK
>Humanized5F9_VH_DNA-SEQ ID NO: 78
GAGGTACAACTGGTAGAATCAGGGGGGGGACTGGTACAGCCCGGGGGCTCCCTGAGACTGAGTTG
CGCCGCCTCTGGCTTTACTTTCTCCGATTACGGTATGGCATGGGTTAGACAAGCCCCCGGAAAAG
GGCTCGAATGGGTGTCATTCATCACATCACTGGCCTACATTATCTATTATGCCGACTCCGTTAAG
GGGCGGTTCACCATTAGCCGTGACAACGCAAAGAATAGTCTTTACCTGCAAATGAACTCTCTCAG
GGCAGAAGATACAGCCGTCTATTACTGTGCCAGAAACTATGGTAGTTCTCCTTATTACTTTGATT
ACTGGGGACAAGGAACTTTGGTTACCGTGAGCTCA
>Humanized5F9_VL_DNA-SEQ ID NO: 79
GATATTCAGATGACCCAAAGTCCATCATCCCTTAGTGCCTCCGTGGGGGATAGAGTAACTATCAC
ATGCCGCGCTAGTCAAGATATAAACAAATTTTTGTCCTGGTATCAGCAGAAGCCTGGTAAAGCTC
CAAAATTGCTGATCCATTATACCTCTTCCCTGCAATCTGGGGTACCTTCCCGTTTCTCTGGGTCC
GGATCAGGTACAGACTTCACTCTTACCATAAGCTCACTCCAACCAGAAGATTTCGCAACCTATTA
CTGTCTCCAGTACGATAACCTGTATACTTTTGGCCAGGGAACTAAGTTGGAGATTAAG
>CD4001003-SEQ ID NO: 80
EPPTACREKQYLINSQCCSLCQPGQKLVSDCTEFTETECLPCGESEFLDTWNRETRCHQHKYCDP
NLGLRVQQKGTSETDTICTCEEGLHCTSESCESCVPHRSCLPGFGVKQIATGVSDTICEPCPVGF
FSNVSSAFEKCRPWTSCETKDLVVQQAGTNKTDVVCGPQDRQRSSSEPRGPTIKPCPPCKCPAPN
LLGGPSVFIFPPKIKDVLMISLSPIVTCVVVDVSEDDPDVQISWFVNNVFVHTAQTQTHREDYNS
TLRVVSALPIQHQDWMSGKEFKCKVNNKDLPAPIERTISKPKGSVRAPQVYVLPPPEEEMTKKQV
TLTCMVTDFMPEDIYVEWTNNGKTELNYKNTEPVLDSDGSYFMYSKLRVEKKNWVERNSYSCSVV
HEGLHNHHTTKSFSRTPGK
>CD4001004-SEQ ID NO: 81
EPPTACREKQYLINSQCCSLCQPGQKLVSDCTEFTETECLPCGESEFLDTWNRETRCHQHKYCDP
NLGLRVQQKGTSETDTICTCEEGLHCTSESCESCVPHRSCLPGFGVKQIATGVSDTICEPCPVGF
FSNVSSAFEKCRPWTSCETKDLVVQQAGTNKTDVVCGPQDRQRHHHHHHHHHHGLNDIFEAQKIE
WHEEPEA
>CD4001005-SEQ ID NO: 82
EPPTACREKQYLINSQCCSLCQPGQKLVSDCTEFTETECLPCGESEFLDTWNRETHCHQHKYCDP
NLGLRVQQKGTSETDTICTCEEGWHCTSEACESCVLHRSCSPGFGVKQIATGVSDTICEPCPVGF
FSNVSSAFEKCHPWTSCETKDLVVQQAGTNKTDVVCGPQDRLSSSEPRGPTIKPCPPCKCPAPNL
LGGPSVFIFPPKIKDVLMISLSPIVTCVVVDVSEDDPDVQISWFVNNVFVHTAQTQTHREDYNST
LRVVSALPIQHQDWMSGKEFKCKVNNKDLPAPIERTISKPKGSVRAPQVYVLPPPEEEMTKKQVT
LTCMVTDFMPEDIYVEWTNNGKTELNYKNTEPVLDSDGSYFMYSKLRVEKKNWVERNSYSCSVVH
EGLHNHHTTKSFSRTPGK
>CD4001006-SEQ ID NO: 83
EPPTACREKQYLINSQCCSLCQPGQKLVSDCTEFTETECLPCGESEFLDTWNRETHCHQHKYCDP
NLGLRVQQKGTSETDTICTCEEGWHCTSEACESCVLHRSCSPGFGVKQIATGVSDTICEPCPVGF
FSNVSSAFEKCHPWTSCETKDLVVQQAGTNKTDVVCGPQDRLHHHHHHHHHHGLNDIFEAQKIEW
HEEPEA
>CD4001012-SEQ ID NO: 84
EPPTACREKQYLINSQCCSLCQPGQKLVSDCTEFTETECLPCGESEFLDTWNRETHCHQHKYCDP
NLGLRVQQKGTSETDTICTCEEGWHCTSEACESCVLHRSCSPGFGVKQIATGVSDTICEPCPVGF
FSNVSSAFEKCHPWTSCETKDLVVQQAGTNKTDVVCGPQDRLRALVVIPIIFGILFAILLVIVFI
KKVAKKPTNKAPHPKQEPQEINFPDDLPGSNTAAPVQETLHGCQPVTQEDGKESRISVQERQ
>CD4001013-SEQ ID NO: 85
EPPTACREKQYLINSQCCSLCQPGQKLVSDCTEFTETECLPCGESEFLDTWNRETRCHQHKYCDP
NLGLRVQQKGTSETDTICTCEEGLHCTSESCESCVPHRSCLPGFGVKQIATGVSDTICEPCPVGF
FSNVSSAFEKCRPWTSCETKDLVVQQAGTNKTDVVCGPQDRQRALVVIPICLGILFVILLLVLVF
IKKVAKKPNDKVPHPKQEPQEINFPDDLPGSNPAAPVQETLHGCQPVTQEDGKESRISVQERQ
>CD4001014-SEQ ID NO: 86
EVQLQQSGPELVKPGASMKISCKASGYSITGYTMNWVKQSHGKNLEWIGLINTYTGGTTYNQKFK
GKATLTVDKSSSTAYMELLSLTSEDSAVYYCAGTGTGAMDYWGQGTSVTVSSGGGGSGGGGSGGG
GSGGGGSQIVLTQSPAIMSSSPGFKVTMTCSASSSVSYIHWYQQKSGTSPKRWIYDTSKLASGVP
ARFSGSGSGTSYSLTINSVEAEDAATYYCQQWRTNPYTFGGGTKLEIKEPKSSDKTHTCPPCPAP
ELLGGPSVFLFPPKPKDTLMISRTPEVTCVVVDVSHEDPEVKFNWYVDGVEVHNAKIKPREEQYN
STYRVVSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVFSCSV
MHEALHNHYTQKSLSLSPGGGGSPSEVQLVESGGGLVKPGGSLRLSCAASGFTFSDYGMAWVRQA
PGKGLEWVAFITSLAYIIYYADSVKGRFTISRDNAKNSLYLQMNSLRAEDTAVYYCARNYGSSPY
YFDYWGQGTLVTVSSGGGGSGGGGSGGGGSGGGGSDIQMTQSPSSLSASVGDRVTITCRASQDIN
KFLSWYQQKPGKAPKLLIHYTSSLQPGVPSRFSGSGSGTDFTLTISSLQPEDFATYYCLQYDNLY
TFGQGTKLEIK
>CD4001015-SEQ ID NO: 88
DVQLVESGGGLVQPGGSRKLSCAASGFTFSTFGMHWVRQAPEKGLEWVAYISSGSNTIYYADTLK
GRFTISRDNPRNTLFLQMISIRSEDTAIYYCTRRPIPGSMDYWGQGTSVTVSSGGGGSGGGGSGG
GGSGGGGSQIVLTQSPALISAFPGEKVTMTCSASSSVTYMHWYQQKPRSSPKSWIYLTSNLASGV
PTRFSGSGSGTSYSLTISSMEAEDAATYFCQQWSTNSLTFGAGTKLEIKEPKSSDKTHTCPPCPA
PELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVFSCS
VMHEALHNHYTQKSLSLSPGGGGSPSEVQLVESGGGLVKPGGSLRLSCAASGFTFSDYGMAWVRQ
APGKGLEWVAFITSLAYIIYYADSVKGRFTISRDNAKNSLYLQMNSLRAEDTAVYYCARNYGSSP
YYFDYWGQGTLVTVSSGGGGSGGGGSGGGGSGGGGSDIQMTQSPSSLSASVGDRVTITCRASQDI
NKFLSWYQQKPGKAPKLLIHYTSSLQPGVPSRFSGSGSGTDFTLTISSLQPEDFATYYCLQYDNL
YTFGQGTKLEIK
>CD4001016-SEQ ID NO: 90
QVQLQQSGAELMNPGASVRISCKATGYTFSTSWIEWIKQRPGHGLEWIGEILPGSGSTNYNEKFK
GKATFTADTSSNTAYMQLSSLTFEDSAVYYCARGDDGSYVRWYFDVWGAGTTVTVSSGGGGSGGG
GSGGGGSGGGGSDIVMSQSPSSLAVSVGEKVTMSCKSSQSLLFSVNQKNYLAWYQQKPGQSPKLL
IYWASTRFSGVPDRFTGSGSGTDFTLTIGSVKAEDLAVYYCQHYYSYPWTFGGGTKLEIKEPKSS
DKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDKS
RWQQGNVFSCSVMHEALHNHYTQKSLSLSPGGGGSPSEVQLVESGGGLVKPGGSLRLSCAASGFT
FSDYGMAWVRQAPGKGLEWVAFITSLAYIIYYADSVKGRFTISRDNAKNSLYLQMNSLRAEDTAV
YYCARNYGSSPYYFDYWGQGTLVTVSSGGGGSGGGGSGGGGSGGGGSDIQMTQSPSSLSASVGDR
VTITCRASQDINKFLSWYQQKPGKAPKLLIHYTSSLQPGVPSRFSGSGSGTDFTLTISSLQPEDF
ATYYCLQYDNLYTFGQGTKLEIK
>CD4001017-SEQ ID NO: 92
DVQLVESGGGLVQPGGSRKLSCAASGFTFSTFGMHWVRQAPEKGLEWVAYISSGSNTIYYADTLK
GRFTISRDNPRNTLFLQMTSLRSEDTAIYYCTRRPIPGAMDYWGQGTSVTVSSGGGGSGGGGSGG
GGSGGGGSQIVLTQSPALISAFPGEKVTMTCSASSSVTYMHWYQQKPRSSPKSWIYLTSNLASGV
PSRFSGSGSGTSYSLTISSMEAEDAATYFCQQWSTNSLTFGAGTKLEIKEPKSSDKTHTCPPCPA
PELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVFSCS
VMHEALHNHYTQKSLSLSPGGGGSPSEVQLVESGGGLVKPGGSLRLSCAASGFTFSDYGMAWVRQ
APGKGLEWVAFITSLAYIIYYADSVKGRFTISRDNAKNSLYLQMNSLRAEDTAVYYCARNYGSSP
YYFDYWGQGTLVTVSSGGGGSGGGGSGGGGSGGGGSDIQMTQSPSSLSASVGDRVTITCRASQDI
NKFLSWYQQKPGKAPKLLIHYTSSLQPGVPSRFSGSGSGTDFTLTISSLQPEDFATYYCLQYDNL
YTFGQGTKLEIK
>CD4001018-SEQ ID NO: 94
EVMLVESGGGLVKPGGSLKLSCAASGFTFSSYAMSWVRQTPERRLEWVASISSAGTYIYYPDSVK
GRFTISRDNAKNILYLQMNSLRSEDTAMYYCARHGNGYDPLWYFDVWGAGTTVTVSSGGGGSGGG
GSGGGGSGGGGSDVVMTQTPLSLPVSLGDQASISCRSSQSLVHYNGYTYLHWYLQKPGQSPKLLI
YKVSNRFSGVPDRFSGSGSGTDFTLKISRVEAEDLGVYFCSQTTHVPLTFGAGTKLELREPKSSD
KTHTCPPCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSR
WQQGNVFSCSVMHEALHNHYTQKSLSLSPGGGGSPSEVQLVESGGGLVKPGGSLRLSCAASGFTF
SDYGMAWVRQAPGKGLEWVAFITSLAYIIYYADSVKGRFTISRDNAKNSLYLQMNSLRAEDTAVY
YCARNYGSSPYYFDYWGQGTLVTVSSGGGGSGGGGSGGGGSGGGGSDIQMTQSPSSLSASVGDRV
TITCRASQDINKFLSWYQQKPGKAPKLLIHYTSSLQPGVPSRFSGSGSGTDFTLTISSLQPEDFA
TYYCLQYDNLYTFGQGTKLEIK
>CD4001019-SEQ ID NO: 96
DVQLVESGGGLVQPGGSRKLSCAASGFAFSSFGIHWVRQSPEKGLEWVAYISGGSSTIYYADSLK
GRFTISRDNPKNTLFLQMTSLRSEDTAIYYCVRRPIPGAMDYWGQGISVTVSSGGGGSGGGGSGG
GGSGGGGSQIVLTQSPALMSASPGFKVTMTCRASSRVSYIHWYQQKPRSSPKSWIYLTSNLASGV
PSRFSGSGSGTSYSLTISSMEAEDAATYYCQQWSSNSLTFGAGTKLELKEPKSSDKTHTCPPCPA
PELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVFSCS
VMHEALHNHYTQKSLSLSPGGGGSPSEVQLVESGGGLVKPGGSLRLSCAASGFTFSDYGMAWVRQ
APGKGLEWVAFITSLAYIIYYADSVKGRFTISRDNAKNSLYLQMNSLRAEDTAVYYCARNYGSSP
YYFDYWGQGTLVTVSSGGGGSGGGGSGGGGSGGGGSDIQMTQSPSSLSASVGDRVTITCRASQDI
NKFLSWYQQKPGKAPKLLIHYTSSLQPGVPSRFSGSGSGTDFTLTISSLQPEDFATYYCLQYDNL
YTFGQGTKLEIK
>CD4001020-SEQ ID NO: 98
DVQLVESGGGLVQPGGSRKLSCAASGFTFSSFGMHWVRQAPEKGLEWVAYISSGSITIYYADTLK
GRFTISRDNPKNTLFLQMTSLRSEDTAMYYCTRRPIPGAMDYWGQGTSVTVSSGGGGSGGGGSGG
GGSGGGGSQIVLTQSPALMSASPGFKVTMTCSASSSVTYMHWYQQKPRSSPKSWIYLTSNLASGV
PARFSGSGSGTSYSLTISSMEAEDAATYYCQQWSSDSLTFGAGTKLELKEPKSSDKTHTCPPCPA
PELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVFSCS
VMHEALHNHYTQKSLSLSPGGGGSPSEVQLVESGGGLVKPGGSLRLSCAASGFTFSDYGMAWVRQ
APGKGLEWVAFITSLAYIIYYADSVKGRFTISRDNAKNSLYLQMNSLRAEDTAVYYCARNYGSSP
YYFDYWGQGTLVTVSSGGGGSGGGGSGGGGSGGGGSDIQMTQSPSSLSASVGDRVTITCRASQDI
NKFLSWYQQKPGKAPKLLIHYTSSLQPGVPSRFSGSGSGTDFTLTISSLQPEDFATYYCLQYDNL
YTFGQGTKLEIK
>CD4001021-SEQ ID NO: 100
EVLLQQSGPELVKPGASVKISCKASGYKFNDYNIDWVKQSQGKSLEWIGNINPDNGGTIYNQKFK
GKATLTVDKSSSTPYMELRSLTSEDTAVYFCARERDNRYDRWSAYWGQGTLVTVSAGGGGSGGGG
SGGGGSGGGGSDIVMTQSQKFMSTSVGDRVSVTCKASQNVGANVAWYQQKPGQSPKALIYSASYR
YSGVPDRFTGSGSGTDFTLTISNVHSDDLAEYFCQQYNTYPFTFGSGTKLEIKEPKSSDKTHTCP
PCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRWQQGNV
FSCSVMHEALHNHYTQKSLSLSPGGGGSPSEVQLVESGGGLVKPGGSLRLSCAASGFTFSDYGMA
WVRQAPGKGLEWVAFITSLAYIIYYADSVKGRFTISRDNAKNSLYLQMNSLRAEDTAVYYCARNY
GSSPYYFDYWGQGTLVTVSSGGGGSGGGGSGGGGSGGGGSDIQMTQSPSSLSASVGDRVTITCRA
SQDINKFLSWYQQKPGKAPKLLIHYTSSLQPGVPSRFSGSGSGTDFTLTISSLQPEDFATYYCLQ
YDNLYTFGQGTKLEIK
>CD4001022-SEQ ID NO: 102
EVLLQQSGPEVVKPGASVKISCKASGYKFNDYNIDWVKQSHGKSLDWIGNINPNNGGTIYNQKFK
GKATLTVDKSSSTPYMDLRSLTSEDTAVYFCSRERDNRYDRWSAYWGQGTLVTVSAGGGGSGGGG
SGGGGSGGGGSDIVMTQSQKFMSTSVGDRVSVTCKASQNVGANVAWYQHKPGQSPKALIYSASYW
NSGVPDRFTGSGSGTDFTLTISSVHSDDLAEYFCQQYNSYPFAFGSGTKLEIKEPKSSDKTHTCP
PCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRWQQGNV
FSCSVMHEALHNHYTQKSLSLSPGGGGSPSEVQLVESGGGLVKPGGSLRLSCAASGFTFSDYGMA
WVRQAPGKGLEWVAFITSLAYIIYYADSVKGRFTISRDNAKNSLYLQMNSLRAEDTAVYYCARNY
GSSPYYFDYWGQGTLVTVSSGGGGSGGGGSGGGGSGGGGSDIQMTQSPSSLSASVGDRVTITCRA
SQDINKFLSWYQQKPGKAPKLLIHYTSSLQPGVPSRFSGSGSGTDFTLTISSLQPEDFATYYCLQ
YDNLYTFGQGTKLEIK
>CD4001023-SEQ ID NO: 104
EVQLQQSGPELVKPGASMKISCKASGYSFTGYTMNWVKQSHGKNLEWIGLVNVYHGGTTYNQKFK
GKATLTVDKSSSTAYMELLSLTSEDSAVYYCAGTGTGALDYWGQGTSVTVSSGGGGSGGGGSGGG
GSGGGGSQIVLTQSPAIMSASPGFKVTMTCSASSSVSYMHWYQQKSGPSPKRWIYDTSKLASGVP
ARFSGSGSGTSYSLTISSMEAEDAATYYCQQWRSNPYTFGGGTKLEIKEPKSSDKTHTCPPCPAP
ELLGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVFSCSV
MHEALHNHYTQKSLSLSPGGGGSPSEVQLVESGGGLVKPGGSLRLSCAASGFTFSDYGMAWVRQA
PGKGLEWVAFITSLAYIIYYADSVKGRFTISRDNAKNSLYLQMNSLRAEDTAVYYCARNYGSSPY
YFDYWGQGTLVTVSSGGGGSGGGGSGGGGSGGGGSDIQMTQSPSSLSASVGDRVTITCRASQDIN
KFLSWYQQKPGKAPKLLIHYTSSLQPGVPSRFSGSGSGTDFTLTISSLQPEDFATYYCLQYDNLY
TFGQGTKLEIK
>CD4001024-SEQ ID NO: 106
DVQLVESGGGLVQPGGSRKLSCAASGFTFSTFGMHWVRQAPEKGLEWVAYISSGSNTIYYADTLK
GRFTISRDNPRNTLFLQMTSLRSEDTAIYYCTRRPIPGAMDYWGQGTSVTVSSGGGGSGGGGSGG
GGSGGGGSQIVLTQSPALISAFPGERVTMTCSASSSVTYIHWYQQKPRSSPKSWIYLTSNLASGV
PTRFSGSGSGTSYSLTISSMEAEDAATYFCQQWSTNSLTFGAGTKLEIKEPKSSDKTHTCPPCPA
PELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVFSCS
VMHEALHNHYTQKSLSLSPGGGGSPSEVQLVESGGGLVKPGGSLRLSCAASGFTFSDYGMAWVRQ
APGKGLEWVAFITSLAYIIYYADSVKGRFTISRDNAKNSLYLQMNSLRAEDTAVYYCARNYGSSP
YYFDYWGQGTLVTVSSGGGGSGGGGSGGGGSGGGGSDIQMTQSPSSLSASVGDRVTITCRASQDI
NKFLSWYQQKPGKAPKLLIHYTSSLQPGVPSRFSGSGSGTDFTLTISSLQPEDFATYYCLQYDNL
YTFGQGTKLEIK
>CD4001036-SEQ ID NO: 108
QVQLQQSGAELMKPGASVKIPCKATGYTFSSYWIEWVKQRPGHGLEWIGEVLPGSGSTNYNEKFK
GKATFTADTSSNTAYMQLSSLTSEDSAVYYCARPYYRYDVGAWFAYWGQGTLVTVSAGGGGSGGG
GSGGGGSGGGGSDIVMSQSPSSLAVSVGEKVTMSCKSSQSLLYSGNQKNYLAWYQQKPGQSPKLL
IYWASTRFSGVPDRFTGSGSGTDFTLTISSVKAEDLAVYYCQQYYSYPYTFGGGTKLEIKEPKSS
DKTHTCPPCPAPELLGGPSVFLFPPKPKDTLMISRIPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDKS
RWQQGNVFSCSVMHEALHNHYTQKSLSLSPGGGGSPSEVQLVESGGGLVKPGGSLRLSCAASGFT
FSDYGMAWVRQAPGKGLEWVAFITSLAYIIYYADSVKGRFTISRDNAKNSLYLQMNSLRAEDTAV
YYCARNYGSSPYYFDYWGQGTLVTVSSGGGGSGGGGSGGGGSGGGGSDIQMTQSPSSLSASVGDR
VTITCRASQDINKFLSWYQQKPGKAPKLLIHYTSSLQPGVPSRFSGSGSGTDFTLTISSLQPEDF
ATYYCLQYNDLYTFGQGTKLEIK
>CD4001038-SEQ ID NO: 110
QVQLQQSGAELAKPGASVKMSCKASGYAFTTYWMHWVKQRPGQGLEWIGYINPTTGYTDYNQKFK
DKATLTADKSSSTAYMQLSSLTSEDSAVYYCARWGLPPFAYWGQGTLVTVSAGGGGSGGGGSGGG
GSGGGGSDIVMTQSHKFMSTSVGDRVSITCKASQDVNTAVAWYQQKPGQSPKLLIYSASYRYTGV
PDRFTGSGSGTDFTFTISSVQAEDLAVYYCQQHYSTPWTFGGGTKLEIKEPKSSDKTHTCPPCPA
PELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVFSCS
VMHEALHNHYTQKSLSLSPGGGGSPSEVQLVESGGGLVKPGGSLRLSCAASGFTFSDYGMAWVRQ
APGKGLEWVAFITSLAYIIYYADSVKGRFTISRDNAKNSLYLQMNSLRAEDTAVYYCARNYGSSP
YYFDYWGQGTLVTVSSGGGGSGGGGSGGGGSGGGGSDIQMTQSPSSLSASVGDRVTITCRASQDI
NKFLSWYQQKPGKAPKLLIHYTSSLQPGVPSRFSGSGSGTDFTLTISSLQPEDFATYYCLQYDNL
YTFGQGTKLEIK
>CD4001040-SEQ ID NO: 112
QVQLKESGPGLVAPSQSLSITCTVSGFSLSRYSIQWVRQPPGKGLEWLGMIWGGGSTDYNSALKS
RLSISKDNSKSQVFLKMNSLQTDDTAMYYCARNQGGYDVWFAYWGQGTLVTVSAGGGGSGGGGSG
GGGSGGGGSDIVMSQSPSSLAVSVGEKVTMNCKSSQSLLYSGNQKKYLAWYQQKPGQSPKLLIYW
ASTRFSGVPDRFTGSGSGTDFTLTISSVKAEDLAIYYCQQYYSYPFTFGGGTKLEIKEPKSSDKT
HTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGGGGSPSEVQLVESGGGLVKPGGSLRISCAASGFTFSD
YGMAWVRQAPGKGLEWVAFITSLAYIIYYADSVKGRFTISRDNAKNSLYLQMNSLRAEDTAVYYC
ARNYGSSPYYFDYWGQGTLVTVSSGGGGSGGGGSGGGGSGGGGSDIQMTQSPSSLSASVGDRVTI
TCRASQDINKFLSWYQQKPGKAPKLLIHYTSSLQPGVPSRFSGSGSGTDFTLTISSLQPEDFATY
YCLQYDNLYTFGQGTKLEIK
>CD4001042-SEQ ID NO: 114
QVQLQQSGTELMKPGAAVKISCKATGYTISSYWIEWVKQRPGHGLEWIGEILPGSGSINYNEKFK
GKATFTADTSSNTAYIQLSSLTSEDSAVYYCARGGIYYGPLYAMDYWGQGISVTVSSGGGGSGGG
GSGGGGSGGGGSDIVMSQSPSSLAVSVGEKVTMSCKSSQSLLYSGNQKNYLAWYQQKPGQSPKLL
IYWASTRFSGVPDRFTGSGSGTDFTLTISSVKAEDLAVYYCQQYYSYPYTFGGGTKLEIKEPKSS
DKTHTCPPCPAPELLGGPSVFLFPPKPKDTLMISRIPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDKS
RWQQGNVFSCSVMHEALHNHYTQKSLSLSPGGGGSPSEVQLVESGGGLVKPGGSLRLSCAASGFT
FSDYGMAWVRQAPGKGLEWVAFITSLAYIIYYADSVKGRFTISRDNAKNSLYLQMNSLRAEDTAV
YYCARNYGSSPYYFDYWGQGTLVTVSSGGGGSGGGGSGGGGSGGGGSDIQMTQSPSSLSASVGDR
VTITCRASQDINKFLSWYQQKPGKAPKLLIHYTSSLQPGVPSRFSGSGSGTDFTLTISSLQPEDF
ATYYCLQYDNLYTFGQGTKLEIK
>CD4001044-SEQ ID NO: 116
EVKLVESGGGLVQPGGALRLSCATSGFTFTDYYMSWVRQPPGKALEWLGFIRNKANGYTTEYSAS
VKGRFTISRDNSQSILYLQMNTLRAEDSATYYCASHYYGRAWFAYWGQGTLVTVSAGGGGSGGGG
SGGGGSGGGGSQIVLTQSPTIMSASPGFKVTMTCSASSSVSNMHWYQQKSGTSPKRWIYDTSTLA
SGVPARFSGSGSGTSYSLTISSMEAEDVATYYCQQWSSNPPTFGGGTTLEIKEPKSSDKTHTCPP
CPAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQQGNVF
SCSVMHEALHNHYTQKSLSLSPGGGGSPSEVQLVESGGGLVKPGGSLRLSCAASGFTFSDYGMAW
VRQAPGKGLEWVAFITSLAYIIYYADSVKGRFTISRDNAKNSLYLQMNSLRAEDTAVYYCARNYG
SSPYYFDYWGQGTLVTVSSGGGGSGGGGSGGGGSGGGGSDIQMTQSPSSLSASVGDRVTITCRAS
QDINKFLSWYQQKPGKAPKLLIHYTSSLQPGVPSRFSGSGSGTDFTLTISSLQPEDFATYYCLQY
DNLYTFGQGTKLEIK
>CD4001027-SEQ ID NO: 118
DIVMSQSPSSLAVSVGEKVTMSCKSSQSLLFSVNQKNYLAWYQQKPGQSPKLLIYWASTRFSGVP
DRFTGSGSGTDFTLTIGSVKAEDLAVYYCQHYYSYPWTFGGGTKLEIKGGGGSGGGGSGGGGSGG
GGSQVQLQQSGAELMNPGASVRISCKATGYTFSTSWIEWIKQRPGHGLEWIGEILPGSGSTNYNE
KFKGKATFTADTSSNTAYMQLSSLTFEDSAVYYCARGDDGSYVRWYFDVWGAGTTVTVSSEPKSS
DKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDKS
RWQQGNVFSCSVMHEALHNHYTQKSLSLSPGGGGSPSEVQLVESGGGLVKPGGSLRLSCAASGFT
FSDYGMAWVRQAPGKGLEWVAFITSLAYIIYYADSVKGRFTISRDNAKNSLYLQMNSLRAEDTAV
YYCARNYGSSPYYFDYWGQGTLVTVSSGGGGSGGGGSGGGGSGGGGSDIQMTQSPSSLSASVGDR
VTITCRASQDINKFLSWYQQKPGKAPKLLIHYTSSLQPGVPSRFSGSGSGTDFTLTISSLQPEDF
ATYYCLQYDNLYTFGQGTKLEIK
>CD4001052-SEQ ID NO: 119
DYKDDDDKDYKDDDDKGGSGGEPPTACREKQYLINSQCCSLCQPGQKLVSDCTEFTETECLPCGE
SEFLDTWNRETHCHQHKYCDPNLGLRVQQKGTSETDTICTCEEGWHCTSEACESCVLHRSCSPGF
GVKQIATGVSDTICEPCPVGFFSNVSSAFEKCHPWTSCETKDLVVQQAGTNKTDVVCGPQDRLRA
LVVIPIIFGILFAILLVLVFIKKVAKKPTNKAPHPKQEPQEINFPDDLPGSNTAAPVQETLHGCQ
PVTQEDGKESRISVQERQ
>CD4001053-SEQ ID NO: 120
DYKDDDDKDYKDDDDKGGSGGEPPTACSDKQYLHDGQCCDLCQPGSRLTSHCTALEKTQCHPCDS
GEFSAQWNREIRCHQHKYCDPNLGLRVQQKGTSETDTICTCEEGWHCTSEACESCVLHRSCSPGF
GVKQIATGVSDTICEPCPVGFFSNVSSAFEKCHPWTSCETKDLVVQQAGTNKTDVVCGPQDRLRA
LVVIPIIFGILFAILLVLVFIKKVAKKPTNKAPHPKQEPQEINFPDDLPGSNTAAPVQETLHGCQ
PVTQEDGKESRISVQERQ
>CD4001054-SEQ ID NO: 121
DYKDDDDKDYKDDDDKGGSGGEPPTACREKQYLINSQCCSLCQPGQKLVSDCTEFTETECLPCGE
SEFLDTWNRETHCHQHRHCEPNQGLRVKKEGTAESDTVCTCKEGQHCTSKDCEACAQHRSCSPGF
GVKQIATGVSDTICEPCPVGFFSNVSSAFEKCHPWTSCETKDLVVQQAGTNKTDVVCGPQDRLRA
LVVIPIIFGILFAILLVLVFIKKVAKKPTNKAPHPKQEPQEINFPDDLPGSNTAAPVQETLHGCQ
PVTQEDGKESRISVQERQ
>CD4001055-SEQ ID NO: 122
DYKDDDDKDYKDDDDKGGSGGEPPTACREKQYLINSQCCSLCQPGQKLVSDCTEFTETECLPCGE
SEFLDTWNRETHCHQHKYCDPNLGLRVQQKGTSETDTICTCEEGWHCTSEACESCVLHTPCIPGF
GVMEMATETTDTVCHPCPVGFFSNQSSLFEKCYPWTSCEDKNLEVLQKGTSQTNVICGLKSRMRA
LVVIPIIFGILFAILLVLVFIKKVAKKPTNKAPHPKQEPQEINFPDDLPGSNTAAPVQETLHGCQ
PVTQEDGKESRISVQERQ
>CD4001056-SEQ ID NO: 123
DYKDDDDKDYKDDDDKGGSGGEPPTACSDKQYLHDGQCCDLCQPGSRLTSHCTALEKTQCHPCDS
GEFSAQWNREIRCHQHRHCEPNQGLRVKKEGTAESDTVCTCKEGQHCTSKDCEACAQHTPCIPGF
GVMEMATETTDTVCHPCPVGFFSNQSSLFEKCYPWTSCEDKNLEVLQKGTSQTNVICGLKSRMRA
LVVIPIIFGILFAILLVLVFIKKVAKKPTNKAPHPKQEPQEINFPDDLPGSNTAAPVQETLHGCQ
PVTQEDGKESRISVQERQ
>CD4001238-SEQ ID NO: 124
DYKDDDDKDYKDDDDKGGSGGEPPTACREKQYLINSQCCSLCQPGQKLVSDCTEFTETECLPCGE
SEFLDTWNRETHCHQHKYCDPNLGLRVQQKGTSETDTICTCKEGQHCTSKDCEACAQHRSCSPGF
GVKQIATETTDTICEPCPVGFFSNVSSAFEKCHPWTSCETKDLVVQQAGTNKTDVVCGPQDRLRA
LVVIPIIFGILFAILLVLVFIKKVAKKPTNKAPHPKQEPQEINFPDDLPGSNTAAPVQETLHGCQ
PVTQEDGKESRISVQERQ
>CD4001239-SEQ ID NO: 125
DYKDDDDKDYKDDDDKGGSGGEPPTACREKQYLINSQCCSLCQPGQKLVSDCTEFTETECLPCGE
SEFLDTWNRETHCHQHKYCDPNLGLRVQQKGTSETDTICTCEEGWHCTSEACESCVLHTPCIPGF
GVKQIATGVSDTICEPCPVGFFSNQSSLFEKCHPWTSCETKDLVVQQAGTSQTDVVCGPQDRLRA
LVVIPIIFGILFAILLVLVFIKKVAKKPTNKAPHPKQEPQEINFPDDLPGSNTAAPVQETLHGCQ
PVTQEDGKESRISVQERQ
>CD4001240-SEQ ID NO: 126
DYKDDDDKDYKDDDDKGGSGGEPPTACREKQYLINSQCCSLCQPGQKLVSDCTEFTETECLPCGE
SEFLDTWNRETHCHQHKYCDPNLGLRVQQKGTSETDTICTCEEGWHCTSEACESCVLHRSCSPGF
GVMEMATETTDTVCHPCPVGFFSNVSSAFEKCYPWTSCETKDLVVQQAGTNKTDVVCGPQDRLRA
LVVIPIIFGILFAILLVLVFIKKVAKKPTNKAPHPKQEPQEINEPDDLPGSNTAAPVQETLHGCQ
PVTQEDGKESRISVQERQ
>CD4001241-SEQ ID NO: 127
DYKDDDDKDYKDDDDKGGSGGEPPTACREKQYLINSQCCSLCQPGQKLVSDCTEFTETECLPCGE
SEFLDTWNRETHCHQHKYCDPNLGLRVQQKGTSETDTICTCEEGWHCTSEACESCVLHRSCSPGF
GVKQIATGVSDTICEPCPVGFFSNVSSAFEKCYPWTSCEDKNLEVLQKGTSQINVICGLKSRMRA
LVVIPIIFGILFAILLVLVFIKKVAKKPTNKAPHPKQEPQEINFPDDLPGSNTAAPVQETLHGCQ
PVTQEDGKESRISVQERQ
>CD4001047-SEQ ID NO: 128
DIVMTQSHKFMSTSVGDRVSITCKASQDVNTAVAWYQQKPGQSPKLLIYSASYRYTGVPDRFTGS
GSGTDFTFTISSVQAEDLAVYYCQQHYSTPWTFGGGTKLEIKGGGGSGGGGSGGGGSGGGGSQVQ
LQQSGAELAKPGASVKMSCKASGYAFTTYWMHWVKQRPGQGLEWIGYINPTTGYTDYNQKFKDKA
TLTADKSSSTAYMQLSSLISEDSAVYYCARWGLPPFAYWGQGTLVTVSAEPKSSDKTHTCPPCPA
PPAAAPSVFLFPPKPKDTLMISRTPEVTCVVVDVSHEDPEVKFNWYVDGVEVHNAKTKPREEQYN
STYRVVSVLTVLHQDWLNGKEYKCAVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVFSCSV
MHEALHNHYTQKSLSLSPGGGGSPSEVQLVESGGGLVKPGGSLRLSCAASGFTFSDYGMAWVRQA
PGKGLEWVAFITSLAYIIYYADSVKGRFTISRDNAKNSLYLQMNSLRAEDTAVYYCARNYGSSPY
YFDYWGQGTLVTVSSGGGGSGGGGSGGGGSGGGGSDIQMTQSPSSLSASVGDRVTITCRASQDIN
KFLSWYQQKPGKAPKLLIHYTSSLQPGVPSRFSGSGSGTDFTLTISSLQPEDFATYYCLQYDNLY
TFGQGTKLEIK
>CD4001048-SEQ ID NO: 129
DVVMTQTPLSLPVSLGDQASISCRSSQSLVHYNGYTYLHWYLQKPGQSPKLLIYKVSNRFSGVPD
RFSGSGSGTDFTLKISRVEAEDLGVYFCSQTTHVPLTFGAGTKLELRGGGGSGGGGSGGGGSGGG
GSEVMLVESGGGLVKPGGSLKLSCAASGFTFSSYAMSWVRQTPERRLEWVASISSAGTYIYYPDS
VKGRFTISRDNAKNILYLQMNSLRSEDTAMYYCARHGNGYDPLWYFDVWGAGTTVTVSSEPKSSD
KTHTCPPCPAPPAAAPSVFLFPPKPKDTLMISRTPEVTCVVVDVSHEDPEVKFNWYVDGVEVINA
KTKPREEQYNSTYRVVSVLTVLHQDWLNGKEYKCAVSNKALPAPIEKTISKAKGQPREPQVYTLP
PSRDELTKNQVSLTCLVKGFYPSDIAVEWESNGQPENNYKITPPVLDSDGSFFLYSKLTVDKSRW
QQGNVFSCSVMHEALHNHYTQKSLSLSPGGGGSPSEVQLVESGGGLVKPGGSLRISCAASGFTFS
DYGMAWVRQAPGKGLEWVAFITSLAYIIYYADSVKGRFTISRDNAKNSLYLQMNSLRAEDTAVYY
CARNYGSSPYYFDYWGQGTLVTVSSGGGGSGGGGSGGGGSGGGGSDIQMTQSPSSLSASVGDRVT
ITCRASQDINKFLSWYQQKPGKAPKLLIHYTSSLQPGVPSRFSGSGSGTDFTLTISSLQPEDFAT
YYCLQYDNLYTFGQGTKLEIK
>CD4001049-SEQ ID NO: 130
DIVMSQSPSSLAVSVGEKVTMSCKSSQSLLFSVNQKNYLAWYQQKPGQSPKLLIYWASTRFSGVP
DRFTGSGSGTDFTLTIGSVKAEDLAVYYCQHYYSYPWTFGGGTKLEIKGGGGSGGGGSGGGGSGG
GGSQVQLQQSGAELMNPGASVRISCKATGYTFSTSWIEWIKQRPGHGLEWIGEILPGSGSTNYNE
KFKGKATFTADTSSNTAYMQLSSLTFEDSAVYYCARGDDGSYVRWYFDVWGAGTTVTVSSEPKSS
DKTHTCPPCPAPPAAAPSVFLFPPKPKDTLMISRTPEVTCVVVDVSHEDPEVKFNWYVDGVEVHN
AKTKPREEQYNSTYRVVSVLTVLHQDWLNGKEYKCAVSNKALPAPIEKTISKAKGQPREPQVYTL
PPSRDELTKNQVSLTCLVKGFYPSDIAVEWESNGQPENNYKTTPPVLDSDGSFFLYSKLTVDKSR
WQQGNVFSCSVMHEALHNHYTQKSLSLSPGGGGSPSEVQLVESGGGLVKPGGSLRLSCAASGFTF
SDYGMAWVRQAPGKGLEWVAFITSLAYIIYYADSVKGRFTISRDNAKNSLYLQMNSLRAEDTAVY
YCARNYGSSPYYFDYWGQGTLVTVSSGGGGSGGGGSGGGGSGGGGSDIQMTQSPSSLSASVGDRV
TITCRASQDINKFLSWYQQKPGKAPKLLIHYTSSLQPGVPSRFSGSGSGTDFTLTISSLQPEDFA
TYYCLQYDNLYTFGQGTKLEIK
>CD4001064-SEQ ID NO: 131
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDELMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSC
SVMHEALHNHYTQKSLSLSPGGGGSPSDIVMTQSHKFMSTSVGDRVSITCKASQDVNTAVAWYQQ
KPGQSPKLLIYSASYRYTGVPDRFTGSGSGTDFTFTISSVQAEDLAVYYCQQHYSTPWTFGGGTK
LEIKGGGGSGGGGSGGGGSGGGGSQVQLQQSGAELAKPGASVKMSCKASGYAFTTYWMHWVKQRP
GQGLEWIGYINPTTGYTDYNQKFKDKATLTADKSSSTAYMQLSSLTSEDSAVYYCARWGLPPFAY
WGQGTLVTVSA
>CD4001065-SEQ ID NO: 132
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPSRDELTK
NQVSLTCLVKGFYPSDIAVEWESNGQPENNYKTTPPVLDSDGSFFLYSKLIVDKSRWQQGNVFSC
SVMHEALHNHYTQKSLSLSPGGGGSPSDVVMTQTPLSLPVSLGDQASISCRSSQSLVHYNGYTYL
HWYLQKPGQSPKLLIYKVSNRFSGVPDRFSGSGSGTDFTLKISRVEAEDLGVYFCSQTTHVPLTF
GAGTKLELRGGGGSGGGGSGGGGSGGGGSEVMLVESGGGLVKPGGSLKLSCAASGFTFSSYAMSW
VRQTPERRLEWVASISSAGTYIYYPDSVKGRFTISRDNAKNILYLQMNSLRSEDTAMYYCARHGN
GYDPLWYFDVWGAGTTVTVSS
>CD4001066-SEQ ID NO: 133
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSC
SVMHEALHNHYTQKSLSLSPGGGGSPSDIVMSQSPSSLAVSVGEKVTMSCKSSQSLLFSVNQKNY
LAWYQQKPGQSPKLLIYWASTRFSGVPDRFTGSGSGTDFTLTIGSVKAEDLAVYYCQHYYSYPWT
FGGGTKLEIKGGGGSGGGGSGGGGSGGGGSQVQLQQSGAELMNPGASVRISCKATGYTFSTSWIE
WIKQRPGHGLEWIGEILPGSGSTNYNEKFKGKATFTADTSSNTAYMQLSSLTFEDSAVYYCARGD
DGSYVRWYFDVWGAGTTVTVSS
>CD4001067_Chain1-SEQ ID NO: 134
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPSRDELTK
NQVSLWCLVKGFYPSDIAVEWESNGQPENNYKTTPPVLDSDGSFFLYSKLTVDKSRWQQGNVFSC
SVMHEALHNHYTQKSLSLSPG
>CD4001067_Chain2-SEQ ID NO: 135
DIVMTQSHKFMSTSVGDRVSITCKASQDVNTAVAWYQQKPGQSPKLLIYSASYRYTGVPDRFTGS
GSGTDFTFTISSVQAEDLAVYYCQQHYSTPWTFGGGTKLEIKGGGGSGGGGSGGGGSGGGGSQVQ
LQQSGAELAKPGASVKMSCKASGYAFTTYWMHWVKQRPGQGLEWIGYINPTTGYTDYNQKFKDKA
TLTADKSSSTAYMQLSSLTSEDSAVYYCARWGLPPFAYWGQGTLVTVSAEPKSSDKTHTCPPCPA
PPAAAPSVFLFPPKPKDTLMISRTPEVTCVVVDVSHEDPEVKFNWYVDGVEVHNAKTKPREEQYN
STYRVVSVLTVLHQDWLNGKEYKCAVSNKALPAPIEKTISKAKGQPREPQVYTLPPSRDELTKNQ
VSLSCAVKGFYPSDIAVEWESNGQPENNYKTTPPVLDSDGSFFLVSKLTVDKSRWQQGNVFSCSV
MHEALHNRFTQKSLSLSPG
>CD4001068_Chain1-SEQ ID NO: 136
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGGGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPSRDELTK
NQVSLWCLVKGFYPSDIAVEWESNGQPENNYKTTPPVLDSDGSFFLYSKLTVDKSRWQQGNVFSC
SVMHEALHNHYTQKSLSLSPG
>CD4001068_Chain2-SEQ ID NO: 137
DVVMTQTPLSLPVSLGDQASISCRSSQSLVHYNGYTYLHWYLQKPGQSPKLLIYKVSNRFSGVPD
RFSGSGSGTDFTLKISRVEAEDLGVYFCSQTTHVPLIEGAGTKLELRGGGGSGGGGSGGGGSGGG
GSEVMLVESGGGLVKPGGSLKLSCAASGFTFSSYAMSWVRQTPERRLEWVASISSAGTYIYYPDS
VKGRFTISRDNAKNILYLQMNSLRSEDTAMYYCARHGNGYDPLWYFDVWGAGTTVTVSSEPKSSD
KTHTCPPCPAPPAAAPSVFLFPPKPKDTLMISRTPEVTCVVVDVSHEDPEVKFNWYVDGVEVHNA
KTKPREEQYNSTYRVVSVLTVLHQDWLNGKEYKCAVSNKALPAPIEKTISKAKGQPREPQVYTLP
PSRDELTKNQVSLSCAVKGFYPSDIAVEWESNGQPENNYKTTPPVLDSDGSFFLVSKLTVDKSRW
QQGNVFSCSVMHEALHNRETQKSLSLSPG
>CD4001069_Chain1-SEQ ID NO: 138
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPSRDELTK
NQVSLWCLVKGFYPSDIAVEWESNGQPENNYKTTPPVLDSDGSFFLYSKLTVDKSRWQQGNVFSC
SVMHEALHNHYTQKSLSLSPG
>CD4001069_Chain2-SEQ ID NO: 139
DIVMSQSPSSLAVSVGEKVTMSCKSSQSLLFSVNQKNYLAWYQQKPGQSPKLLIYWASTRFSGVP
DRFTGSGSGTDFTLTIGSVKAEDLAVYYCQHYYSYPWTFGGGTKLEIKGGGGSGGGGSGGGGSGG
GGSQVQLQQSGAELMNPGASVRISCKATGYTFSTSWIEWIKQRPGHGLEWIGEILPGSGSTNYNE
KFKGKATFTADTSSNTAYMQLSSLTFEDSAVYYCARGDDGSYVRWYFDVWGAGTTVTVSSEPKSS
DKTHTCPPCPAPPAAAPSVFLFPPKPKDTLMISRTPEVTCVVVDVSHEDPEVKFNWYVDGVEVHN
AKTKPREEQYNSTYRVVSVLTVLHQDWLNGKEYKCAVSNKALPAPIEKTISKAKGQPREPQVYTL
PPSRDELTKNQVSLSCAVKGFYPSDIAVEWESNGQPENNYKTTPPVLDSDGSFFLVSKLTVDKSR
WQQGNVFSCSVMHEALHNRFTQKSLSLSPG
>CD4001070_Chain1-SEQ ID NO: 140
EPKSSDKTHTCPPCPAPPAAAPSVFLFPPKPKDTLMISRTPEVTCVVVDVSHEDPEVKFNWYVDG
VEVHNAKTKPREEQYNSTYRVVSVLTVLHQDWLNGKEYKCAVSNKALPAPIEKTISKAKGQPREP
QVYTLPPSRDELTKNQVSLWCLVKGFYPSDIAVEWESNGQPENNYKTTPPVLDSDGSFFLYSKLT
VDKSRWQQGNVFSCSVMHEALHNHYTQKSLSLSPGGGGSPSEVQLVESGGGLVKPGGSLRLSCAA
SGFTFSDYGMAWVRQAPGKGLEWVAFITSLAYIIYYADSVKGRFTISRDNAKNSLYLQMNSLRAE
DTAVYYCARNYGSSPYYFDYWGQGTLVTVSSGGGGSGGGGSGGGGSGGGGSDIQMTQSPSSLSAS
VGDRVTITCRASQDINKFLSWYQQKPGKAPKLLIHYTSSLQPGVPSRFSGSGSGTDFTLTISSLQ
PEDFATYYCLQYDNLYTFGQGTKLEIK
>CD4001070_Chain2-SEQ ID NO: 141
DIVMTQSHKFMSTSVGDRVSITCKASQDVNTAVAWYQQKPGQSPKLLIYSASYRYTGVPDRFTGS
GSGTDFTFTISSVQAEDLAVYYCQQHYSTPWTFGGGTKLEIKGGGGSGGGGSGGGGSGGGGSQVQ
LQQSGAELAKPGASVKMSCKASGYAFTTYWMHWVKQRPGQGLEWIGYINPTTGYTDYNQKFKDKA
TLTADKSSSTAYMQLSSLTSEDSAVYYCARWGLPPFAYWGQGTLVTVSAEPKSSDKTHTCPPCPA
PPAAAPSVFLFPPKPKDTLMISRTPEVTCVVVDVSHEDPEVKFNWYVDGVEVHNAKTKPREEQYN
STYRVVSVLTVLHQDWLNGKEYKCAVSNKALPAPIEKTISKAKGQPREPQVYTLPPSRDELTKNQ
VSLSCAVKGFYPSDIAVEWESNGQPENNYKTTPPVLDSDGSFFLVSKLTVDKSRWQQGNVFSCSV
MHEALHNRFTQKSLSLSPGGGGSPSEVQLVESGGGLVKPGGSLRLSCAASGFTFSDYGMAWVRQA
PGKGLEWVAFITSLAYIIYYADSVKGRFTISRDNAKNSLYLQMNSLRAEDTAVYYCARNYGSSPY
YFDYWGQGTLVTVSSGGGGSGGGGSGGGGSGGGGSDIQMTQSPSSLSASVGDRVTITCRASQDIN
KFLSWYQQKPGKAPKLLIHYTSSLQPGVPSRFSGSGSGTDFTLTISSLQPEDFATYYCLQYDNLY
TFGQGTKLEIK
>CD4001071_Chain1-SEQ ID NO: 142
EPKSSDKTHTCPPCPAPPAAAPSVFLFPPKPKDTLMISRTPEVTCVVVDVSHEDPEVKFNWYVDG
VEVHNAKTKPREEQYNSTYRVVSVLTVLHQDWLNGKEYKCAVSNKALPAPIEKTISKAKGQPREP
QVYTLPPSRDELTKNQVSLWCLVKGFYPSDIAVEWESNGQPENNYKTTPPVLDSDGSFFLYSKLT
VDKSRWQQGNVFSCSVMHEALHNHYTQKSLSLSPGGGGSPSEVQLVESGGGLVKPGGSLRISCAA
SGFTFSDYGMAWVRQAPGKGLEWVAFITSLAYIIYYADSVKGRFTISRDNAKNSLYLQMNSLRAE
DTAVYYCARNYGSSPYYFDYWGQGTLVTVSSGGGGSGGGGSGGGGSGGGGSDIQMTQSPSSLSAS
VGDRVTITCRASQDINKFLSWYQQKPGKAPKLLIHYTSSLQPGVPSRFSGSGSGTDFTLTISSLQ
PEDFATYYCLQYDNLYTFGQGTKLEIK
>CD4001071_Chain2-SEQ ID NO: 143
DVVMTQTPLSLPVSLGDQASISCRSSQSLVHYNGYTYLHWYLQKPGQSPKLLIYKVSNRFSGVPD
RFSGSGSGTDFTLKISRVEAEDLGVYFCSQTTHVPLIFGAGTKLELRGGGGSGGGGSGGGGSGGG
GSEVMLVESGGGLVKPGGSLKLSCAASGFTFSSYAMSWVRQTPERRLEWVASISSAGTYIYYPDS
VKGRFTISRDNAKNILYLQMNSLRSEDTAMYYCARHGNGYDPLWYFDVWGAGTTVTVSSEPKSSD
KTHTCPPCPAPPAAAPSVFLFPPKPKDTLMISRIPEVTCVVVDVSHEDPEVKFNWYVDGVEVHNA
KTKPREEQYNSTYRVVSVLTVLHQDWLNGKEYKCAVSNKALPAPIEKTISKAKGQPREPQVYTLP
PSRDELTKNQVSLSCAVKGFYPSDIAVEWESNGQPENNYKTTPPVLDSDGSFFLVSKLTVDKSRW
QQGNVFSCSVMHEALHNRFTQKSLSLSPGGGGSPSEVQLVESGGGLVKPGGSLRLSCAASGFTFS
DYGMAWVRQAPGKGLEWVAFITSLAYIIYYADSVKGRFTISRDNAKNSLYLQMNSLRAEDTAVYY
CARNYGSSPYYFDYWGQGTLVTVSSGGGGSGGGGSGGGGSGGGGSDIQMTQSPSSLSASVGDRVT
ITCRASQDINKFLSWYQQKPGKAPKLLIHYTSSLQPGVPSRFSGSGSGTDFTLTISSLQPEDFAT
YYCLQYDNLYTFGQGTKLEIK
>CD4001072_Chain1-SEQ ID NO: 144
EPKSSDKTHTCPPCPAPPAAAPSVFLFPPKPKDTLMISRTPEVTCVVVDVSHEDPEVKFNWYVDG
VEVHNAKTKPREEQYNSTYRVVSVLTVLHQDWLNGKEYKCAVSNKALPAPIEKTISKAKGQPREP
QVYTLPPSRDELTKNQVSLWCLVKGFYPSDIAVEWESNGQPENNYKTTPPVLDSDGSFFLYSKLT
VDKSRWQQGNVFSCSVMHEALHNHYTQKSLSLSPGGGGSPSEVQLVESGGGLVKPGGSLRLSCAA
SGFTFSDYGMAWVRQAPGKGLEWVAFITSLAYIIYYADSVKGRFTISRDNAKNSLYLQMNSLRAE
DTAVYYCARNYGSSPYYFDYWGQGTLVTVSSGGGGSGGGGSGGGGSGGGGSDIQMTQSPSSLSAS
VGDRVTITCRASQDINKFLSWYQQKPGKAPKLLIHYTSSLQPGVPSRFSGSGSGTDFTLTISSLQ
PEDFATYYCLQYDNLYTFGQGTKLEIK
>CD4001072_Chain2-SEQ ID NO: 145
DIVMSQSPSSLAVSVGEKVTMSCKSSQSLLFSVNQKNYLAWYQQKPGQSPKLLIYWASTRFSGVP
DRFTGSGSGTDFTLTIGSVKAEDLAVYYCQHYYSYPWTFGGGTKLEIKGGGGSGGGGSGGGGSGG
GGSQVQLQQSGAELMNPGASVRISCKATGYTFSTSWIEWIKQRPGHGLEWIGEILPGSGSTNYNE
KFKGKATFTADTSSNTAYMQLSSLTFEDSAVYYCARGDDGSYVRWYFDVWGAGTTVTVSSEPKSS
DKTHTCPPCPAPPAAAPSVFLFPPKPKDTLMISRTPEVTCVVVDVSHEDPEVKFNWYVDGVEVHN
AKTKPREEQYNSTYRVVSVLTVLHQDWLNGKEYKCAVSNKALPAPIEKTISKAKGQPREPQVYTL
PPSRDELTKNQVSLSCAVKGFYPSDIAVEWESNGQPENNYKTTPPVLDSDGSFFLVSKLTVDKSR
WQQGNVFSCSVMHEALHNRFTQKSLSLSPGGGGSPSEVQLVESGGGLVKPGGSLRLSCAASGFTF
SDYGMAWVRQAPGKGLEWVAFITSLAYIIYYADSVKGRFTISRDNAKNSLYLQMNSLRAEDTAVY
YCARNYGSSPYYFDYWGQGTLVTVSSGGGGSGGGGSGGGGSGGGGSDIQMTQSPSSLSASVGDRV
TITCRASQDINKFLSWYQQKPGKAPKLLIHYTSSLQPGVPSRFSGSGSGTDFTLTISSLQPEDFA
TYYCLQYDNLYTFGQGTKLEIK
>CD4001073_Chain1-SEQ ID NO: 146
DIVMTQSHKFMSTSVGDRVSITCKASQDVNTAVAWYQQKPGQSPKLLIYSASYRYTGVPDRFTGS
GSGTDFTFTISSVQAEDLAVYYCQQHYSTPWTFGGGTKLEIKGGGGSGGGGSGGGGSGGGGSQVQ
LQQSGAELAKPGASVKMSCKASGYAFTTYWMHWVKQRPGQGLEWIGYINPTTGYTDYNQKFKDKA
TLTADKSSSTAYMQLSSLTSEDSAVYYCARWGLPPFAYWGQGTLVTVSAEPKSSDKTHTCPPCPA
PPAAAPSVFLFPPKPKDTLMISRTPEVTCVVVDVSHEDPEVKFNWYVDGVEVHNAKIKPREEQYN
STYRVVSVLTVLHQDGLNGKEYKCAVSNKALPAPIEKTISKAKGQPREPQVYTLPPSRDELTKNQ
VSLWCLVKGFYPSDIAVEWESNGQPENNYKTTPPVLDSDGSFFLYSKLTVDKSRWQQGNVFSCSV
MHEALHNHYTQKSLSLSPG
>CD4001073_Chain2-SEQ ID NO: 147
DIVMTQSHKFMSTSVGDRVSITCKASQDVNTAVAWYQQKPGQSPKLLIYSASYRYTGVPDRFTGS
GSGTDFTFTISSVQAEDLAVYYCQQHYSTPWTFGGGTKLEIKGGGGSGGGGSGGGGSGGGGSQVQ
LQQSGAELAKPGASVKMSCKASGYAFTTYWMHWVKQRPGQGLEWIGYINPTTGYTDYNQKFKDKA
TLTADKSSSTAYMQLSSLTSEDSAVYYCARWGLPPFAYWGQGTLVTVSAEPKSSDKTHTCPPCPA
PPAAAPSVFLFPPKPKDTLMISRTPEVTCVVVDVSHEDPEVKFNWYVDGVEVHNAKTKPREEQYN
STYRVVSVLTVLHQDGLNGKEYKCAVSNKALPAPIEKTISKAKGQPREPQVYTLPPSRDELTKNQ
VSLSCAVKGFYPSDIAVEWESNGQPENNYKTTPPVLDSDGSFFLVSKLTVDKSRWQQGNVFSCSV
MHEALHNRFTQKSLSLSPGGGGSPSEVQLVESGGGLVKPGGSLRLSCAASGFTFSDYGMAWVRQA
PGKGLEWVAFITSLAYIIYYADSVKGRFTISRDNAKNSLYLQMNSLRAEDTAVYYCARNYGSSPY
YFDYWGQGTLVTVSSGGGGSGGGGSGGGGSGGGGSDIQMTQSPSSLSASVGDRVTITCRASQDIN
KFLSWYQQKPGKAPKLLIHYTSSLQPGVPSRFSGSGSGTDFTLTISSLQPEDFATYYCLQYDNLY
TFGQGTKLEIK
>CD4001074_Chain1-SEQ ID NO: 148
DVVMTQTPLSLPVSLGDQASISCRSSQSLVHYNGYTYLHWYLQKPGQSPKLLIYKVSNRFSGVPD
RFSGSGSGTDFTLKISRVEAEDLGVYFCSQTTHVPLTFGAGTKLELRGGGGSGGGGSGGGGSGGG
GSEVMLVESGGGLVKPGGSLKLSCAASGFTFSSYAMSWVRQTPERRLEWVASISSAGTYIYYPDS
VKGRFTISRDNAKNILYLQMNSLRSEDTAMYYCARHGNGYDPLWYFDVWGAGTTVTVSSEPKSSD
KTHTCPPCPAPPAAAPSVFLFPPKPKDTLMISRTPEVTCVVVDVSHEDPEVKFNWYVDGVEVHNA
KTKPREEQYNSTYRVVSVLTVLHQDGLNGKEYKCAVSNKALPAPIEKTISKAKGQPREPQVYTLP
PSRDELTKNQVSLWCLVKGFYPSDIAVEWESNGQPENNYKTTPPVLDSDGSFFLYSKLTVDKSRW
QQGNVFSCSVMHEALHNHYTQKSLSLSPG
>CD4001074_Chain2-SEQ ID NO: 149
DVVMTQTPLSLPVSLGDQASISCRSSQSLVHYNGYTYLHWYLQKPGQSPKLLIYKVSNRFSGVPD
RFSGSGSGTDFTLKISRVEAEDLGVYFCSQTTHVPLTFGAGTKLELRGGGGSGGGGSGGGGSGGG
GSEVMLVESGGGLVKPGGSLKLSCAASGFTFSSYAMSWVRQTPERRLEWVASISSAGTYIYYPDS
VKGRFTISRDNAKNILYLQMNSLRSEDTAMYYCARHGNGYDPLWYFDVWGAGTTVTVSSEPKSSD
KTHTCPPCPAPPAAAPSVFLFPPKPKDTLMISRTPEVTCVVVDVSHEDPEVKFNWYVDGVEVHNA
KTKPREEQYNSTYRVVSVLTVLHQDWLNGKEYKCAVSNKALPAPIEKTISKAKGQPREPQVYTLP
PSRDELTKNQVSLSCAVKGFYPSDIAVEWESNGQPENNYKTTPPVLDSDGSFFLVSKLTVDKSRW
QQGNVFSCSVMHEALHNRFTQKSLSLSPGGGGSPSEVQLVESGGGLVKPGGSLRLSCAASGFTFS
DYGMAWVRQAPGKGLEWVAFITSLAYIIYYADSVKGRFTISRDNAKNSLYLQMNSLRAEDTAVYY
CARNYGSSPYYFDYWGQGTLVTVSSGGGGSGGGGSGGGGSGGGGSDIQMTQSPSSLSASVGDRVT
ITCRASQDINKFLSWYQQKPGKAPKLLIHYTSSLQPGVPSRFSGSGSGTDFTLTISSLQPEDFAT
YYCLQYDNLYTFGQGTKLEIK
>CD4001075_Chain1-SEQ ID NO: 150
DIVMSQSPSSLAVSVGEKVTMSCKSSQSLLFSVNQKNYLAWYQQKPGQSPKLLIYWASTRFSGVP
DRFTGSGSGTDFTLTIGSVKAEDLAVYYCQHYYSYPWTFGGGTKLEIKGGGGSGGGGSGGGGSGG
GGSQVQLQQSGAELMNPGASVRISCKATGYTFSTSWIEWIKQRPGHGLEWIGEILPGSGSTNYNE
KFKGKATFTADTSSNTAYMQLSSLTFEDSAVYYCARGDDGSYVRWYFDVWGAGTTVTVSSEPKSS
DKTHTCPPCPAPPAAAPSVFLFPPKPKDTLMISRTPEVTCVVVDVSHEDPEVKFNWYVDGVEVHN
AKTKPREEQYNSTYRVVSVLTVLHQDGLNGKEYKCAVSNKALPAPIEKTISKAKGQPREPQVYTL
PPSRDELTKNQVSLWCLVKGFYPSDIAVEWESNGQPENNYKTTPPVLDSDGSFFLYSKLTVDKSR
WQQGNVFSCSVMHEALHNHYTQKSLSLSPG
>CD4001075_Chain2-SEQ ID NO: 151
DIVMSQSPSSLAVSVGEKVTMSCKSSQSLLFSVNQKNYLAWYQQKPGQSPKLLIYWASTRFSGVP
DRFTGSGSGTDFTLTIGSVKAEDLAVYYCQHYYSYPWTFGGGTKLEIKGGGGSGGGGSGGGGSGG
GGSQVQLQQSGAELMNPGASVRISCKATGYTFSTSWIEWIKQRPGHGLEWIGEILPGSGSTNYNE
KFKGKATFTADTSSNTAYMQLSSLTFEDSAVYYCARGDDGSYVRWYFDVWGAGTTVTVSSEPKSS
DKTHTCPPCPAPPAAAPSVFLFPPKPKDTLMISRTPEVTCVVVDVSHEDPEVKFNWYVDGVEVHN
AKTKPREEQYNSTYRVVSVLTVLHQDGLNGKEYKCAVSNKALPAPIEKTISKAKGQPREPQVYTL
PPSRDELTKNQVSLSCAVKGFYPSDIAVEWESNGQPENNYKTTPPVLDSDGSFFLVSKLTVDKSR
WQQGNVFSCSVMHEALHNRFTQKSLSLSPGGGGSPSEVQLVESGGGLVKPGGSLRLSCAASGFTF
SDYGMAWVRQAPGKGLEWVAFITSLAYIIYYADSVKGRFTISRDNAKNSLYLQMNSLRAEDTAVY
YCARNYGSSPYYFDYWGQGTLVTVSSGGGGSGGGGSGGGGSGGGGSDIQMTQSPSSLSASVGDRV
TITCRASQDINKFLSWYQQKPGKAPKLLIHYTSSLQPGVPSRFSGSGSGTDFTLTISSLQPEDFA
TYYCLQYDNLYTFGQGTKLEIK
>CD4001076_Chain1-SEQ ID NO: 152
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDGLNGKEYKCAVSNKALPAPIEKTISKAKGQPREPQVYTLPPSRDELTK
NQVSLWCLVKGFYPSDIAVEWESNGQPENNYKTTPPVLDSDGSFFLYSKLTVDKSRWQQGNVFSC
SVMHEALHNHYTQKSLSLSPG
>CD4001076_Chain2-SEQ ID NO: 153
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPSRDELTK
NQVSLSCAVKGFYPSDIAVEWESNGQPENNYKTTPPVLDSDGSFFLVSKLTVDKSRWQQGNVFSC
SVMHEALHNRFTQKSLSLSPGGGGSPSDIVMTQSHKFMSTSVGDRVSITCKASQDVNTAVAWYQQ
KPGQSPKLLIYSASYRYTGVPDRFTGSGSGTDFTFTISSVQAEDLAVYYCQQHYSTPWTFGGGTK
LEIKGGGGSGGGGSGGGGSGGGGSQVQLQQSGAELAKPGASVKMSCKASGYAFTTYWMHWVKQRP
GQGLEWIGYINPTTGYTDYNQKFKDKATLTADKSSSTAYMQLSSLTSEDSAVYYCARWGLPPFAY
WGQGTLVTVSA
>CD4001077_Chain1-SEQ ID NO: 154
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDGLNGKEYKCAVSNKALPAPIEKTISKAKGQPREPQVYTLPPSRDELTK
NQVSLWCLVKGFYPSDIAVEWESNGQPENNYKTTPPVLDSDGSFFLYSKLTVDKSRWQQGNVFSC
SVMHEALHNHYTQKSLSLSPG
>CD4001077_Chain2-SEQ ID NO: 155
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPSRDELTK
NQVSLSCAVKGFYPSDIAVEWESNGQPENNYKTTPPVLDSDGSFFLVSKLTVDKSRWQQGNVFSC
SVMHEALHNRFTQKSLSLSPGGGGSPSDVVMTQTPLSLPVSLGDQASISCRSSQSLVHYNGYTYL
HWYLQKPGQSPKLLIYKVSNRFSGVPDRFSGSGSGTDFTLKISRVEAEDLGVYFCSQTTHVPLTF
GAGTKLELRGGGGSGGGGSGGGGSGGGGSEVMLVESGGGLVKPGGSLKLSCAASGFTFSSYAMSW
VRQTPERRLEWVASISSAGTYIYYPDSVKGRFTISRDNAKNILYLQMNSLRSEDTAMYYCARHGN
GYDPLWYFDVWGAGTTVTVSS
>CD4001078_Chain1-SEQ ID NO: 156
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPSRDELTK
NQVSLWCLVKGFYPSDIAVEWESNGQPENNYKTTPPVLDSDGSFFLYSKLTVDKSRWQQGNVFSC
SVMHEALHNHYTQKSLSLSPG
>CD4001078_Chain2-SEQ ID NO: 157
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDILMISRTPEVTCVVVDVSHEDPEVKFNWYVDGVEVHNAKTKPREEQ
YNSTYRVVSVLTVLHQDGLNGKEYKCAVSNKALPAPIEKTISKAKGQPREPQVYTLPPSRDELTK
NQVSLSCAVKGFYPSDIAVEWESNGQPENNYKTTPPVLDSDGSFFLVSKLTVDKSRWQQGNVFSC
SVMHEALHNRFTQKSLSLSPGGGGSPSDIVMSQSPSSLAVSVGEKVTMSCKSSQSLLFSVNQKNY
LAWYQQKPGQSPKLLIYWASTRFSGVPDRFTGSGSGTDFTLTIGSVKAEDLAVYYCQHYYSYPWT
FGGGTKLEIKGGGGSGGGGSGGGGSGGGGSQVQLQQSGAELMNPGASVRISCKATGYTFSTSWIE
WIKQRPGHGLEWIGEILPGSGSTNYNEKFKGKATFTADTSSNTAYMQLSSLTFEDSAVYYCARGD
DGSYVRWYFDVWGAGTTVTVSS
>CD4001079_Chain1-SEQ ID NO: 158
EPKSSDKTHTCPPCPAPPAAAPSVFLFPPKPKDTLMISRTPEVTCVVVDVSHEDPEVKFNWYVDG
VEVHNAKTKPREEQYNSTYRVVSVLTVLHQDGLNGKEYKCAVSNKALPAPIEKTISKAKGQPREP
QVYTLPPSRDELTKNQVSLWCLVKGFYPSDIAVEWESNGQPENNYKTTPPVLDSDGSFFLYSKLT
VDKSRWQQGNVFSCSVMHEALHNHYTQKSLSLSPGGGGSPSDIVMTQSHKFMSTSVGDRVSITCK
ASQDVNTAVAWYQQKPGQSPKLLIYSASYRYTGVPDRFTGSGSGTDFTFTISSVQAEDLAVYYCQ
QHYSTPWTFGGGTKLEIKGGGGSGGGGSGGGGSGGGGSQVQLQQSGAELAKPGASVKMSCKASGY
AFTTYWMHWVKQRPGQGLEWIGYINPTTGYTDYNQKFKDKATLTADKSSSTAYMQLSSLTSEDSA
VYYCARWGLPPFAYWGQGTLVTVSA
>CD4001079_Chain2-SEQ ID NO: 159
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDGLNGKEYKCAVSNKALPAPIEKTISKAKGQPREPQVYTLPPSRDELTK
NQVSLSCAVKGFYPSDIAVEWESNGQPENNYKTTPPVLDSDGSFFLVSKLTVDKSRWQQGNVFSC
SVMHEALHNRFTQKSLSLSPGGGGSPSDIVMTQSHKFMSTSVGDRVSITCKASQDVNTAVAWYQQ
KPGQSPKLLIYSASYRYTGVPDRFTGSGSGTDFTFTISSVQAEDLAVYYCQQHYSTPWTFGGGTK
LEIKGGGGSGGGGSGGGGSGGGGSQVQLQQSGAELAKPGASVKMSCKASGYAFTTYWMHWVKQRP
GQGLEWIGYINPTTGYTDYNQKFKDKATLTADKSSSTAYMQLSSLISEDSAVYYCARWGLPPFAY
WGQGTLVTVSA
>CD4001080_Chain1-SEQ ID NO: 160
EPKSSDKTHTCPPCPAPPAAAPSVFLFPPKPKDTLMISRTPEVTCVVVDVSHEDPEVKFNWYVDG
VEVHNAKTKPREEQYNSTYRVVSVLTVLHQDWLNGKEYKCAVSNKALPAPIEKTISKAKGQPREP
QVYTLPPSRDELTKNQVSLWCLVKGFYPSDIAVEWESNGQPENNYKTTPPVLDSDGSFFLYSKLT
VDKSRWQQGNVFSCSVMHEALHNHYTQKSLSLSPGGGGSPSDVVMTQTPLSLPVSLGDQASISCR
SSQSLVHYNGYTYLHWYLQKPGQSPKLLIYKVSNRFSGVPDRFSGSGSGTDFTLKISRVEAEDLG
VYFCSQTTHVPLTFGAGTKLELRGGGGSGGGGSGGGGSGGGGSEVMLVESGGGLVKPGGSLKLSC
AASGFTFSSYAMSWVRQTPERRLEWVASISSAGTYIYYPDSVKGRFTISRDNAKNILYLQMNSLR
SEDTAMYYCARHGNGYDPLWYFDVWGAGTTVTVSS
>CD4001080_Chain2-SEQ ID NO: 161
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGGGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPSRDELTK
NQVSLSCAVKGFYPSDIAVEWESNGQPENNYKTTPPVLDSDGSFFLVSKLTVDKSRWQQGNVFSC
SVMHEALHNRETQKSLSLSPGGGGSPSDVVMTQTPLSLPVSLGDQASISCRSSQSLVHYNGYTYL
HWYLQKPGQSPKLLIYKVSNRFSGVPDRFSGSGSGTDFTLKISRVEAEDLGVYFCSQTTHVPLTF
GAGTKLELRGGGGSGGGGSGGGGSGGGGSEVMLVESGGGLVKPGGSLKLSCAASGFTFSSYAMSW
VRQTPERRLEWVASISSAGTYIYYPDSVKGRFTISRDNAKNILYLQMNSLRSEDTAMYYCARHGN
GYDPLWYFDVWGAGTTVTVSS
>CD4001081_Chain1-SEQ ID NO: 162
EPKSSDKTHTCPPCPAPPAAAPSVFLFPPKPKDTLMISRTPEVTCVVVDVSHEDPEVKFNWYVDG
VEVHNAKTKPREEQYNSTYRVVSVLTVLHQDWLNGKEYKCAVSNKALPAPIEKTISKAKGQPREP
QVYTLPPSRDELTKNQVSLWCLVKGFYPSDIAVEWESNGQPENNYKTTPPVLDSDGSFFLYSKLT
VDKSRWQQGNVFSCSVMHEALHNHYTQKSLSLSPGGGGSPSDIVMSQSPSSLAVSVGEKVTMSCK
SSQSLLFSVNQKNYLAWYQQKPGQSPKLLIYWASTRFSGVPDRFTGSGSGTDFTLTIGSVKAEDL
AVYYCQHYYSYPWTFGGGTKLEIKGGGGSGGGGSGGGGSGGGGSQVQLQQSGAELMNPGASVRIS
CKATGYTFSTSWIEWIKQRPGHGLEWIGEILPGSGSTNYNEKFKGKATFTADTSSNTAYMQLSSL
TFEDSAVYYCARGDDGSYVRWYFDVWGAGTTVTVSS
>CD4001081_Chain2-SEQ ID NO: 163
EVQLVESGGGLVKPGGSLRLSCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDGLNGKEYKCAVSNKALPAPIEKTISKAKGQPREPQVYTLPPSRDELTK
NQVSLSCAVKGFYPSDIAVEWESNGQPENNYKTTPPVLDSDGSFFLVSKLTVDKSRWQQGNVFSC
SVMHEALANRFTQKSLSLSPGGGGSPSDIVMSQSPSSLAVSVGEKVTMSCKSSQSLLFSVNQKNY
LAWYQQKPGQSPKLLIYWASTRFSGVPDRFTGSGSGTDFTLTIGSVKAEDLAVYYCQHYYSYPWT
FGGGTKLEIKGGGGSGGGGSGGGGSGGGGSQVQLQQSGAELMNPGASVRISCKATGYTFSTSWIE
WIKQRPGHGLEWIGEILPGSGSTNYNEKFKGKATFTADTSSNTAYMQLSSLTFEDSAVYYCARGD
DGSYVRWYFDVWGAGTTVTVSS
>PQC01043-SEQ ID NO: 164
EVQLQESGPGLVKPSETLSLTCTVSGYSITSNYYWNWIRQPPGKGLEWMGYIRYDGSNNYNPSLK
NRVTISRDTSKNQFSLKLSSVTAADTAVYYCARLDYWGQGTTVTVSSDLSGGGGSGGGGSGGGGS
GGGGSTGDAVMTQTPLSLSVTPGQPASISCRSSQSLENINGNTFLNWYLQKPGQSPQLLIYRVSN
RFSGVPDRFSGSGSGTDFTLKISRVEAEDVGVYYCLQVTHVPFTFGQGTKLEIKEPKSSDKTHTC
PPCPAPPAAAPSVFLFPPKPKDTLMISRTPEVTCVVVDVSHEDPEVKFNWYVDGVEVHNAKTKPR
EEQYNSTYRVVSVLTVLHQDWLNGKEYKCAVSNKALPAPIEKTISKAKGQPREPQVYTLPPSRDE
LIKNQVSLTCLVKGFYPSDIAVEWESNGQPENNYKTTPPVLDSDGSFFLYSKLTVDKSRWQQGNV
FSCSVMHEALHNHYTQKSLSLSPGGGGSPSDIQMTQSPSSLSASVGDRVTITCRASQDVSTAVAW
YQQKPGKAPKLLIYSASFLYSGVPSRFSGSGSGTDFTLTISSLQPEDFATYYCQQYLYHPATFGQ
GTKVEIKGGGGSGGGGSGGGGSEVQLVESGGGLVQPGGSLRLSCAASGFTFSDSWIHWVRQAPGK
GLEWVAWISPYGGSTYYADSVKGRFTISADTSKNTAYLQMNSLRAEDTAVYYCARRHWPGGFDYW
GQGTLVTVSS
>PDL01029-SEQ ID NO: 165
DIQMTQSPSSLSASVGDRVTITCRASQDVSTAVAWYQQKPGKAPKLLIYSASFLYSGVPSRFSGS
GSGTDFTLTISSLQPEDFATYYCQQYLYHPATFGQGTKVEIKGGGGSGGGGSGGGGSEVQLVESG
GGLVQPGGSLRLSCAASGFTFSDSWIHWVRQAPGKGLEWVAWISPYGGSTYYADSVKGRFTISAD
TSKNTAYLQMNSLRAEDTAVYYCARRHWPGGFDYWGQGTLVTVSSEPKSSDKTHTCPPCPAPELL
GGPSVFLFPPKPKDTLMISRTPEVTCVVVDVSHEDPEVKFNWYVDGVEVHNAKTKPREEQYNSTY
RVVSVLTVLHQDGLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHE
ALHNHYTQKSLSLSPGK
>PDL01085-SEQ ID NO: 166
EVQLVESGGGLVKPGGSLRISCAASGFTFSDYGMAWVRQAPGKGLEWVAFITSLAYIIYYADSVK
GRFTISRDNAKNSLYLQMNSLRAEDTAVYYCARNYGSSPYYFDYWGQGTLVTVSSGGGGSGGGGS
GGGGSGGGGSDIQMTQSPSSLSASVGDRVTITCRASQDINKFLSWYQQKPGKAPKLLIHYTSSLQ
PGVPSRFSGSGSGTDFTLTISSLQPEDFATYYCLQYDNLYTFGQGTKLEIKSSSEPKSSDKTHTC
PPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGN
VFSCSVMHEALHNHYTQKSLSLSPGK
>PDL01127_LightChain-SEQ ID NO: 167
EIVLTQSPGTLSLSPGFRATLSCRASQRVSSSYLAWYQQKPGQAPRLLIYDASSRATGIPDRFSG
SGSGTDFTLTISRLEPEDFAVYYCQQYGSLPWTFGQGTKVEIKRTVAAPSVFIFPPSDEQLKSGT
ASVVCLLNNFYPREAKVQWKVDNALQSGNSQESVTEQDSKDSTYSLSSTLTLSKADYEKHKVYAC
EVTHQGLSSPVTKSFNRGEC
>PDL01127 HeavyChain-SEQ ID NO: 168
EVQLVESGGGLVQPGGSLRLSCAASGFTFSRYWMSWVRQAPGKGLEWVANIKQDGSEKYYVDSVK
GRFTISRDNAKNSLYLQMNSLRAEDTAVYYCAREGGWFGELAFDYWGQGTLVTVSSASTKGPSVE
PLAPCSRSTSESTAALGCLVKDYFPEPVTVSWNSGALTSGVHTEPAVLQSSGLYSLSSVVTVPSS
SLGTKTYTCNVDHKPSNTKVDKRVESKYGPPCPPCPAPEFLGGPSVFLFPPKPKDTLMISRTPEV
TCVVVDVSQEDPEVQFNWYVDGVEVHNAKTKPREEQFNSTYRVVSVLTVLHQDGLNGKEYKCKVS
NKGLPSSIEKTISKAKGQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQKSLSLSLGKGGGGSGG
GGSNPQIAAHVISEASSKITSVLQWAEKGYYTMSNNLVTLENGKQLTVKRQGLYYIYAQVTFCSN
REASSQAPFIASLWLKSPGRFERILLRAANTHSSAKPCGQQSIHLGGVFELQPGASVEVNVIDPS
QVSHGTGFTSFGLLKLGGGGSGGGSQIAAHVISEASSKITSVLQWAEKGYYTMSNNLVTLENGKQ
LTVKRQGLYYIYAQVTFCSNREASSQAPFIASLWLKSPGRFERILLRAANTHSSAKPCGQQSIHL
GGVFELQPGASVFVNVTDPSQVSHGTGFTSFGLLKLGGGGSGGGSQIAAHVISEASSKTTSVLQW
AEKGYYTMSNNLVTLENGKQLTVKRQGLYYIYAQVTFCSNREASSQAPFIASLWLKSPGRFERIL
LRAANTHSSAKPCGQQSIHLGGVFELQPGASVFVNVTDPSQVSHGTGFTSFGLLKL
>PDL01153-SEQ ID NO: 169
SSEPKSSDKTHTCPPCPAPPAAAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPR
EPQVYTLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGGGGSPSEVQLVESGGGLVKPGGSLRLSC
AASGFTFSDYGMAWVRQAPGKGLEWVAFITSLAYIIYYADSVKGRFTISRDNAKNSLYLQMNSLR
AEDTAVYYCARNYGSSPYYFDYWGQGTLVTVSSGGGGSGGGGSGGGGSGGGGSDIQMTQSPSSLS
ASVGDRVTITCRASQDINKFLSWYQQKPGKAPKLLIHYTSSLQPGVPSRFSGSGSGTDFTLTISS
LQPEDFATYYCLQYDNLYTFGQGTKLEIK
>CD4001086-SEQ ID NO: 170
EVVMTQSPGTLSLSPGFRATLSCRSSQSLVHYNGYTYLHWYQQKPGQAPRLLIYKVSNRFSGIPA
RFSGSGSGTDFTLTISRLEPEDLAVYFCSQTTHVPLTFGQGTKVEIKGGGGSGGGGSGGGGSGGG
GSEVQLVESGGGLVQPGRSLRLSCAASGFTFSSYAMSWVRQAPGKGLEWVASISSAGTYIYYADS
VQGRFTISRDNGKNSLYLQMNSLRAEDTALYYCARHGNGYDPLWYFDVWGQGTMVTVSS
>CD4001087-SEQ ID NO: 171
EVVMTQSPDTLSVSPGFRATLSCRSSQSLVHYNGYTYLHWYQQKPGQVPRLLIYKVSNRFSGVPA
RFTGSGSGTEFTLTISSLQSEDFAVYFCSQTTHVPLTFGQGTKLEIKGGGGSGGGGSGGGGGGGG
GSEVQLVESGGGLVQPGRSLRLSCAASGFTFSSYAMSWVRQAPGKGLEWVASISSAGTYIYYADS
VQGRFTISRDNGKNSLYLQMNSLRAEDTALYYCARHGNGYDPLWYFDVWGQGTMVTVSS
>CD4001088-SEQ ID NO: 172
EVVMTQSPDFQSVTPKFKVTITCRSSQSLVHYNGYTYLHWYQQKPDQSPKLLIYKVSNRFSGVPS
RFSGSGSGTDFTLTINSLEAEDAAAYFCSQTTHVPLTFGPGTKVDIKGGGGSGGGGSGGGGSGGG
GSEVQLVESGGGLVQPGRSLRISCAASGFTFSSYAMSWVRQAPGKGLEWVASISSAGTYIYYADS
VQGRFTISRDNGKNSLYLQMNSLRAEDTALYYCARHGNGYDPLWYFDVWGQGTMVTVSS
>CD4001089-SEQ ID NO: 173
DVQMTQSPILLSASVGDRVTITCRSSQSLVHYNGYTYVHWYQQRTNGSPRLLIYKVSNRFSGVPD
RFSGSGSGTDFTLTINSLEAEDAAAYFCSQTTHVPLTFGPGTKVDIKGGGGSGGGGSGGGGSGGG
GSEVQLVESGGGLVQPGRSLRISCAASGFTFSSYAMSWVRQAPGKGLEWVASISSAGTYIYYADS
VQGRFTISRDNGKNSLYLQMNSLRAEDTALYYCARHGNGYDPLWYFDVWGQGTMVTVSS
>CD4001090-SEQ ID NO: 174
EVVMTQSPDFQSVTPKFKVTITCRSSQSLVHYNGYTYLHWYQQKPDQSPKLLIYKVSNRFSGVPS
RFSGSGSGTDFTLTINSLEAEDAAAYFCSQTTHVPLTFGPGTKVDIKGGGGSGGGGSGGGGSGGG
GSEVQLVESGGGVVQPGRSLRLSCAASGFTFSSYAMSWVRQAPGKGLEWVASISSAGTYIYYADS
VKGRFTISRDNSKNTLYLQMNGLRAEDTAVYYCARHGNGYDPLWYFDVWGQGTLVTVSS
>CD4001091-SEQ ID NO: 175
DVQMTQSPILLSASVGDRVTITCRSSQSLVHYNGYTYVHWYQQRTNGSPRLLIYKVSNRFSGVPD
RFSGSGSGTDFTLTISSLQPEDIADYFCQQTTHVPLTFGAGTKVEIKGGGGSGGGGSGGGGSGGG
GSEVQLVESGGGVVQPGRSLRLSCAASGFTFSSYAMSWVRQAPGKGLEWVASISSAGTYIYYADS
VKGRFTISRDNSKNTLYLQMNGLRAEDTAVYYCARHGNGYDPLWYFDVWGQGTLVTVSS
>CD4001092-SEQ ID NO: 176
DVVMTQSPDSLAVSLGERATINCRSSQSLVHYNGYTYLHWYQQKPGQPPKLLIYKVSNRFSGVPD
RFSGSGSGTDFTLTISSLQAEDVAVYFCSQTTHVPLTFGAGTKLELRGGGGSGGGGSGGGGSGGG
GSEVQLVESGGGVVQPGRSLRLSCAASGFTFSSYAMSWVRQAPGKGLEWVASISSAGTYIYYADS
VKGRFTISRDNSKNTLYLQMNGLRAEDTAVYYCARHGNGYDPLWYFDVWGQGTLVTVSS
>CD4001093-SEQ ID NO: 177
DVVMTQSPDTLSLSPGFRATLSCRSSQSLVHYNGYTYLHWYQQQPGQAPRLLIYKVSNRFSGIPD
RFSGSGSGTDFTLTISRLEPEDFAVYFCSQTTHVPLTFGQGTKLEIKGGGGSGGGGSGGGGSGGG
GSEVQLLFSGGGLVQPGGSLRLSCAASGFTFSSYAMSWVRQAPGKGLEWVASISSAGTYIYYTDS
VKGRFSISRDNSKNTLYLQMNSLRAEDTAVYYCARHGNGYDPLWYFDVWGQGTTVTVSS
>CD4001094-SEQ ID NO: 178
DVQMTQSPILLSASVGDRVTITCRSSQSLVHYNGYTYVHWYQQRTNGSPRLLIYKVSNRFSGVPD
RFSGSGSGTDFTLTISSLQPEDIADYFCQQTTHVPLTFGAGTKVEIKGGGGSGGGGSGGGGSGGG
GSEVQLLFSGGGLVQPGGSLRLSCAASGFTFSSYAMSWVRQAPGKGLEWVASISSAGTYIYYTDS
VKGRFSISRDNSKNTLYLQMNSLRAEDTAVYYCARHGNGYDPLWYFDVWGQGTTVTVSS
>CD4001095-SEQ ID NO: 179
DVVMTQSPDTLSLSPGFRATLSCRSSQSLVHYNGYTYLHWYQQQPGQAPRLLIYKVSNRFSGIPD
RFSGSGSGTDFTLTISRLEPEDFAVYFCSQTTHVPLTFGQGTKLEIKGGGGSGGGGSGGGGSGGG
GSQVQLVESGGGLVQPGGSLRLSCAASGFTFSSYAMSWVRQAPGKGLVWVASISSAGTYIYYADS
VKGRFTISRDNAKNTLYLQMTSLRAEDTAIYYCARHGNGYDPLWYFDVWGQGALVTVSS
>CD4001096-SEQ ID NO: 180
DVQMTQSPILLSASVGDRVTITCRSSQSLVHYNGYTYVHWYQQRTNGSPRLLIYKVSNRFSGVPD
RFSGSGSGTDFTLTISSLQPEDIADYFCQQTTHVPLTFGAGTKVEIKGGGGSGGGGSGGGGSGGG
GSQVQLVESGGGLVQPGGSLRLSCAASGFTFSSYAMSWVRQAPGKGLVWVASISSAGTYIYYADS
VKGRFTISRDNAKNTLYLQMTSLRAEDTAIYYCARHGNGYDPLWYFDVWGQGALVTVSS
>CD4001097-SEQ ID NO: 181
EVVMTQSPGTLSLSPGFRATLSCRSSQSLVHYNGYTYLHWYQQKPGQAPRLLIYKVSNRFSGIPA
RFSGSGSGTDFTLTISRLEPEDLAVYFCSQTTHVPLIFGQGTKVEIKGGGGSGGGGSGGGGSGGG
GSQVQLVESGGGLVKPGGSLRLSCAASGFTFSSYAMSWVRQAPGKGLEWVASISSAGTYIYYADS
VKGRFTISRDNAKNSLYLQMNSLRAEDTAVYYCARHGNGYDPLWYFDVWGAGTTVTVSS
>CD4001098-SEQ ID NO: 182
DVQMTQSPILLSASVGDRVTITCRSSQSLVHYNGYTYVHWYQQRTNGSPRLLIYKVSNRFSGVPD
RFSGSGSGTDFTLTISSLQPEDIADYFCQQTTHVPLTFGAGTKVEIKGGGGSGGGGSGGGGSGGG
GSQVQLVESGGGLVKPGGSLRLSCAASGFTFSSYAMSWVRQAPGKGLEWVASISSAGTYIYYADS
VKGRFTISRDNAKNSLYLQMNSLRAEDTAVYYCARHGNGYDPLWYFDVWGAGTTVTVSS
>CD4001099-SEQ ID NO: 183
EVVMTQSPDFQSVTPKFKVTITCRSSQSLVHYNGYTYLHWYQQKPDQSPKLLIYKVSNRFSGVPS
RFSGSGSGTDFTLTINSLEAEDAAAYFCSQTTHVPLTFGPGTKVDIKGGGGSGGGGSGGGGSGGG
GSEVQLVESGGGLEQPGGSLRLSCAASGFTFSSYAMSWVRQAPGKGLEWVASISSAGTYIYYADS
VKGRFTISRDNSKNTLYLQMNSLRAEDTAVYYCARHGNGYDPLWYFDVWGQGTTVTVSS
>CD4001100-SEQ ID NO: 184
DVQMTQSPILLSASVGDRVTITCRSSQSLVHYNGYTYVHWYQQRTNGSPRLLIYKVSNRFSGVPD
RFSGSGSGTDFTLTISSLQPEDIADYFCQQTTHVPLTFGAGTKVEIKGGGGSGGGGSGGGGGGGG
GSEVQLVESGGGLEQPGGSLRLSCAASGFTFSSYAMSWVRQAPGKGLEWVASISSAGTYIYYADS
VKGRFTISRDNSKNTLYLQMNSLRAEDTAVYYCARHGNGYDPLWYFDVWGQGTTVTVSS
>CD4001132-SEQ ID NO: 185
DVQMTQSPILLSASVGDRVTITCRSSQSLVHYNGYTYVHWYQQKPGKAPKLLIYKVSNLESGVPS
RFSGSGSGTDFTLTISSLQPEDIADYFCQQTTHVPLTFGAGTKVEIKGGGGSGGGGGGGGSGGG
GSEVQLVESGGGLVQPGGSLRLSCAASGFTFSSYAMSWVRQAPGKGLEWVSAISSAGTYIYYADS
VKGRFTISRDNSKNTLYLQMNSLRAEDTAVYYCARHGNGYDPLWYFDVWGQGTTVTVSS
>CD4001133_Humanized5B9_VH-SEQ ID NO: 186
EVQLVESGGGLVQPGGSLRLSCAASGFTFSSYAMSWVRQAPGKGLEWVSAISSAGTYIYYADSVK
GRFTISRDNSKNTLYLQMNSLRAEDTAVYYCARIGNGYDPLWYFDVWGQGTTVTVSS
>Humanized5B9_HCDR1-SEQ ID NO: 187-SYAMS
>Humanized5B9_HCDR2-SEQ ID NO: 188-AISSAGTYIYYADSVKG
>Humanized5B9_HCDR3-SEQ ID NO: 189-HGNGYDPLWFVD
>CD4001133_Humanized5B9_VL-SEQ ID NO: 190
DIQMTQSPSSLSASVGDRVTITCRASQSLVHYNGYTYLNWYQQKPGKAPKLLIYKVSNRFSGVPD
RFSGSGSGTDFTLTISSLQPEDFATYYCQQTTHVPLTFGQGTKVEIK
>Humanized5B9_LCDR1-SEQ ID NO: 191-RASQSLVHYNGYTYLN
>Humanized5B9_LCDR2-SEQ ID NO: 192-KVSNRFS
>Humanized5B9_LCDR3-SEQ ID NO: 193-QQTTHVPLT
>CD4001209-SEQ ID NO: 194
SSEPKSSDKTHTCPPCPAPPAAAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPR
EPQVYTLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGGGGSPSDIQMTQSPSSLSASVGDRVTIT
CRASQSLVHYNGYTYLNWYQQKPGKAPKLLIYKVSNRFSGVPDRFSGSGSGTDFTLTISSLQPED
FATYYCQQTTHVPLTFGQGTKVEIKGGGGSGGGGSGGGGSGGGGSEVQLVESGGGLVQPGGSLRL
SCAASGFTFSSYAMSWVRQAPGKGLEWVSAISSAGTYIYYADSVKGRFTISRDNSKNTLYLQMNS
LRAEDTAVYYCARHGNGYDPLWYFDVWGQGTTVTVSSS
>PDL01157_Chain1-SEQ ID NO: 195
DIQMTQSPSSLSASVGDRVTITCRASQDINKFLSWYQQKPGKAPKLLIHYTSSLQSGVPSRFSGS
GSGTDFTLTISSLQPEDFATYYCLQYDNLYTFGQGTKLEIKGGGGSGGGGSGGGGSGGGGSEVQL
VESGGGLVQPGGSLRLSCAASGFTFSDYGMAWVRQAPGKGLEWVSFITSLAYIIYYADSVKGRFT
ISRDNAKNSLYLQMNSLRAEDTAVYYCARNYGSSPYYFDYWGQGTLVTVSSSEPKSSDKTHTCPP
CPAPPAAAPSVFLFPPKPKDTLMISRTPEVTCVVVDVSHEDPEVKFNWYVDGVEVHANKTKPREE
QYNSTYRVVSVLTVLHQDWLNGKEYKCAVSNKALPAPIEKTISKAKGQPREPQVYTLPPSRDELT
KNQVSLWCLVKGFYPSDIAVEWESNGQPENNYKTTPPVLDSDGSFFLYSKLTVDKSRWQQGNVFS
CSVMHEALHNHYTQKSLSLSPGGGGSPSQVQLVQSGAEVKKPGASVKVSCKASGYTFTRSTMHWV
RQAPGQGLEWIGYINPSSAYTNYAQKFQGRVTLTADKSTSTAYMELSSLRSEDTAVYYCASPQVH
YDYNGFPYWGQGTLVTVSSGGGGSGGGGSGGGGSGGGGSDIQMTQSPSSLSASVGDRVTITCRAS
SSVSYMNWYQQKPGKAPKRWIYDSSKLASGVPSRFSGSGSGTDFTLTISSLQPEDFATYYCQQWS
RNPPTFGQGTKVEIKRS
>PDL01157_Chain2-SEQ ID NO: 196
DIQMTQSPSSLSASVGDRVTITCRASQDINKFLSWYQQKPGKAPKLLIHYTSSLQSGVPSRFSGS
GSGTDFTLTISSLQPEDFATYYCLQYDNLYTFGQGTLKEIKGGGGSGGGGSGGGGSGGGGSEVQL
VESGGGLVQPGGSLRLSCAASGFTFSDYGMAWVRQAPGKGLEWVSFITSLAYIIYYADSVKGRFT
ISRDNAKNSLYLQMNSLRAEDTAVYYCARNYGSSPYYFDYWGQGTLVTVSSSEPKSSDKTHTCPP
CPAPPAAAPSVFLFPPKPKDTLMISRTPEVTCVVVDVSHEDPEVKFNWYVDGVEVHNAKTKPREE
QYNSTYRVVSVLTVLHQDWLNGKEYKCAVSNKALPAPIEKTISKAKGQPREPQVYTLPPSRDELT
KNQVSLSCAVKGFYPSDIAVEWESNGQPENNYKTTPPVLDSDGSFFLVSKLTVDKSRWQQGNVFS
CSVMHEALHNRFTQKSLSLSPG
>PDL01158_Chain1-SEQ ID NO: 197
DIQMTQSPSSLSASVGDRVTITCRASQDINKFLSWYQQKPGKAPKLLIHYTSSLQSGVPSRFSGS
GSGTDFTLTISSLQPEDFATYYCLQYDNLYTFGQGTKLEIKGGGGSGGGGSGGGGSGGGGSEVQL
VESGGGLVQPGGSLRLSCAASGFTFSDYGMAWVRQAPGKGLEWVSFITSLAYIIYYADSVKGRFT
ISRDNAKNSLYLQMNSLRAEDTAVYYCARNYGSSPYYFDYWGQGTLVTVSSSEPKSSDKTHTCPP
CPAPPAAAPSVFLFPPKPKDTLMISRTPEVTCVVVDVSHEDPEVKFNWYVDGVEVHNAKTKPREE
QYNSTYRVVSVLTVLHQDWLNGKEYKCAVSNKALPAPIEKTISKAKGQPREPQVYTLPPSRDELT
KNQVSLWCLVKGFYPSDIAVEWESNGQPENNYKTTPPVLDSDGSFFLYSKLTVDKSRWQQGNVFS
CSVMHEALHNHYTQKSLSLSPGGGGSPSQVQLVQSGPEVKKPGSSVKVSCKASGYTFSRSTMHWV
RQAPGQGLEWIGYINPSSAYTNYNQKFKDRVTITADKSTSTAYMELSSLRSEDTAVYYCARPQVH
YDYNGFPYWGQGTLVTVSSGGGGSGGGGSGGGGSGGGGSDIQMTQSPSTLSASVGDRVTMTCSAS
SSVSYMNWYQQKPGKAPKRWIYDSSKLASGVPSRFSGSGSGTDYTLTISSLQPDDFATYYCQQWS
RNPPTFGGGTKVEIKRS
>PDL01158_Chain2-SEQ ID NO: 198
DIQMTQSPSSLSASVGDRVTITCRASQDINKFLSWYQQKPGKAPKLLIHYTSSLQSGVPSRFSGS
GSGTDFTLTISSLQPEDFATYYCLQYDNLYTFGQGTKLEIKGGGGSGGGGSGGGGSGGGGSEVQL
VESGGGLVQPGGSLRLSCAASGFTFSDYGMAWVRQAPGKGLEWVSFITSLAYIIYYADSVKGRFT
ISRDNAKNSLYLQMNSLRAEDTAVYYCARNYGSSPYYFDYWGQGTLVTVSSSEPKSSDKTHTCPP
CPAPPAAAPSVFLFPPKPKDTLMISRTPEVTCVVVDVSHEDPEVKFNWYVDGVEVHNAKTKPREE
QYNSTYRVVSVLTVLHQDWLNGKEYKCAVSNKALPAPIEKTISKAKGQPREPQVYTLPPSRDELT
KNQVSLWCLVKGFYPSDIAVEWESNGQPENNYKTTPPVLDSDGSFFLYSKLTVDKSRWQQGNVFS
CSVMHEALHNHYTQKSLSLSPGGGGSPSQVQLVQSGAEVKKPGASVKVSCKASGYTFTRSTMHWV
RQAPGQGLEWIGYINPSSAYTNYAQKFQGRVTLTADKSTSTAYMELSSLRSEDTAVYYCASPQVH
YDYNGFPYWGQGTLVTVSSGGGGSGGGGSGGGGSGGGGSDIQMTQSPSSLSASVGDRVTITCRAS
SSVSYMNWYQQKPGKAPKRWIYDSSKLASGVPSRFSGSGSGTDFTLTISSLQPEDFATYYCQQWS
RNPPTFGQGTKVEIKRS
>PC401020-SEQ ID NO: 199
DIQMTQSPSSLSASVGDRVTITCRASQDINKFLSWYQQKPGKAPKLLIHYTSSLQSGVPSRFSGS
GSGTDFTLTISSLQPEDFATYYCLQYDNLYTFGQGTKLEIKGGGGSGGGGSGGGGSGGGGSEVQL
VESGGGLVQPGGSLRLSCAASGFTFSDYGMAWVRQAPGKGLEWVSFITSLAYIIYYADSVKGRFT
ISRDNAKNSLYLQMNSLRAEDTAVYYCARNYGSSPYYFDYWGQGTLVTVSSEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPSRDELTK
NQVSLTCLVKGFYPSDIAVEWESNGQPENNYKITPPVLDSDGSFFLYSKLTVDKSRWQQGNVFSC
SVMHEALHNHYTQKSLSLSPGGGGSPSQVQLVQSGPEVKKPGSSVKVSCKASGYTFSRSTMHWVR
QAPGQGLEWIGYINPSSAYTNYNQKFKDRVTITADKSTSTAYMELSSLRSEDTAVYYCARPQVHY
DYNGFPYWGQGTLVTVSSGGGGSGGGGSGGGGSGGGGSDIQMTQSPSTLSASVGDRVTMTCSASS
SVSYMNWYQQKPGKAPKRWIYDSSKLASGVPSRFSGSGSGIDYTLTISSLQPDDFATYYCQQWSR
NPPTFGGGTKVEIKRS
>PC401021-SEQ ID NO: 200
DIQMTQSPSSLSASVGDRVTITCRASQDINKFLSWYQQKPGKAPKLLIHYTSSLQSGVPSRFSGS
GSGTDFTLTISSLQPEDFATYYCLQYDNLYTFGQGTKLEIKGGGGSGGGGSGGGGSGGGGSEVQL
VESGGGLVQPGGSLRLSCAASGFTFSDYGMAWVRQAPGKGLEWVSFITSLAYIIYYADSVKGRFT
ISRDNAKNSLYLQMNSLRAEDTAVYYCARNYGSSPYYFDYWGQGTLVTVSSEPKSSDKTHTCPPC
PAPPAAAPSVFLFPPKPKDTLMISRIPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSC
SVMHEALHNHYTQKSLSLSPGGGGSPSEVQLVQSGAEVKKPGASVKVSCKASGYTETSYWMNWVR
QAPGQGLEWMGNIYPSGGSTNYAQKFQGRVTMTVDTSTSTVYMELSSLRSEDTAVYYCASESDGY
YAYAMDYWGQGTLVTVSSGGGGSGGGGSGGGGSGGGGSEIVMTQSPATLSLSPGFRATLSCRASQ
SVSSYLNWYQQKPGQAPRLLIYYASRRHTGIPARFSGSGSGTDFTLTISSLQPEDFAVYYCQQGY
NLPYTFGQGTKVEIK
>PC401026-SEQ ID NO: 201
EVQLVQSGAEVKKPGASVKVSCKASGYTFTSYWMNWVRQAPGQGLEWMGNIYPSGGSTNYAQKFQ
GRVTMTVDTSTSTVYMELSSLRSEDTAVYYCASFSDGYYAYAMDYWGQGTLVTVSSGGGGSGGGG
SGGGGSGGGGSEIVMTQSPATLSLSPGFRATLSCRASQSVSSYLNWYQQKPGQAPRLLIYYASRR
HTGIPARFSGSGSGTDFTLTISSLQPEDFAVYYCQQGYNLPYTFGQGTKVEIKEPKSSDKTHTCP
PCPAPPAAAPSVFLFPPKPKDTLMISRTPEVTCVVVDVSHEDPEVKFNWYVDGVEVHNAKTKPRE
EQYNSTYRVVSVLTVLHQDGLNGKEYKCAVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQQGNVF
SCSVMHEALHNHYTQKSLSLSPGGGGSPSDIQMTQSPSSVSASVGDRVTITCRASQSLLFSVNQK
NYLAWYQQKPGKAPKLLIYWASSLQSGVPSRFSGSGSGTDFTLTISSLQPEDFATYYCQHYYSYP
WTFGQGTKVEIKGGGGSGGGGSGGGGSGGGGSQVQLVQSGAEVKKPGSSVKVSCKASGYTFSTSW
IEWIRQAPGQGLEWMGSILPGSGSTNYAQKFQGRVTITADTSTSTAYMELSSLRSEDTAVYYCAR
GDAGSYVRWYFDHWGQGTLVTVSS
>CD4001101-SEQ ID NO: 202
DIQMTQSPSTLSASVGDRVTITCKSSQSLLFSVNQKNYLAWYQQKPGKAPKLLIYWASTRFSGVP
ARFSGSGSGTEFTLTISSLQPDDFATYYCQHYYSYPWTFGQGTKVEVKGGGGSGGGGSGGGGSGG
GGSQVQLVQSGAEVKKPGASVKVSCKASGYTFSTSWIEWIKQAPGQGLEWIGEILPGSGSTNYNE
NFRNKATFTADTSISTAYMELSRLRSDDTAVYYCARGDDGSYVRWYFDVWGQGTLVTVSS
>CD4001102-SEQ ID NO: 203
DIQMTQSPSSLSASVGDRVTITCKSSQSLLFSVNQKNYLAWYQQKPGKAPKLLIYWASTRFSGVP
SRFSGSGSGTDFTFTISSLQPEDIATYYCQHYYSYPWTFGGGTKVEIKGGGGSGGGGSGGGGSGG
GGSQVQLVQSGAEVKKPGASVKVSCKASGYTFSTSWIEWIKQAPGQGLEWIGEILPGSGSTNYNE
NFRNKATFTADTSISTAYMELSRLRSDDTAVYYCARGDDGSYVRWYFDVWGQGTLVTVSS
>CD4001103-SEQ ID NO: 204
QIVMTQSPGTLSLSPGFRATMTCKSSQSLLFSVNQKNYLAWYQQKPGQAPRLLIYWASTRFSGVP
DRFSGSGSGTDFTLTISRLEPEDAATYYCQHYYSYPWTFGAGTKLEIKGGGGSGGGGSGGGGSGG
GGSQVKLVQSGAEVKKPGASVKVSCKASGYTFSTSWIEWIRQAPGQGLEWIGEILPGSGSTNYAQ
KFQGRATFTADTSTSTAYMELSSLRSEDTAVYYCARGDDGSYVRWYFDVWGQGTTVTVSS
>CD4001134-SEQ ID NO: 205
DIVMTQSPDSLALSLGERATMNCKSSQSLLFSVNQKNYLAWYQQKPGQPPKLLIYWASTRFSGVP
DRFSGSGSGTDFTLTISSLQAEDVAVYYCQHYYSYPWTFGQGTKLEIKGGGGSGGGGSGGGGSGG
GGSQVKLVQSGAEVKKPGASVKVSCKASGYTFSTSWIEWIRQAPGQGLEWIGEILPGSGSTNYAQ
KFQGRATFTADISTSTAYMELSSLRSEDTAVYYCARGDDGSYVRWYFDVWGQGTTVTVSS
>CD4001135-SEQ ID NO: 206
DIVMTQSPDSLALSLGERATMNCKSSQSLLFSVNQKNYLAWYQQKPGQPPKLLIYWASTRFSGVP
DRFSGSGSGTDFTLTISSLQAEDVAVYYCQHYYSYPWTFGQGTKLEIKGGGGSGGGGSGGGGSGG
GGSQVQLVQSGAEVKKPGASVKVSCKASGYTFSTSWIEWVRQAPGQGLEWIGEILPGSGSTNYAQ
KFQGRATFTADTSTSTAYMELSSLRSEDTAVYYCARGDDGSYVRWYFDVWGQGTTVTVSS
>CD4001136-SEQ ID NO: 207
DIVMTQSPDSLALSLGERATMNCKSSQSLLFSVNQKNYLAWYQQKPGQPPKLLIYWASTRFSGVP
DRFSGSGSGTDFTLTISSLQAEDVAVYYCQHYYSYPWTFGQGTKLEIKGGGGSGGGGSGGGGSGG
GGSQVQLVQSGAEVKKPGASVKVSCKASGYTFSTSWMEWVRQAPGQGLEWMGEILPGSGSTNYAQ
KFQGRVTMTRDTSTSTVYMELSSLRSEDTAVYYCARGDDGSYVRWYFDVWGQGTTVTVSS
>CD4001137-SEQ ID NO: 208
QIVMTQSPGTLSLSPGFRATMTCKSSQSLLFSVNQKNYLAWYQQKPGQAPRLLIYWASTRFSGVP
DRFSGSGSGTDFTLTISRLEPEDAATYYCQHYYSYPWTFGAGTKLEIKGGGGSGGGGSGGGGSGG
GGSQVQLVQSGAEVKKPGASVKVSCKASGYTFSTSWMEWVRQAPGQGLEWMGEILPGSGSTNYAQ
KFQGRVTMTRDTSTSTVYMELSSLRSEDTAVYYCARGDDGSYVRWYFDVWGQGTTVTVSS
>CD4001138-SEQ ID NO: 209
DIVMTQSPDSLAVSLGERATINCKSSQSLLFSVNQKNYLAWYQQKPGQPPKLLIYWASTRFSGVP
DRFSGSGSGTDFTLTISSLQAEDVAVYYCQHYYSYPWTFGQGTKVEIKGGGGSGGGGSGGGGSGG
GGSQVQLVQSGAEVKKPGASVKVSCKASGYTFSTSWMEWVRQAPGQGLEWMGEILPGSGSTNYAQ
KFQGRVTMTRDISTSTVYMELSSLRSEDTAVYYCARGDDGSYVRWYFDVWGQGTTVTVSS
>CD4001152-SEQ ID NO: 210
QIVMTQSPDSLALSLGERATMNCKSSQSLLFSVNQKNYLAWYQQKPGQAPRLLIYWASTRFSGVP
DRFSGSGSGTDFTLTISRLEPEDVATYYCQHYYSYPWTFGAGTKLEIKGGGGSGGGGSGGGGSGG
GGSQVQLVQSGAEVKKPGASVKVSCKASGYTFSTSWIEWVRQAPGQGLEWIGEILPGSGSTNYAQ
KFQGRATFTADTSTSTAYMELSSLRSEDTAVYYCARGDDGSYVRWYFDVWGQGTTVTVSS
>CD4001153-SEQ ID NO: 211
DIVMTQSPDSLALSLGERATMNCKSSQSLLFSVNQKNYLAWYQQKPGQAPRLLIYWASTRFSGVP
DRFSGSGSGTDFTLTISRLEPEDVATYYCQHYYSYPWTFGAGTKLEIKGGGGSGGGGSGGGGSGG
GGSQVQLVQSGAEVKKPGASVKVSCKASGYTFSTSWIEWVRQAPGQGLEWIGEILPGSGSTNYAQ
KFQGRATFTADTSTSTAYMELSSLRSEDTAVYYCARGDDGSYVRWYFDVWGQGTTVTVSS
>CD4001154-SEQ ID NO: 212
QIVMTQSPDSLALSLGERATMNCKSSQSLLFSVNQKNYLAWYQQKPGQPPKLLIYWASTRFSGVP
DRFSGSGSGTDFTLTISRLEPEDVATYYCQHYYSYPWTFGAGTKLEIKGGGGSGGGGSGGGGSGG
GGSQVQLVQSGAEVKKPGASVKVSCKASGYTFSTSWIEWVRQAPGQGLEWIGEILPGSGSTNYAQ
KFQGRATFTADISTSTAYMELSSLRSEDTAVYYCARGDDGSYVRWYFDVWGQGTTVTVSS
>CD4001155-SEQ ID NO: 213
QIVMTQSPDSLALSLGERATMNCKSSQSLLFSVNQKNYLAWYQQKPGQAPRLLIYWASTRFSGVP
DRFSGSGSGTDFTLTISRLEPEDVAVYYCQHYYSYPWTFGQGTKLEIKGGGGSGGGGSGGGGSGG
GGSQVQLVQSGAEVKKPGASVKVSCKASGYTFSTSWIEWVRQAPGQGLEWIGEILPGSGSTNYAQ
KFQGRATFTADTSTSTAYMELSSLRSEDTAVYYCARGDDGSYVRWYFDVWGQGTTVTVSS
>CD4001156-SEQ ID NO: 214
QIVMTQSPDSLALSLGERATMNCKSSQSLLFSVNQKNYLAWYQQKPGQAPRLLIYWASTRFSGVP
DRFSGSGSGTDFTLTISSLQAEDVATYYCQHYYSYPWTFGAGTKLEIKGGGGSGGGGSGGGGSGG
GGSQVQLVQSGAEVKKPGASVKVSCKASGYTFSTSWIEWVRQAPGQGLEWIGEILPGSGSTNYAQ
KFQGRATFTADISTSTAYMELSSLRSEDTAVYYCARGDDGSYVRWYFDVWGQGTTVTVSS
>CD4001157-SEQ ID NO: 215
QIVMTQSPDSLAVSLGERATINCKSSQSLLFSVNQKNYLAWYQQKPGQAPRLLIYWASTRFSGVP
DRFSGSGSGTDFTLTISRLEPEDVATYYCQHYYSYPWTFGAGTKVEIKGGGGSGGGGSGGGGSGG
GGSQVQLVQSGAEVKKPGASVKVSCKASGYTFSTSWIEWVRQAPGQGLEWIGEILPGSGSTNYAQ
KFQGRATFTADTSTSTAYMELSSLRSEDTAVYYCARGDDGSYVRWYFDVWGQGTTVTVSS
>CD4001158-SEQ ID NO: 216
QIVMTQSPDSLALSLGERATMNCKSSQSLLFSVNQKNYLAWYQQKPGQAPRLLIYWASTRFSGVP
DRFSGSGSGTDFTLTISRLEPEDVATYYCQHYYSYPWTFGAGTKLEIKGGGGSGGGGSGGGGSGG
GGSQVQLVQSGAEVKKPGASVKVSCKASGYTFSTSWMEWVRQAPGQGLEWMGEILPGSGSTNYAQ
KFQGRVTMTRDISTSTVYMELSSLRSEDTAVYYCARGDDGSYVRWYFDVWGQGTTVTVSS
>CD4001160-SEQ ID NO: 217
QIVMTQSPDSLALSLGERATMNCKSSQSLLFSVNQKNYLAWYQQKPGQPPKLLIYWASTRFSGVP
DRFSGSGSGTDFTLTISRLEPEDVATYYCQHYYSYPWTFGAGTKLEIKGGGGSGGGGSGGGGSGG
GGSQVQLVQSGAEVKKPGASVKVSCKASGYTFSTSWMEWVRQAPGQGLEWMGEILPGSGSTNYAQ
KFQGRVTMTRDTSTSTVYMELSSLRSEDTAVYYCARGDDGSYVRWYFDVWGQGTTVTVSS
>CD4001161-SEQ ID NO: 218
QIVMTQSPDSLALSLGERATMNCKSSQSLLFSVNQKNYLAWYQQKPGQAPRLLIYWASTRFSGVP
DRFSGSGSGTDFTLTISRLEPEDVAVYYCQHYYSYPWTFGQGTKLEIKGGGGSGGGGSGGGGSGG
GGSQVQLVQSGAEVKKPGASVKVSCKASGYTFSTSWMEWVRQAPGQGLEWMGEILPGSGSTNYAQ
KFQGRVTMTRDTSTSTVYMELSSLRSEDTAVYYCARGDDGSYVRWYFDVWGQGTTVTVSS
>CD4001162-SEQ ID NO: 219
QIVMTQSPDSLALSLGERATMNCKSSQSLLFSVNQKNYLAWYQQKPGQAPRELIYWASTRFSGVP
DRFSGSGSGTDFTLTISSLQAEDVATYYCQHYYSYPWTFGAGTKLEIKGGGGSGGGGSGGGGSGG
GGSQVQLVQSGAEVKKPGASVKVSCKASGYTFSTSWMEWVRQAPGQGLEWMGEILPGSGSTNYAQ
KFQGRVTMTRDTSTSTVYMELSSLRSEDTAVYYCARGDDGSYVRWYFDVWGQGTTVTVSS
>CD4001163-SEQ ID NO: 220
QIVMTQSPDSLAVSLGERATINCKSSQSLLFSVNQKNYLAWYQQKPGQAPRLLIYWASTRFSGVP
DRFSGSGSGTDFTLTISRLEPEDVATYYCQHYYSYPWTFGAGTKVEIKGGGGSGGGGSGGGGSGG
GGSQVQLVQSGAEVKKPGASVKVSCKASGYTFSTSWMEWVRQAPGQGLEWMGEILPGSGSTNYAQ
KFQGRVTMTRDTSTSTVYMELSSLRSEDTAVYYCARGDDGSYVRWYFDVWGQGTTVTVSS
>CD4001164-SEQ ID NO: 221
QIVMTQSPGTLSLSPGFRATMTCKSSQSLLFSVNQKNYLAWYQQKPGQAPRLLIYWASTRFSGVP
DRFSGSGSGTDFTLTISRLEPEDAATYYCQHYYSYPWTFGAGTKLEIKGGGGSGGGGSGGGGSGG
GGSQVKLVQSGAEVKKPGASVKVSCKASGYTFSTSWIEWIRQAPGQGLEWIGEILPGSGSTNYAQ
KFQGRATFTADTSTSTAYMELSSLRSEDTAVYYCARGDDGSYVRWYFDVWGQGTTVTVSS
>CD4001171-SEQ ID NO: 222
DIQMTQSPSTLSASVGDRVTITCKSSQSLLFSVNQKNYLAWYQQKPGKAPKLLIYWASTRFSGVP
SRFSGSGSGTDFTLTISSLQPDDEATYYCQHYYSYPWTFGQGTKVEVKGGGGSGGGGSGGGGSGG
GGSQVQLVQSGAELKKPGSSVKVSCKASGYTFSTSWIEWIKQAPGQGLEWIGRILPGSGSVHYNQ
DFKDKATFTADTSINTAYMELSSLRSEDTAVYYCARGDDGSYVRWYFDVWGQGTLVTVSS
>CD4001172-SEQ ID NO: 223
DIVMTQSPDSLAVSLGERATINCKSSQSLLFSVNQKNYLAWYQQKPGQPPKLLIYWASYRFSGVP
DRFSGSGSGTDFTLTISSLQAEDVAVYYCQHYYSYPWTFGGGTKVEIKGGGGSGGGGSGGGGSGG
GGSQVQLVQSGAEVKKPGASVKVSCKASGYTFSTSWIEWIRQAPGQGLEWIGNILPGSGSTNYNE
KFKNRATFTADTSTSTAYMELSSLRSEDTAVYYCARGDDGSYVRWYFDVWGQGTLVTVSS
>CD4001173-SEQ ID NO: 224
EIVMTQSPATLSLSPGFRATLSCRSSQSLLFSVNQKNYIAWYQQKPGQAPRLLIYWASTRFSGVP
DRFSGSGSGTDFTLTISSLEPEDFAVYYCQHYYSYPWTFGQGTKLEIKGGGGSGGGGSGGGGSGG
GGSQVQLVQSGAEVKKPGASVKVSCKATGYTFSTSWIEWIRQAPGQRLEWIGRILPGSGSTNYNE
KFKGKATFTADTSASTAYMELSSLRSEDTAVYYCARGDDGSYVRWYFDFWGQGTLVTVSS
>CD4001174-SEQ ID NO: 225
DIQMTQSPSSLSASVGDRVTITCQSSQSLLFSVNQKNYLAWYQQKPGKAPKLLIYWASTRFSGVP
DRFSGSGSGTDFTFTISSLQPEDIATYYCQHYYSYPWTFGGGTKVEIKGGGGSGGGGSGGGGSGG
GGSQVKLVQSGAEVKKPGASVKVSCKATGYTFSTSWIEWIRQAPGQGLEWIGEILPGSGSTNYNE
KFKGKATFTADTSTSTAYMELSSLRSEDTAVYYCARGDDGSYVRWYFDVWGQGTTVTVSS
>CD4001175-SEQ ID NO: 226
DIQMTQSPSSLSASVGDRVTITCRSSQSLLFSVNQKNYVAWYQQKPGKAPKLLIYWASTRFSGVP
DRFSGSGSGTDFTLTISSLQPEDFATYYCQHYYSYPWTFEQGTKVEIKGGGGSGGGGSGGGGSGG
GGSEVQLVESGGGLVQPGGSLRLSCAATGYTFSTSWIEWIRQAPGKGLEWIGLILPGSGSTNYNE
KFKGKATFSANTSKNTAYLQMNSLRAEDTAVYYCARGDDGSYVRWYFDYWGQGTLVTVSS
>CD4001176-SEQ ID NO: 227
DIQMTQSPSSLSASVGDRVTITCRSSQSLLFSVNQKNYVAWYQQKPGKAPKLLIYWASFLYSGVP
SRFSGSGSGTDFTLTISSLQPEDFATYYCQHYYSYPWTFGQGTKVEIKGGGGSGGGGSGGGGSGG
GGSEVQLVESGGGLVQPGGSLRLSCAASGYTFSTSWIEWIRQAPGKGLEWIGRILPGSGSTNYAD
SVKGRATFSADTSKNTAYLQMNSLRAEDTAVYYCARGDDGSYVRWYFDVWGQGTLVTVSS
>CD4001177-SEQ ID NO: 228
EIVMTQSPDFQSVTPKFKVTITCRSSQSLLFSVNQKNYLAWYQQKPDQSPKLLIYWASQSESGVP
SRFSGSGSGTDFTLTINSLEAEDAAAYYCQHYYSYPWTFGPGTKVDIKGGGGSGGGGSGGGGSGG
GGSQVQLVESGGGVVQPGRSLRLSCAASGYTFSTSWIEWIRQAPAKGLEWIGIILPGSGSTYYAD
SVKGRATFSADTSKNTAYLQMNGLRAEDTAVYYCARGDDGSYVRWYFDVWGQGTLVTVSS
>CD4001178-SEQ ID NO: 229
DIQMTQSPSSLSASVGDRVTITCKSSQSLLFSVNQKNYVAWYQQKPGKAPKLLIYWASYRYTGVP
SRFSGSGSGTDFTLTISSLQPEDFATYYCQHYYSYPWTFGQGTKVEIKGGGGSGGGGSGGGGSGG
GGSEVQLVESGGGLVQPGGSLRLSCAASGYTFSTSWIEWIRQAPGKGLEWIGDILPGSGSTIYNQ
RFKGRATFSADTSKNTAYLQMNSLRAEDTAVYYCARGDDGSYVRWYFDVWGQGTLVTVSS
>CD4001179-SEQ ID NO: 230
DIVMTQSPDSLAVSLGERATINCKSSQSLLFSVNQKNYLAWYQQKPGQPPKLLIYWASTRFSGVP
DRFSGSGSGTDFTLTISSLQAEDVAVYYCQHYYSYPWTFGGGTKLEIKGGGGSGGGGSGGGGSGG
GGSQVQLVQSGAEVKKPGSSVKVSCKASGYTFSTSWIEWIRQAPGQGLEWIGGILPGSGSTNYAQ
KFQGRATFTADTSTSTAYMELSSLRSEDTAVYYCARGDDGSYVRWYFDVWGAGTTVTVSS
>CD4001180-SEQ ID NO: 231
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKATGYTFSTSWIEWIRQAPGQGLEWIGSILPGSGSTNYNE
KFKGKATFTADTSTSTAYMELSSLRSEDTAVYYCARGDDGSYVRWYFDHWGQGTLVTVSS
>CD4001181-SEQ ID NO: 232
DIQMTQSPSSVSASVGDRVTITCRASQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKATGYTFSTSWIEWIRQAPGQGLEWIGSILPGSGSTNYNE
KFKGKATFTADTSTSTAYMELSSLRSEDTAVYYCARGDDGSYVRWYFDHWGQGTLVTVSS
>CD4001182-SEQ ID NO: 233
DIQMTQSPSSVSASVGDRVTITCRSSQSLLFSVNQKNYLAWYQQKPGKAPKLLIYWASSRQSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKATGYTFSTSWIEWIRQAPGQGLEWIGSILPGSGSTNYNE
KFKGKATFTADTSTSTAYMELSSLRSEDTAVYYCARGDDGSYVRWYFDHWGQGTLVTVSS
>CD4001183-SEQ ID NO: 234
DIQMTQSPSSVSASVGDRVTITCRSSQSLLFSVNQKNYLAWYQQKPGKAPKLLIYWASTLESGVP
SRFSGSGSGTDFTLTISSLQPEDFATYYCQHYYSYPWTFGQGTKVEIKGGGGSGGGGSGGGGSGG
GGSQVQLVQSGAEVKKPGSSVKVSCKATGYTFSTSWIEWIRQAPGQGLEWIGSILPGSGSTNYNE
KFKGKATFTADTSTSTAYMELSSLRSEDTAVYYCARGDDGSYVRWYFDHWGQGTLVTVSS
>CD4001184-SEQ ID NO: 235
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWIEWIRQAPGQGLEWIGSILPGSGSTNYNE
KFKGKATFTADTSTSTAYMELSSLRSEDTAVYYCARGDDGSYVRWYFDHWGQGTLVTVSS
>CD4001185-SEQ ID NO: 236
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWISWVRQAPGQGLEWIGGILPGSGSTNYNE
KFKGKATFTADTSTSTAYMELSSLRSEDTAVYYCARGDDGSYVRWYFDHWGQGTLVTVSS
>CD4001186-SEQ ID NO: 237
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWIEWIRQAPGQGLEWMGSILPGSGSTNYNE
KFKGKVTITADTSTSTAYMELSSLRSEDTAVYYCARGDDGSYVRWYFDHWGQGTLVTVSS
>CD4001187-SEQ ID NO: 238
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWIEWIRQAPGQGLEWIGSILPGSGSTNYAE
KFKGKATFTADTSTSTAYMELSSLRSEDTAVYYCARGDDGSYVRWYFDHWGQGTLVTVSS
>CD4001188-SEQ ID NO: 239
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWIEWIRQAPGQGLEWIGSILPGSGSTNYNQ
KFQGRATFTADISTSTAYMELSSLRSEDTAVYYCARGDDGSYVRWYFDHWGQGTLVTVSS
>CD4001189-SEQ ID NO: 240
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWIEWIRQAPGQGLEWIGSILPGSGSTNYNE
KFKGKATFTADESTSTAYMELSSLRSEDTAVYYCARGDDGSYVRWYFDHWGQGTLVTVSS
>CD4001190-SEQ ID NO: 241
DIQMTQSPSSVSASVGDRVTITCRASQSLLFSVNQKNYLAWYQQKPGKAPKLLIYWASSRQSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKATGYTFSTSWIEWIRQAPGQGLEWIGSILPGSGSTNYNE
KFKGKATFTADISTSTAYMELSSLRSEDTAVYYCARGDDGSYVRWYFDHWGQGTLVTVSS
>CD4001191-SEQ ID NO: 242
DIQMTQSPSSVSASVGDRVTITCRSSQSLLFSVNQKNYLAWYQQKPGKAPKLLIYWASSLQSGVP
SRFSGSGSGTDFTLTISSLQPEDFATYYCQHYYSYPWTFGQGTKVEIKGGGGSGGGGSGGGGSGG
GGSQVQLVQSGAEVKKPGSSVKVSCKATGYTFSTSWIEWIRQAPGQGLEWIGSILPGSGSTNYNE
KFKGKATFTADTSTSTAYMELSSLRSEDTAVYYCARGDDGSYVRWYFDHWGQGTLVTVSS
>CD4001192-SEQ ID NO: 243
DIQMTQSPSSVSASVGDRVTITCRASQSLLFSVNQKNYLAWYQQKPGKAPKLLIYWASTLESGVP
SRFSGSGSGTDFTLTISSLQPEDFATYYCQHYYSYPWTFGQGTKVEIKGGGGSGGGGSGGGGSGG
GGSQVQLVQSGAEVKKPGSSVKVSCKATGYTFSTSWIEWIRQAPGQGLEWIGSILPGSGSTNYNE
KFKGKATFTADISTSTAYMELSSLRSEDTAVYYCARGDDGSYVRWYFDHWGQGTLVTVSS
>CD4001193-SEQ ID NO: 244
DIQMTQSPSSVSASVGDRVTITCRASQSLLFSVNQKNYLAWYQQKPGKAPKLLIYWASSLQSGVP
SRFSGSGSGTDFTLTISSLQPEDFATYYCQHYYSYPWTFGQGTKVEIKGGGGSGGGGSGGGGSGG
GGSQVQLVQSGAEVKKPGSSVKVSCKATGYTFSTSWIEWIRQAPGQGLEWIGSILPGSGSTNYNE
KFKGKATFTADISTSTAYMELSSLRSEDTAVYYCARGDDGSYVRWYFDHWGQGTLVTVSS
>CD4001194-SEQ ID NO: 245
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWISWVRQAPGQGLEWMGGILPGSGSTNYNE
KFKGKVTITADISTSTAYMELSSLRSEDTAVYYCARGDDGSYVRWYEDHWGQGTLVTVSS
>CD4001195-SEQ ID NO: 246
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWISWVRQAPGQGLEWIGGILPGSGSTNYAE
KFKGKATFTADISTSTAYMELSSLRSEDTAVYYCARGDDGSYVRWYFDHWGQGTLVTVSS
>CD4001196-SEQ ID NO: 247
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWISWVRQAPGQGLEWIGGILPGSGSTNYNQ
KFQGRATFTADISTSTAYMELSSLRSEDTAVYYCARGDDGSYVRWYFDHWGQGTLVTVSS
>CD4001197-SEQ ID NO: 248
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWISWVRQAPGQGLEWIGGILPGSGSTNYNE
KFKGKATFTADESTSTAYMELSSLRSEDTAVYYCARGDDGSYVRWYFDHWGQGTLVTVSS
>CD4001198-SEQ ID NO: 249
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWIEWIRQAPGQGLEWMGSILPGSGSTNYAE
KFKGKVTITADTSTSTAYMELSSLRSEDTAVYYCARGDDGSYVRWYFDHWGQGTLVTVSS
>CD4001199-SEQ ID NO: 250
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWIEWIRQAPGQGLEWMGSILPGSGSTNYNQ
KFQGRVTITADTSTSTAYMELSSLRSEDTAVYYCARGDDGSYVRWYFDHWGQGTLVTVSS
>CD4001200-SEQ ID NO: 251
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWIEWIRQAPGQGLEWIGSILPGSGSTNYAQ
KFQGRATFTADTSTSTAYMELSSLRSEDTAVYYCARGDDGSYVRWYFDHWGQGTLVTVSS
>CD4001201-SEQ ID NO: 252
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWIEWIRQAPGQGLEWIGSILPGSGSTNYAE
KFKGKATFTADESTSTAYMELSSLRSEDTAVYYCARGDDGSYVRWYFDHWGQGTLVTVSS
>CD4001202-SEQ ID NO: 253
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWIEWIRQAPGQGLEWIGSILPGSGSTNYNQ
KFQGRATFTADESTSTAYMELSSLRSEDTAVYYCARGDDGSYVRWYFDHWGQGTLVTVSS
>CD4001203-SEQ ID NO: 254
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWISWVRQAPGQGLEWMGGILPGSGSTNYAE
KFKGKVTITADISTSTAYMELSSLRSEDTAVYYCARGDDGSYVRWYFDHWGQGTLVTVSS
>CD4001204-SEQ ID NO: 255
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWISWVRQAPGQGLEWMGGILPGSGSTNYNQ
KFQGRVTITADTSTSTAYMELSSLRSEDTAVYYCARGDDGSYVRWYFDHWGQGTLVTVSS
>CD4001205-SEQ ID NO: 256
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWISWVRQAPGQGLEWMGGILPGSGSTNYNE
KFKGKVTITADESTSTAYMELSSLRSEDTAVYYCARGDDGSYVRWYFDHWGQGTLVTVSS
>CD4001206-SEQ ID NO: 257
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWIEWIRQAPGQGLEWMGSILPGSGSTNYAQ
KFQGRVTITADTSTSTAYMELSSLRSEDTAVYYCARGDDGSYVRWYEDHWGQGTLVTVSS
>CD4001207-SEQ ID NO: 258
DIQMTQSPSSVSASVGDRVTITCRSSQSLLFSVNQKNYLAWYQQKPGKAPKLLIYWASTRFSGVP
DRFSGSGSGTDFTLTISSLQPEDFATYYCQHYYSYPWTFGQGTKVEIKGGGGSGGGGSGGGGSGG
GGSQVQLVQSGAEVKKPGSSVKVSCKASGYTFSTSWIEWIRQAPGQGLEWMGSILPGSGSTNYAE
KFKGKVTITADESTSTAYMELSSLRSEDTAVYYCARGDDGSYVRWYFDHWGQGTLVTVSS
>Humanized1D5_VH_DNA-SEQ ID NO: 259
CAGGTTCAACTGGTGCAAAGCGGGGCTGAGGTAAAAAAGCCCGGTAGCTCTGTGAAGGTGTCTTG
TAAAGCCAGCGGATATACCTTTTCAACTTCCTGGATTGAGTGGATAAGGCAAGCCCCAGGGCAGG
GGCTGGAGTGGATGGGCTCTATTCTGCCTGGGAGCGGATCAACTAATTATGCCCAGAAATTTCAA
GGTCGGGTTACTATAACAGCAGACACCTCTACAAGTACCGCCTACATGGAGCTGAGTTCCTTGCG
ATCTGAGGATACAGCCGTATATTATTGTGCAAGGGGTGACGACGGAAGCTACGTCCGATGGTACT
TCGATCATTGGGGACAGGGAACTCTCGTAACCGTATCATCT
>Humanized1D5_VL_DNA-SEQ ID NO: 260
GATATACAGATGACCCAATCCCCTAGTTCCGTCTCAGCTTCAGTTGGCGACAGAGTAACTATTAC
ATGCCGTGCTAGCCAATCCCTTCTTTTCTCAGTGAATCAGAAAAACTATCTGGCATGGTATCAAC
AAAAGCCCGGCAAGGCCCCCAAGCTCCTCATTTACTGGGCTAGCTCATTGCAGTCAGGAGTTCCA
AGCCGTTTTTCCGGGTCAGGGTCTGGCACCGATTTTACTTTGACCATCAGTTCTTTGCAACCCGA
AGACTTTGCTACATATTACTGTCAACATTACTATAGTTATCCCTGGACATTTGGACAAGGAACCA
AAGTCGAGATTAAA
>PDL01041_2C11LH-SEQ ID NO: 261
DIQMTQSPASLSASVGETVTITCRASENIYSFLAWYQQKQGKSPQLLVYKAKTLTEGVPSRFSGS
GSGTQFSLKINSLQPEDFGSYYCQHHYGSPPETFGGGTKVEIKGGGGSGGGGSGGGGSGGGGSDV
KLVESGGGLVKPGGSLKLSCAASGFTFSSYTMSWVRQTPEKRLEWVATISSGGNYTYYPDSVKGR
FTISRDNAKNTLYLQLSSLKSEDTAMYYCTRDQAYYGNLFTYWGQGTLVTVSA
>PDL01042_3G5LH-SEQ ID NO: 262
DIQMTQTTSSLSASLGDRVTISCSASQGIRNYLNWYQQKPDGTVKLLIYYTSTLHSGVPSRFSGS
GSGIDYSLTISNLEPEDIATYYCQQYSKLPWTFGGGTRLEIKGGGGSGGGGSGGGGSGGGGSQIQ
LQQSGPELVKPGASVKISCKASGYTFTDYYINWVKQKPEQGLEWIGWTFPGSINTKYNEKFKGKA
TLTVDTSSSTAYMQINSLTSEDTAVYFCARSPYEFDYWGQGTTLTVSS
>PDL01043_3G11LH-SEQ ID NO: 263
DIQMTQTTSSLSASLGDRVTISCSASQGIRNYLNWYQQKPDGTVKLLIYYTSTLHSGVPSRFSGS
GSGTDYSLTISNLEPEDIATYYCQQYSKLPWTFGGGTRLEIKGGGGSGGGGSGGGGSGGGGSEVQ
LQESGPSLVKPSQTLSLTCSVTGDSITSGYWNWIRKFPGKKLEYMGYISYTGSTYYNPSLKSRIS
IARDTSKNQYFLQLNSVTTEDTATYYCTRGENWDGGYFDYWGQGTTLTVSS
>PDL01045_6F6LH-SEQ ID NO: 264
DIVMTQSHKFMSTSVGDRVTITCKASQDVGTAVAWYQKKPGQSPKLLISWASTRHTGVPDRFTGS
GSGTDFTLTISNVQSEDLADYFCQQYSSYPYTFGGGTNLEIKGGGGSGGGGSGGGGSGGGGSQGQ
MQQSGAELVKPGASVKLSCKTSGFTFSSSYITWLKQKPGQSLEWIAWIYVGTGGISYNQKFTGKA
QLTVDTSSSTAYMQFSSLTTEDSAIYYCARHGRYHWYFDVWGAGTTVTVSS
>PDL01047_7A3-2LH-SEQ ID NO: 265
DVQITQSPSYLAASPGFTITINCRASKSISKYLAWYQEKPGKINKLLIYSGSTLQSGIPSRFSGS
GSGTDFTLTISSLEPEDFAMYYCQQHNEYPFTFGAGTKLEIKGGGGSGGGGSGGGGSGGGGSEVQ
LQESGPSLVKPSQTLSLTCSVTGDSITSGYWNWIRKFPGNRLEYMGFRSYSGTTYYNPSLKSRIS
ITRDTSKNQYYLQLNSVTTEDTATYYCARESDFGSSENTMDYWGQGTSVTVSS
>PDL01048_7H11LH-SEQ ID NO: 266
DIVLTQSPASLAVSLGQRATISCRASKSVSKSGYSYLHWYQQKPGQTPKLLIYLASNLESGVPAR
FSGSGSGTDFTLNIHPVEEEDAATYYCQHSWELPYTFGGGTKLEIKGGGGSGGGGSGGGGSGGGG
SEVQLQESGPSLVKPSQTLSLTCSVTGDSITSGYWNWIRKFPGKKLEYMGYISYTGSTYYNPSLK
SRISIARDTSKNQYFLQLNSVTTEDTATYYCTRGENWDGGYFDYWGQGTTLTVSS
>PDL01049_10B2LH-SEQ ID NO: 267
DIQMTQTTSSLSASLGDRVTISCRASQDIRNYLNWYQQKPDGTVKLLIYYTSRLQSGVPSRFSGS
GSGTDYSLTITTLEQEDFATYFCQQGNMFPYTFGGGTKLEIKGGGGSGGGGSGGGGSGGGGSKVQ
LQQSGAEVVKPGASVKLSCKASGYNLTEYIIHWVKQRSGQGLEWIGWFYPGSGSIKYNEKFKDKA
TLTADKSSTTVYMDLSRLTSEDSAVYFCARHGLYYGFPYWGQGTSVTVSS
>PDL01050_LightChain_5F11chimeric_mAb-SEQ ID NO: 268
DIQMTQSPSSLSASLGGKVTITCKASQDINKYIAWYQHKPGKGPRLLIHYTSILQPGIPSRFSGS
GSGRDYSFSISNLEPEDIATYYCLQYDNLRTFGGGTKLEIKRIVAAPSVFIFPPSDEQLKSGTAS
VVCLLNNFYPREAKVQWKVDNALQSGNSQESVTEQDSKDSTYSLSSTLTLSKADYEKHKVYACEV
THQGLSSPVTKSFNRGEC
>PDL01050_HeavyChain_5F11chimeric_mAb-SEQ ID NO: 269
EVKFEESGGGLVQPGGSMKLSCFASGFTFSYYWMNWVRQSPEKGLEWIAEIRLKSNNYATHYAES
VKGRFTISRDDSKSSVYLEMNNLRAEDTGIYHCTRPPIYYGNYEAYWGQGTLVTVSAASTKGPSV
FPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSLSLSPGK
>PDL01051_LightChain_7A3-1chimeric_mAb-SEQ ID NO: 270
DIVMTQSHKFMSTSVGDRVTITCKASQDVGTAVAWYQKKPGQSPKLLISWASTRHTGVPDRFTGS
GSGTDFTLTISNVQSEDLADYFCQQYSSYPYTFGGGTNLEIKRTVAAPSVFIFPPSDEQLKSGTA
SVVCLLNNFYPREAKVQWKVDNALQSGNSQESVTEQDSKDSTYSLSSTLTLSKADYEKHKVYACE
VTHQGLSSPVTKSFNRGEC
>PDL01051_HeavyChain_7A3-1chimeric_mAb-SEQ ID NO: 271
EVQLQESGPSLVKPSQTLSLTCSVTGDSITSGYWNWIRKFPGNRLEYMGFRSYSGTTYYNPSLKS
RISITRDTSKNQYYLQLNSVTTEDTATYYCARFSDFGSSENTMDYWGQGTSVTVSSASTKGPSVF
PLAPSSKSTSGGTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
>PC401025-SEQ ID NO: 272
DIQMTQSPSSLSASVGDRVTINCQASQSIDSNLAWFQQKPGQPPKLLIYRASNLASGVPDRFSGS
GSGTDFTLTISSLEAEDVATYYCLGGVGAVSYRTSFGGGTKVEIKGGGGSGGGGSGGGGSGGGGS
EVQLVESGGGLVQPGRSLRLSCTASGSDINDYPITWVRQAPGQGLEWIGFINSGGSTWYASWVKG
RFTISRDDSKSIAYLQMNSLKTEDTAVYYCARGYSTYYRDFNIWGQGTLVTVSSSEPKSSDKTHT
CPPCPAPPAAAPSVFLFPPKPKDTLMISRTPEVTCVVVDVSHEDPEVKFNWYVDGVEVHNAKTKP
REEQYNSTYRVVSVLTVLHQDGLNGKEYKCAVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGN
VFSCSVMHEALHNHYTQKSLSLSPGGGGSPSDIQMTQSPSSVSASVGDRVTITCRASQSLLFSVN
QKNYLAWYQQKPGKAPKLLIYWASSLQSGVPSRFSGSGSGTDFTLTISSLQPEDFATYYCQHYYS
YPWTFGQGTKVEIKGGGGSGGGGSGGGGSGGGGSQVQLVQSGAEVKKPGSSVKVSCKASGYTFST
SWIEWIRQAPGQGLEWMGSILPGSGSTNYAQKFQGRVTITADTSTSTAYMELSSLRSEDTAVYYC
ARGDAGSYVRWYFDHWGQGTLVTVSS
>PC401027-SEQ ID NO: 273
DILLTQSPVILSVSPGFRVSFSCRASQSLGTNIHWYQQRTNGSPRLLIKYASESISGIPSRFSGS
GSGTDFTLSINSVESEDIADYYCQQNNNWPTTFGAGTKLELKGGGGSGGGGSGGGGSQVQLKQSG
PGLVQPSQSLSITCTVSGFSLTNYGVHWVRQSPGKGLEWLGVIWSGGNTDYNTPFTSRLSINKDN
SKSQVFFKMNSLQSNDTAIYYCARALTYYDYEFAYWGQGTLVTVSSSEPKSSDKTHTCPPCPAPP
AAAPSVFLFPPKPKDTLMISRTPEVTCVVVDVSHEDPEVKFNWYVDGVEVHNAKTKPREEQYNST
YRVVSVLTVLHQDGLNGKEYKCAVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSVMH
EALHNHYTQKSLSLSPGGGGSPSDIQMTQSPSSVSASVGDRVTITCRASQSLLFSVNQKNYLAWY
QQKPGKAPKLLIYWASSLQSGVPSRFSGSGSGTDFTLTISSLQPEDFATYYCQHYYSYPWTFGQG
TKVEIKGGGGSGGGGSGGGGSGGGGSQVQLVQSGAEVKKPGSSVKVSCKASGYTFSTSWIEWIRQ
APGQGLEWMGSILPGSGSTNYAQKFQGRVTITADTSTSTAYMELSSLRSEDTAVYYCARGDAGSY
VRWYFDHWGQGTLVTVSS
>PC401035-SEQ ID NO: 274
DIQMTQSPSSLSASVGDRVTINCQASQSIDSNLAWFQQKPGQPPKLLIYRASNLASGVPDRFSGS
GSGTDFTLTISSLEAEDVATYYCLGGVGAVSYRTSFGGGTKVEIKGGGGSGGGGSGGGGSGGGGS
EVQLVESGGGLVQPGRSLRLSCTASGSDINDYPITWVRQAPGQGLEWIGFINSGGSTWYASWVKG
RFTISRDDSKSIAYLQMNSLKTEDTAVYYCARGYSTYYRDENIWGQGTLVTVSSSEPKSSDKTHT
CPPCPAPPAAAPSVFLFPPKPKDTLMISRTPEVTCVVVDVSHEDPEVKFNWYVDGVEVHNAKTKP
REEQYNSTYRVVSVLTVLHQDGLNGKEYKCAVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGN
VFSCSVMHEALHNHYTQKSLSLSPGGGGSPSDIQMTQSPSSVSASVGDRVTITCRASQSLLFSVN
QKNYLAWYQQKPGKAPKLLIYWASSLQSGVPSRFSGSGSGTDFTLTISSLQPEDFATYYCQHYYS
YPWTFGQGTKVEIKGGGGSGGGGSGGGGSGGGGSQVQLVQSGAEVKKPGSSVKVSCKASGYTFST
SWIEWIRQAPGQGLEWMGSILPGSGSTNYAQKFQGRVTITADTSTSTAYMELSSLRSEDTAVYYC
ARGDDGSYVRWYFDHWGQGTLVTVSS
>PC401037-SEQ ID NO: 275
DILLTQSPVILSVSPGFRVSFSCRASQSLGINIHWYQQRTNGSPRLLIKYASESISGIPSRFSGS
GSGTDFTLSINSVESEDIADYYCQQNNNWPTTFGAGTKLELKGGGGSGGGGSGGGGSQVQLKQSG
PGLVQPSQSLSITCTVSGFSLINYGVHWVRQSPGKGLEWLGVIWSGGNTDYNTPFTSRLSINKDN
SKSQVFFKMNSLQSNDTAIYYCARALTYYDYEFAYWGQGTLVTVSSSEPKSSDKTHTCPPCPAPP
AAAPSVFLFPPKPKDTLMISRTPEVTCVVVDVSHEDPEVKFNWYVDGVEVHNAKTKPREEQYNST
YRVVSVLTVLHQDGLNGKEYKCAVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSVMH
EALHNHYTQKSLSLSPGGGGSPSDIQMTQSPSSVSASVGDRVTITCRASQSLLFSVNQKNYLAWY
QQKPGKAPKLLIYWASSLQSGVPSRFSGSGSGTDFTLTISSLQPEDFATYYCQHYYSYPWTFGQG
TKVEIKGGGGSGGGGSGGGGSGGGGSQVQLVQSGAEVKKPGSSVKVSCKASGYTFSTSWIEWIRQ
APGQGLEWMGSILPGSGSTNYAQKFQGRVTITADISTSTAYMELSSLRSEDTAVYYCARGDDGSY
VRWYFDHWGQGTLVTVSS
>PC401015-SEQ ID NO: 490
DIQMTQSPSSLSASVGDRVTITCRASQDINKFLSWYQQKPGKAPKLLIHYTSSLQSGVPSRFSGS
GSGTDFTLTISSLQPEDFATYYCLQYDNLYTFGQGTKLEIKGGGGSGGGGSGGGGSGGGGSEVQL
VESGGGLVQPGGSLRLSCAASGFTFSDYGMAWVRQAPGKGLEWVSFITSLAYIIYYADSVKGRFT
ISRDNAKNSLYLQMNSLRAEDTAVYYCARNYGSSPYYFDYWGQGTLVTVSSEPKSSDKTHTCPPC
PAPPAAAPSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSC
SVMHEALHNHYTQKSLSLSPG
>CD4001272-SEQ ID NO: 491
EPKSSDKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKITPPVLDSDGSFFLYSKL
TVDKSRWQQGNVFSCSVMHEALHNHYTQKSLSLSPGGGGSPSDIQMTQSPSSVSASVGDRVTITC
RASQSLLFSVNQKNYLAWYQQKPGKAPKLLIYWASSLQSGVPSRFSGSGSGTDFTLTISSLQPED
FATYYCQHYYSYPWTFGQGTKVEIKGGGGSGGGGSGGGGSGGGGSQVQLVQSGAEVKKPGSSVKV
SCKASGYTFSTSWIEWIRQAPGQGLEWMGSILPGSGSTNYAQKFQGRVTITADTSTSTAYMELSS
LRSEDTAVYYCARGDDGSYVRWYFDHWGQGTLVTVSS
>CD4001242-SEQ ID NO: 492
EPKSSDKTHTCPPCPAPPAAAPSVFLFPPKPKDTLMISRTPEVTCVVVDVSHEDPEVKFNWYVDG
VEVHNAKTKPREEQYNSTYRVVSVLTVLHQDWLNGKEYKCAVSNKALPAPIEKTISKAKGQPREP
QVYTLPPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPGGGGSPSDIQMTQSPSSVSASVGDRVTITCR
ASQSLLFSVNQKNYLAWYQQKPGKAPKLLIYWASSLQSGVPSRFSGSGSGTDFTLTISSLQPEDF
ATYYCQHYYSYPWTFGQGTKVEIKGGGGSGGGGSGGGGSGGGGSQVQLVQSGAEVKKPGSSVKVS
CKASGYTFSTSWIEWIRQAPGQGLEWMGSILPGSGSTNYAQKFQGRVTITADTSTSTAYMELSSL
RSEDTAVYYCARGDDGSYVRWYFDHWGQGTLVTVSS
>CD4001085_Light-Chain-SEQ ID NO: 493
DIQMTQSPSSVSASVGDRVTITCRASQGIYSWLAWYQQKPGKAPNLLIYTASTLQSGVPSRFSGS
GSGTDFTLTISSLQPEDFATYYCQQANIFPLTFGGGTKVEIKRTVAAPSVFIFPPSDEQLKSGTA
SVVCLLNNFYPREAKVQWKVDNALQSGNSQESVTEQDSKDSTYSLSSTLTLSKADYEKHKVYACE
VTHQGLSSPVTKSFNRGEC
>CD4001085_Heavy-Chain-SEQ ID NO: 494
QVQLVQSGAEVKKPGASVKVSCKASGYTFTGYYMHWVRQAPGQGLEWMGWINPDSGGTNYAQKFQ
GRVTMTRDTSISTAYMELNRLRSDDTAVYYCARDQPLGYCTNGVCSYFDYWGQGTLVTVSSASTK
GPSVFPLAPCSRSTSESTAALGCLVKDYFPEPVTVSWNSGALTSGVHTFPAVLQSSGLYSLSSVV
TVPSSNFGTQTYTCNVDHKPSNTKVDKTVERKCCVECPPCPAPPVAGPSVFLFPPKPKDTLMISR
TPEVTCVVVDVSHEDPEVQFNWYVDGVEVHNAKTKPREEQFNSTERVVSVLTVVHQDWLNGKEYK
CKVSNKGLPAPIEKTISKTKGQPREPQVYTLPPSREEMTKNQVSLTCLVKGFYPSDIAVEWESNG
QPENNYKTTPPMLDSDGSFFLYSKLTVDKSRWQQGNVFSCSVMHEALHNHYTQKSLSLSPGK

Provided herein is a CD40 binding polypeptide that binds specifically to human CD40; wherein the CD40 binding polypeptide comprises, in order from amino terminus to carboxyl terminus or from carboxyl terminus to amino terminus, (a) a first binding domain; (b) a hinge region; (c) an immunoglobulin constant region; and (d) a second binding domain; wherein the first binding domain is a CD40 binding domain and the second binding domain binds a tumor-associated antigen; or wherein the first binding domain binds a tumor-associated antigen and the second binding domain is a CD40 binding domain. The first binding domain, the second binding domain, or both binding domains may be an scFv. In some embodiments, the tumor-associated antigen is PD-L1, ROR1, or EGFR.

In some embodiments, the CD40 binding domain comprises (i) an immunoglobulin heavy chain variable region (VH) comprising HCDR1, HCDR2, and HCDR3; and (ii) an immunoglobulin light chain variable region (VL) comprising LCDR1, LCDR2, and LCDR3. Exemplary combinations of regions of CD40 binding domain sequences are provided in Table 3.

In some embodiments, (a) the HCDR1 comprises SEQ ID NO: 10, the HCDR2 comprises SEQ ID NO: 11, and the HCDR3 comprises SEQ ID NO: 12, the LCDR1 comprises SEQ ID NO: 14, the LCDR2 comprises SEQ ID NO: 15, and the LCDR3 comprises SEQ ID NO: 16; (b) the HCDR1 comprises SEQ ID NO: 10, the HCDR2 comprises SEQ ID NO: 23, and the HCDR3 comprises SEQ ID NO: 12, the LCDR1 comprises SEQ ID NO: 14, the LCDR2 comprises SEQ ID NO: 15, and the LCDR3 comprises SEQ ID NO: 16; (c) the HCDR1 comprises SEQ ID NO: 10, the HCDR2 comprises SEQ ID NO: 11, and the HCDR3 comprises SEQ ID NO: 31, the LCDR1 comprises SEQ ID NO: 14, the LCDR2 comprises SEQ ID NO: 15, and the LCDR3 comprises SEQ ID NO: 16; (d) the HCDR1 comprises SEQ ID NO: 10, the HCDR2 comprises SEQ ID NO: 23, and the HCDR3 comprises SEQ ID NO: 31, the LCDR1 comprises SEQ ID NO: 14, the LCDR2 comprises SEQ ID NO: 15, and the LCDR3 comprises SEQ ID NO: 16; (e) the HCDR1 comprises SEQ ID NO: 10, the HCDR2 comprises SEQ ID NO: 11, and the HCDR3 comprises SEQ ID NO: 12, the LCDR1 comprises SEQ ID NO: 37, the LCDR2 comprises SEQ ID NO: 15, and the LCDR3 comprises SEQ ID NO:16; (f) the HCDR1 comprises SEQ ID NO: 10, the HCDR2 comprises SEQ ID NO: 11, and the HCDR3 comprises SEQ ID NO: 12, the LCDR1 comprises SEQ ID NO: 41, the LCDR2 comprises SEQ ID NO: 15, and the LCDR3 comprises SEQ ID NO: 16; (g) the HCDR1 comprises SEQ ID NO: 10, the HCDR2 comprises SEQ ID NO: 45, and the HCDR3 comprises SEQ ID NO: 12, the LCDR1 comprises SEQ ID NO: 14, the LCDR2 comprises SEQ ID NO: 15, and the LCDR3 comprises SEQ ID NO: 16; (h) the HCDR1 comprises SEQ ID NO: 10, the HCDR2 comprises SEQ ID NO: 49, and the HCDR3 comprises SEQ ID NO: 12, the LCDR1 comprises SEQ ID NO: 14, the LCDR2 comprises SEQ ID NO: 15, and the LCDR3 comprises SEQ ID NO: 16; or (i) the HCDR1 comprises SEQ ID NO: 10, the HCDR2 comprises SEQ ID NO: 23, and the HCDR3 comprises SEQ ID NO: 31, the LCDR1 comprises SEQ ID NO: 37, the LCDR2 comprises SEQ ID NO: 15, and the LCDR3 comprises SEQ ID NO: 16.

In some embodiments, (a) the VH comprises SEQ ID NO: 13; and the VL comprises SEQ ID NO: 17; (b) the VH comprises SEQ ID NO: 24; and the VL comprises SEQ ID NO: 17; (c) the VH comprises SEQ ID NO: 27; and the VL comprises SEQ ID NO: 28; (d) the VH comprises SEQ ID NO: 32; and the VL comprises SEQ ID NO: 17; (e) the VH comprises SEQ ID NO:34; and the VL comprises SEQ ID NO: 17; (f) the VH comprises SEQ ID NO: 13; and the VL comprises SEQ ID NO: 38; (g) the VH comprises SEQ ID NO: 13; and the VL comprises SEQ ID NO: 42; (h) the VH comprises SEQ ID NO: 46; and the VL comprises SEQ ID NO: 17; (i) the VH comprises SEQ ID NO. 50, and the VL comprises SEQ ID NO: 17; (j) the VH comprises SEQ ID NO: 484; and the VL comprises SEQ ID NO: 485; or (k) the VH comprises SEQ ID NO: 484; and the VL comprises SEQ ID NO: 28.

In some embodiments, the CD40 binding domain comprises SEQ ID NO: 18, SEQ ID NO: 25. SEQ ID NO: 29. SEQ ID NO: 33. SEQ ID NO: 35, SEQ ID NO: 39, SEQ ID NO: 43. SEQ ID NO: 47, SEQ ID NO: 51; SEQ ID NO: 486, or SEQ ID NO: 488. In some embodiments, the CD40 binding domain comprises an amino acid sequence having at least 95%, 96%, 97%, 98%, or 99% sequence identity to any one of SEQ ID NO: 18, SEQ ID NO: 25, SEQ ID NO: 29, SEQ ID NO: 33, SEQ ID NO: 35, SEQ ID NO; 39, SEQ ID NO: 43. SEQ ID NO: 47. SEQ ID NO: 51; SEQ ID NO; 486, and SEQ ID NO: 488.

In some embodiments, the second binding domain comprises (i) an immunoglobulin heavy chain variable region (VH) comprising HCDR1, HCDR2, and HCDR3; and (ii) an immunoglobulin light chain variable region (VL) comprising LCDR1, LCDR2, and LCDR3.

TABLE 3
SEQ ID NOs of CD40 binding domain sequence regions

| Name | HCDR1 | HCDR2 | HCDR3 | VH | LCDR1 | LCDR2 | LCDR3 | VL |
|---|---|---|---|---|---|---|---|---|
| PC401003 | 10 | 11 | 12 | 13 | 14 | 15 | 16 | 17 |
| PC401119 | 10 | 23 | 12 | 24 | 14 | 15 | 16 | 17 |
| PC401120 | 10 | 11 | 12 | 27 | 14 | 15 | 16 | 28 |
| PC401122 | 10 | 11 | 31 | 32 | 14 | 15 | 16 | 17 |
| PC401124 | 10 | 23 | 31 | 34 | 14 | 15 | 16 | 17 |
| PC401128 | 10 | 11 | 12 | 13 | 37 | 15 | 16 | 38 |
| PC401129 | 10 | 11 | 12 | 13 | 41 | 15 | 16 | 42 |
| PC401132 | 10 | 45 | 12 | 46 | 14 | 15 | 16 | 17 |
| PC401133 | 10 | 49 | 12 | 50 | 14 | 15 | 16 | 17 |
| PC401144 | 10 | 23 | 31 | 484 | 37 | 15 | 16 | 485 |
| PC401145 | 10 | 23 | 31 | 484 | 14 | 15 | 16 | 28 |

Provided herein are binding polypeptides that bind specifically to human PD-L1 and human CD40. Provided herein is a binding polypeptide that binds specifically to human PD-L1 and human CD40; wherein the binding polypeptide comprises, in order from amino terminus to carboxyl terminus or from carboxyl terminus to amino terminus, (a) a first binding domain; (b) a hinge region; (c) an immunoglobulin constant region; and (d) a second binding domain; wherein the first binding domain is a PD-L1 binding domain and the second binding domain is a CD40 binding domain; or wherein the first binding domain is a CD40 binding domain and the second binding domain is a PD-L1 binding domain.

Provided herein is a binding polypeptide that binds specifically to human PD-L1 and human CD40; wherein the binding polypeptide comprises, in order from amino terminus to carboxyl terminus, (a) a first binding domain; (b) a hinge region; (c) an immunoglobulin constant region; and (d) a second binding domain; wherein the first binding domain is a PD-L1 binding domain, and the second binding domain is a CD40 binding domain.

The CD40 binding domain, the PD-L1 binding domain, or both binding domains may be a scFv.

Provided herein is a binding polypeptide that binds specifically to human PD-L 1 and human CD40; wherein the binding polypeptide comprises, in order from amino-terminus to carboxyl-terminus: (i) a first binding domain, (ii) a hinge region, (iii) an immunoglobulin constant region, (iv) a Fc-binding domain linker, and (v) a second binding domain wherein the first binding domain is a PD-L1 binding domain, and the second binding domain is a CD40 binding domain. In some embodiments, an Fc-binding domain linker is GGGGSPS (SEQ ID NO: 21).

Provided herein is a binding polypeptide that binds specifically to human PD-L1 and human CD40; wherein the binding polypeptide comprises, in order from amino-terminus to carboxyl-terminus: (i) a PD-L1 binding domain; (ii) an Fc region; and (iii) a CD40 binding domain. In some embodiments, an Fc region comprises or consists of SEQ ID NO 22.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO: 10, a HCDR2 comprising SEQ ID NO: 11, and a HCDR3 comprising SEQ ID NO: 12; and a VL comprising a LCDR1 comprising SEQ ID NO:14, a LCDR2 comprising SEQ ID NO: 15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO: 10, a HCDR2 comprising SEQ ID NO:23, and a HCDR3 comprising SEQ ID NO: 12; and a VL comprising a LCDR1 comprising SEQ ID NO:14, a LCDR2 comprising SEQ ID NO: 15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO:10, a HCDR2 comprising SEQ ID NO: 11, and a HCDR3 comprising SEQ ID NO: 31; and a VL comprising a LCDR1 comprising SEQ ID NO:14, a LCDR2 comprising SEQ ID NO: 15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO: 10, a HCDR2 comprising SEQ ID NO:23, and a HCDR3 comprising SEQ ID NO: 31; and a VL comprising a LCDR1 comprising SEQ ID NO:14, a LCDR2 comprising SEQ ID NO: 15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO:10, a HCDR2 comprising SEQ ID NO: 11, and a HCDR3 comprising SEQ ID NO:12; and a VL comprising a LCDR1 comprising SEQ ID NO:37, a LCDR2 comprising SEQ ID NO: 15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO: 10, a HCDR2 comprising SEQ ID NO: 11, and a HCDR3 comprising SEQ ID NO: 12; and a VL comprising a LCDR1 comprising SEQ ID NO:41, a LCDR2 comprising SEQ ID NO:15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO:10, a HCDR2 comprising SEQ ID NO: 45, and a HCDR3 comprising SEQ ID NO: 12; and a VL comprising a LCDR1 comprising SEQ ID NO:14, a LCDR2 comprising SEQ ID NO: 15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO:10, a HCDR2 comprising SEQ ID NO: 49, and a HCDR3 comprising SEQ ID NO: 12; and a VL comprising a LCDR1 comprising SEQ ID NO:14, a LCDR2 comprising SEQ ID NO: 15, and a LCDR3 comprising SEQ ID NO: 16.

Provided herein is a binding polypeptide, wherein the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and the CD40 binding domain comprises a VH comprising a HCDR1 comprising SEQ ID NO:10, a HCDR2 comprising SEQ ID NO: 23, and a HCDR3 comprising SEQ ID NO: 31; and a VL comprising a LCDR1 comprising SEQ ID NO:37, a LCDR2 comprising SEQ ID NO: 15, and a LCDR3 comprising SEQ ID NO: 16.

Provided herein is a binding polypeptide, wherein (a) the PD-L1 binding domain comprises a VH comprising SEQ ID NO: 4 and a VL comprising SEQ ID NO: 8; and the CD40 binding domain comprises a VH comprising SEQ ID NO: 13 and a VL comprising SEQ ID NO: 17; (b) the PD-L1 binding domain comprises a VH comprising SEQ ID NO: 4 and a VL comprising SEQ ID NO: 8; and the CD40 binding domain comprises a VH comprising SEQ ID NO: 24 and a VL comprising SEQ ID NO: 17; (c) the PD-L1 binding domain comprises a VH comprising SEQ ID NO: 4 and a VL comprising SEQ ID NO: 8; and the CD40 binding domain comprises a VH comprising SEQ ID NO: 27 and a VL comprising SEQ ID NO: 28; (d) the PD-L1 binding domain comprises a VH comprising SEQ ID NO: 4 and a VL comprising SEQ ID NO: 8, and the CD40 binding domain comprises a VH comprising SEQ ID NO: 32 and a VL comprising SEQ ID NO: 17; (e) the PD-L1 binding domain comprises a VH comprising SEQ ID NO: 4 and a VL comprising SEQ ID NO: 8; and the CD40 binding domain comprises a VH comprising SEQ ID NO: 34 and a VL comprising SEQ ID NO: 17; (f) the PD-L1 binding domain comprises a VH comprising SEQ ID NO: 4 and a VL comprising SEQ ID NO: 8; and the CD40 binding domain comprises a VH comprising SEQ ID NO: 13 and a VL comprising SEQ ID NO: 38; (g) the PD-L1 binding domain comprises a VH comprising SEQ ID NO: 4 and a VL comprising SEQ ID NO: 8; and the CD40 binding domain comprises a VH comprising SEQ ID NO: 13 and a VL comprising SEQ ID NO: 42; (h) the PD-L1 binding domain comprises a VH comprising SEQ ID NO: 4 and a VL comprising SEQ ID NO: 8; and the CD40 binding domain comprises a VH comprising SEQ ID NO: 46 and a VL comprising SEQ ID NO: 17; (i) the PD-L1 binding domain comprises a VH comprising SEQ ID NO: 4 and a VL comprising SEQ ID NO: 8; and the CD40 binding domain comprises a VH comprising SEQ ID NO: 50 and a VL comprising SEQ ID NO: 17; (e) the PD-L1 binding domain comprises a VH comprising SEQ ID NO: 4 and a VL comprising SEQ ID NO: 8; and the CD40 binding domain comprises a VH comprising SEQ ID NO: 484 and a VL comprising SEQ ID NO: 485; or (k) the PD-L1 binding domain comprises a VH comprising SEQ ID NO: 4 and a VL comprising SEQ ID NO: 8; and the CD40 binding domain comprises a VH comprising SEQ ID NO: 484 and a VL comprising SEQ ID NO: 28.

Provided herein is a binding polypeptide, wherein (a) the PD-L1 binding domain comprises SEQ ID NO: 9; and the CD40 binding domain comprises SEQ ID NO: 18; (b) the PD-L1 binding domain comprises SEQ ID NO: 9; and the CD40 binding domain comprises SEQ ID NO: 25; (c) the PD-L1 binding domain comprises SEQ ID NO: 9; and the CD40 binding domain comprises SEQ ID NO: 29; (d) the PD-L1 binding domain comprises SEQ ID NO: 9; and the CD40 binding domain comprises SEQ ID NO: 33; (e) the PD-L1 binding domain comprises SEQ ID NO: 9; and the CD40 binding domain comprises SEQ ID NO: 35; (f) the PD-L1 binding domain comprises SEQ ID NO: 9; and the CD40 binding domain comprises SEQ ID NO: 39; (g) the PD-L1 binding domain comprises SEQ ID NO: 9; and the CD40 binding domain comprises SEQ ID NO: 43; (h) the PD-L1 binding domain comprises SEQ ID NO: 9; and the CD40 binding domain comprises SEQ ID NO: 47; (1) the PD-L1 binding domain comprises SEQ ID NO: 9; and the CD40 binding domain comprises SEQ ID NO: 51; (j) the PD-L1 binding domain comprises SEQ ID NO: 9; and the CD40 binding domain comprises SEQ ID NO: 486; (k) the PD-L1 binding domain comprises SEQ ID NO: 9; and the CD40 binding domain comprises SEQ ID NO: 488.

Provided herein is a binding polypeptide comprising SEQ ID NO: 19, SEQ ID NO: 26, SEQ ID NO: 30, SEQ ID NO: 280, SEQ ID NO: 36, SEQ ID NO: 40, SEQ ID NO: 44, SEQ ID NO: 48, SEQ ID NO: 52, SEQ ID NO: 487, or SEQ ID NO: 489. Provided herein is a binding polypeptide comprising an amino acid sequence having at least 95%, 96%, 97%, 98%, or 99% sequence identity to any one of SEQ ID NO: 19, SEQ ID NO: 26, SEQ ID NO: 30, SEQ ID NO: 280, SEQ ID NO: 36, SEQ ID NO: 40, SEQ ID NO: 44, SEQ ID NO: 48, SEQ ID NO: 52, SEQ ID NO: 487, and SEQ ID NO: 489.

Provided herein is a binding polypeptide comprising the amino acid sequence of the molecule PC401003, as well as its variants PC401119, PC401120, PC401122, PC401124, PC401128, PC401129, PC401132, PC401133, PC401144, or PC401145.

Compared to PC401003, PC401119 comprises an A to N mutation in anti-CD40 HCDR2, PC401120 comprises a Q to C mutation in anti-CD40 LFW4 and G to C mutation in anti-CD40 HFW2, PC401122 comprises a V to N mutation in anti-CD40 HCDR3, PC401124 comprises an A to N mutation in anti-CD40 HCDR2 and a V to N mutation in anti-CD40 HCDR3, PC401128 comprises a L to T mutation in anti-CD40 LCDR1, PC401129 comprises a L to N mutation in anti-CD40 LCDR1, PC401132 comprises a L to N mutation in anti-CD40 HCDR2, PC401133 comprises a L to Q mutation in anti-CD40 HCDR2, PC401144 comprises an A to N mutation in anti-CD40 HCDR2, a V to N mutation in anti-CD40 HCDR3, and a L to T mutation in anti-CD40 LCDR1, and PC401145 comprises an A to N mutation in anti-CD40 HCDR2 and a V to N mutation in anti-CD40 HCDR3. These amino acid substitution mutations are shown in Table 1.

Provided herein is a binding polypeptide comprising a CD40 binding domain, wherein the CD40 binding domain is a variant of the PC401003 CD40 binding domain that comprises 2, 3, 4, 5, 6, or 7 of the amino acid substitutions found in PC401119, PC401120, PC401122, PC401124, PC401128, PC401129, PC401132, PC401133. PC401144, or PC401145.

The binding domains described herein may comprise or consist of a single chain variable fragment (scFv). In some embodiments, the scFv may be in the V_{H}-V_{L} orientation or the V_{L}-V_{H} orientation. In some embodiments, the VH may be carboxy-terminal to the VL. In some embodiments, the VL may be carboxy-terminal to the VH.

In some embodiments, the scFv may comprise a linker between the V_{H} and V_{L}, regions (i.e., a scFv linker). In some embodiments, the linker may comprise GGGGSGGGGSGGGGSGGGGS (SEQ ID NO: 20).

Binding polypeptides provided herein may comprise an immunoglobulin Fc (Fc) region. The Fc regions described herein may comprise IgG CH2 and CH3 domains, e.g., CH2 and CH3 domains of IgG1, IgG2, IgG3, IgG4, IgA1, IgA2 or IgD. In certain embodiments, the Fc region does not comprise a CH1 domain. In certain embodiments, the sequences making up the Fc region are human or derived from human sequences. In some embodiments, the Fc region comprises a human IgG1 CH2 domain comprising an amino acid substitution at one or more of the following residues, according to the EU numbering system: E233, L234, L235, G236, G237, E318, K320, and K322. In some embodiments, the amino acid substitution at residue E233 is E233P. In some embodiments, the amino acid substitution at residue L234 is selected from the group consisting of L234A and L234V. In some embodiments, the amino acid substitution at residue L235 is L235A. In some embodiments, the amino acid substitution at residue G237 is G237A. In some embodiments, the amino acid substitution at E318 is E318A. In some embodiments, the amino acid substitution at K320 is K320A. In some embodiments, the amino acid substitution at K322 is K322A. In some embodiments, one or more of E233, L234, L235, G236, G237, E318, K320, and K322 is deleted. In particular embodiments, residue G236 is deleted.

In some embodiments, the Fc domain is derived from IgG1. In some embodiments, the Fc domain that is derived from IgG1 comprises two or more mutations that prevent the polypeptide from depleting PD-L1 and/or CD40 expressing cells when administered to a patient. In some embodiments, the two or more mutations in the IgG1 Fc domain prevent or substantially reduce signaling through Fc-mediated cross-linking.

In some embodiments, the Fc comprises SEQ ID NO: 22

In some embodiments, a binding polypeptide comprises a hinge that is an immunoglobulin hinge. The hinge regions described herein may be derived from an IgG. In some embodiments, the hinge region has one or more mutated cysteine residues.

In some embodiments, a binding polypeptide provided herein is a single-chain polypeptide.

In some embodiments, a binding polypeptide is a dimer, for example a homodimer or a heterodimer. In some embodiments, a dimeric polypeptide comprises at least one of the binding polypeptides of the disclosure. In some embodiments, a dimeric polypeptide comprises two binding polypeptides of the disclosure. In some embodiments, a binding polypeptide of the disclosure may be dimerized to an identical binding polypeptide. In some embodiments, a bispecific single chain binding polypeptide exists primarily in a homodimeric form. In some embodiments, a binding polypeptide of the disclosure may be dimerized to a non-identical second polypeptide.

### Polynucleotides and Methods of Protein Expression

The disclosure also includes nucleic acids (e.g., DNA or RNA) encoding the polypeptides of the present disclosure (e.g., bispecific PD-L1 binding polypeptides, bispecific CD40 binding polypeptides, or bispecific PD-L1×CD40 binding polypeptides) or one or more polypeptide chains of a polypeptide as described herein. Nucleic acids of the disclosure also include complementary nucleic acids. In some instances, the sequences will be fully complementary (no mismatches) when aligned. In other instances, there can be up to about a 20% mismatch in the sequences. In some embodiments of the disclosure are provided nucleic acids encoding both first and second polypeptide chains of a bispecific protein of the disclosure. The nucleic acid sequences provided herein can be exploited using codon optimization, degenerate sequence, silent mutations, and other DNA techniques to optimize expression in a particular host, and the present disclosure encompasses such sequence modifications.

The disclosure relates to an isolated nucleic acid molecule encoding polypeptides of the present disclosure (e.g., PD-L1 binding domains, CD40 binding domains, and bispecific PD-L1×CD40 binding polypeptides). Provided herein is a nucleic acid molecule comprising a nucleotide sequence that comprises SEQ ID NO: 78, SEQ ID NO: 79, SEQ ID NO: 259, or SEQ ID NO: 260.

Polynucleotide molecules comprising a desired polynucleotide sequence are propagated by placing the molecule in a vector. Viral and non-viral vectors are used. In some embodiments, the vector is a viral vector. In some embodiments, the viral vector is a lentivirus, a retrovirus, an adenovirus, or an adeno-associated virus (AAV). In some embodiments, the vector is a plasmid. The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially. The partial or full-length polynucleotide is inserted into a vector typically by means of DNA ligase attachment to a cleaved restriction enzyme site in the vector. Alternatively, the desired nucleotide sequence can be inserted by homologous recombination in vivo. Typically, this is accomplished by attaching regions of homology to the vector on the flanks of the desired nucleotide sequence. Regions of homology are added by ligation of oligonucleotides, or by polymerase chain reaction using primers comprising both the region of homology and a portion of the desired nucleotide sequence, for example.

For expression, an expression cassette or system may be employed. To express a nucleic acid encoding a polypeptide disclosed herein, a nucleic acid molecule encoding the polypeptide, operably linked to regulatory sequences that control transcriptional expression in an expression vector, is introduced into a host cell. The transcriptional regulatory sequences may include promoters and/or enhancers, and the promoters may be constitutive or inducible. In addition to transcriptional regulatory sequences, expression vectors can include translational regulatory sequences and a marker gene which is suitable for selection of cells that carry the expression vector. The gene product encoded by a polynucleotide of the disclosure is expressed in any convenient expression system, including, for example, bacterial, yeast, insect, amphibian and mammalian systems. In the expression vector, the polypeptide-encoding polynucleotide is linked to a regulatory sequence as appropriate to obtain the desired expression properties. These can include promoters, enhancers, terminators, operators, repressors, and inducers. The promoters can be regulated (e.g., the promoter from the steroid inducible pIND vector (Invitrogen)) or constitutive (e.g., promoters from CMV, SV40, Elongation Factor, or LTR sequences). These are linked to the desired nucleotide sequence using the techniques described above for linkage to vectors. Any techniques known in the art can be used. Accordingly, the expression vector will generally provide a transcriptional and translational initiation region, which can be inducible or constitutive, where the coding region is operably linked under the transcriptional control of the transcriptional initiation region, and a transcriptional and translational termination region.

The vectors described herein may be contained within a host cell, such as a CHO cell or a HEK cell. In some embodiments, the host cell is stably transfected with the vector, e.g., a stably transfected CHO or HEK cell.

An expression cassette (“expression unit”) can be introduced into a variety of vectors, e.g., plasmid, BAC, YAC, bacteriophage such as lambda, P1, M13, etc., plant or animal viral vectors (e.g., retroviral-based vectors, adenovirus vectors), and the like, where the vectors are normally characterized by the ability to provide selection of cells comprising the expression vectors. The vectors can provide for extrachromosomal maintenance, particularly as plasmids or viruses, or for integration into the host chromosome. Where extrachromosomal maintenance is desired, an origin sequence is provided for the replication of the plasmid, which can be low- or high copy-number. A wide variety of markers are available for selection, particularly those which protect against toxins, more particularly against antibiotics. The particular marker that is chosen is selected in accordance with the nature of the host, where, in some cases, complementation can be employed with auxotrophic hosts. Introduction of the DNA construct can use any convenient method, including. e.g., conjugation, bacterial transformation, calcium-precipitated DNA, electroporation, fusion, transfection, infection with viral vectors, biolistics, and the like.

Accordingly, proteins for use within the present disclosure can be produced in genetically engineered host cells according to conventional techniques. Suitable host cells are those cell types that can be transformed or transfected with exogenous DNA and grown in culture, and include bacteria, fungal cells, and cultured higher eukaryotic cells (including cultured cells of multicellular organisms), particularly cultured mammalian cells. Techniques for manipulating cloned DNA molecules and introducing exogenous DNA into a variety of host cells are disclosed by Sambrook and Russell, Molecular Cloning: A Laboratory Manual (3rd ed., Cold Spring Harbor Laboratory Press, Cold Spring Harbor, NY, 2001), and Ausubel et al., Short Protocols in Molecular Biology (4th ed., John Wiley & Sons, 1999).

For example, for recombinant expression of a homodimeric binding protein comprising two identical binding polypeptides as described herein, an expression vector will generally include a nucleic acid segment encoding the binding polypeptide, operably linked to a promoter. For recombinant expression of a heterodimeric binding protein comprising different first and second polypeptide chains, the first and second polypeptide chains can be co-expressed from separate vectors in the host cell for expression of the entire heterodimeric protein. Alternatively, for the expression of heterodimeric binding proteins the first and second polypeptide chains are co-expressed from separate expression units in the same vector in the host cell for expression of the entire heterodimeric protein. The expression vector(s) are transferred to a host cell by conventional techniques, and the transfected cells are then cultured by conventional techniques to produce the encoded polypeptide(s) to produce the corresponding binding proteins (e.g., PD-L1×CD40 polypeptides).

To direct a recombinant protein into the secretory pathway of a host cell, a secretory signal sequence (also known as a leader sequence) is provided in the expression vector. The secretory signal sequence can be that of the native form of the recombinant protein, or can be derived from another secreted protein or synthesized de novo. The secretory signal sequence is operably linked to the polypeptide-encoding DNA sequence. i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences can be positioned elsewhere in the DNA sequence of interest (see. e.g., U.S. Pat. Nos. 5,037,743 and 5,143,830).

Cultured mammalian cells are suitable hosts for production of recombinant polypeptides and proteins of the present disclosure (e.g., PD-L1×CD40 polypeptides) for use within the present disclosure. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., supra), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, U.S. Pat. Nos. 4,713,339; 4,784,950; 4,579,821; and 4,656,134. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61, CHO DG44; CHO DXB11 (Hyclone, Logan, UT); see also, e.g., Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658). Additional suitable cell lines are known in the art and available from public depositories such as the American Type Culture Collection, Manassas, Virginia. Strong transcription promoters can be used, such as promoters from SV-40 or cytomegalovirus. See. e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants.” Cells that have been cultured in the presence of the selective agent and are able to pass the gene of interest to their progeny are referred to as “stable transfectants.” Exemplary selectable markers include a gene encoding resistance to the antibiotic neomycin, which allows selection to be carried out in the presence of a neomycin-type drug, such as G-418 or the like; the gpt gene for xanthine-guanine phosphoribosyl transferase, which permits host cell growth in the presence of mycophenolic acid/xanthine; and markers that provide resistance to zeocin, bleomycin, blastocidin, and hygromycin (see, e.g., Gatignol et al., Mol. Gen. Genet. 207:342, 1987; Drocourt et al., Nucl. Acids Res. 18:4009, 1990). Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. An exemplary amplifiable selectable marker is dihydrofolate reductase, which confers resistance to methotrexate. Other drug resistance genes (e.g., hygromycin resistance, multi-drug resistance, puromycin acetyltransferase) can also be used.

Other higher eukaryotic cells can also be used as hosts, including insect cells, plant cells and avian cells. The use of Agrobacterium rhizogenes as a vector for expressing genes in plant cells has been reviewed by Sinkar et al, J. Biosci. (Bangalore) 11:47-58, 1987. Transformation of insect cells and production of foreign polypeptides therein is disclosed in U.S. Pat. No. 5,162,222 and PCT Publication No. WO 94/06463.

Insect cells can be infected with recombinant baculovirus, commonly derived from Autographa californica nuclear polyhedrosis virus (AcNPV). See King and Possee, The Baculovirus Expression System: A Laboratory Guide (Chapman & Hall, London); O'Reilly et al., Baculovirus Expression Vectors: A Laboratory Manual (Oxford University Press., New York 1994); and Baculovirus Expression Protocols. Methods in Molecular Biology (Richardson ed., Humana Press, Totowa, NJ, 1995). Recombinant baculovirus can also be produced through the use of a transposon-based system described by Luckow et al. (J. Virol. 67:4566-4579, 1993). This system, which utilizes transfer vectors, is commercially available in kit form (BAC-TO-BAC kit; Life Technologies, Gaithersburg, MD). The transfer vector (e.g., PFASTBAC1; Life Technologies) contains a Tn7 transposon to move the DNA encoding the protein of interest into a baculovirus genome maintained in E. coli as a large plasmid called a “bacmid.” See Hill-Perkins and Possee, J. Gen. Virol. 71:971-976, 1990; Bonning et al., J. Gen. Virol. 75:1551-1556, 1994; and Chazenbalk and Rapoport, J. Biol. Chem. 270:1543-1549, 1995. In addition, transfer vectors can include an in-frame fusion with DNA encoding a polypeptide extension or affinity tag as disclosed above. Using techniques known in the art, a transfer vector containing a protein-encoding DNA sequence is transformed into E. coli host cells, and the cells are screened for bacmids which contain an interrupted lacZ gene indicative of recombinant baculovirus. The bacmid DNA containing the recombinant baculovirus genome is isolated, using common techniques, and used to transfect Spodoptera frugiperda cells, such as Sf9 cells. Recombinant virus that expresses the protein or interest is subsequently produced. Recombinant viral stocks are made by methods commonly used in the art.

For protein production, the recombinant virus is used to infect host cells, typically a cell line derived from the fall armyworm, Spodoptera frugiperda (e.g., Sf9 or Sf21 cells) or Trichoplusia ni (e.g., HIGH FIVE™ cells; Invitrogen, Carlsbad. CA). See generally Glick and Pasternak, Molecular Biotechnology, Principles & Applications of Recombinant DNA (ASM Press, Washington, D.C., 1994). See also U.S. Pat. No. 5,300,435. Serum-free media are used to grow and maintain the cells. Suitable media formulations are known in the art and can be obtained from commercial suppliers. The cells are grown up from an inoculation density of approximately 2-5×10⁵ cells to a density of 1-2×10⁶ cells, at which time a recombinant viral stock is added at a multiplicity of infection (MOI) of 0.1 to 10, more typically near 3. Procedures used are generally described in available laboratory manuals (see. e.g., King and Possee, supra; O'Reilly er al., supra; Richardson, supra).

Fungal cells, including yeast cells, can also be used within the present disclosure to produce the polypeptides of the present disclosure (e.g., PD-L1×CD40 polypeptides). Yeast species of in this regard include, e.g., Saccharomyces cerevisiae, Pichia pastoris, and Pichia methanolica. Methods for transforming S. cerevisiae cells with exogenous DNA and producing recombinant polypeptides therefrom are disclosed by, for example, U.S. Pat. Nos. 4,599,311; 4,931,373; 4,870,008; 5,037,743; and 4,845,075. Transformed cells are selected by phenotype determined by the selectable marker, commonly drug resistance or the ability to grow in the absence of a particular nutrient (e.g., leucine). An exemplary vector system for use in Saccharomyces cerevisiae is the POTI vector system disclosed by Kawasaki er al. (U.S. Pat. No. 4,931,373), which allows transformed cells to be selected by growth in glucose-containing media. Suitable promoters and terminators for use in yeast include those from glycolytic enzyme genes (see, e.g., U.S. Pat. Nos. 4,599,311; 4,615,974; and 4,977,092) and alcohol dehydrogenase genes. See also U.S. Pat. Nos. 4,990,446; 5,063,154; 5,139,936; and 4,661,454. Transformation systems for other yeasts, including Hansenula polymorpha, Schizosaccharomyces pombe, Kluyveromyces lactis. Kluyveromyres fragilis. Ustilago maydis, Pichia pastoris, Pichia methanolica, Pichia guillermondii, and Candida maltosa are known in the art. See. e.g., Gleeson et al., J. Gen. Microbiol. 132:3459-3465, 1986; U.S. Pat. No. 4,882,279; and Raymond et al., Yeast 14:11-23, 1998. Aspergillus cells can be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. Production of recombinant proteins in Pichia methanolica is disclosed in U.S. Pat. Nos. 5,716,808; 5,736,383; 5,854,039; and 5,888,768.

Prokaryotic host cells, including strains of the bacteria Escherichia coli, Bacillus, and other genera are also useful host cells within the present disclosure to produce, for example, PD-L1 binding polypeptides, CD40 binding polypeptides, and/or multispecific binding proteins including PDL1×CD40 binding polypeptides. Techniques for transforming these hosts and expressing foreign DNA sequences cloned therein are well-known in the art (see, e.g., Sambrook and Russell, supra). When expressing a recombinant protein in bacteria such as E. coli, the protein can be retained in the cytoplasm, typically as insoluble granules, or can be directed to the periplasmic space by a bacterial secretion sequence. In the former case, the cells are lysed, and the granules are recovered and denatured using, for example, guanidine isothiocyanate or urea. The denatured protein can then be refolded and dimerized by diluting the denaturant, such as by dialysis against a solution of urea and a combination of reduced and oxidized glutathione, followed by dialysis against a buffered saline solution. In the alternative, the protein can be recovered from the cytoplasm in soluble form and isolated without the use of denaturants. The protein is recovered from the cell as an aqueous extract in, for example, phosphate buffered saline. To capture the protein of interest, the extract is applied directly to a chromatographic medium, such as an immobilized antibody or heparin-Sepharose column. Secreted proteins can be recovered from the periplasmic space in a soluble and functional form by disrupting the cells (by, for example, sonication or osmotic shock) to release the contents of the periplasmic space and recovering the protein, thereby obviating the need for denaturation and refolding. Antibodies, including single-chain antibodies, can be produced in bacterial host cells according to known methods. See. e.g., Bird et al., Science 242:423-426, 1988; Huston et al, Proc. Natl. Acad. Sci. USA 85:5879-5883, 1988; and Pantoliano et al., Biochem. 30:10117-10125, 1991.

Transformed or transfected host cells to produce the polypeptides and proteins of the present disclosure (e.g., PD-L1 binding polypeptides, CD40 binding polypeptides, or PD-L1×CD40 polypeptides) are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media can also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

The proteins and polypeptides of the present disclosure (e.g., PD-L1×CD40 polypeptides) may be purified by conventional protein purification methods, typically by a combination of chromatographic techniques. See generally Affinity Chromatography: Principles & Methods (Pharmacia LKB Biotechnology, Uppsala, Sweden, 1988); Scopes, Protein Purification: Principles and Practice (Springer-Verlag, New York 1994). Proteins comprising an immunoglobulin Fc region can be purified by affinity chromatography on immobilized protein A or protein G. Additional purification steps, such as gel filtration, can be used to obtain the desired level of purity or to provide for desalting, buffer exchange, and the like.

### Compositions and Methods of Use

For administration to a subject, a protein of the present disclosure (e.g., bispecific PD-L1 binding polypeptides, bispecific CD40 binding polypeptides, or bispecific PD-L1×CD40 binding polypeptides, or dimers thereof) may be formulated as a pharmaceutical composition. A pharmaceutical composition may comprise: (i) a binding polypeptide of the disclosure, and/or a dimer thereof; and (ii) a pharmaceutically acceptable carrier, diluent or excipient. A pharmaceutical composition comprising a binding polypeptide of the disclosure can be formulated according to known methods to prepare pharmaceutically useful compositions, whereby the therapeutic molecule is combined in a mixture with a pharmaceutically acceptable carrier, diluent or excipient. A carrier is said to be a “pharmaceutically acceptable carrier” if its administration can be tolerated by a recipient patient. Sterile phosphate-buffered saline is one example of a pharmaceutically acceptable carrier. Other suitable carriers, diluents or excipients are well-known to those in the art. (See. e.g., Gennaro (ed.), Remington's Pharmaceutical Sciences (Mack Publishing Company, 19th ed. 1995).) Formulations can further include one or more excipients, preservatives, solubilizers, buffering agents, albumin to prevent protein loss on vial surfaces, etc. In certain embodiments, a pharmaceutical composition comprises a bispecific PD-L1×CD40 binding polypeptide that is a homodimer or a heterodimer. A “homodimer” may be a dimer formed from two identical polypeptides.

Thus, in some embodiments, a pharmaceutical composition may comprise a PD-L1 binding polypeptide, a bispecific CD40 binding polypeptide, or a bispecific PD-L1×CD40 binding polypeptides of the disclosure dimerized to an identical binding polypeptide and a pharmaceutically acceptable buffer or excipient.

A pharmaceutical composition comprising a bispecific PD-L1 binding polypeptide, a bispecific CD40 binding polypeptide, or a bispecific PD-L1×CD40 binding polypeptides of the disclosure may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, and an intracerebral unit dosage form. The oral unit dosage form may be selected from the group consisting of: tablets, pills, pellets, capsules, powders, lozenges, granules, solutions, suspensions, emulsions, syrups, elixirs, sustained-release formulations, aerosols, and sprays.

A pharmaceutical composition comprising polypeptide or protein described herein (e.g., a bispecific PD-L1 binding polypeptide, a bispecific CD40 binding polypeptide, or a bispecific PD-L1×CD40 binding polypeptide, or dimer thereof) may be administered to a subject in a therapeutically effective amount. According to the methods of the present disclosure, polypeptide or protein described herein can be administered to subjects by a variety of administration modes, including, for example, by intramuscular, subcutaneous, intravenous, intra-atrial, intra-articular, parenteral, intranasal, intrapulmonary, transdermal, intrapleural, intrathecal, and oral routes of administration. For prevention and treatment purposes, an antagonist can be administered to a subject in a single bolus delivery, via continuous delivery (e.g., continuous transdermal delivery) over an extended time period, or in a repeated administration protocol (e.g., on an hourly, daily, weekly, or monthly basis).

Thus, in some embodiments, the disclosure provides a method of treating a subject comprising administering to the subject a therapeutically effective dose of a pharmaceutical composition of the disclosure. In some embodiments, the subject suffers from cancer. In some embodiments, the cancer expresses or overexpresses PD-L1. In some embodiments, the cancer is a solid tumor. In some embodiments, the cancer is a carcinoma. In some embodiments, the cancer is head and neck cancer, melanoma, lung cancer, brain cancer, thymus cancer, breast cancer, liver cancer, pancreatic cancer, kidney cancer, ovarian cancer, bladder cancer, gastrointestinal tract cancer or colon cancer. In some embodiments, the patient suffers from head and neck squamous cell carcinoma, melanoma, or carcinoma of the lung, brain, thymus, breast, liver, pancreas, kidney, ovary, bladder, gastrointestinal tract or colon.

In some embodiments, the disclosure provides a method of treating or ameliorating a symptom of a cancer (e.g., a cancer characterized by expression or overexpression of PD-L1) in a subject, comprising administering to the subject a pharmaceutical composition comprising a bispecific polypeptide of the disclosure.

Provided herein are the binding polypeptides (e.g., a bispecific PD-L1 binding polypeptide, a bispecific CD40 binding polypeptide, or a bispecific PD-L1×CD40 binding polypeptide), dimeric proteins, and compositions disclosed herein for use as a medicament.

Determination of effective dosages in this context is typically based on animal model studies followed up by human clinical trials and is guided by determining effective dosages and administration protocols that significantly reduce the occurrence or severity of the subject disorder in model subjects. Effective doses of the compositions of the present disclosure vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. Usually, the patient is a human, but in some diseases, the patient can be a nonhuman mammal. Typically, dosage regimens are adjusted to provide an optimum therapeutic response, i.e., to optimize safety and efficacy. Accordingly, a therapeutically effective amount is also one in which any undesired collateral effects are outweighed by the beneficial effects of administering a bispecific PD-L1 binding polypeptide, a bispecific CD40 binding polypeptide, or a bispecific PD-L1×CD40 binding polypeptide of the disclosure, as described herein.

In some embodiments, a dosage of the pharmaceutical composition may range from about 0.1 μg to 100 about mg/kg or about 1 μg/kg to about 50 mg/kg, and more usually about 10 μg to about 5 mg/kg of the subject's body weight. In more specific embodiments, an effective amount of the agent is between about 1 μg/kg and about 20 mg/kg, between about 10 μg/kg and about 10 mg/kg, or between about 0.1 mg/kg and about 5 mg/kg. In particular embodiments, a pharmaceutical composition is administered at a dose of about 100 μg/kg or less. In some embodiments, a pharmaceutical composition comprising a bispecific PD-L1 binding polypeptide, a bispecific CD40 binding polypeptide, or a bispecific PD-L1×CD40 binding polypeptide of the disclosure is administered to a patient by intravenous injection at a dose of about 20 μg/kg, about 40 μg/kg, about 80 μg/kg or about 200 μg/kg.

Dosages within this range can be achieved by single or multiple administrations, including. e.g., multiple administrations per day or daily, weekly, bi-weekly, or monthly administrations. For example, in certain variations, a regimen consists of an initial administration followed by multiple, subsequent administrations at weekly or bi-weekly intervals. Another regimen consists of an initial administration followed by multiple, subsequent administrations at monthly or bi-monthly intervals. Alternatively, administrations can be on an irregular basis as indicated by monitoring clinical symptoms of the disorder.

Dosage of the pharmaceutical composition comprising a polypeptide or protein described herein can be varied by the attending clinician to maintain a desired concentration at a target site. For example, if an intravenous mode of delivery is selected, local concentration of the agent in the bloodstream at the target tissue can be between about 0.01-50 nanomoles of the composition per liter, sometimes between about 1.0 nanomole per liter and 10, 15, or 25 nanomoles per liter depending on the subject's status and projected measured response. Higher or lower concentrations can be selected based on the mode of delivery, e.g., trans-epidermal delivery versus delivery to a mucosal surface. Dosage should also be adjusted based on the release rate of the administered formulation, e.g., nasal spray versus powder, sustained release oral or injected particles, transdermal formulations, etc. To achieve the same serum concentration level, for example, slow-release particles with a release rate of 5 nanomolar (under standard conditions) would be administered at about twice the dosage of particles with a release rate of 10 nanomolar.

The proteins and polypeptides described herein may also be administered at a daily dosage of from about 0.001 to about 10 milligrams (mg) per kilogram (mpk) of body weight, preferably given as a single daily dose or in divided doses about two to six times a day. For administration to a human adult patient, the therapeutically effective amount may be administered in doses in the range of about 0.2 mg to about 800 mg per dose, including but not limited to 0.2 mg per dose, 0.5 mg per dose, 1 mg per dose, 5 mg per dose, 10 mg per dose, 25 mg per dose, 100 mg per dose, 200 mg per dose, and 400 mg per dose, and multiple, usually consecutive daily doses may be administered in a course of treatment. The proteins and polypeptides described herein can be administered at different times of the day. In one embodiment the optimal therapeutic dose can be administered in the evening. In another embodiment the optimal therapeutic dose can be administered in the morning. The total daily dosage of the proteins and polypeptides described herein thus can in one embodiment range from about 1 mg to about 2 g, and often ranges from about 100 mg to about 1.5 g, and most often ranges from about 200 mg to about 1200 mg. In the case of a typical 70 kg adult human, the total daily dose of the anti-5T4 therapeutic can range from about 2 mg to about 1200 mg and will often range, as noted above, from about 0.2 mg to about 800 mg.

Subjects for administration of a protein of the present disclosure include patients at high risk for developing a particular cancer as well as patients presenting with an existing cancer. Typically, the subject has been diagnosed as having the cancer for which treatment is sought. Further, subjects can be monitored during the course of treatment for any change. Also, in some variations, the subject does not suffer from another disease or disorder requiring treatment.

In prophylactic applications, pharmaceutical compositions or medicaments comprising a protein of the present disclosure are administered to a patient susceptible to, or otherwise at risk of, a particular disorder in an amount sufficient to eliminate or reduce the risk or delay the onset of the disorder. In therapeutic applications, compositions or medicaments comprising a protein of the present disclosure are administered to a patient suspected of, or already suffering from such a disorder in an amount sufficient to cure, or at least partially arrest, the symptoms of the disorder and its complications. An amount adequate to accomplish this is referred to as a therapeutically effective dose or amount. In both prophylactic and therapeutic regimes, agents are usually administered in several dosages until a sufficient response (e.g., inhibition of inappropriate angiogenesis activity) has been achieved. Typically, the response is monitored and repeated dosages are given if the desired response starts to fade.

To identify subject patients for treatment according to the methods of the disclosure, accepted screening methods can be employed to determine risk factors associated with specific disorders or to determine the status of an existing disorder identified in a subject. Such methods can include, for example, determining whether an individual has relatives who have been diagnosed with a particular disorder. Screening methods can also include, for example, conventional work-ups to determine familial status for a particular disorder known to have a heritable component. For example, various cancers are also known to have certain inheritable components. Inheritable components of cancers include, for example, mutations in multiple genes that are transforming (e.g., Ras, Raf, EGFR cMet, and others), the presence or absence of certain HLA and killer inhibitory receptor (KIR) molecules, or mechanisms by which cancer cells are able to modulate immune suppression of cells like NK cells and T-cells, either directly or indirectly (see, e.g., Ljunggren and Malmberg, Nature Rev. Immunol. 7:329-339, 2007; Boyton and Altmann, Clin. Exp. Immunol. 149:1-8, 2007). Toward this end, nucleotide probes can be routinely employed to identify individuals carrying genetic markers associated with a particular disorder of interest. In addition, a wide variety of immunological methods are known in the art that are useful to identify markers for specific disorder. For example, various ELISA immunoassay methods are available and well-known in the art that employ monoclonal antibody probes to detect antigens associated with specific tumors. Screening can be implemented as indicated by known patient symptomology, age factors, related risk factors, etc. These methods allow the clinician to routinely select patients in need of the methods described herein for treatment. In accordance with these methods, targeting an inflammatory disease or disorder as described herein can be implemented as an independent treatment program or as a follow-up, adjunct, or coordinate treatment regimen to other treatments.

Pharmaceutical compositions comprising the proteins and polypeptides described herein (e.g., a bispecific PD-L1 binding polypeptide, a bispecific CD40 binding polypeptide, or a bispecific PD-L1×CD40 binding polypeptide of the disclosure) can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously.

The disclosure will be further clarified by the following examples, which are intended to be purely exemplary of the disclosure and in no way limiting.

### EXAMPLES

### Example 1. Generation of PD-L1-Expressing CHO Cells and Recombinant Extracellular Domain Proteins

The protein sequences defining the human, non-human primate and murine PD-L1 full length and extracellular domains (ECDs) were obtained from the Genbank database and are listed in Table 4. AFH protein sequence consisting of AviTag, FLAGtag and His tag and represents a set of C-terminal tags for purification, detection, and biotin-based labeling purposes. mFc represents a protein sequence of murine IgG2a hinge and Fc region. PD-L1 hu-IgV mu-IgC ECD is a hybrid of human and murine PD-L1 ECD.

TABLE 4
SEQ IDs of PD-L1 constructs for
production of cell lines and recombinant proteins

| Construct Name | Construct Description | SEQ ID NO |
|---|---|---|
| PDL01001 | huPD-L1 ECD AFH | 53 |
| PDL01002 | huPD-L1 ECD mFc | 54 |
| PDL01003 | cyPD-L1 ECD AFH | 55 |
| PDL01004 | cyPD-L1 ECD mFc | 56 |
| PDL01011 | PD-L1 hu-IgV mu-IgC ECD AFH | 57 |
| PDL01017 | PD-L1 hu-IgV mu-IgC ECD mFc | 58 |
| PDL01021 | Human PD-L1 full-length | 59 |
| PDL01022 | Cyno PD-L1 full-length | 60 |
| PDL01026 | Human PD-1 full-length | 61 |

The DNA containing the nucleotide sequences encoding proteins listed in Table 4 was synthesized at IDT, Coralville IA, inserted either into an expression vector appropriate for mammalian cell expression and secretion or into an expression vector appropriate for cell-surface expression that included the ability to apply selective pressure to generate stable transfectants. These reagents were used to assess the cross reactivity and binding strength of anti-PD-L1 binding domains to human PD-L1 and the species to be used in potential toxicology assessments. The DNA expression vectors encoding AFH tags were used to transiently transfect human embryonic kidney fibroblast (HEK)-293 cells grown in suspension culture. After several days in culture, the conditioned media was clarified via centrifugation and sterile filtration. Protein purification was performed utilizing a combination of Immobilized Metal Affinity Chromatography (IMAC) followed by size exclusion chromatography (SEC). SEC removed aggregated and clipped product and other host cell contaminants. SEC was also used to buffer-exchange the protein into phosphate-buffered saline (PBS). Final purity was determined by analytical SEC and typically exceeded 90%. Protein batches were sterile-filtered and stored at 4° C. if the intent was to use within the next week or were frozen in aliquots in a −80° C. freezer.

Plasmid DNA encoding full-length constructs was digested with a restriction enzyme and ethanol precipitated, then dissolved in ultrapure water, then Maxcyte Electroporation Buffer. Linearized DNA was transfected into Chinese Hamster Ovary (CHO)-K1SV cells (CDACF-CHO-K1SV cells (ID code 269-W3), Lonza Biologics) by electroporation. Transfected cells were transferred from the electroporation cuvette to a T75 culture flask, rested, and then gently resuspended in 15 mL of CD CHO media supplemented with 6 mM L-Glutamine in the T150 flask. The flask was put in a 37° C., 5% CO₂ incubator and allowed to recover for 24 hours prior to placing in the selection conditions. On the day following transfection, the cells were centrifuged for 5 minutes at 1000 RPM and resuspended in CD CHO medium with 1×GS supplement and 50 μM MSX. After the bulk populations were recovered from initial selection, cells were evaluated for surface expression with commercially available reagents, and representative vials were frozen. To obtain clones with varying levels of expression, cells were sorted by flow cytometry, plated by limiting dilution, and allowed to grow for 2 weeks. Wells were imaged with a Clone Select Imager during the incubation to identify growth positive wells. Only wells with good quality images were selected for further expansion and characterization for surface expression by flow cytometry. All clones were frozen in banks at up to 30 vials per clone.

### Example 2. General Expression and Purification of PD-L1- and CD40-Binding Molecules and Antibodies

Monospecific and bispecific PD-L1- and CD40-binding molecules disclosed herein were produced by transient transfection of either HEK293 or CHO cells. Cultures were clarified of cells, cell debris, and insoluble matter by centrifugation and/or filtration. Recombinant homodimeric proteins were captured from the clarified, conditioned media using Protein A affinity chromatography (ProA). Preparative Size exclusion chromatography (Prep SEC) was typically performed to further purify the protein to homogeneity and buffer-exchange into PBS. Protein purity was verified by analytical size exclusion chromatography (analytical SEC) on an Agilent HPLC after each of the ProA and Prep SEC purification steps.

Heteromeric proteins in which two or more peptide chains assemble to form a soluble protein complex were expressed using transiently transfected CHO cells using separate plasmids for each peptide chain. In some instances, the plasmids were transfected in equal ratios. If it was observed that one peptide chain expressed significantly better than the other(s), the plasmid ratio was altered to transfect a greater quantity of the lower-expressing plasmid. The protein was captured from cell culture supernatant using ProA with a wash step and low pH elution step. Prep SEC was used to remove aggregated protein and exchange the sample into PBS. In some instances, a second ProA chromatography step was performed. After washing the column with PBS, the protein was eluted using a decreasing pH gradient (from neutral to acidic). In some cases, cation exchange chromatography was used to further purify heterodimers to remove low MW, homodimer and unpaired peptide chain contaminants.

In most instances, final protein batches were buffer-exchanged into PBS as part of the SEC purification process, adjusted to 1 mg/mL, sterile-filtered and stored at 4° C. until needed or otherwise specified. Protein concentration was determined from the absorbance at 280 nm using the theoretical extinction coefficient calculated from the amino acid sequence.

Endotoxin levels were determined with the Endosafe PTS instrument, using the manufacturer's instructions. This assured that the in vitro activity assay results would not be confounded by the presence of endotoxin. Analytical SEC was used along with peak area integration to quantify the purity of the samples. In some instances, the resolving power of analytical SEC was insufficient to separate the desired heterodimeric product from product-related contaminants. Capillary Electrophoresis-Sodium Dodecyl Sulphate (CE-SDS) was used as a secondary method to assess product purity. Reduced and non-reduced SDS-PAGE (Sodium Dodecyl Sulfate-PolyAcrylamide Gel Electrophoresis) gels were run along with molecular weight (MW) standards to confirm the purity and estimate MW of the product.

### Example 3. Generation of PD-L1 Antibodies by Immunization of Wild-Type Mice

PD-L1-specific antibodies were isolated from a hybridoma library generated after immunizing BALB/c mice with recombinant hybrid human/mouse antigen PDL01017 at Precision Antibody Columbia, MD. Supernatants from hybridoma clones were assayed by ELISA, and identified wells were confirmed for specific binding using flow cytometry on CHO cells transfected with human and cynomolgus PD-L1 (PDL01001 and 03). Positive clones were selected for expansion, and viable cells were frozen for RNA extraction and variable domain analysis. Supernatants were saved for additional analyses.

The variable heavy (VH) and light (VL) domain sequences for selected hybridoma clones were obtained by RT-PCR after isolating total RNA. Briefly, total RNA was isolated from the hybridoma clone cell banks using Qiagen's RNeasy Plus Kit (Qiagen, Venlo Netherlands), and 400 ng of total RNA were used in a First Stand cDNA synthesis reaction using oligo dT and Superscript IV (Thermo Fisher Scientific, Waltham, MA), following manufacturer's protocol. Following cDNA synthesis, the variable region cDNA was amplified using 1 μL of cDNA and a series of primer mixes for mouse IgG VH, Vκ, and Vλ (Novagen Mouse Ig-Primer Set, EMD Millipore, Temecula, CA). PCR products for each clone were directly sequenced using the reverse (constant domain) PCR primer and standard Sanger sequencing methods. DNA sequence analysis identified 10 different antibodies: 2C11, 3G5, 3G1, 5F9, 5F11, 6F6, 7A3-1, 7A3-2, 7H11 and 10B8. Sequences were then converted to scFv format in VH-VL and VH-VL orientation with 4×G4S linker (for list of constructs see Table 5.) by amplifying the variable domains using specific primers that contain overlapping sequences and were assembled as scFv-Fc with human IgG1 WT Fc and into a mammalian expression vector using NEBuilder HiFi DNA Assembly Cloning Kit (New England Biolabs, Beverly MA). Positive control anti-PD-L1 antibodies F11 and 7A3 were constructed as chimeric an bodies, see PDL01050 and PDL71051.

TABLE 5
SEQ ID NOs of anti-PD-L1 hybridoma-derived
scFv-Fc constructs and positive controls

| | | SEQ |
|---|---|---|
| Construct | | ID |
| Name | Construct Description | NO |
| PDL01031 | 2C11HL x human IgG1 | 62 |
| | WT Fc | |
| PDL01032 | 3G5HL x human IgG1 | 63 |
| | WT Fc | |
| PDL01033 | 3G11HL x human IgG1 | 64 |
| | WT Fc | |
| PDL01034 | 5F9HL x human IgG1 | 65 |
| | WT Fc | |
| PDL01035 | 5F11HL x human IgG1 | 69 |
| | WT Fc | |
| PDL01036 | 6F6HL x human IgG1 | 70 |
| | WT Fc | |
| PDL01037 | 7A3-1HL x human IgG1 | 71 |
| | WT Fc | |
| PDL01038 | 7A3-2HL x human IgG1 | 72 |
| | WT Fc | |
| PDL01039 | 7H11HL x human IgG1 | 73 |
| | WT Fc | |
| PDL01040 | 10B2HL x human IgG1 | 74 |
| | WT Fc | |
| PDL01041 | 2C11LH x human IgG1 | 261 |
| | WT Fc | |
| PDL01042 | 3G5LH x human IgG1 | 262 |
| | WT Fc | |
| PDL01043 | 3G11(F)LH x human | 263 |
| | IgG1 WT Fc | |
| PDL01045 | 6F6LH x human IgG1 | 264 |
| | WT Fc | |
| PDL01047 | 7A3-2LH x human IgG1 | 265 |
| | WT Fc | |
| PDL01048 | 7H11LH x human IgG1 | 266 |
| | WT Fc | |
| PDL01049 | 10B2LH x human IgG1 | 267 |
| | WT Fc | |
| PDL01050 | 5F11 chimeric mAb | 268 |
| | | (light chain) |
| | | 269 |
| | | (heavy chain) |
| PDL01051 | 7A3 chimeric mAb | 270 |
| | | (light chain) |
| | | 271 |
| | | (heavy chain) |

### Example 4. Humanization of PD-L1-Specific Clone 5F9 in scFv Format

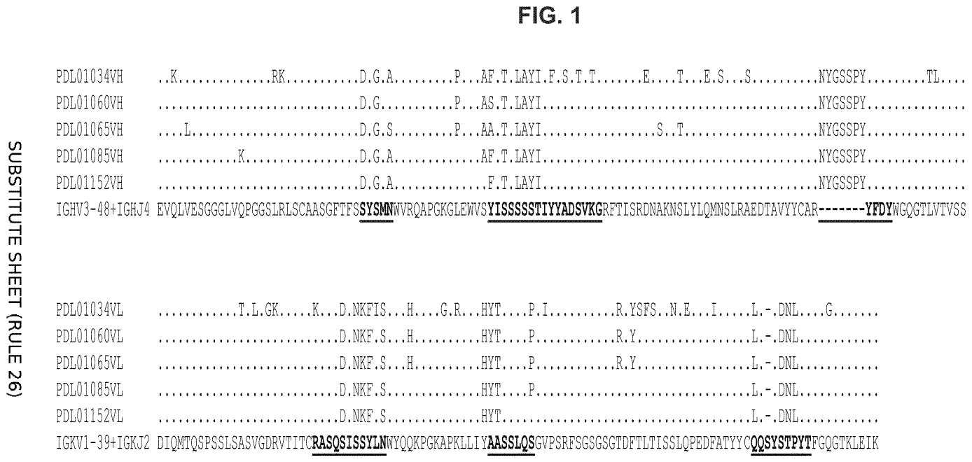

After evaluation of the hybridoma derived antibodies (described in Examples 6, 7 and 8), clone 5F9 was selected for full humanization and optimization. The primary purpose of humanization was to eliminate as much of the mouse derived sequence as possible to minimize potential immunogenicity and optimize the binding and stability properties of the binding domain at the same time. Clone 5F9 anti-PD-L1 murine monoclonal antibody in scFv-hFc format (PDL01034; VH SEQ ID NO:276; VL SEQ ID NO:277) was humanized in multiple rounds of CDR grafting and refinements using BioLuminate software package (Schrodinger, LLC, New York, USA). A homology models of mouse clone 5F9 were created based on PDB IDs 5B3J, 5VYF and 6HHC, and the most geometrically suitable and homologous human frameworks for CDR grafting were identified using the software's default and modified settings. Seven CDR-grafted molecules were produced in small scale and tested unpurified for binding to cells expressing full length human- or cyno-PD-L1. After scale-up and purification, their binding kinetics to recombinant human PD-L1 antigen were measured using Biacore T200 (GE Healthcare Life Sciences, USA), stability by measuring Tₘ and T_{agg} using the Uncle instrument (Unchained Labs, USA) and expression levels and initial aggregate levels were noted (data not shown). In Stage 2, molecules PDL01060 and PDL01065 were further germlined and molecule PDL01085 was shown to have similar binding properties to the parental mAb 5F9 and good thermal stability (data not shown). In Stage 3, additional framework residues were mutated in sets and combinations of sets to convert mouse residues of PDL01085 to human germline sequences IGHV3-48*01 and IGHJ4*01 for heavy chain and IGKV1-39*01 and IGKJ2*01 for light chain. Molecule PDL01152 was identified to carry the best combination of binding, functional, and developability properties. All non-germline residues in frameworks are essential for either binding or stability. The progression of humanization from mouse to humanized sequences is shown as an amino acid alignment in FIG. 1 (from mouse anti-PD-L1 clone 5F9 (PDL01034) to humanized PDL01152).

All antibody protein engineering was performed at the protein sequence level. Genes corresponding to designed proteins were synthesized by Integrated DNA Technologies Inc., Coralville, IA, USA using their online gene design tool for optimized expression in mammalian system. Synthetic genes were combined with each other or with expression vectors using either NEBuilder HiFi DNA Assembly Cloning Kit (New England Biolabs, Beverly. MA) or using standard molecular biology techniques and methods generally disclosed in, e.g., PCT Application Publication No. WO 2007/146968, U.S. Patent Application Publication No. 2006/0051844, PCT Application Publication No. WO 2010/040105, PCT Application Publication No. WO 2010/003108, and U.S. Pat. No. 7,166,707. DNA sequences were verified using Sanger sequencing at GENEWIZ, South Plainfield, NJ, USA.

### Example 5. Production and Evaluation of the Biophysical Characteristics of the Anti-PD-L1 scFv Variants

Different humanized versions of the Clone 5F9 scFv were produced as monospecific DNA constructs by attaching the scFv sequence to the N-terminus of a wildtype IgG1 Fc region. Following transient expression and purification of constructs with sufficient quantity, these constructs were characterized for expression, thermal stability by differential scanning fluorimetry (DSF), and binding affinity to human PD-L1 ECD by SPR (as described in Example 6).

The mid-point of the first melting transition (Tₘ₁) was measured using DSF. Tₘ₁ was used to reflect the temperature required to unfold the first, or least-stable, binding domain in the construct. DSF was run using the Uncle instrument from Unchained Laboratories. Samples were analyzed at 1 mg/mL in PBS using intrinsic fluorescence (no additional dyes were used to assess protein unfolding). PDL01034, the parent construct, had the lowest recorded Tₘ₁ of 55.1° C., whereas the derivative constructs all had values greater than 63° C., indicating that all are more thermostable.

As shown in Table 6, examination of binding to human PD-L1 ECD by SPR revealed varying effects on affinity during humanization. Two variants (PDL01085 and PDL01152) retained similar binding affinity to the parent (~1 nM KD), while two (PDL01060 and PDL01065) had decreased affinity, exhibited as a higher KDs (217 and 16 nM, respectively). Additionally, a significant increase in expression was obtained with three variants (PDL01060, PDL01085, and PDL01152) compared to the unmodified parent construct (PDL01034). The improved expression and Tₘ₁ values suggest that the variants have improved stability and solubility, which are considered beneficial properties therapeutic protein drugs, while maintaining similar binding affinity to the parent binding domain.

TABLE 6
Summary of expression, thermostability, and affinity of
preferred anti-PD-L1 variants compared to the parent
sequence

| | Expres- | | Affinity to human PD-L1 | | |
|---|---|---|---|---|---|
| Construct | sion | DSF | ECD by SPR | | |
| Details | Titer | Tₘ₁ | Ka | Kd | KD |
| Name | μg/mL | (° C.) | (1/Ms) | (1/s) | (nM) |
| PDL01034 | 25.6 | 55.1 | 1.4E+06 | 2.4E−04 | 0.2 |
| (Parent) | | | | | |
| PDL01060 | 220.7 | ND | 1.1E+02 | 2.2E−07 | 217 |
| PDL01065 | 15 | ND | 8.3E+04 | 1.6E−08 | 16 |
| PDL01085 | 166.8 | 67.3 | 9.1E+04 | 9.5E−04 | 1 |
| PDL01152 | 220.5 | 63.1 | 1.1E+06 | 1.2E−03 | 1.1 |

### Example 6. Methodology for Using Surface Plasmon Resonance (SPR) to Determine the Binding Affinity of Anti-PD-L1 Binding Domains

SPR binding affinity studies of mono- and bispecific proteins binding to recombinant human PD-L1 ectodomain (ECD) were conducted at 25° C. in HBS-EP+ buffer (Cytiva, BR100826) with 0.1% bovine serum albumin (BSA) on a Biacore 8K or Biacore T200 system. Mouse anti-human IgG (Cytiva, BR100839) at 25 μg/ml in 10 mM sodium acetate pH 5.0 was immobilized at a density of 2,000-4,000 response units (RU) onto each flow cell of a CM5 research-grade sensor chip (Cytiva, BR100530) by standard amine coupling chemistry. Each anti-PD-L1 protein at approximately 40 nM in HBS-EP+ with 0.1% BSA buffer was captured in a flow cell with the immobilized anti-human IgG at a flow rate of 10 L/min for up to 30 seconds, leaving one flow cell surface unmodified as the reference. Using a multi-cycle kinetics mode, a buffer blank and five different concentrations of ECD ranging from 0.5 nM to 243 nM were sequentially injected through each flow cell at 30 L/min with association times varying from 300-600 seconds and dissociation times varying from 600-1200 seconds. Regeneration was achieved by injection of 3 M MgCl₂ at a flow rate of 30 L/min for up to 40 seconds followed by HBS-EP+ with 0.1% BSA buffer stabilization for 1 min.

Sensorgrams obtained from kinetic SPR measurements were analyzed using the double subtraction method. The signal from the reference flow cell was subtracted from the analyte binding response obtained from flow cells with captured ligands. The buffer blank response was then subtracted from analyte binding responses and the final double-referenced data were analyzed with Biacore T200 Evaluation software (v2.0, Cytiva), globally fitting data to derive kinetic parameters. All sensorgrams were fitted using a simple one-to-one binding model.

Several monospecific anti-PD-L1 scFv-Fc proteins were evaluated for their binding affinity to monomeric PD-L1 ECD using SPR (see Table 6).

### Example 7: Binding of PD-L1-Binding Molecules to Human and Cynomolgus PD-L1-Expressing CHO Cell Lines

PD-L1 scFv binding domains were evaluated using a MesoScale Discovery (MSD) assay. 50,000 cells (CHOK1SV/huPDL1, CHOK1SV/cynoPDL1 and parental CHOK1SV) were plated on multi-array 96-well high binding plates (MSD) and then blocked with 40% FBS/PBS for 1 hour at 37° C. Plates were washed and titrated test samples were added and incubated for 1 hour at RT. Plates were again washed and 0.5 μg/ml goat anti-human Sulfo-TAG detection antibody (MSD) was added and incubated for 1 hour at RT. Plates were washed and 1× surfactant-free MSD Read Buffer was added and read on a Meso QuickPlex SQ120 plate reader using Discovery Workbench software (MSD).

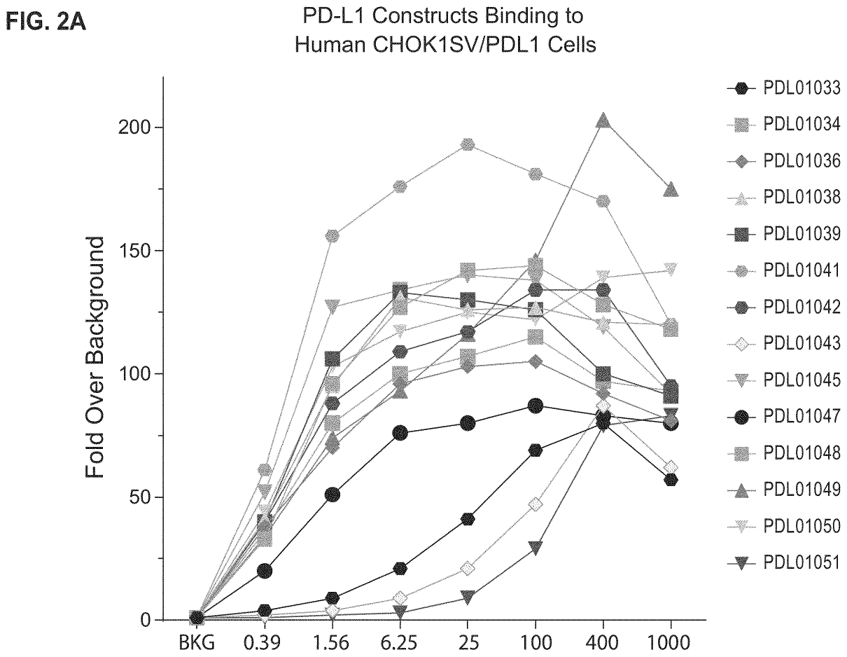

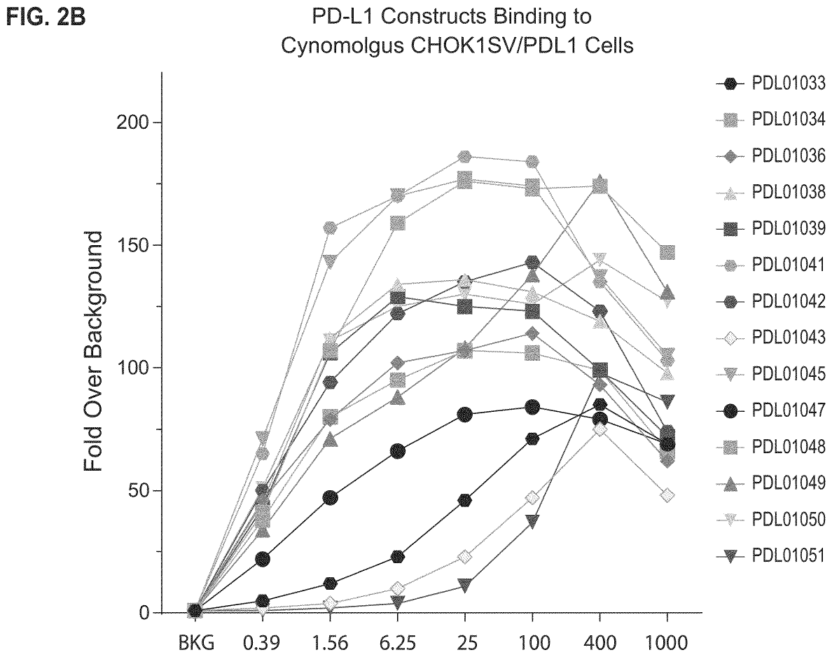

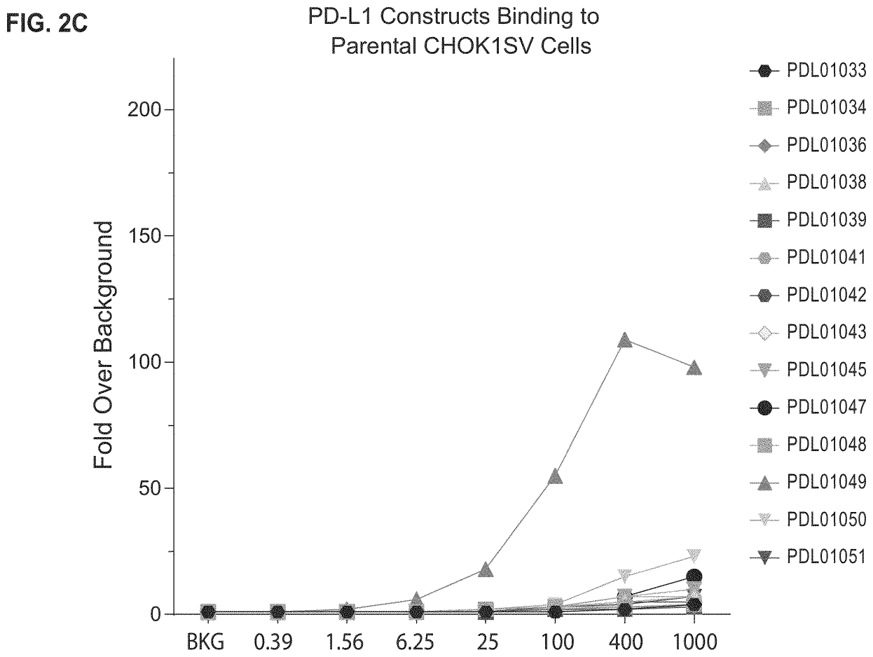

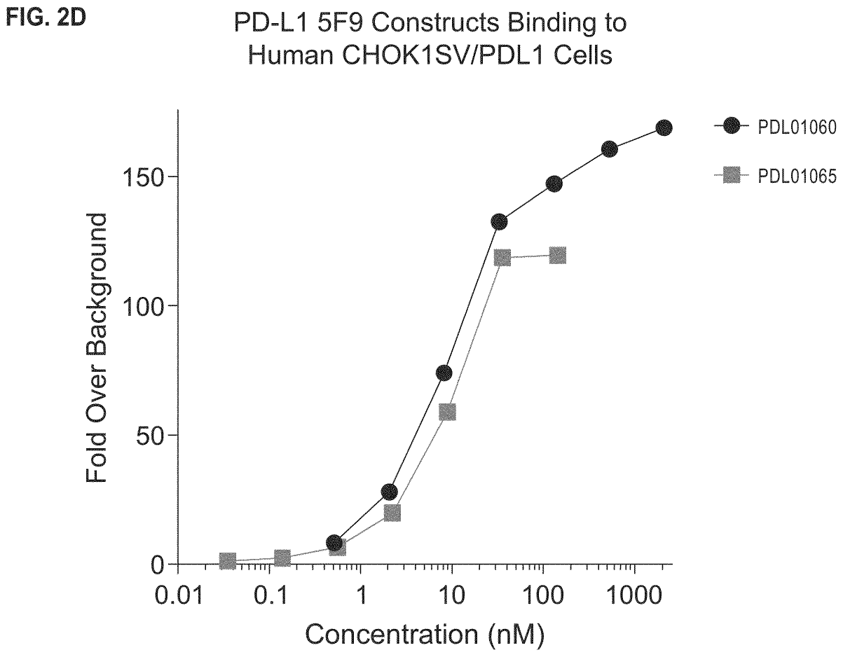

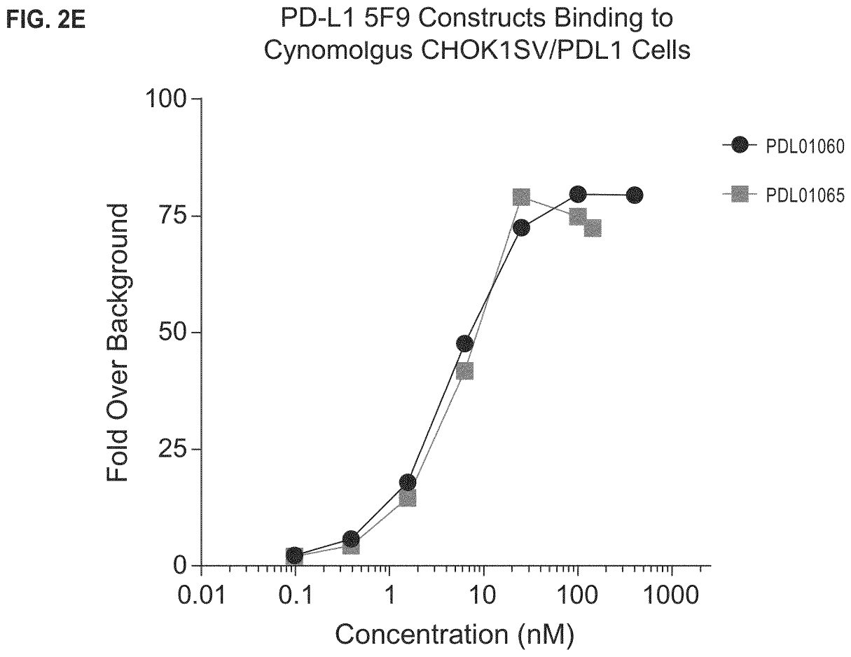

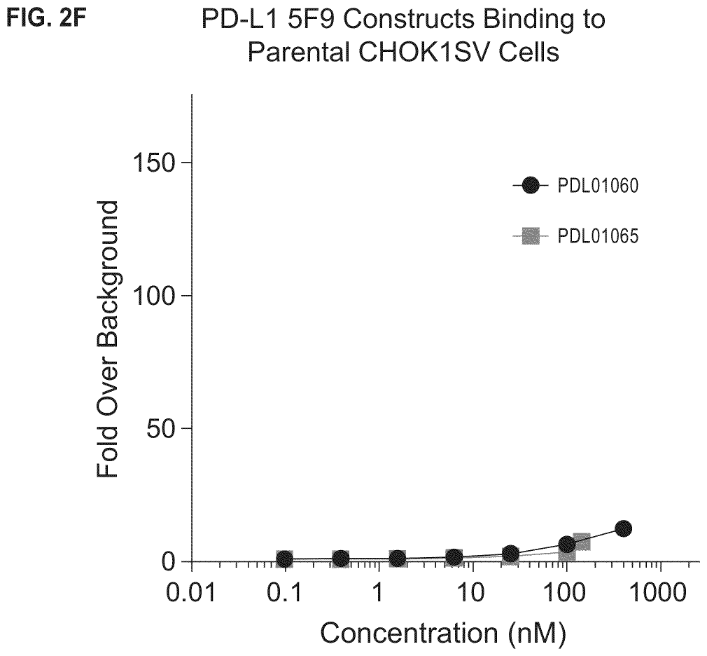

As shown in FIG. 2A-FIG. 2C, all PD-L1 antibodies bound to both human (FIG. 2A) and cynomolgus (FIG. 2B) PD-L1-expressing cells. Only one antibody, PDL01049, had increased non-specific binding to parental CHOK1SV cells (FIG. 2C) and was excluded from further testing.

After further evaluation, 5F9 was the desired PD-L1 binding domain and humanization was done. FIG. 2D-FIG. 2F reveal the PD-L1 two leads, PDL01060 and PDL01065, binding to human (FIG. 2D), cynomolgus (FIG. 2E) and parental CHOK1SV (FIG. 2F) cells using the MSD platform assay. Both humanized molecules had good binding to PD-L1 and did not show non-specific binding to parental cells.

### Example 8: Binding of PD-L1-Binding Molecules to Various Human PD-L1 Expressing Cell Lines

Cell binding studies were completed to demonstrate that the PD-L1 scFv binding domains bound sufficiently to cells expressing PD-L1 (CHOK1SV/huPD-L1. CHOK1SV/cynoPD-L1, K562 and KG-1), but not to cells without expression of PD-L1 (Jurkat, U-937, C4-2B or parental CHOK1SV). Binding studies were performed using the sensitive MSD assay platform.

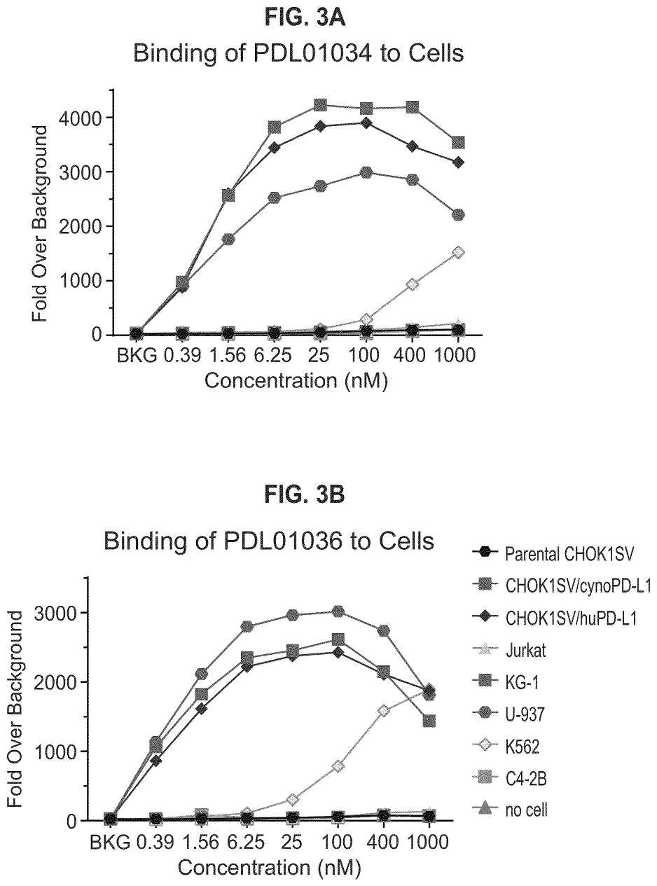

FIG. 3A-FIG. 3B graphically represent the binding of 5F9 clone PDL01034 (FIG. 3A) and 6F6 clone PDL0136 (FIG. 3B) to these various cell lines. Both molecules strongly bind, as expected, to human and cynomolgus PD-L1-expressing CHOK1SV and KG-1 that all express high levels of PD-L1. There was additional weak binding to K562 which express low levels of PD-L1. Of importance, neither molecule bound to negative control cell lines.

### Example 9: Generation of CD40-Expressing CHO Cells and Recombinant Extracellular Domain Proteins

The protein sequences defining the human and non-human primate CD40 full length and extracellular domains (ECDs) were obtained from the Genbank database and are listed in Table 7. HAC protein sequence consisting of His tag, AviTag and C-tag and represents a set of C-terminal tags for purification, detection, and biotin-based labeling purposes. mFc represents a protein sequence of murine IgG2a hinge and Fc region.

TABLE 7
SEQ ID NOs of CD40 constructs for production
of cell lines and recombinant proteins

| | | SEQ |
|---|---|---|
| Construct | | ID |
| Name | Construct Description | NO |
| CD4001003 | Cyno CD40 ECD-mFc | 80 |
| CD4001004 | Cyno CD40 ECD-HAC tag | 81 |
| CD4001005 | human CD40 ECD-mFc | 82 |
| CD4001006 | human CD40 ECD- HAC tag | 83 |
| CD4001012 | human CD40 full-length | 84 |
| CD4001013 | Cyno CD40 full-length | 85 |

The DNA containing the nucleotide sequences for proteins listed in Table 7 was synthesized by Integrated DNA Technologies Inc., Coralville, IA, USA and inserted either into an expression vector appropriate for mammalian cell expression and secretion or into an expression vector appropriate for cell-surface expression that included the ability to apply selective pressure to generate stable transfectants. These reagents were used to assess the cross reactivity and binding strength of anti-CD40 binding domains to Human CD40 and the species to be used in potential toxicology assessments. The DNA expression vectors encoding HAC tags were used to transiently transfect human embryonic kidney fibroblast (HEK)-293 cells grown in suspension culture. After several days in culture, the conditioned media was clarified via centrifugation and sterile filtration. Protein purification was performed utilizing a combination of Immobilized Metal Affinity Chromatography (IMAC) followed by size exclusion chromatography (SEC). SEC removed aggregated and clipped product and other host cell contaminants. SEC was also used to buffer-exchange the protein into phosphate-buffered saline (PBS). Final purity was determined by analytical SEC and typically exceeded 90%. Protein batches were sterile-filtered and stored at 4° C. if the intent was to use within the next week or were frozen in aliquots in a −80° C. freezer.

Plasmid DNA encoding full-length constructs was digested with a restriction enzyme and ethanol precipitated, then dissolved in ultrapure water, then Maxcyte Electroporation Buffer. Linearized DNA was transfected into CHO-K1SV cells (CDACF-CHO-K1SV cells (ID code 269-W3), Lonza Biologics) by electroporation. Transfected cells were transferred from the electroporation cuvette to a T75 culture flask, rested, and then gently resuspended in 15 mL of CD CHO media supplemented with 6 mM L-Glutamine in the T150 flask. The flask was put in a 37° C., 5% C02 incubator and allowed to recover for 24 hours prior to placing in the selection conditions. On the day following transfection, the cells were centrifuged for 5 minutes at 1000 RPM and resuspended in CD CHO medium with 1×GS supplement and 50 μM MSX. After the bulk populations were recovered from initial selection, cells were evaluated for surface expression with commercially available reagents, and representative vials were frozen. To obtain clones with varying levels of expression, cells were sorted by flow cytometry, plated by limiting dilution, and allowed to grow for 2 weeks. Wells were imaged with a Clone Select Imager during the incubation to identify growth positive wells. Only wells with good quality images were selected for further expansion and characterization for surface expression by flow cytometry. All clones were frozen in banks at up to 30 vials per clone.

### Example 10. Generation of CD40 Antibodies by Immunization of Wild-Type Mice

CD40-specific antibodies were isolated from a hybridoma library generated after immunizing BALB/c mice with recombinant human antigen CD4001005 at Precision Antibody, Columbia, MD. Supernatants from hybridoma clones were assayed by ELISA, and identified wells were confirmed for specific binding using flow cytometry on CHO cells transfected with human and cynomolgus CD40. Positive clones were selected for expansion, and viable cells were frozen for RNA extraction and variable domain analysis. Supernatants were saved for additional analyses.

The variable heavy (VH) and light (VL) domain sequences for selected hybridoma clones were obtained by RT-PCR after isolating total RNA. Briefly, total RNA was isolated from the hybridoma clone cell banks using Qiagen's RNeasy Plus Kit (Qiagen, Venlo Netherlands), and 400 ng of total RNA were used in a First Stand cDNA synthesis reaction using oligo dT and Superscript IV (Thermo Fisher Scientific. Waltham, MA), following manufacturer's protocol. Following cDNA synthesis, the variable region cDNA was amplified using 1 μL of cDNA and a series of primer mixes for mouse IgG VH, Vic, and VX (Novagen Mouse Ig-Primer Set, EMD Millipore, Temecula. CA). PCR products for each clone were directly sequenced using the reverse (constant domain) PCR primer and standard Sanger sequencing methods. DNA sequence analysis identified 16 different antibodies: 1A7, 1B12, 1D5, 4G10, 5B9, 5E3, 6A4, 6B3, 7A8, 7G9, 71-112, (CD4001014 to 24) 1G4, 2H7, 6B12, 9A6 and 9F1 (CD4001036, 38, 40, 42, 44) (see Table 2). Sequences were then converted to scFv in both VH-VL and VL-VH orientation by amplifying the variable domains using specific primers that contain overlapping sequences. The scFv-coding fragments of antiCD40 binding domains were fused to N-terminus of human WT IgG1 Fc with antiPDL1 scFv 5F9 fused to C-terminus. These constructs were assembled into a mammalian expression vector using NEBuilder HiFi DNA Assembly Cloning Kit (New England Biolabs, Beverly, MA).

TABLE 8
SEQ ID NOs of anti-CD40 hybridoma-derived
scFv-Fc constructs

| | | SEQ |
|---|---|---|
| Construct | | ID |
| Name | Construct Description | NO |
| CD4001014 | 1A7HL x IgG1 WT Fc x 5F9 | 86 |
| CD4001015 | 1B12HL x IgG1 WT Fc x 5F9 | 88 |
| CD4001016 | ID5HL x IgG1 WT Fc x 5F9 | 90 |
| CD4001017 | 4G10HL x IgG1 WT Fc x 5F9 | 92 |
| CD4001018 | 5B9HL x IgG1 WT Fc x 5F9 | 94 |
| CD4001019 | 5E3HL x IgG1 WT Fc x 5F9 | 96 |
| CD4001020 | 6A4HL x IgG1 WT Fc x 5F9 | 98 |
| CD4001021 | 6B3HL x IgG1 WT Fc x 5F9 | 100 |
| CD4001022 | 7A8HL x IgG1 WT Fc x 5F9 | 102 |
| CD4001023 | 7G9HL x IgG1 WT Fc x 5F9 | 104 |
| CD4001024 | 7H12HL x IgG1 WT Fc x 5F9 | 106 |
| CD4001027 | ID5LH x IgG1 WT Fc x 5F9 | 118 |
| CD4001036 | 1G4HL x IgG1 WT Fc x 5F9 | 108 |
| CD4001038 | 2H7HL x IgG1 WT Fc x 5F9 | 110 |
| CD4001040 | 6B12HL x IgG1 WT Fc x 5F9 | 112 |
| CD4001042 | 9A6HL x IgG1 WT Fc x 5F9 | 114 |
| CD4001044 | 9F1HL x IgG1 WT Fc x 5F9 | 116 |

### Example 11. CD40 ECD Domain Mapping of Hybridoma Clones

Taking advantage of no binding activity of hybridoma antiCD40 clones to murine CD40 and modular domain structure of CD49 ECD we designed a set of human/murine domain swapped hybrid constructs. Human CD40 ECD was divided to 3 domains with junctions at His78 and His122. These constructs were expressed as full-length hybrid CD40 proteins and transiently expressed on the surface of CHO cells N-terminal 2×FLAG tag was used to determine expression level of all constructs and for normalization of binding signal. Transmembrane and intracellular domains are human CD40-derived for all the constructs. Table 9 describes the constructs used in the domain mapping.

TABLE 9
SEQ IDs of CD40 ECD domain swap constructs
for domain mapping of hybridoma clones

| | | SEQ |
|---|---|---|
| Construct | | ID |
| Name | Construct Description | NO |
| CD4001052 | 2xFLAG-huCD40 full-length | 119 |
| CD4001053 | 2xFLAG-hu_mulCD40 full-length | 120 |
| CD4001054 | 2xFLAG-hu_mu2CD40 full-length | 121 |
| CD4001055 | 2xFLAG-hu_mu3CD40 full-length | 122 |
| CD4001056 | 2xFLAG-muECD_huCD40 full-length | 123 |

6 hybridoma clones in scFv format, 1A7, 2H7, 5B39, 1D5, 1G4 and 9A7 were tested for binding to human/murine hybrid CD40 ECDs expressed on CHO cells by flow cytometry. Anti-FLAG antibody stained all cells carrying hybrid construct strongly as shown in Table 10.

TABLE 10
Relative expression of hybrid CD40 on surface of CHO cells

| | huCD40 | hu/mu1CD40 | hu/mu2CD40 | hu/mu3CD40 | muCD40 | vector |
|---|---|---|---|---|---|---|
| FLAG signal | 100 | 92 | 46 | 48 | 81 | 3 |
| [%] | | | | | | |

Signals from the hybridoma clones were normalized both to FLAG signal and to huCD40 signal and are shown in Table 11. The data shows that 1A7 binds to domain 2, 2H7 binds primarily to domain 2 and domain 1, 5B9 binds to domain 1, and 1D5, 1G4 and 9A7 hybridoma clones bind to domain 3.

TABLE 11
Relative % binding of hybridoma clones to human/
murine hybrid CD40 on surface of CHO cells

| | | hu/ | hu/ | hu/ | |
|---|---|---|---|---|---|
| | huCD40 | mu1CD40 | mu2CD40 | mu3CD40 | muCD40 |
| 1A7 | 100 | 95 | 2 | 285 | 1 |
| 2H7 | 100 | 39 | 4 | 137 | 2 |
| 5B9 | 100 | 3 | 221 | 351 | 1 |
| 1D5 | 100 | 100 | 221 | 11 | 6 |
| 1G4 | 100 | 105 | 178 | 11 | 6 |
| 9A7 | 100 | 111 | 163 | 14 | 9 |

### Example 12. CD40/CD40l Interaction Blocking by Hybridoma Clones in scFv Format

To investigate ability of hybridoma antibody clones 1D5, 2H7 and 5B9 (constructs CD4001066, 64 and 65) to block CD40 receptor-CD40 ligand binding on cells, two sets of experiments were carried out. In the first, CD40 expressing CHO cells were saturated by CD40L followed by binding of antibodies and in the second, CD40 expressing CHO cells were saturated by antibodies followed by CD40L. Both experiments show that 1D5 and 2H7 block CD40/CD40L interaction while 5B9 does not. All 3 hybridoma clones were in ADAPTIR format with anti-PD-L1 scFv on N-terminus and anti-CD40 scFv on C-terminus.

For both assays 100,000 CD40-expressing HEK293 cells were plated. In the first assay, His-tagged CD40L (BPS Bioscience) dilutions were added to the cell and incubated on ice for 1 hour. The cells were washed and 100 nM anti-CD40 antibodies were added, incubated on ice for 30 minutes and then repeatedly washed. Cells were incubated with PE-labelled goat-anti-hulgG F(ab′)2 (Jackson). Following a 30-minute incubation, cells were washed and analyzed by flow cytometry.

In the second assay, serially diluted anti-CD40 antibodies were added and incubated on ice for 1 hour. The cells were washed and 100 nM CD40L was added for 30 minutes. After repeated washes, PE-labelled anti-His (Biolegend). Following a 30-minute incubation, cells were washed and analyzed by flow cytometry. All incubations and washes were done in staining buffer. All samples were collected using an BD™ LSR-II flow and analyzed by FlowJo flow cytometry analysis software. Mean fluorescence intensity (MFI) of bound molecules on cells was determined after exclusion of doublets.

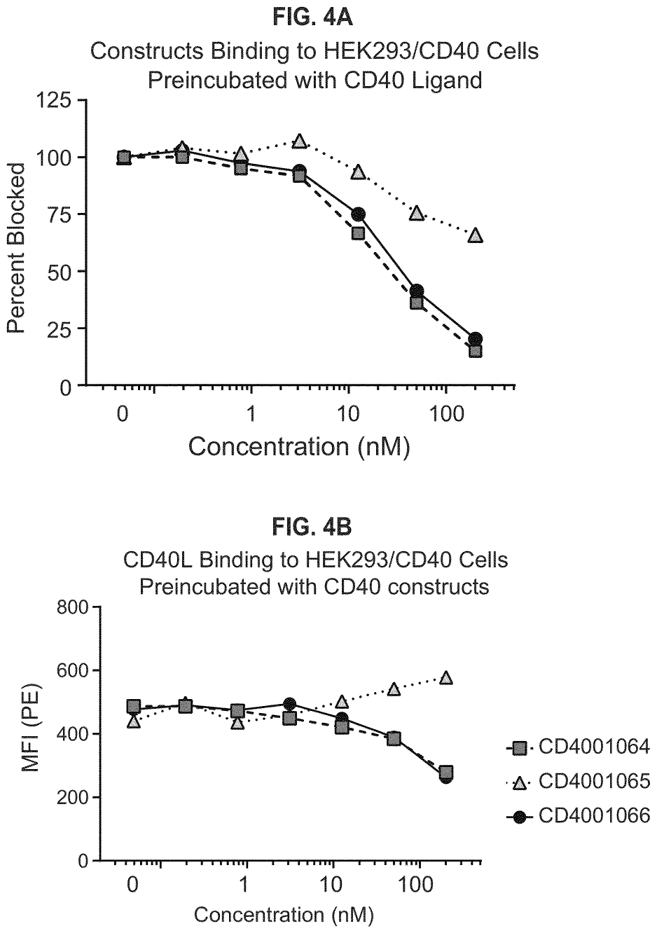

FIG. 4A shows the percentage of anti-CD40 antibodies that are blocked by preincubating with CD40L. Both CD4001064 (2H7) and CD4001066 (1D5) are blocked by CD40L. In contrast, CD4001065 (5B9) does not have significant reduction in binding to CD40 when competing with CD40L binding. Alternatively, FIG. 4B shows the MFI of CD40L bound to CD40-expressing cells following preincubation with anti-CD40 antibodies. Here. CD4001064 (2H7) and CD4001066 (1D5) block CD40L binding starting at 10 nM.

### Example 13. Anti-PD-L1 and Anti-CD40 Bispecific Proteins with Different Structures and Binding Valency

While the optimal distance and geometry to form an immune synapse between a PD-L1 expressing tumor cell and CD40 receptor expressing cell is unknown, and epitopes of our anti-PD-L1-specific and anti-CD40-specific binding domains are pre-determined and immovable, testing of different bispecific structures was performed to achieve the optimal functional activity. Binding valency was also investigated by producing and testing molecules with heterodimeric structures in ADAPTIR-FLEX format, that contained one or two binding domains against PD-L1 and against CD40 and located on either the N- or C-terminal positions on the Fc region (see Table 12). Additionally, the effect of changing the order of the scFv domains (VH-VL versus VL-VH) was examined. These structural changes, in addition to impacting the binding affinity and functional performance of ADAPTIR and ADAPTIR-FLEX, can also have a significant impact on the expression levels and stability of the bispecific protein and were examined as part of this work.

TABLE 12
SEQ ID NOs and descriptions of bispecific constructs
with different structures and biding valency

| | | SEQ |
|---|---|---|
| Construct | | ID |
| Name | Construct Description | NO |
| CD4001047 | CD40_2H7 x hFc x PDL_5F9 | 128 |
| CD4001048 | CD40_5B9 x hFc x PDL_5F9 | 129 |
| CD4001049 | CD40_1D5 x hFc x PDL_5F9 | 130 |
| CD4001064 | PDL_5F9 x hFc x CD40_2H7 | 131 |
| CD4001065 | PDL_5F9 x hFc x CD40_5B9 | 132 |
| CD4001066 | PDL_5F9 x hFc x CD40_1D5 | 133 |
| CD4001067 | PDL_5F9 x Fc-Knob; | 134, |
| | CD40_2H7 x Fc-Hole | 135 |
| CD4001068 | PDL_5F9 x Fc-Knob; | 136, |
| | CD40_5B9 x Fc-Hole | 137 |
| CD4001069 | PDL_5F9 x Fc-Knob; | 138, |
| | CD40_1D5 x Fc-Hole | 139 |
| CD4001070 | Fc-Knob x PDL_5F9; CD40_ | 140, |
| | 2H7 x Fc-Hole x PDL_5F9 | 141 |
| CD4001071 | Fc-Knob x PDL_5F9; CD40_ | 142, |
| | 5B9 x Fc-Hole x PDL_5F9 | 143 |
| CD4001072 | Fc-Knob x PDL_5F9; CD40_ | 144, |
| | 1D5 x Fc-Hole x PDL_5F9 | 145 |
| CD4001073 | CD40_2H7 x Fc-Knob; CD40_ | 146, |
| | 2H7 x Fc-Hole x PDL_5F9 | 147 |
| CD4001074 | CD40_5B9 x Fc-Knob; CD40_ | 148, |
| | 5B9 x Fc-Hole x PDL_5F9 | 149 |
| CD4001075 | CD40_1D5 x Fc-Knob; CD40_ | 150, |
| | 1D5 x Fc-Hole x PDL_5F9 | 151 |
| CD4001076 | PDL_5F9 x Fc-Knob; PDL_ | 152, |
| | 5F9 x Fc-Hole x CD40_2H7 | 153 |
| CD4001077 | PDL_5F9 x Fc-Knob; PDL_ | 154, |
| | 5F9 x Fc-Hole x CD40_5B9 | 155 |
| CD4001078 | PDL_5F9 x Fc-Knob; PDL_ | 156, |
| | 5F9 x Fc-Hole x CD40_1D5 | 157 |
| CD4001079 | Fc-Knob x CD40_2H7; PDL_ | 158, |
| | 5F9 x Fc-Hole x CD40_2H7 | 159 |
| CD4001080 | Fc-knob x CD40_5B9; PDL_ | 160, |
| | 5F9 x Fc-Hole x CD40_5B9 | 161 |
| CD4001081 | Fc-knob x CD40_1D5; PDL_ | 162, |
| | 5F9 x Fc-Hole x CD40_1D5 | 163 |
| PDL01127 | Positive control: bivalent | 167, |
| | PDL1-1 x Fc x hexavalent | 168 |
| | CD40 | |
| PDL01029 | Positive control: bivalent | 165 |
| | PDL 1-2 x Fc | |
| PDL01085 | PDL_5F9 x Fc | 166 |
| PDL01153 | Fc x PDL_5F9 | 169 |
| POC01043 | Positive control: bivalent | 164 |
| | CD40 x Fc x bivalent PDL1-2 | |

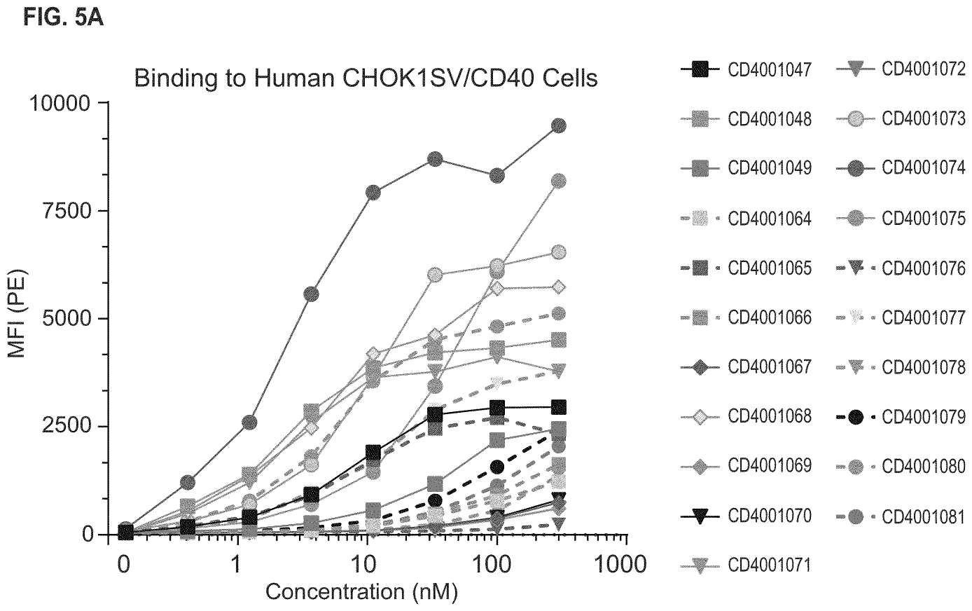

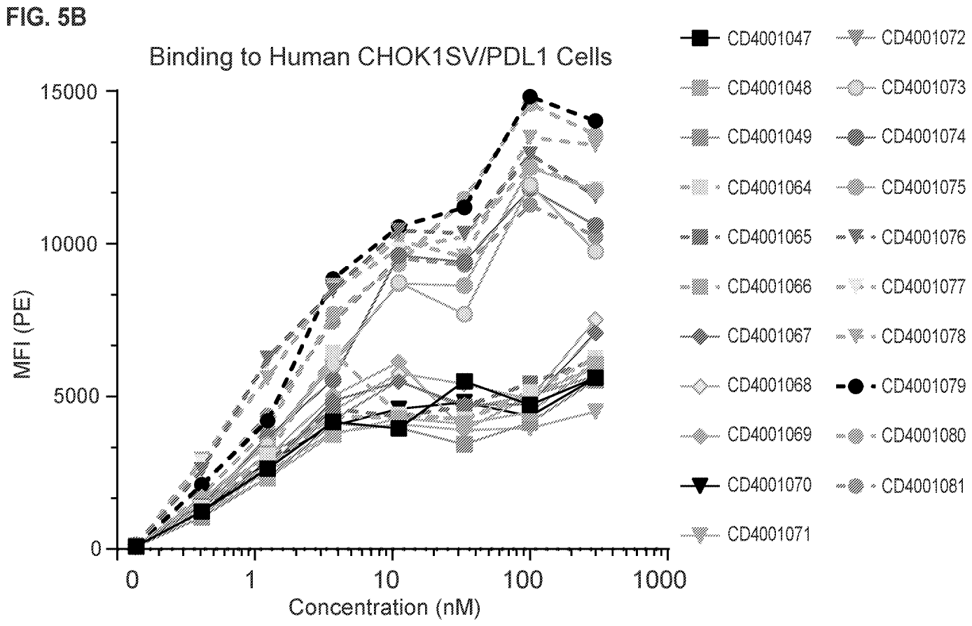

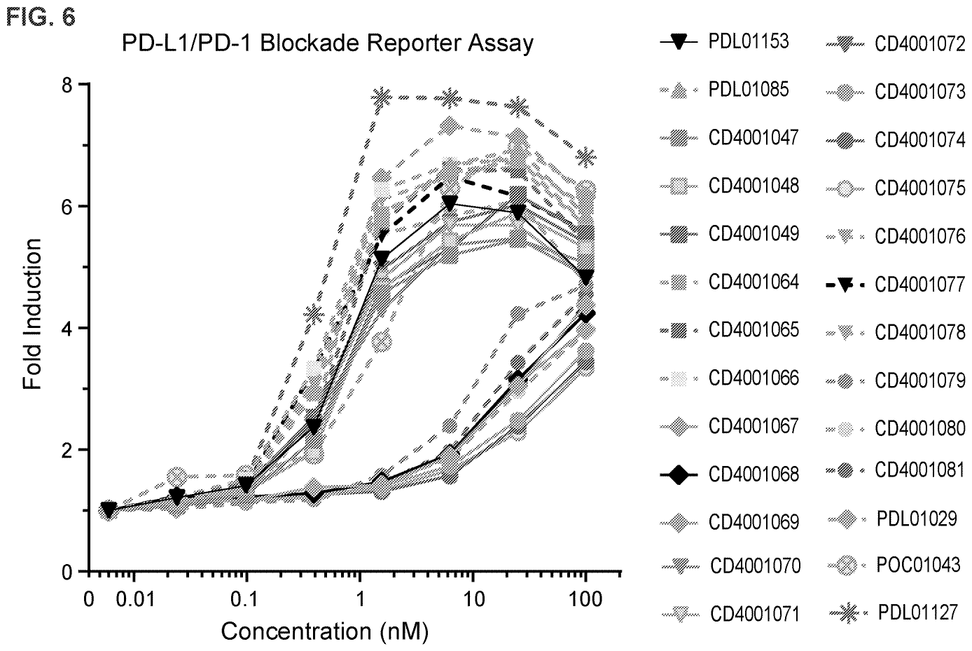

To evaluate the effect on binding domains, geometry and binding valency, the combinations of molecules were first tested for binding to both CD40- and PD-L1-expressing CHOK1SV cells via flow cytometry (FIG. 5A and FIG. 5B, respectively). The experimental methods and analysis were similar to descriptions above. Several overall observations were determined during this matrix evaluation. First, there was a drop in CD40 binding when the anti-CD40 binding domain was on the C-terminus whether as a monovalent or bivalent binding domain. Overall, monovalent CD40 binding was significantly lower than bivalent binding. In general, monovalent PD-L1 in combination with bivalent CD40 and bivalent PD-L1 on the N-terminus had higher PD-L1 binding than the other combinations.

To quantitate PD-L1/PD-1 blockade activity, the human PD-1 NFAT luciferase reporter Jurkat line (BPS) was incubated with TCR Activator PD-L1-expressing CHO target cells (BPS). Thirty-five thousand target cells were plated overnight in a 96-well assay plate. The following day diluted bispecific samples were added and incubated for 30 minutes. Eighty thousand reporter cells were then added and incubated for 6 hours at 37° C. The plate is then equilibrated to RT and 100 ml of RT Bio-Glo reagent (Promega) was added and incubated for 15 minutes. Luminescence was read on a MicroBeta 2450 Microplate Counter (Perkin Elmer). In the PD-L1/PD-1 blockade assay shown in FIG. 6 bivalent PD-L1 molecules were definitively better at blocking the PD-L1/PD-1 as measured by light production compared to molecules containing monovalent PD-L1. The effect was not dependent on the orientation of either binding domain or the valency of the anti-CD40 binding domain.

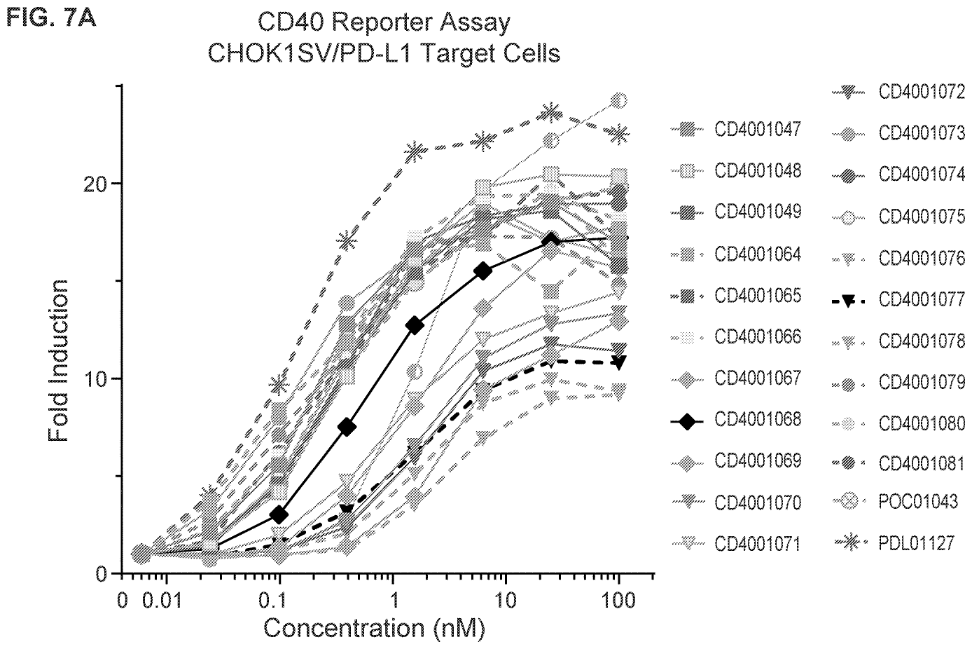

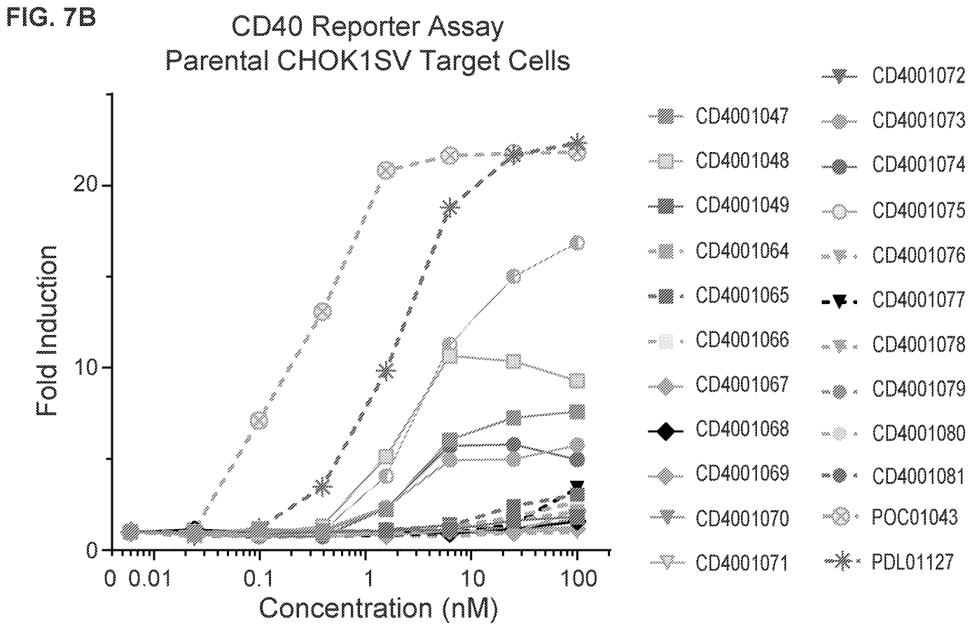

To examine CD40 activity, the human CD40/NFκB HEK293 luciferase reporter cells (BPS) are incubated with PD-L1-expressing target cells. Thirty thousand reporter cells were plated overnight in a 96-well assay plate. The following day diluted bispecific samples and 50,000 target cells were added incubated for 6 hours at 37° C. The plate is then equilibrated to RT and 100 ml of RT Bio-Glo reagent (Promega) was added and incubated for 10 minutes. Using the CD40 reporter assay shown in FIG. 7A, it was determined that bivalent CD40 molecules induced higher downstream signaling than monovalent CD40 molecules. Additionally, when monovalent, binding was reduced further when on the C-terminus. Overall, 5B9 binding domains generated the highest activity, followed by 1D5. The 2H7 binding domain generally had the lowest induction of CD40 activity. When using parental CHOK1SV cells (FIG. 7B), eliminating crosslinking through PD-L1, higher levels of CD40 activity were seen with 2H7 and 5B9 bivalent binding domains, which was more pronounced on the N-terminus.

### Example 14. Humanization of CD40-Specific Clone 1D5 in scFv Format

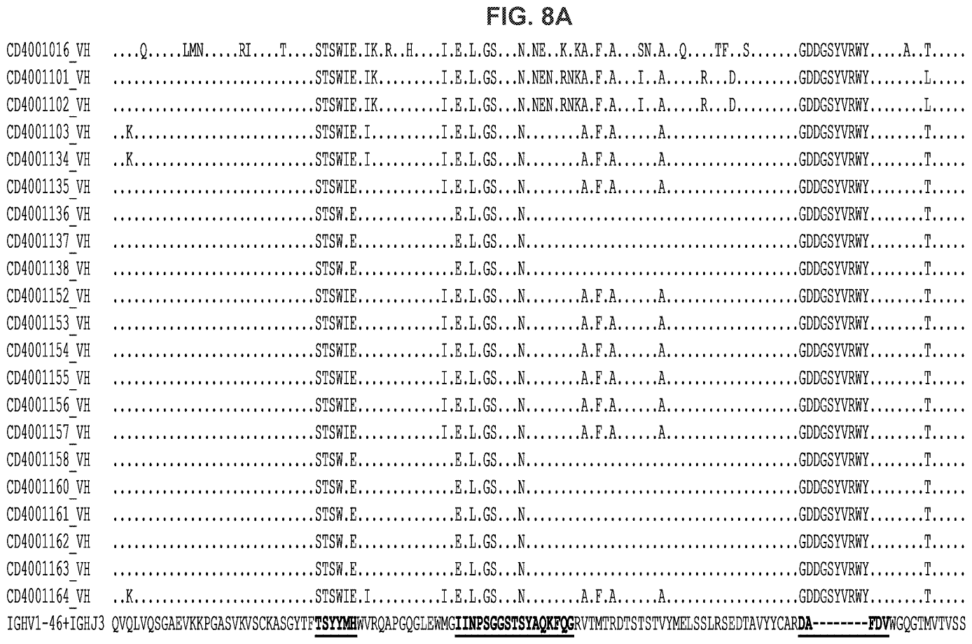

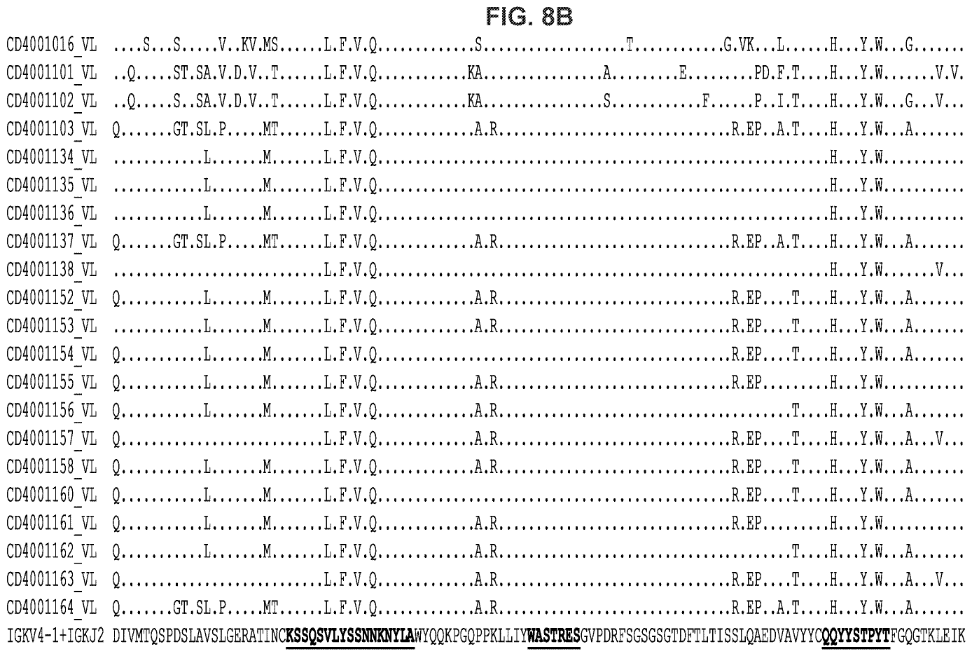

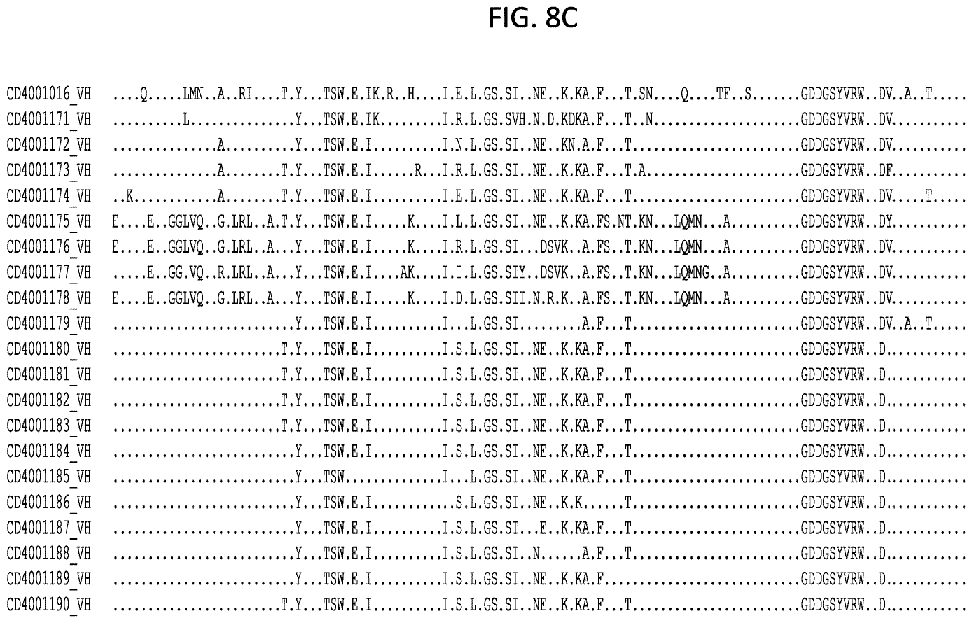

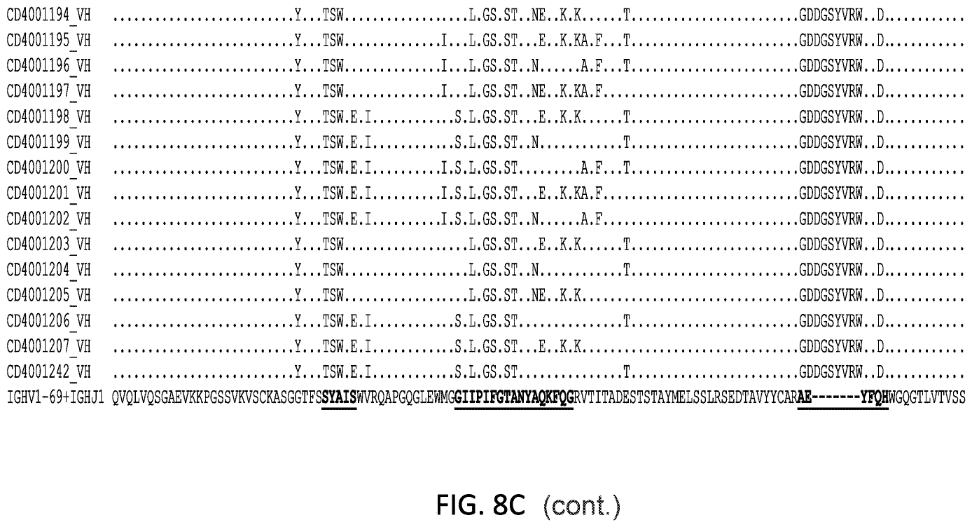

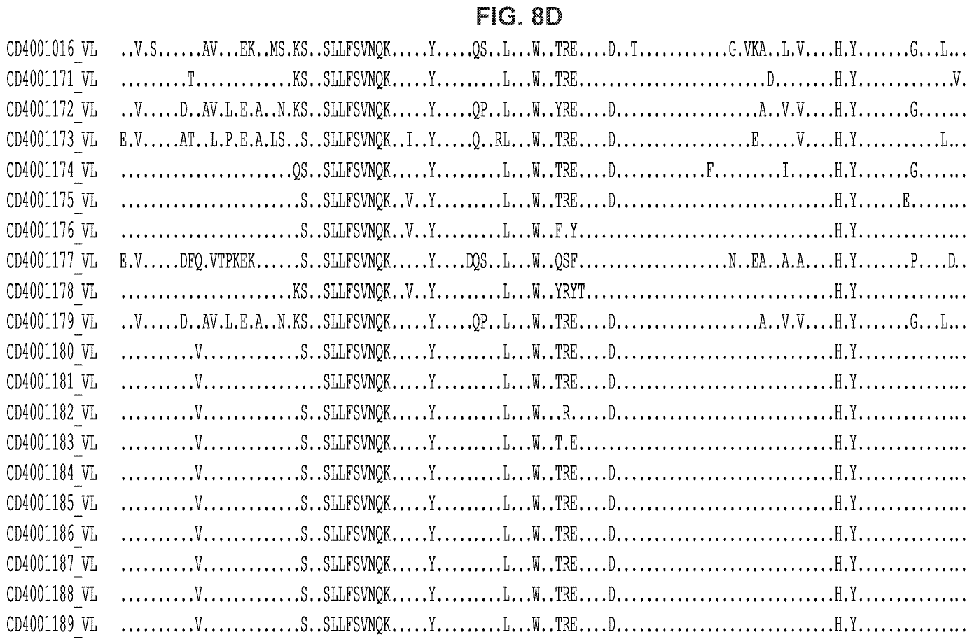

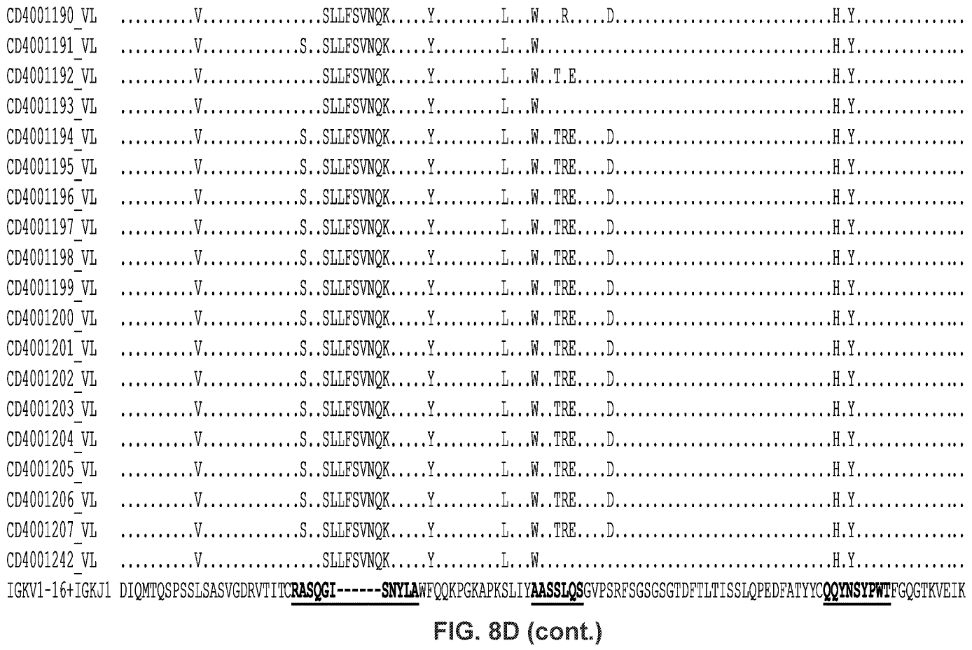

After evaluation of the hybridoma derived antibodies, clone ID5 was selected for full humanization and optimization. The primary purpose of humanization was to eliminate as much of the mouse derived sequence as possible to minimize potential immunogenicity and optimize the binding and stability properties of the binding domain at the same time. Humanization of clone 1D5 anti-CD40 murine monoclonal antibody was performed in scFv-hFc format in multiple rounds of CDR grafting and refinements using BioLuminate software package (Schrodinger, LLC, New York, USA). A homology models of mouse clone ID5 were created based on PDB ID 5KVD, and the most geometrically suitable and homologous human frameworks for CDR grafting were identified using the software's default and modified settings. These were 6U6U, 5N7W, 6OKM, 1T3F, 1CE1, 2WUB and 3NFS. 28 CDR-grafted molecules were produced in small scale and tested unpurified for binding to cells expressing full length human- or cyno-CD40. Clones with parent-like binding (CD4001101-03) were scaled-up and purified, their binding affinity by on-cell binding, stability by measuring Tₘ and T_{agg} using the Uncle instrument (Unchained Labs, USA) and expression levels and initial aggregate levels were noted. In Stage 2, molecule CD4001103 was further germlined and the humanized variants CD4041134-38 were tested similarly. Several additional sets of mutations were tested CD4001152-64, but none of the variants retained its binding activity in Fc-scFv format. Progression of humanization is described in FIG. 8A for VH and in FIG. 8B for VL chain.

New humanization of clone 1D5 anti-CD40 murine monoclonal antibody was performed in hFc-scFv format in multiple rounds of CDR grafting and refinements using BioLuminate software package (Schrodinger, LLC, New York, USA). A homology models of mouse clone 1D5 were created based on PDB IDs 5KVD, 4M61, 4M7K, 5HDQ and 4BZ1, and the most geometrically suitable and homologous human frameworks for CDR grafting were identified using the software's default and modified settings. 31 CDR-grafted molecules were produced in small scale and tested unpurified for binding to cells expressing full length human- or cyno-CD40. 10 clones with matching binding of parental mouse 1D5 were scaled-up and purified, their binding affinity by on-cell binding, stability by measuring Tₘ and T_{agg} using the Uncle instrument (Unchained Labs, USA) and expression levels and initial aggregate levels were noted (CD4001171-1180) In Stage 2, molecule CD4001180 was further germlined and the humanized variants CD4041181-1207 were tested similarly. Final humanized sequence of 1D5 antibody has VL from CD4001193 and VH from CD4001206. Progression of humanization is described in FIG. 8C for VH and in FIG. 8D for VL chain.

### Example 15. Production and Biophysical Evaluation of Partial Humanized Versions of Anti-CD40 Clone 1D5

Different humanized versions of the Clone 1D5 scFv were produced as monospecific DNA constructs by attaching the scFv sequence to the C-terminus of a wildtype IgG1 Fc region. Following transient expression and purification of constructs with sufficient quantity, these constructs were characterized for expression and thermal stability by differential scanning fluorimetry (DSF).

The mid-point of the first melting transition (Tₘ₁) was measured using DSF. Tₘ₁ was used to reflect the temperature required to unfold the first, or least-stable, binding domain in the construct. DSF was run using the Uncle instrument from Unchained Laboratories. Samples were analyzed at 1 mg/mL in PBS using intrinsic fluorescence (no additional dyes were used to assess protein unfolding).

These data showed that expression and thermal stability were sometimes negatively impacted by elimination of the mouse sequence (see Table 13). Many constructs exhibited Tₘ₁ greater than 60° C., indicating high thermal stability, while others were substantially decreased and excluded from further development. Expression was similarly variable with measured titers ranging from 44.0 up to 360.6 μg/mL. Binding affinity was determined by on-cell binding (Example 21). Combined, these data were used to look for general trends along with degree of human sequence incorporated as described in Example 15.

TABLE 13
Summary of expression and thermostability of
partially humanized anti-CD40 variants of
clone 1D5

| Construct | Expression | DSF |
|---|---|---|
| Details | Titer | Tₘ₁ |
| Name | μg/mL | (° C.) |
| CD4001171 | 221.2 | 60.1 |
| CD4001172 | 203.6 | 59.6 |
| CD4001173 | 228.1 | 58.1 |
| CD4001174 | 235.7 | 58.5 |
| CD4001176 | 207.8 | 51.4 |
| CD4001179 | 174.2 | 61.7 |
| CD4001180 | 204.7 | 65.7 |
| CD4001181 | 45.8 | 63.3 |
| CD4001182 | 208.6 | 63.7 |
| CD4001183 | 206.9 | 63.6 |
| CD4001184 | 88.6 | 63.1 |
| CD4001185 | 75.7 | 59.0 |
| CD4001186 | 99.8 | 64.4 |
| CD4001187 | 69.9 | 63.8 |
| CD4001190 | 76.6 | 63.5 |
| CD4001191 | 68.1 | 61.8 |
| CD4001192 | 57.1 | 63.0 |
| CD4001194 | 60.1 | 59.4 |
| CD4001195 | 46.1 | 55.8 |
| CD4001197 | 47.7 | 59.4 |
| CD4001199 | 52.9 | 63.9 |
| CD4001200 | 360.6 | 51.0 |
| CD4001201 | 102.1 | 62.9 |
| CD4001202 | 348.7 | 63.1 |
| CD4001203 | 274.6 | 54.2 |
| CD4001204 | 87.1 | 58.2 |
| CD4001205 | 64.5 | 59.0 |
| CD4001206 | 44.0 | 62.7 |
| CD4001207 | 46.4 | 62.0 |

### Example 16. PTM Removal of CD40-Specific Clone 1D5 by Phage Display

The HCDR3 of 1D5 antiCD40 binding domain contains a DDG sequence which contains 2 iso-aspartate isomerization motifs, DD and DG. To identify PTM de-risked variants, a phage display library was designed to mutate DDG sequence to all 20 possible amino acids. The library was synthesized at IDT, Coralville IA and assembled into a phage display vector using NEBuilder HiFi DNA Assembly Cloning Kit (New England Biolabs, Beverly, MA). Standard phage display library making and panning techniques were used. (Antibody Phage Display: Methods and Protocols, ed. Robert Aitken, 2009 Methods in Molecular Biology, vol 562) We used site-specifically biotinylated humnanCD40 ECD (CD4001006), streptavidin-coated magnetic beads (Dynabeads M-280 Streptavidin, Thermo Fisher Scientific), and KingFisher mL Purification System instrument (Thermo Fisher Scientific) for panning.

After the first round of panning with 200 nM biotin-CD40, there was no enrichment. The second round of panning which used 10 nM biotin-CD40 led to a ten-fold enrichment over background, which was a parallel panning with no biotin-CD40. Round three of panning used 2 nM biotin-CD40 and resulted in a ten-fold enrichment over background. Each subsequent round of panning was performed with longer washing conditions, 10s, 30s and 120s, respectively. Round 3 phage output was analyzed by colony PCR and sequencing of 96 clones. The results are in Table 14. DDG high risk PTM motif was mutated to several other sequences which presumably retained binding to CD40.

TABLE 14
Sequence enrichment analysis from 1D5
HCDR3 DDG library

| Sequence | Frequency | PTM risk |
|---|---|---|
| DGG | 8 | Yes |
| DDG | 6 | Yes |
| DEG | 6 | Yes |
| DNG | 6 | Yes |
| DAG | 3 | No |
| DDL | 3 | Yes |
| DDS | 3 | Yes |
| DSG | 3 | Yes |
| DDM | 2 | Yes |
| DKG | 2 | No |
| DDA | 1 | Yes |
| DDW | 1 | Yes |
| DNM | 1 | Yes |
| DQG | 1 | No |
| DWG | 1 | No |
| SNE | 1 | Yes |

### Example 17. Evaluating the Stability and Manufacturability Consequences of CD40-Specific Clone 1D5 PTM Removal

Following phage display, Example 16, variants of DDG sequence not containing PTM risk were introduced into the parental PC401003 molecule. These constructs were then transiently expressed in CHO cells, purified as described above, and examined for the impact of incorporating alternative sequences to the anti-CD40 HCDR3. These comparisons included evaluation of expression, thermal stability by DSF (described above), and binding affinity to human CD40 ECD by SPR.

Comparisons of the constructs indicate that the transient CHO expression levels are mostly unchanged when the DDG sequence is altered. However, the two constructs with moderately decreased expression levels (DKG and DWG) failed during purification, no further data is reported, indicated by a “-” in Table 15. The parent (PC401003 DDG) and remaining three variants (TDH, DAG, and DQG) successfully purified and analyzed further.

Thermal stability of these four constructs was evaluated by measuring Tm1 using DSF as described previously. Compared to the parent, all PTM variants impacted thermal stability, indicated by a decrease in Tₘ₁ of between 4.2 and 6° C. Additionally, binding affinity to human CD40 for the original PC401003 and three PTM variants was determined. Only TDH was significantly impacted by the sequence change: affinity to CD40 was nearly 8-fold weaker than PC401003.

TABLE 15
Summary of expression, thermal stability, and
CD40 affinity of PDL1 x CD40 bispecific
proteins with PTM removal variants of the
CD40 binding domain

| | Expres- | | Affinity to human CD40 | | |
|---|---|---|---|---|---|
| PTM | sion | DSF | ECD by SPR | | |
| Variant | Titer | Tₘ₁ | Ka | Kd | KD |
| Sequence | μg/mL | (° C.) | (1/Ms) | (1/s) | (nM) |
| DDG | 183 | 61.7 | 8.8E+03 | 4.4E−04 | 50 |
| (Original) | | | | | |
| TDH | 191 | 57.5 | 1.5E+04 | 5.9E−03 | 391 |
| DAG | 192 | 56.7 | 8.7E+04 | 5.3E−04 | 61 |
| DKG | 129 | — | — | — | — |
| DQG | 163 | 55.7 | 1.1E+04 | 8.8E−04 | 78 |
| DWG | 117 | — | — | — | — |

### Example 18. Humanization of CD40-Specific Clone 5B9 in scFv Format

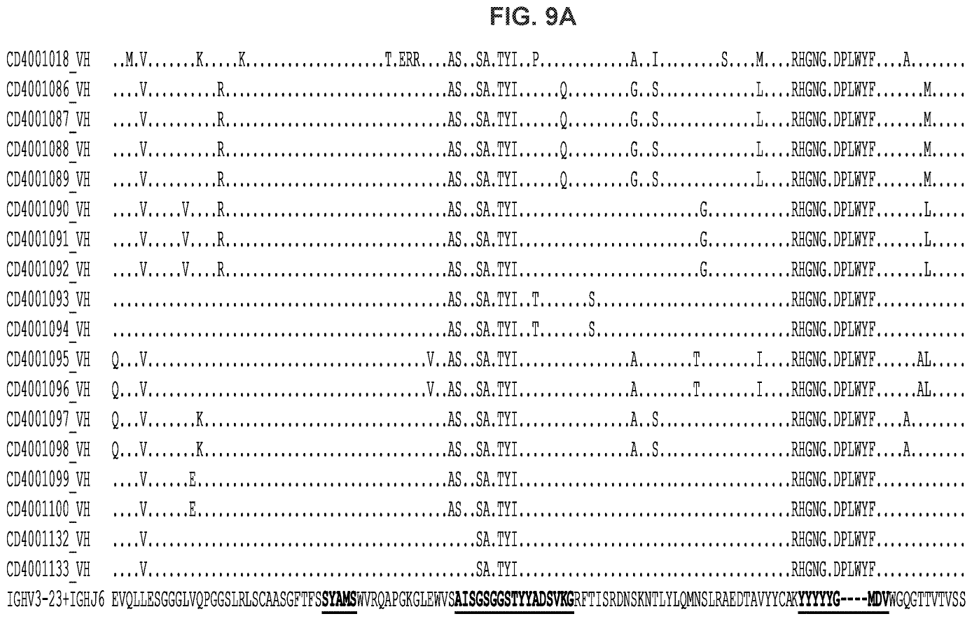

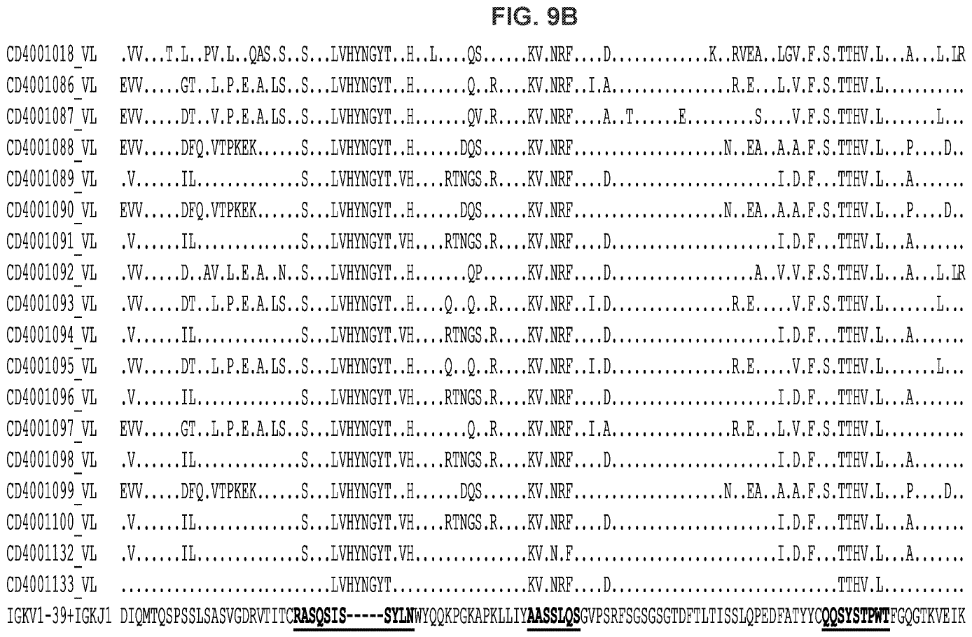

After evaluation of the hybridoma derived antibodies, clone 5B9 was selected for full humanization and optimization. The primary purpose of humanization was to eliminate as much of the mouse derived sequence as possible to minimize potential immunogenicity and optimize the binding and stability properties of the binding domain at the same time. Humanization of clone 5B9 anti-CD40 murine monoclonal antibody was performed in scFv-Fc format in multiple rounds of CDR grafting and refinements using BioLuminate software package (Schrodinger, LLC, New York, USA). A homology models of mouse clone 5B9 were created based on PDB IDs IKFA, 5Y9F, 2DQU and 2A77, and the most geometrically suitable and homologous human frameworks for CDR grafting were identified using the software's default and modified settings. 49 CDR-grafted molecules were produced in small scale and tested unpurified for binding to cells expressing full length human- or cyno-CD40. 15 clones (clone numbers CD4001086-1100) with matching binding of parental mouse 5B9 were scaled-up and purified, their binding kinetics to recombinant human CD40 antigen were measured using Biacore T200 (GE Healthcare Life Sciences, USA) or cell binding, stability by measuring Tm and Tagg using the Uncle instrument (Unchained Labs. USA) and expression levels and initial aggregate levels were noted. In Stage 2, molecule CD4001100 was further germlined to closed human sequence and 30 combinations of mutations were created. These were first screened from a small-scale production and the best 2 clones were scaled up purified and characterized. These were labeled clones CD4001132 and CD4001133. The humanization progress for VH chain is outlined in FIG. 9A and for VL chain in FIG. 9B.

### Example 19. Production and Biophysical Evaluation of Partial Humanized Versions of Anti-CD40 Clone 5B9

Different humanized versions of the Clone 5B9 scFv were produced as monospecific DNA constructs by attaching the scFv sequence to the N-terminus of a wildtype IgG1 Fc region. Following transient expression and purification of constructs with sufficient quantity, these constructs were characterized for expression, thermal stability by differential scanning fluorimetry (DSF), and binding affinity either by SPR to human CD40 ECD (as described in Example 20), or cell binding (shown in Example 21).

The mid-point of the first melting transition (Tₘ₁) was measured using DSF. Tₘ₁ was used to reflect the temperature required to unfold the first, or least-stable, binding domain in the construct. DSF was run using the Uncle instrument from Unchained Laboratories. Samples were analyzed at 1 mg/mL in PBS using intrinsic fluorescence (no additional dyes were used to assess protein unfolding). As shown in Table 16, all constructs exhibited Tₘ₁ above 6° C., indicating high thermal stability.

Additionally, expression levels varied throughout the humanization process ranging from 54.3 to 424.5 μg/mL, although most expressed above 200 g/mL. The predominantly high expression levels combined with Tm values suggest these variants have stability and solubility properties beneficial to therapeutic protein drugs.

TABLE 16
Summary of expression, thermostability, and
affinity of partially humanized anti-CD40
variants of clone 5B9

| Construct | Expres- | | Affinity to human CD40 | | |
|---|---|---|---|---|---|
| | sion | DSF | ECD by SPR | | |
| Details | Titer | Tₘ₁ | Ka | Kd | KD |
| Name | μg/mL | (° C.) | (1/Ms) | (1/s) | (nM) |
| CD4001086 | 247.3 | 69.5 | — | — | — |
| CD4001087 | 155.4 | 69.5 | — | — | — |
| CD4001089 | 54.3 | 67.7 | — | — | — |
| CD4001091 | 221.9 | 67.8 | — | — | — |
| CD4001092 | 332.9 | 70.4 | — | — | — |
| CD4001093 | 181.8 | 70.1 | — | — | — |
| CD4001094 | 229.3 | 67.2 | — | — | — |
| CD4001095 | 284.2 | 69.3 | — | — | — |
| CD4001096 | 316.2 | 66.8 | — | — | — |
| CD4001097 | 396.6 | 68.1 | — | — | — |
| CD4001098 | 424.5 | 67.2 | — | — | — |
| CD4001100 | 187.4 | 67.4 | 2.9E+05 | 6,0E−03 | 21 |
| CD4001132 | 239.6 | 65.7 | 2.4E+05 | 1.0E−02 | 4 |
| CD4001133 | 125.6 | 68.1 | 2.3E+05 | 1.9E−02 | 85 |

### Example 20. Methodology for Using Surface Plasmon Resonance to Determine the Binding Affinity of Anti-CD40 Binding Domains

SPR binding affinity studies of anti-CD40 binding domains to recombinant monomeric human CD40 ectodomain (ECD) were conducted at 25° C. in HBS-EP+ with 0.1% BSA buffer on a Biacore T200 system. Mouse anti-human IgG (Cytiva, BR100839) at 25 μg/ml in 10 mM sodium acetate pH 5.0 was immobilized at a density of 4,000 response units (RU) onto each flow cell of a CM5 research-grade sensor chip (Cytiva, BR100530) by standard amine coupling chemistry. Each anti-CD40 mono- or bispecific protein at 40 nM in HBS-EP+ with 0.1% BSA buffer was captured in a flow cell with the immobilized anti-human IgG at a flow rate of 10 μl/min for 20 sec. leaving one flow cell surface unmodified as the reference. Using a single-cycle kinetics mode, different concentrations of CD40 ECD ranging from 8 nM to 648 nM were sequentially injected through each flow cell at 30 μl/min for up to 400 sec followed by a 600 sec dissociation period. Regeneration was achieved by injection of 3 M MgCl₂ at a flow rate of 30 μl/min for 30 sec followed by HBS-EP+ with 0.1% BSA buffer stabilization for 1 min.

Sensorgrams obtained from kinetic SPR measurements were analyzed by the double subtraction method. The signal from the reference flow cell was subtracted from the analyte binding response obtained from flow cells with immobilized or captured ligands. Buffer reference responses were then averaged from multiple injections. The averaged buffer reference responses were then subtracted from analyte binding responses, and the final double-referenced data were analyzed with Biacore T200 Evaluation software (v2.0. Cytiva), globally fitting data to derive kinetic parameters. All sensorgrams were fitted using a simple one-to-one binding model.

Several monospecific and bispecific anti-CD40 proteins were evaluated for their binding affinity to monomeric human CD40 ECD using SPR (see Tables 15, 16, and 19).

### Example 21. Evaluation of Anti-CD40 Binding to CD40-Expressing CHO Cell Lines During Humanization

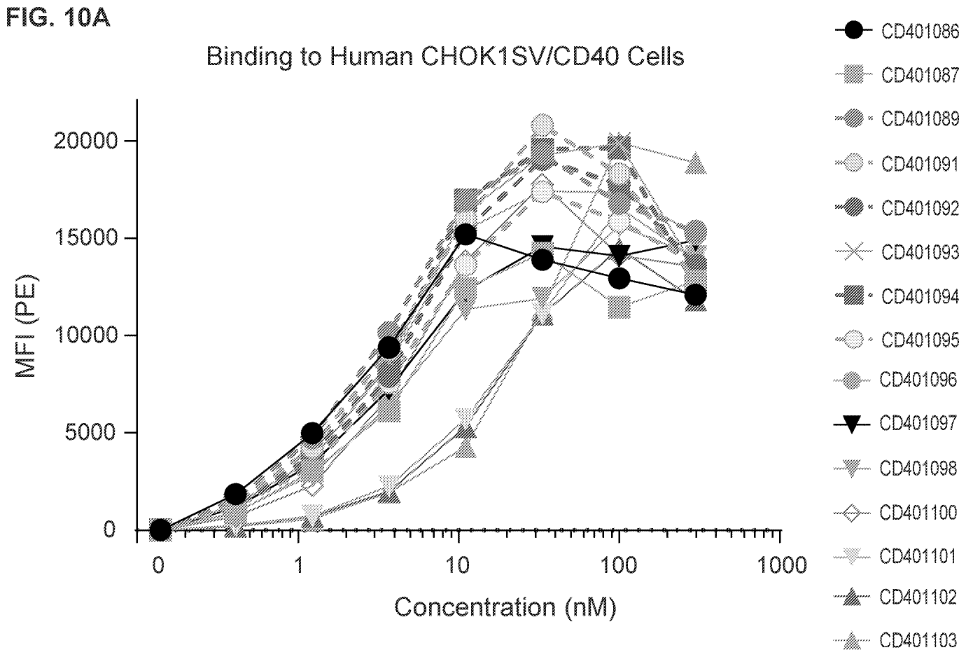

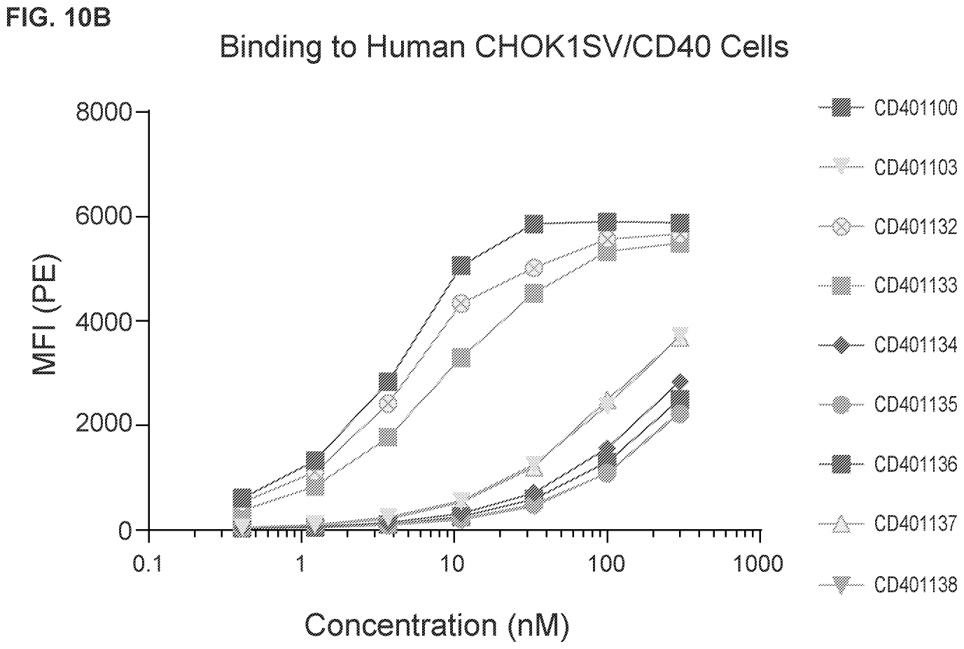

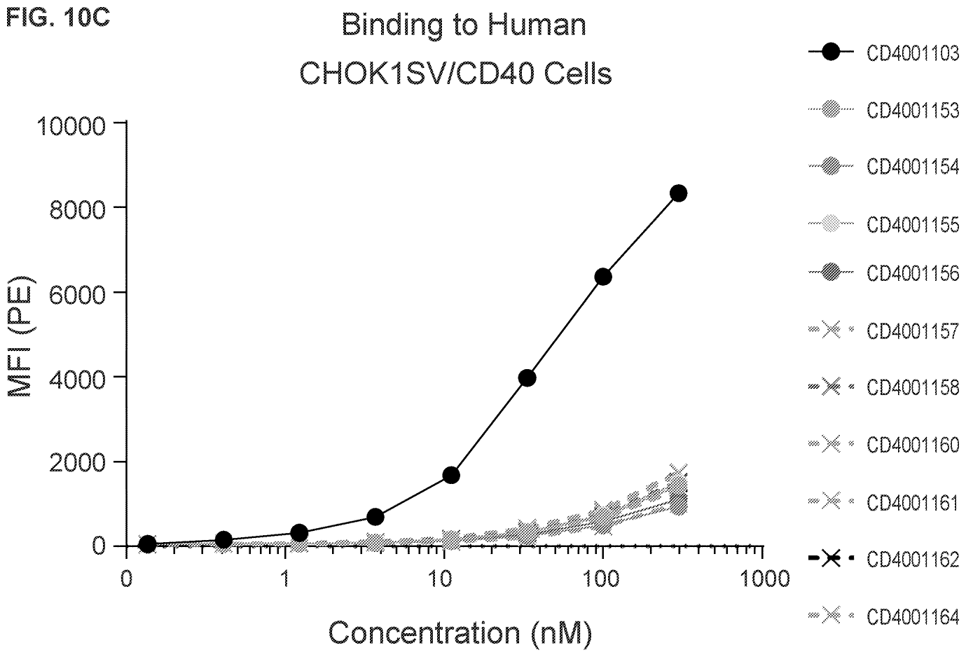

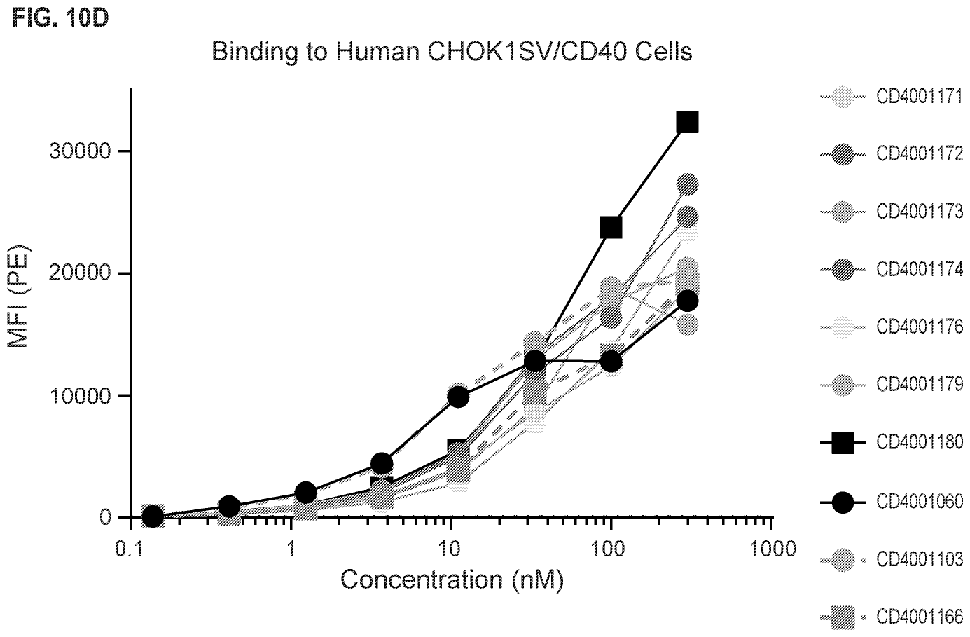

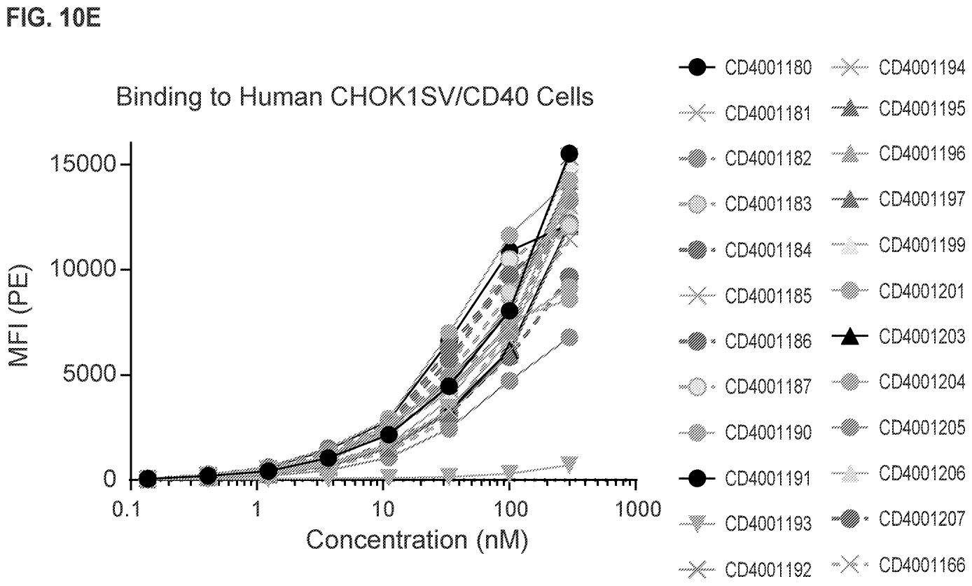

Throughout the anti-CD40 clones 5B9 and 1D5 binding domain humanization, the constructs were evaluated for retained on-cell binding similar to the parental domain. Using human CHOK1SV/CD40 cells, each successive set of changes were tested by flow cytometry as previously described. Humanization on clone 5B9 was initiated first and is shown as part of FIG. 10A and FIG. 10B. All 5B9 modifications retained CD40 binding similarly to the original. FIG. 10B through FIG. 10E all evaluate 1D5 humanization. Reduced 1D5 binding to CD40 was shown with modifications in FIG. 10C but was regained in modifications shown in FIG. 10D and FIG. 10E.

### Example 22. Binding of CD40-Binding Molecules to Human CD40 and PD-L1 Expressing Tumor Cell Lines

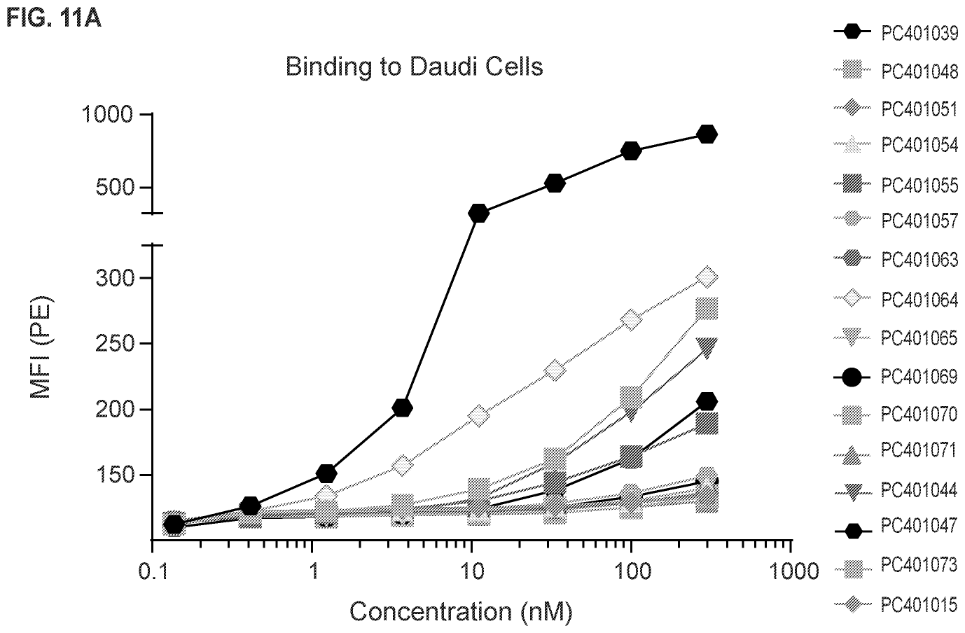

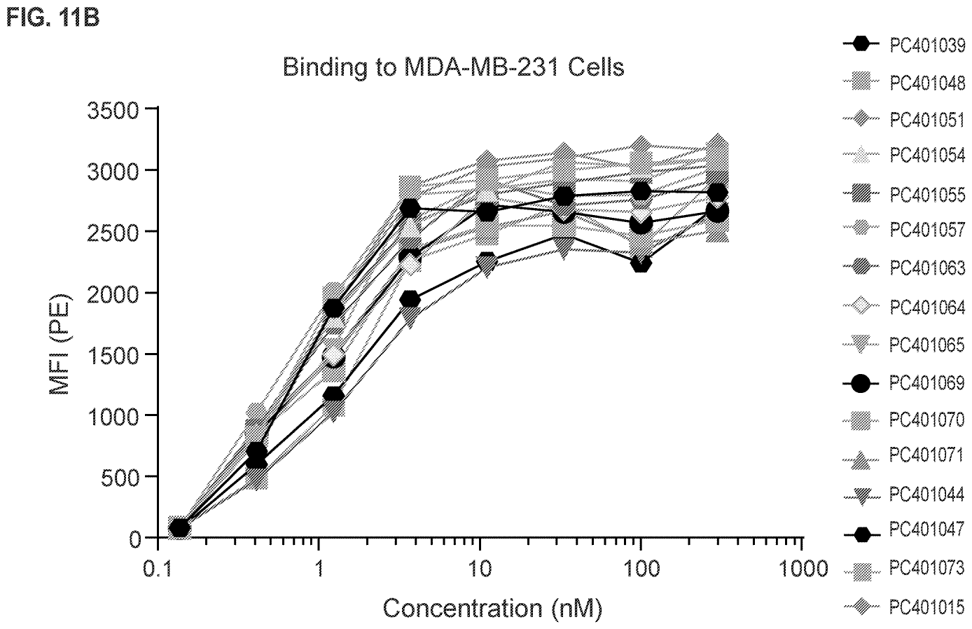

Binding studies were used to confirm binding of anti-PD-L1×ani-CD40 bispecific variants to human tumor cell lines known to express CD40 and/or PD-L1. Flow cytometry was used to examine the binding of each bispecific end. As shown in FIG. 11A, the binding to Daudi tumor cells varied widely depending on the bispecific. In contrast, there were minor differences in binding between the variants on MDA-MB-231 (FIG. 11B).

### Example 23. Assembly and Characterization of Bispecific Anti-PD-L1×Anti-CD40 Constructs with Manufacturability Improving Mutations to the Anti-CD40 scFv

A subset of PD-L1 and CD40 binding domains were combined into bispecific proteins. Individual binding domains were amplified by PCR and assembled with DNA fragments encoding Fc and linearized expression vector using standard molecular biology techniques. After analysis of experimental data and in silico modeling of the CD40 binding domain in the parental bispecific PC401003, several variants were constructed and tested. Mutations were designed to improve manufacturability properties and were identified using BioLuminate (Schrodinger Release 2022-4: BioLuminate, Schrödinger, LLC, New York, NY, 2021). The parental PC401003 amino acid sequence was mutated with single- or double-point mutations listed in Table 17. Bispecific molecules PC401119, PC401120, PC401122, PC401124, PC401128, PC401129, PC401132, and PC401133 (see Table 1) represent set of the variants of the CD40 domain with the most advantageous mutations.

Following transient transfection and purification using methods described above, the additional bispecific proteins were examined for the impact of incorporating additional mutations into the anti-CD40 scFv. The orientation was kept constant (anti-PD-L1 on the N-terminus and anti-CD40 on the C-terminus), and both the PD-L1 scFv and Fe sequence remained unchanged.

TABLE 17
Description of bispecific constructs evaluated for
manufacturability mutations in the anti-CD40 scFv

| | Stabilizing | |
|---|---|---|
| | Mutation(s) | Predicted Effect |
| | in CD40 | of Stabilizing |
| Construct | Binding | Mutation in CD40 |
| Name | Domain | Binding Domain |
| PC401003 | Parent | — |
| PC401119 | H-Ala60Asn | Increase intramolecular |
| | | stabilizing hydrogen bonds |
| PC401120 | L-Gln100Cys ~ + | Decrease tertiary structure |
| | H-Gly44Cys | mobility with extra disulfide |
| | | bond |
| PC401122 | H-Val100aAsn | Increase intramolecular |
| | | stabilizing hydrogen bonds |
| PC401124 | H-Ala60Asn + | Increase intramolecular |
| | H-Val100aAsn | stabilizing hydrogen bonds |
| | | and decrease hydrophobic |
| | | surface area |
| PC401128 | L-Leu30Thr | Decrease hydrophobic |
| | | surface area |
| PC401129 | L-Leu30Asn | Decrease hydrophobic |
| | | surface area |
| PC401132 | H-Leu52Asn | Decrease hydrophobic |
| | | surface area |
| PC401133 | H-Leu52Gln | Decrease hydrophobic |
| | | surface area |

Comparison of the constructs indicates that the transient expression levels are impacted by all stabilizing mutations compared to the parental PC401003. Within this set, PC401120 and PC401122 retained the highest expression levels. Preparative SEC was performed the following Protein A affinity capture step, which removed high molecular weight aggregate (HMW), as well as simultaneously buffer-exchanged the sample into PBS. Samples of each construct were analyzed by analytical HPLC to evaluate product homogeneity. All constructs in Table 18 had high purity levels (greater than 93% MP) with minimal HMW product detected by HPLC.

TABLE 18
Expression level and purity of bispecific anti-PD-L1 x
anti-CD40 constructs with stabilizing mutations

| | | Expres- | Analytical | |
|---|---|---|---|---|
| | | sion | HPLC | |
| Construct Details | | Titer | % | % |
| Name | Mutation | μg/mL | MP | HMW |
| PC401003 | Parent | 183 | 98.7 | 1.3 |
| PC401119 | H-Ala60Asn | 23.5 | 98.9 | 1.1 |
| PC401120 | L-Gln100Cys + | 86.1 | 99.2 | 0.8 |
| | H-Gly44Cys | | | |
| PC401122 | H-Val100aAsn | 62.8 | 99.1 | 0.9 |
| PC401124 | H-Ala60Asn + | 29.9 | 93.6 | 6.4 |
| | H-Val100aAsn | | | |
| PC401128 | L-Leu30Thr | 8.2 | 98.9 | 1.1 |
| PC401129 | L-Leu30Asn | 7.9 | 98.3 | 1.7 |
| PC401132 | H-Leu52Asn | 6.1 | 98.4 | 1.6 |
| PC401133 | H-Leu52Gln | 5.8 | 99.2 | 0.8 |
| PC401144 | L-Leu30Thr + | 42.3 | 99.0 | 1.0 |
| | L-Gln100Cys + | | | |
| | H-Ala60Asn + | | | |
| | H-Val100aAsn + | | | |
| | H-Gly44Cys | | | |
| PC401145 | L-Gln100Cys + | 24.2 | 99.2 | 0.8 |
| | H-Ala60Asn + | | | |
| | H-Val100aAsn + | | | |
| | H-Gly44Cys | | | |

The Tₘ₁ (mid-point of the first melting transition) and L_{agg}, the temperature of onset of aggregation based on dynamic light scattering, was measured for these constructs using the Uncle instrument from Unchained Labs. Most of the constructs shown in Table 24, including the parent PC401003, had Tₘ₁ and T_{agg} values that exceeded 60° C., indicative of having high thermal stability. Only PC401129 had a lower Tₘ₁ of 56.2° C. PC401120 had a nearly a full degree higher Tₘ₁ than the parent PC401003.

Using the BIACORE T200 SPR system, the affinity of a subset of these constructs for human CD40 ECD were determined using the methods described above. Monovalent binding affinity was determined by capturing the bispecific construct on the chip and injecting monovalent ECD of the target at multiple concentrations. The affinity of the CD40 scFv was not measurably impacted by stabilizing mutations, as all KDs remained in the low-nM range (Table 19). Further analysis of binding affinity to CD-40 and PD-L1 by on-cell binding is shown in Example 24.

TABLE 19
Tₘ₁ and T_{agg} values and binding affinity to human
CD40 ECD for bispecific anti-PD-L1 × anti-CD40
constructs with stabilizing mutations

| | | | | Affinity to human | | |
|---|---|---|---|---|---|---|
| | | | | CD40 ECD by SPR | | |
| Construct Details | | Tₘ₁ | T_{agg} | Ka | Kd | KD |
| Name | Mutation | (° C.) | (° C.) | (1/Ms) | (1/s) | (nM) |
| PC401003 | Parent | 61.7 | 61.1 | 8.8E+03 | 4.4E−04 | 50 |
| PC401119 | H-Ala60Asn | 61.1 | 61.5 | 7.0E+03 | 4.0E−04 | 57 |
| PC401120 | L- | 62.6 | 62.5 | 7.2E+03 | 5.4E−04 | 74 |
| | Gln100Cys −+ | | | | | |
| | H-Gly44Cys | | | | | |
| PC401122 | H-Val100aAsn | 61.5 | 61.7 | 8.7E+03 | 6.0E−04 | 69 |
| PC401124 | H-Ala60Asn + | 61.2 | 64.1 | — | — | — |
| | H-Val100aAsn | | | | | |
| PC401128 | L-Leu30Thr | 60.6 | 60.1 | — | — | — |
| PC401129 | L-Leu30Asn | 56.2 | 60.4 | — | — | — |
| PC401132 | H-Leu52Asn | 60.4 | 60.2 | — | — | — |
| PC401133 | H-Leu52Gln | 60.4 | 60.3 | — | — | — |
| PC401144 | L-Leu30Thr + | 62.5 | 64.1 | 7.3E+03 | 5.9E−04 | 81 |
| | L-Gln100Cys + | | | | | |
| | H-Ala60Asn + | | | | | |
| | H- | | | | | |
| | Val100aAsn + | | | | | |
| | H-Gly44Cys | | | | | |
| PC401145 | L-Gln100Cys + | 64.3 | 64.2 | 7.6E+03 | 6.1E−04 | 81 |
| | H-Ala60Asn + | | | | | |
| | H- | | | | | |
| | Val100aAsn + | | | | | |
| | H-Gly44Cys | | | | | |

### Example 24. Binding of Anti-PD-L1× Anti-CD40 Bispecific Proteins to Various Cell Lines

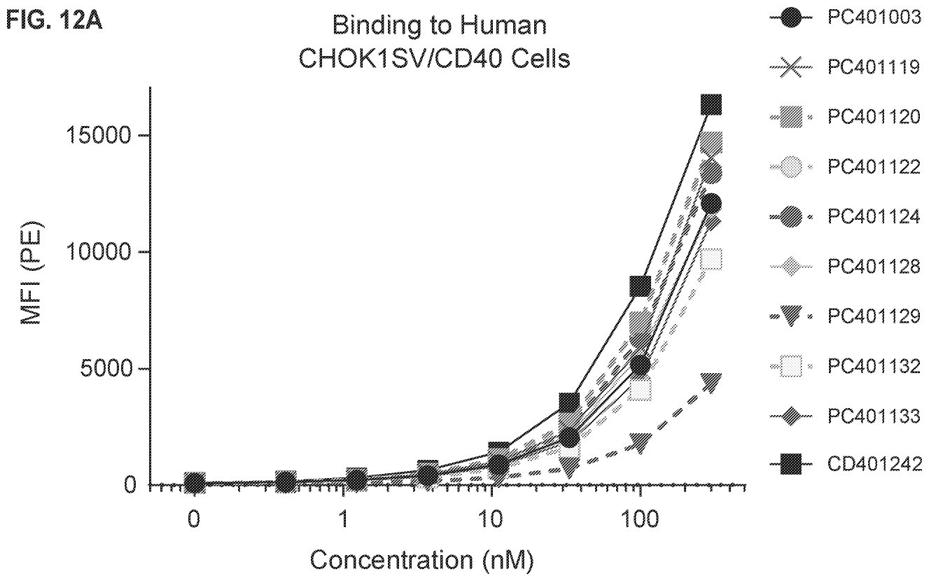

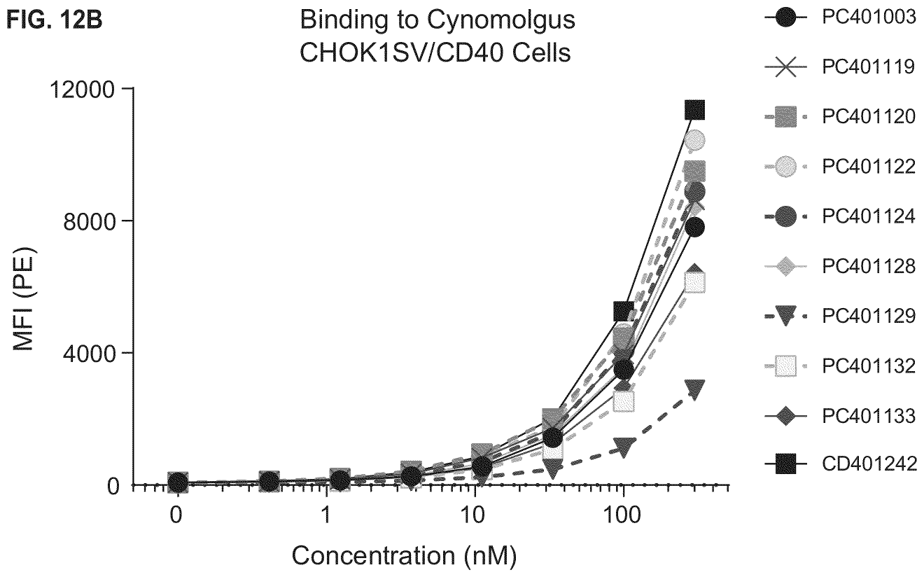

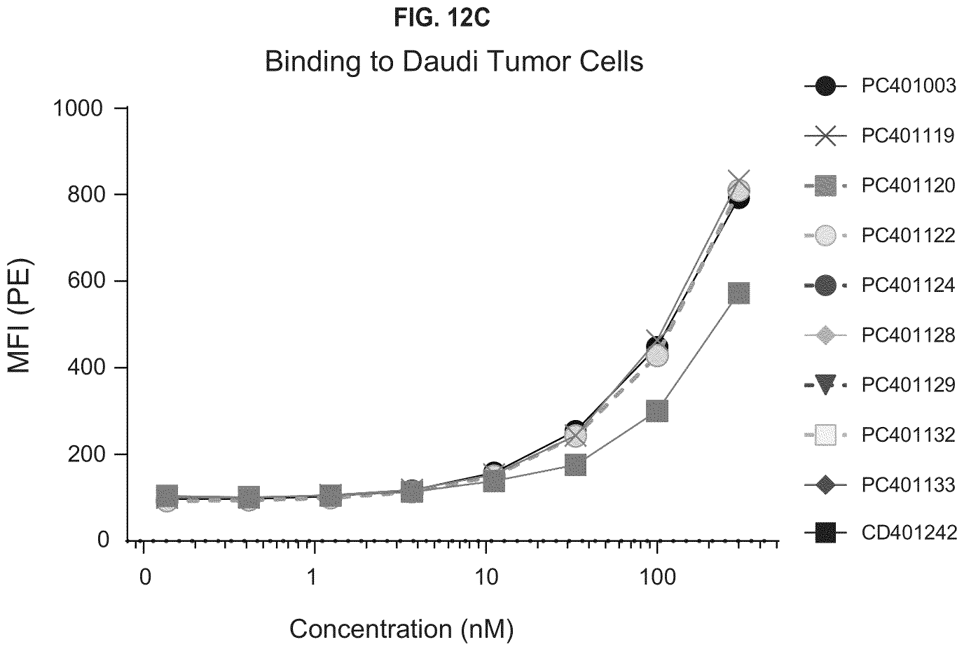

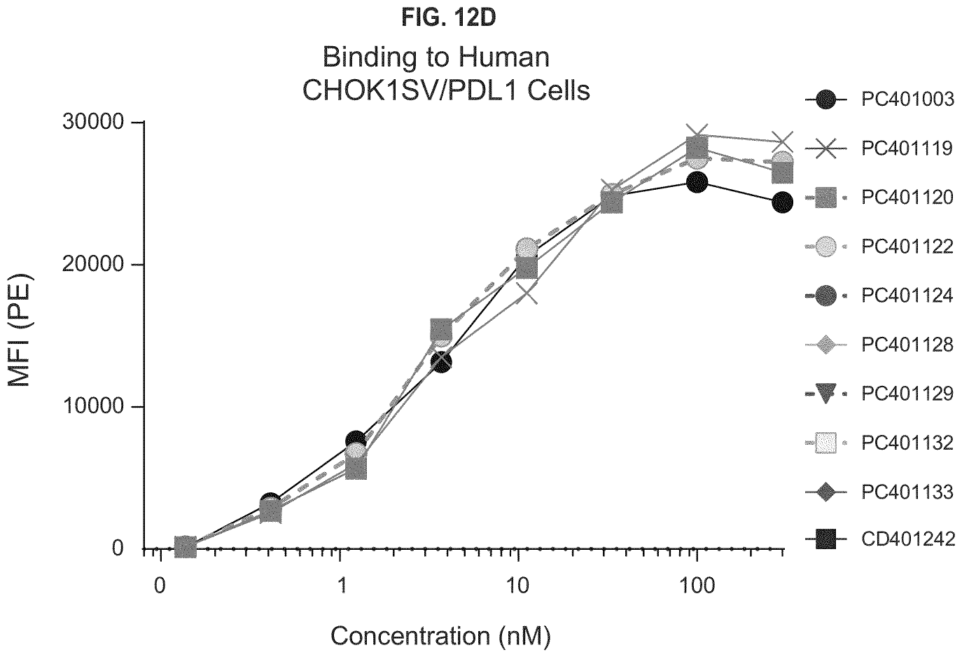

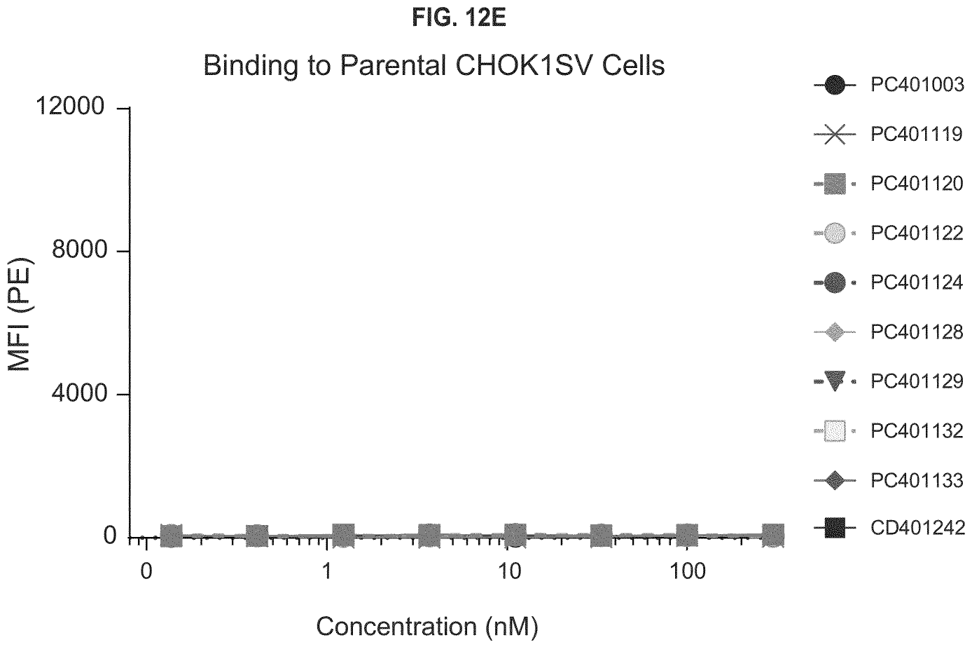

Using flow cytometry, the stabilized CD40 binding domains were evaluated for retained binding to CD40-expressing cell lines. As shown throughout FIG. 12A-FIG. 12E, the majority of sequence modifications had little impact on the original binding of the humanized 1D5 construct. FIG. 12A and FIG. 12B are graphical representations of the binding to CHOK1SV, expressing either human or cynomolgus CD40, respectively. The fully optimized CD40 binding domains were evaluated for binding to Daudi cells, a CD40 endogenously expressing tumor cell line. FIG. 12C shows that PC401120 has slightly lower binding compared to either PC401119 or PC401122. To ensure that binding was not negatively altered for PD-L1, binding to CHOK1SV/PD-L1 was again tested and found to be unchanged (FIG. 12D). Additionally, the full construct was evaluated for non-specific binding to parental CHOK1SV (FIG. 12E) but remained at background levels comparable to the parental ID5 construct PC401003.

### Example 25. Functional Reporter Assays Evaluating Anti-PD-L1× Anti-CD40 Bispecific Proteins

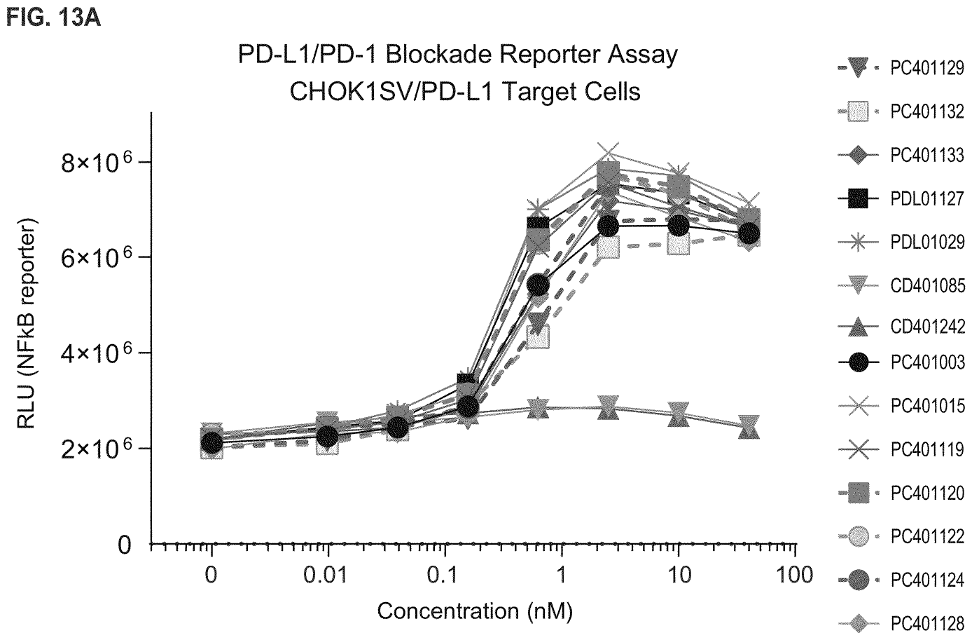

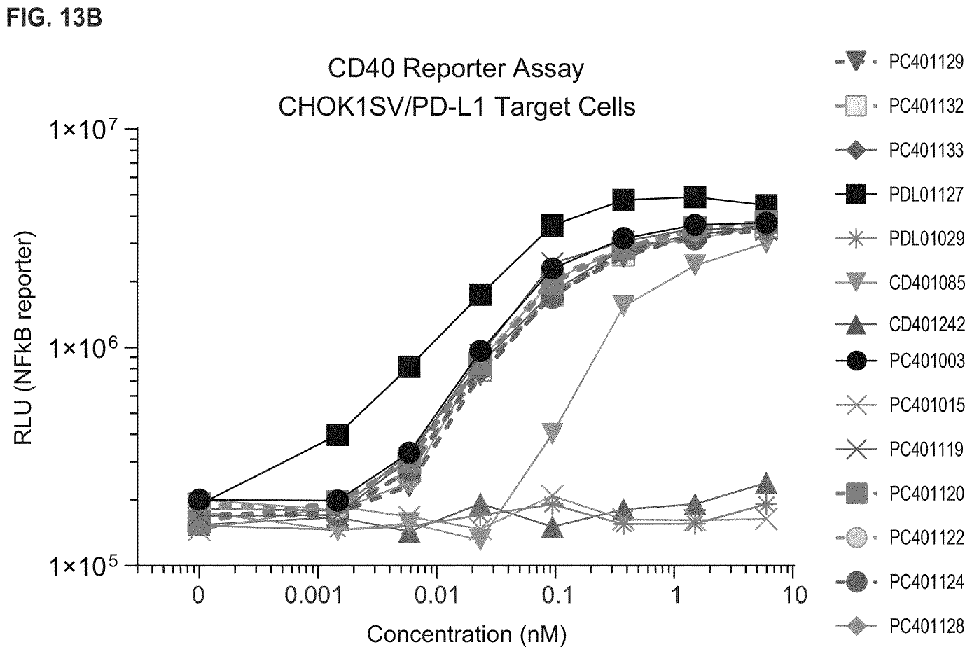

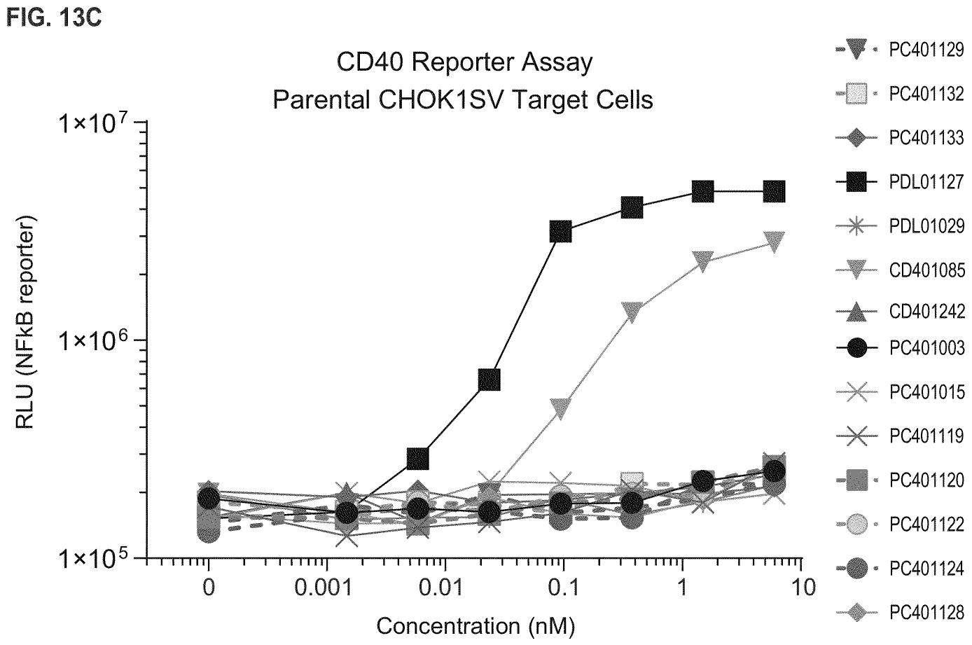

To compare the activity of anti-PD-L1×anti-CD40 bispecific proteins, two luciferase reporter lines were utilized in separate assays. PD-L1-expressing cells were used to bind and induce crosslinking via the anti-receptor binding domain on the other end of the bispecific. To quantitate PD-L1/PD-1 blockade activity, the human PD-1 NFAT luciferase reporter Jurkat line was incubated with TCR Activator PD-L1-expressing CHO target cells. As demonstrated in FIG. 13A, the PD-1/PD-L1 blockade activity of all tested bispecifics had very similar activity and are not impacted by the sequence modifications in the anti-CD40 binding domain. The blocking activity was similar to that of an atezolizumab analogue control (PDL01029). Only CD40 control samples did not enhance the luminescent signal.

Conversely, to examine CD40 activity, the human CD40/NFκB HEK293 luciferase reporter cells are incubated with PD-L1-expressing target cells. As shown in FIG. 13B, the CD40 activity of all tested bispecifics are undistinguishable and again are not impacted by the sequence modifications in the anti-CD40 binding domain. The CD40 reporter assay indicates that the functional activity of our bispecifics is between analogues of selicrelumab (CD401085) and MEDI7526 (PDL01127). Only negative control antibodies did not induce CD40 activity. FIG. 13C shows that the bispecifics require PD-L1 crosslinking for CD40 signaling, as using parental CHOK1SV cells does not increase CD40-mediated luminescence. In contrast, analogues of selicrelumab and MED17526, which do not require crosslinking to function, induce the same levels of CD40 signaling in the absence of PD-L1-expressing cells.

### Example 26. Functional Reporter Assays Evaluating Anti-PD-L1× Anti-CD40 Bispecific Proteins

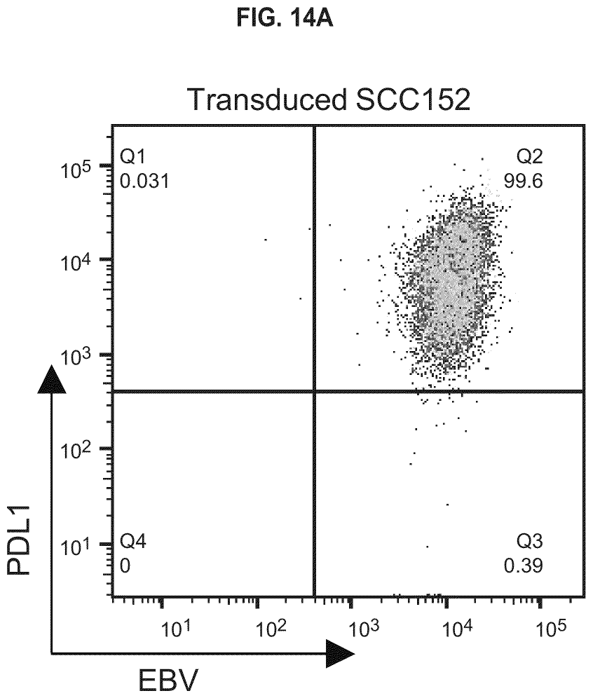

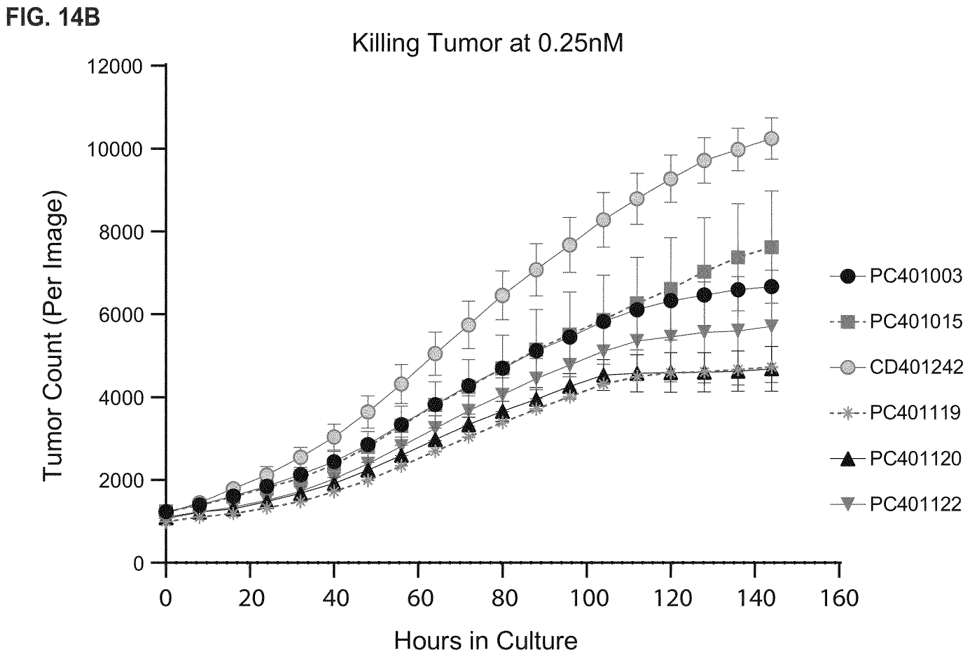

To compare the activity of optimized anti-PD-L1×anti-CD40 bispecific proteins, live cell imaging was used to view tumor cell killing. Initially, the squamous cell carcinoma line SCC152 (ATCC) was transduced to express several proteins. SCC152 cells were transduced with lentivirus Nuclight Orange (NLO, Sartorius) and selected using puromycin. Expanded cells were then transduced with an in-house generated lentivirus human PD-L1 and selected with Zeocin and puromycin. Finally, SCC152 was transduced with lentivirus EBV protein (Vectorbuilder) and selected using Neocin. Zeocin and puromycin. The triple positive cells were shown to be over 99% positive via flow cytometry (FIG. 14A).

To set up the tumor cell killing assay, several primary cell subsets were generated. M2 macrophages, immature DC and EBV-specific T cells are all separately prepared prior to setting up the assay. Two weeks prior to the start of the assay, EBV-expressing SCC152 tumor cells are treated ON with 10 ng/ml IFNγ. The following day, SCC152 tumor cells are treated with 130 KeV for 10 minutes using a Faxitron x-ray irradiator. Ten million PBMC (Bloodworks) are plated with 1e6 irradiated SCC152. Cytokines are added to promote T cell expansion (IL2 (10 ng/ml), 1L7 (10 ng/ml), IL15 (10 ng/ml), and IL2 (10 ng/ml)). T cells are expanded with fresh media and cytokines for 2 weeks before harvesting and assessing for tetramer staining and IFNγ production.

One week prior to assay set up, M2 monocytes and immature DC are generated from adherent PBMC (Bloodworks). For M2 monocytes, 100 ng/ml M-CSF (Peprotech) is added on day 0. Then on day 5 to polarize the cells, IL4 (50 ng/ml) and IL10 (50 ng/ml) are added to the feed media. For DC, IL4 (25 ng/ml) and GM-CSF (100 ng/ml) are added to the culture on days 0 and 5. All cells are harvested on day 7 for use in the killing assay.

The day prior to assay set up, 10,000 adherent SCC152 cells (expressing EBV. NLO and PD-L1) are seeded into 96-well plates and incubated ON. The media is then removed and replaced with 2× titrated PD-L1×CD40 test or control molecules. Immature DC, macrophages and T cells are mixed and transferred to the assay plate for a final cell number per well of 500, 500 and 1000 respectively. The plate is then transferred to the Incucyte reader (Sartorius) and imaged every 8 hours over the course of 6 days. Analysis was completed after the 6-day time course.

FIG. 14B graphically represents the reduced number of live SCC152 tumor cells at 0.25 nM of test or control molecules. The CD40 control molecule PC401242, was unable to enhance tumor cell killing. Both the PD-L1 (PC401015) and parental bispecific (PC401003) were able to enhance SCC152 killing but were less effective than the optimized molecules. PC401122 was slightly better than the parental molecule, but both PC401119 and PC401120 were superior at enhancing tumor killing.

### Example 27. Epitope Mapping of Anti-CD40 Binding Domain 1D5

Domain mapping principles described in EXAMPLE 11 were extended and further refined to map the epitope of anti-CD40 antibody 1D5. Both fully murine and final humanized versions of 1D5 binding domain in scFv format were tested with identical results.

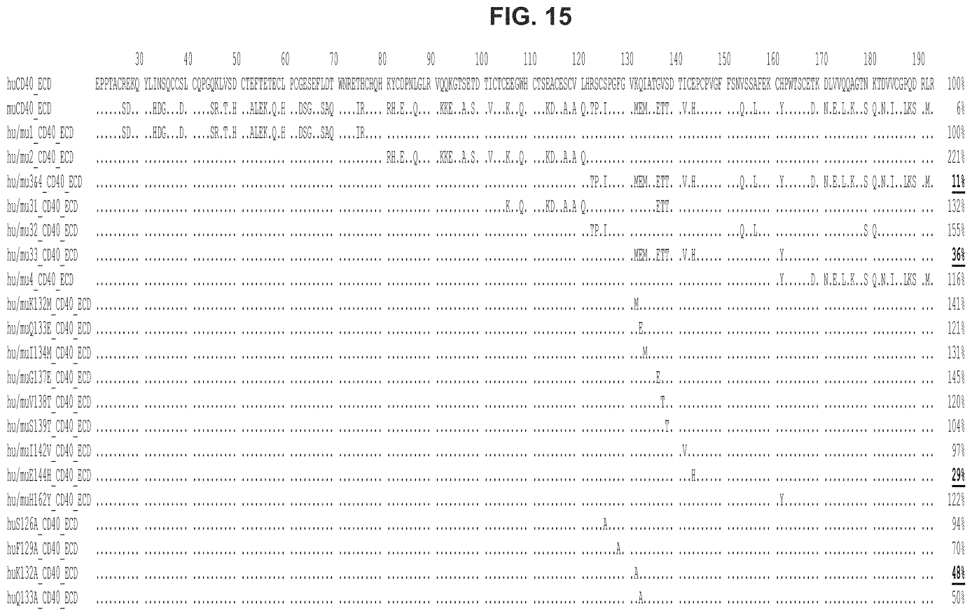

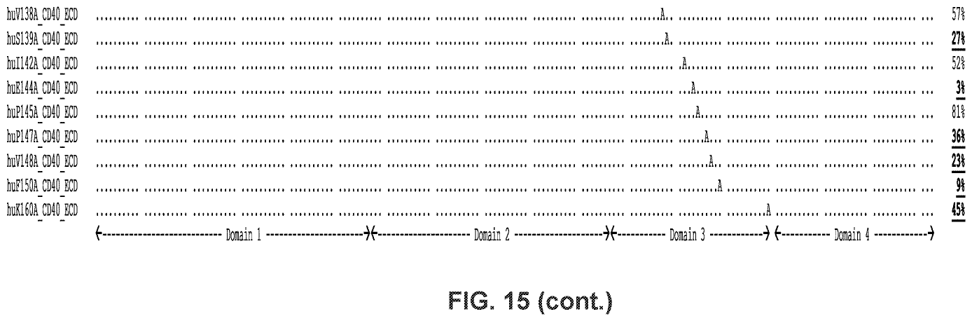

See FIG. 15 (Protein sequence alignment of CD40 extra-cellular domains used for epitope mapping of 1D5 antibody) for protein alignment of all sequences used for epitope mapping. In EXAMPLE 11 we showed that 1D5 binds to region between His122 and Arg193. Using published structure of human CD40, pdb 7P31_I, we designed 3 sets of spatially co-localized amino acids in domain 3 labeled 31, 32 and 33 and a domain 4. Amino acids in these sets representing human CD40 sequence were mutated to their murine CD40 counterparts. When expressed on the surface of CHO cells and tested by flow cytometry with 1D5 binding domain, only one construct hu/mu33_CD40_ECD lost binding (see FIG. 15). Next, we mutated all 9 human amino acids from construct 33 individually to their murine sequence. Only one mutation, E144H had a negative effect on binding. Alanine scanning mutagenesis was performed on 13 selected residues spatially surrounding Glu144, based on the published structures (see FIG. 15). Table 20 summarizes the data. The data confirmed very strong contribution of E144 residue, strong contribution of F150 and significant contributions of F129, Q133, V138, S139, 1142, P147, V148, F150 and K160 residues, to binding of both murine parental and humanized 1D5 binding domain.

TABLE 20
Alanine scanning for identification of critical
human CD40 residues involved in binding to
1D5 antibody

| | % binding of | % binding of |
|---|---|---|
| mutation | CD4001016 | CD4001242 |
| S126A | 106.4 | 94.1 |
| F129A | 88.0 | 70.0 |
| K132A | 79.7 | 48.4 |
| Q133A | 58.8 | 50.2 |
| V138A | 66.1 | 57.4 |
| S139A | 30.8 | 27.6 |
| I142A | 72.6 | 52.7 |
| E144A | 13.7 | 2.8 |
| P145A | 123.0 | 80.7 |
| P147A | 52.2 | 35.9 |
| V148A | 42.0 | 23.0 |
| F150A | 17.1 | 9.4 |
| K160A | 44.5 | 44.8 |

Binding was normalized to FLAG signal and values shown are relative to huCD40.

### Example 28. Surface Plasmon Resonance (SPR) Experiments to Confirm Elimination of Binding to Fcγ Receptors with Modified Fc Domain

The immunoglobulin constant domain (Fc) included in certain bispecific anti-PD-L1× anti-CD40 lead constructs incorporates modifications designed to prevent or reduce Fcγ receptor binding (FcγRI, FcγRIIa, FcγRIIb, FcγRIIa, and FcγRIIIb) compared to a wild-type immunoglobulin constant region, as described in Patent WO2022178114A1. The modified immunoglobulin constant region comprises a human IgG1 CH2 domain comprising the substitutions E233P, L234A, L235A, G237A, and K322A and a deletion of G236 according to the EU numbering system.

Fcγ receptor binding to wild type and modified immunoglobulin constant domain (Fc) protein fragments was measured by Surface Plasmon Resonance on a Biacore 8K instrument at room temperature. A modified Fc protein fragment vas directly immobilized on the surface of a CM5 sensor chip to a surface density of ~1000 RU by standard amine coupling chemistry. A wild type Fc fragment was similarly immobilized to the surface as a positive control and used as a comparator to evaluate the reduction in binding resulting from changes incorporated into the Fc region amino acid sequence. Fcγ receptors (purchased from R&D Systems) were diluted to either 100 nM (FcγRI) or 2 μM (all other Fcγ receptors) in HBS-EP+ buffer before injecting over the surface of the prepared sensorchip at 30 μL/min for 60 seconds. Maximum RU values during association phase of each injection were normalized to the result for wild type Fc and the calculated relative binding values are reported in Table 21. The described Fc modifications significantly reduce binding to all the receptors tested, with some apparent residual binding to Fcγ RIIA and RIIB/C receptors.

TABLE 21
Binding of wild type and modified Fc
fragments to different Fc receptors

| | Relative binding (RU) value during association | | | | | | |
|---|---|---|---|---|---|---|---|
| | | RIIA | RIIA | | | RIIIA | |
| Fc type | RI | R167 | H167 | RIIB/C | RIIIA | V176F | RIIIB |
| wt | 100 | 100 | 100 | 100 | 100 | 100 | 100 |
| modified | 0 | 20 | 3 | 8 | 0 | 0 | 0 |
| Fc | | | | | | | |

### Example 29. Surface Plasmon Resonance (SPR) Experiments to Confirm Retention of Binding Affinity to the Neonatal Fc Receptor (FcRn)

The immunoglobulin constant domain included in certain bispecific anti-PD-L1× anti-CD40 lead constructs incorporates modifications designed to prevent or reduce Fcγ receptor binding but maintain expected neonatal Fc receptor (FcRn) binding compared to a wild-type immunoglobulin constant region, as described in Patent WO2022178114A1. The modified immunoglobulin constant region comprises a human IgG1 CH2 domain comprising the substitutions E233P, L234A, L235A, G237A, and K322A and a deletion of G236 according to the EU numbering system.

SPR binding studies to human neonatal Fc receptor (FcRn, complexed with beta-2-macroglobulin) for bispecific anti-PD-L1×anti-CD40 proteins with modified Fc were conducted at 25° C. on a Biacore 1200 system in a running buffer of phosphate-buffered saline (PBS) with 0.1% BSA and 0.05% Polysorbate-20 adjusted to pH 6.0. Human FcRn/b2m at 10 μg/ml in 10 mM sodium acetate (pH 4.5) was immobilized on a CM5 sensor chip (Cytiva, BR100530) by direct amine coupling chemistry to a level of ~340 RU. A reference flow cell was left blank. Different concentrations of bispecific anti-PD-L1× anti-CD40 proteins from 2 nM to 162 nM diluted in running buffer were sequentially injected at 30 μl/min for 180 sec followed by a 180 sec dissociation period. Optimal regeneration was achieved by twice injection of PBS with 0.1% BSA and 0.05% Polysorbate-20 adjusted to pH 7.4 at a flow rate of 30 μl/min for 30 seconds, followed by running buffer stabilization for 1 min.

Sensorgrams obtained from kinetic SPR measurements were analyzed by the double subtraction method. The signal from the reference flow cell was subtracted from the analyte binding response obtained from flow cell with immobilized ligands. Buffer reference was subtracted from analyte binding responses, and the final double-referenced data were analyzed with Biacore T200 Evaluation software (v2.0. Cytiva), globally fitting data to derive kinetic parameters. All sensorgrams were fitted using two-state reaction model, as described in Weirong Wang et al, Drug Meta Dispos. 2011 September; 39(9): 1469-77. All tested bispecific anti-PD-L1×anti-CD40 proteins maintained expected binding to FcRn.

Several bispecific anti-PD-L1× anti-CD40 proteins were evaluated for their binding affinity to neonatal Fc receptor (FcRn) using SPR (see Table 22).

TABLE 22
SPR affinity measurements to neonatal Fc
receptor (FcRn)

| Construct | Affinity to FcRn, KD |
|---|---|
| Name | (nM) |
| PC401003 | 121 |
| PC401119 | 122 |
| PC401120 | 146 |
| PC401122 | 134 |
| PC401124 | 144 |

### Example 30. Evaluation of Anti-CD40 Bispecifics Targeting Either ROR1 or EGFR Tumor-Associated Antigens

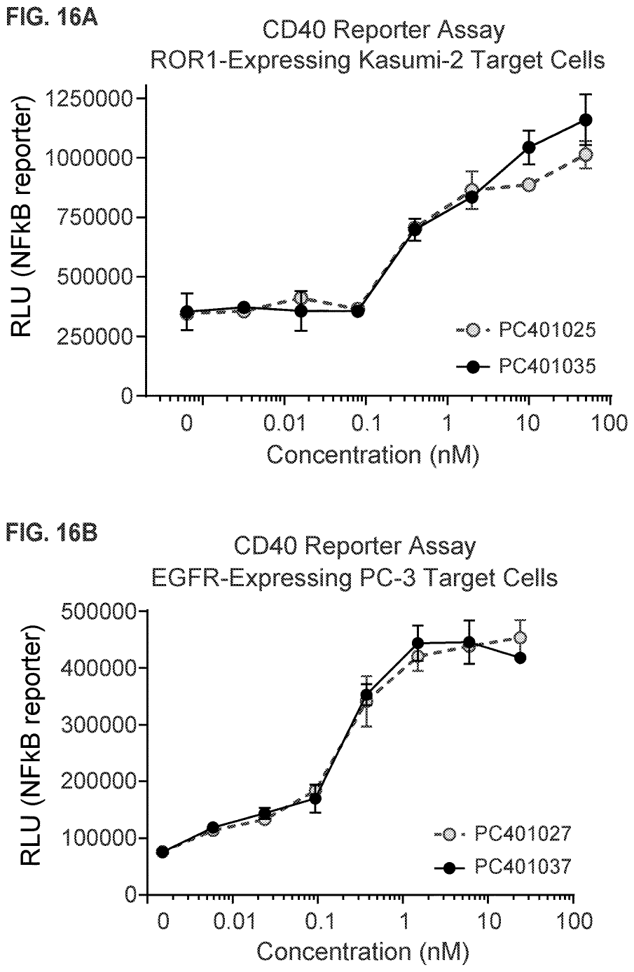

This example demonstrates that the provided CD40 binding domain is capable of being paired with various tumor-associated antigens and utilized to target any number of tumor types. Our humanized 1D5 CD40 binding domain was linked to either ROR1- or EGFR-specific binding domains. These tumor antigens are expressed on Kasumi-2 and PC-3 tumor cells, respectively. The CD40 reporter assay was set up similarly to previous assays, but the target cells were replaced accordingly. As shown in FIG. 16A, the CD40 activity of the tested bispecifics with anti-ROR1 binding are very similar to each other and arm capable of dose-dependent downstream signaling of CD40 as measured by light production. FIG. 16B also shows that the bispecifics utilizing EGFR crosslinking for CD40 signaling induce a dose-dependent increase in CD40-mediated luminescence. Both of these examples infer that additional tumor antigens could be targeted and used for crosslinking dependent CD40 signaling in the bispecific ADAPTIR.

### Example 31. Evaluation of Anti-PD-L1 Bispecifics Used to Activate Other Stimulatory Signaling Pathways

To evaluate the activity of different stimulatory signaling pathways utilizing PD-L1 as a targeting arm of the molecule and assessed in reporter assays. Bispecific molecules were constructed with bivalent anti-PD-L1 on the N-terminus and bivalent OX40, 4-1BB or CD3 binding domains on the other terminus. All reporter assays used 30,000 PD-L1-expressing CHOK1SV target cells in 96-well plates and dilutions of the bispecific constructs. All reporter cell lines were generated in-house and carry a luciferase reporter gene under the control of an NFκB promoter. Fifty thousand reporter cells were co-cultured with the CD3-binding bispecific construct PC401020 to induce target-dependent activation of CD3. For the OX40 reporter assay, 50,000 Jurkat cells transfected to expressed human OX40 and the reporter gene were co-cultured with the OX40-binding bispecific construct PC401022 to induce target-dependent activation of OX40. For the 4-1BB assay, 50,000 Jurkat cells transfected to expressed human 4-1BB and the reporter gene were co-cultured with the 4-1BB-binding bispecific construct PC401021 to induce target-dependent activation of 4-1BB. All plates were incubated at 37° C. 5% CO₂ in humidified incubators for 5 hours. One hundred microliters of Bio-Glo buffer (Promega) was added to each well, mixed, and incubated for 10 minutes. Luminescence was measured in a MicroBeta²2450 Microplate Counter (Perkin Elmer). Nonlinear regression analysis to determine EC₅₀ values was performed in GraphPad Prism 6® graphing and statistics software.

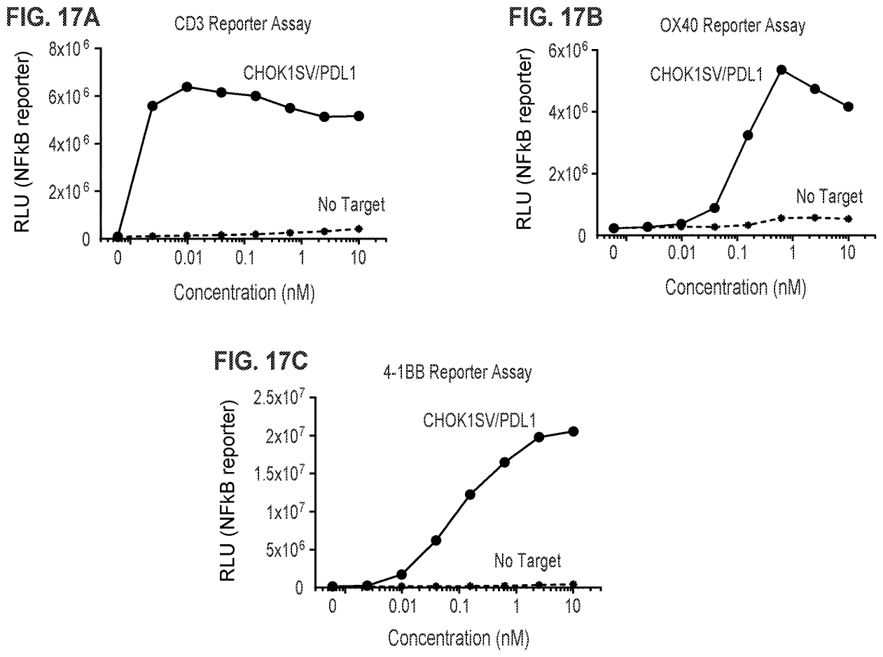

Each of the 3 reporter assays are shown in FIG. 17A-FIG. 17C. The CD3 reporter assay shows robust downstream signaling when CD3 is engaged by the bispecific antibody (FIG. 17A). For both the OX40 and 4-1BB reporter assays, there is a dose dependent correlation between the activity and the concentration of OX40 and 4-1BB molecule, respectively (FIG. 17B and FIG. 17C). In all 3 assays, the stimulatory signaling is mediated by crosslinking through PD-L1 expression on the CHOK1SV cell line, as performing the assay without target cells eliminates all downstream NFκB signaling.

### Example 32. Binding Evaluation of Anti-PD-L1×Anti-CD40 Constructs with Stabilized Binding Domains

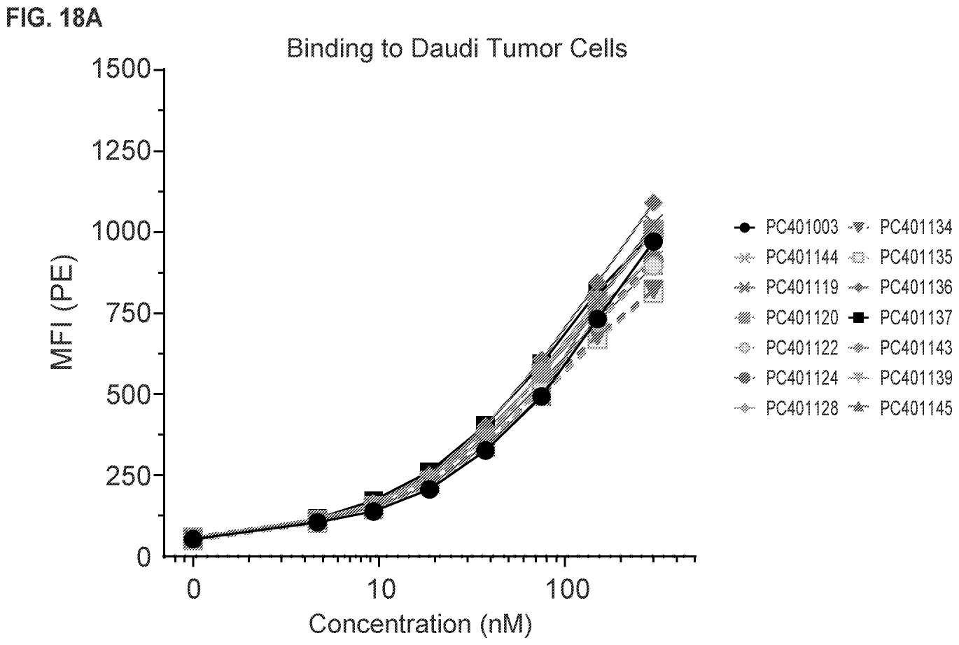

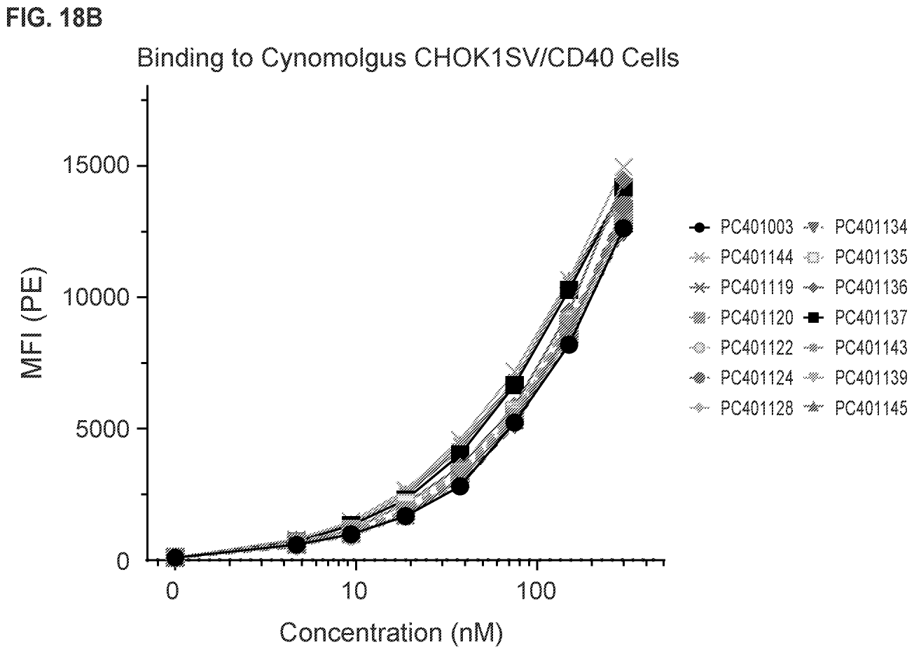

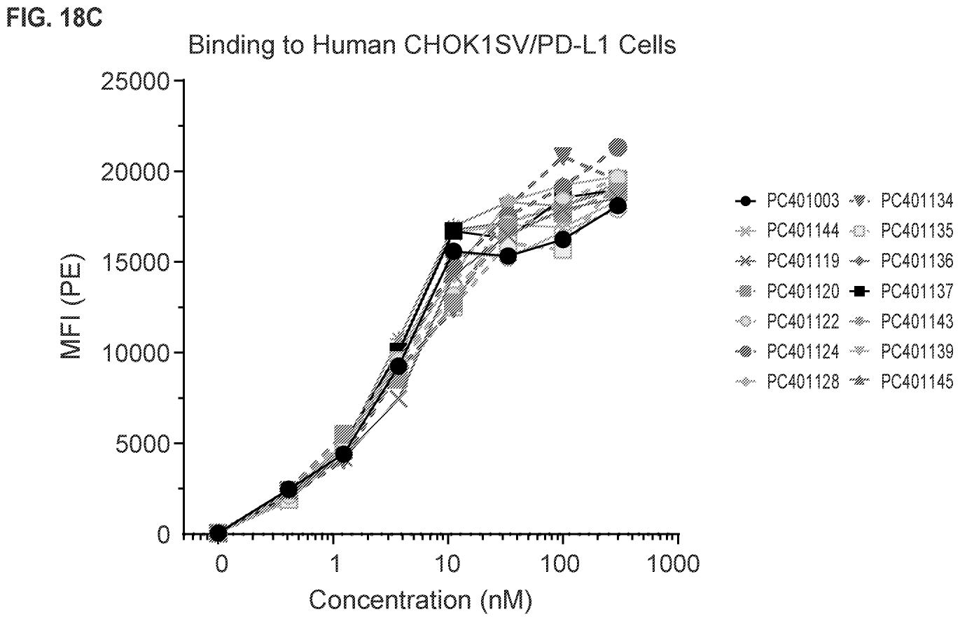

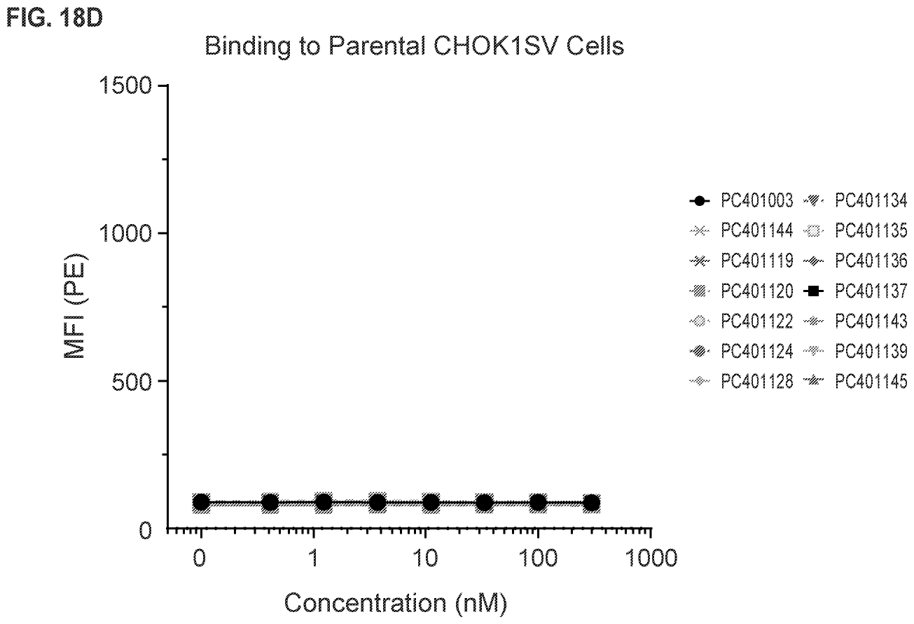

Binding studies were used to confirm binding of anti-PD-L1×anti-CD40 bispecific variants to human tumor cell lines known to express CD40 and/or PD-L1. Flow cytometry was used to examine the binding of each bispecific end. the various changes to stabilize the CD40 binding domain did not alter the binding of the bispecific to Daudi tumor cells (FIG. 18A) or CHOK1SV engineered to express cynomolgus CD40 (FIG. 18B). There were minor differences in binding between the variants' binding to CHOK1SV/PD-L1 cells (FIG. 18C). As shown in FIG. 18D, the background binding to parental CHOK1SV remained very low.

### Example 33. Reporter Evaluation of Anti-PD-L1×Anti-CD40 Constructs with Stabilized Binding Domains

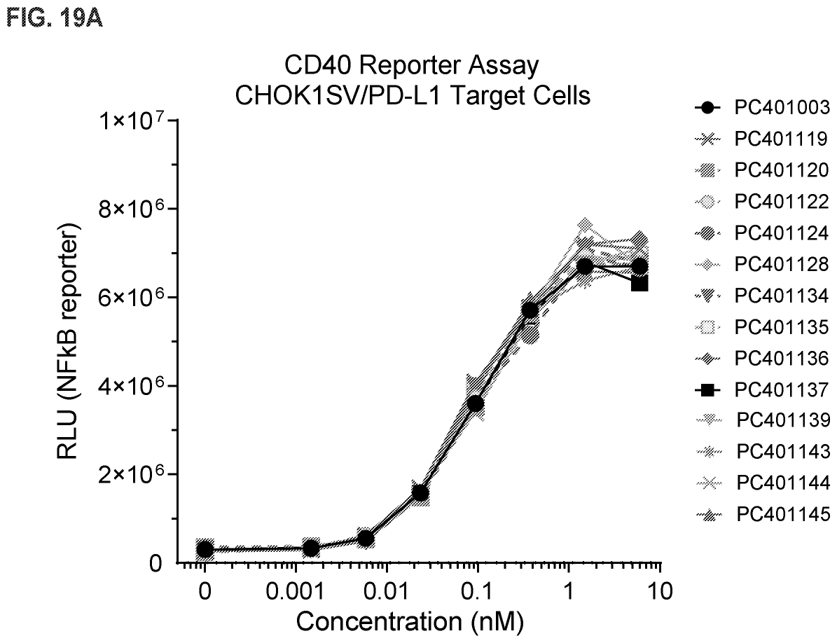

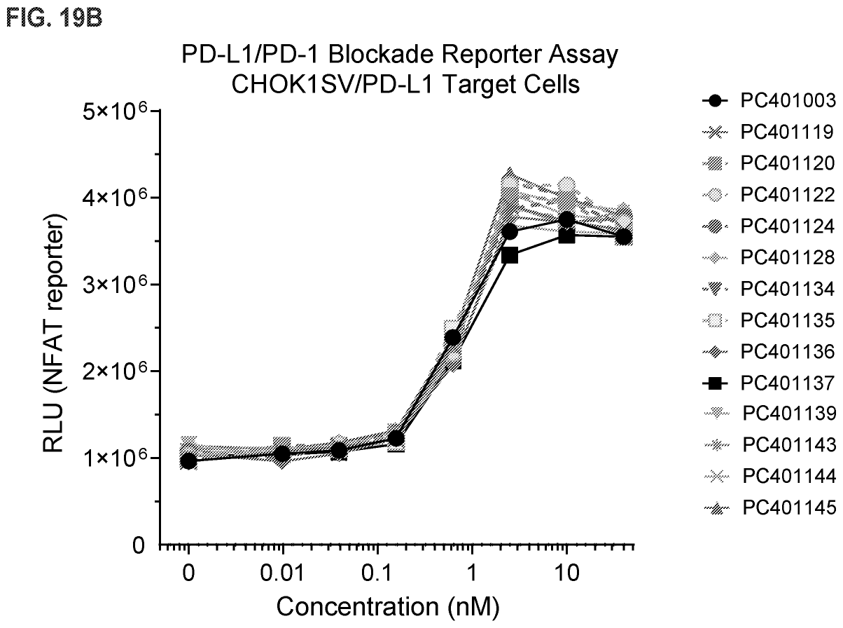

Luciferase reporter assays were used to compare the activity of anti-PD-L1× anti-CD40 bispecific proteins with stabilized binding domains. To examine CD40 activity, the human CD40/NFκB HEK293 luciferase reporter cells are incubated with PD-L1-expressing target cells. As shown in FIG. 19A, the CD40 activity of all tested bispecifics are undistinguishable and again are not impacted by the stabilizing modifications in the anti-CD40 binding domain. To quantitate PD-L1/PD-1 blockade activity, the human PD-1 NFAT luciferase reporter Jurkat line was incubated with TCR Activator PD-L1-expressing CHO target cells. As demonstrated in FIG. 19B, the PD-1/PD-L1 blockade activity of all tested bispecifics had very similar activity and are not impacted by the stabilizing modifications in the anti-CD40 binding domain.

### Example 34. Reporter Evaluation of Anti-PD-L1×Anti-CD40 Constructs with Stabilized Binding Domains Demonstrating the Requirement for Crosslinking

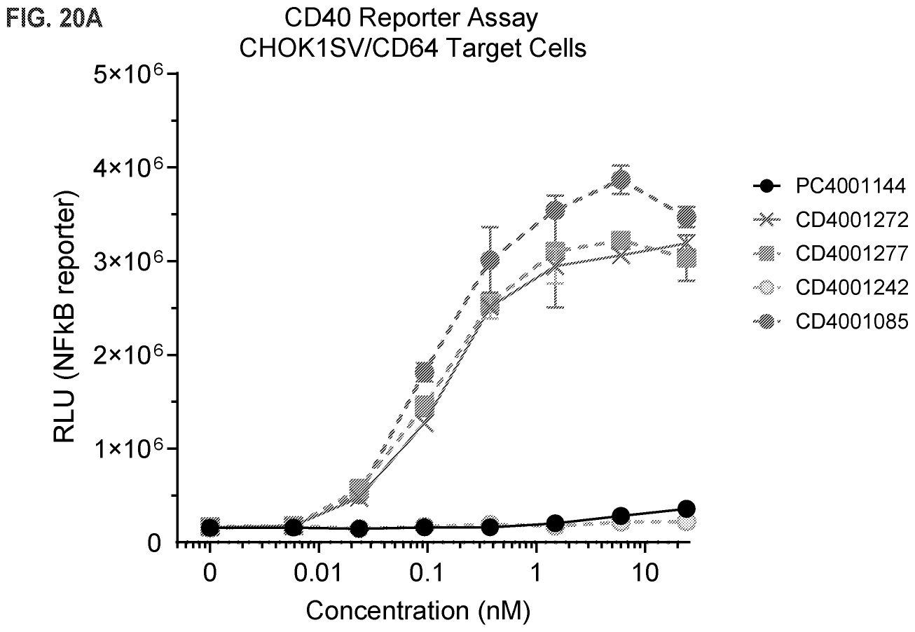

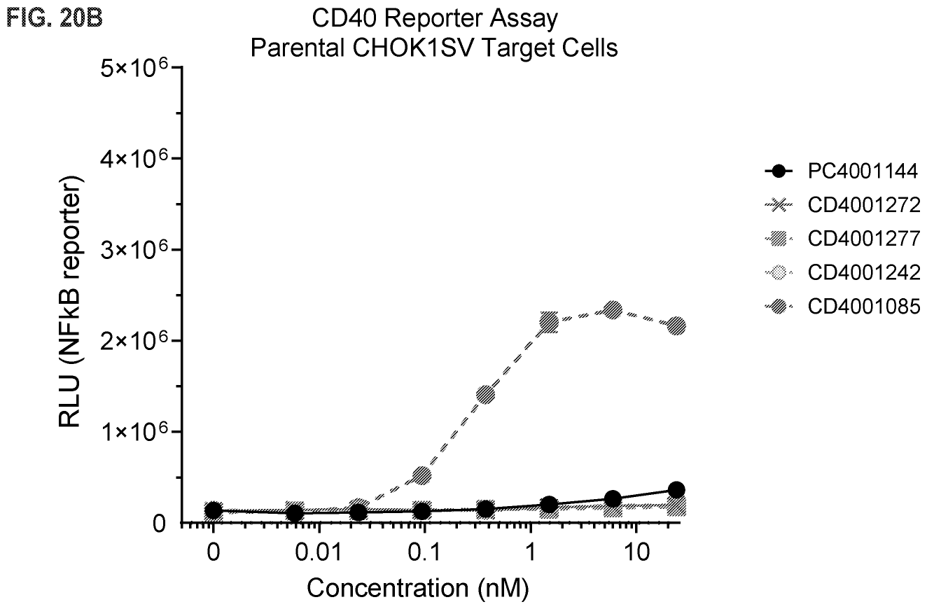

To examine the requirement of crosslinking for CD40 functional activity, bispecifics were constructed that contained either a wildtype Fc or an Fc null mutation to prevent binding to Fey receptors. The human CD40/NFκB HEK293 luciferase reporter cells were incubated with Fey receptor CD64-expressing target cells. As shown in FIG. 20A, when using CD64 to crosslink wildtype Fcs the CD40 activity is not impacted by the stabilizing modifications in the anti-CD40 binding domain as the stabilized domain in CD4001277 has indistinguishable function compared to the derivative binding domain in CD4001272. These are showing slightly lower luciferase induction compared to the selicrelumab analogue (CD4001085). Neither construct containing the Fc null mutations induced CD40 signaling. As demonstrated in FIG. 20B, the stabilizing modifications in the anti-CD40 binding domain do not generate CD40 function without crosslinking provided by the parental CHOK1SV cell line. In contrast, the analogue of selicrelumab, which does not require crosslinking to function, retains the ability to induce CD40 signaling in the absence of CD64-expressing cells.

### Example 35. In Vivo Evaluation of Anti-PD-L1× Anti-CD40 Constructs in Human PD-L1/PD-1/CD40 Knock-In Mice

Female B-hPD-1/hPD-L1/hCD40 triple knock-in mice from Biocytogen, China were acclimated for two weeks before initiation of the study. Animals were checked daily for general health. Treatment of study animals was in accordance with conditions specified in the Guide for the Care and Use of Laboratory Animals, and the study protocol was approved by the Institutional Animal Care and Use Committee (IACUC).

The mouse colon adenocarcinoma cell line MC38-hPD-L1-V1 (Biocytogen) was thawed and expanded in culture. B-hPD-1/hPD-L1/hCD40 mice were challenged on day 0 by injecting 5×10⁵ MC38-hPD-L1 cells subcutaneously on their flank. Starting day 6 after tumor challenge, treatment groups were normalized for tumor burden by ranked random assignment to receive treatment with either vehicle (PBS) or treatment at 3 mg/kg/mouse on days 0. 7, 10, 14 and 17 via IP administration. Tumor growth was observed and measured three times/week with a caliper. Tumor volumes were calculated using the formula: Volume=½[length×(width)²]. The experimental endpoint was reached when the tumor volume was ≥1500 cm³, there was a wound, or the health of the mice was affected.

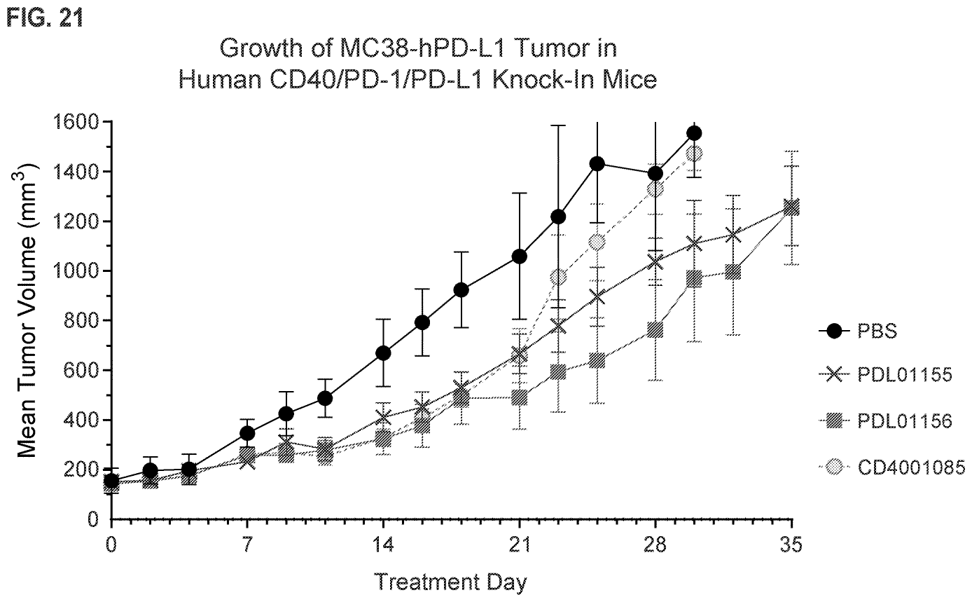

Treatment with either PDL01055 or PDL01056 at a dose of 3 mg/kg/mouse resulted in reduction of MC38-hPD-L1 tumor growth in B-hPD-1/hPD-L1/hCD40 mice (FIG. 21). The level of tumor reduction was better than that seen with the selicrelumab analogue (CD4001085).

### Example 36. In Vitro Evaluation of Anti-PD-L1× Anti-CD40 Bispecific Constructs with Stabilized Binding Domains on DC Maturation

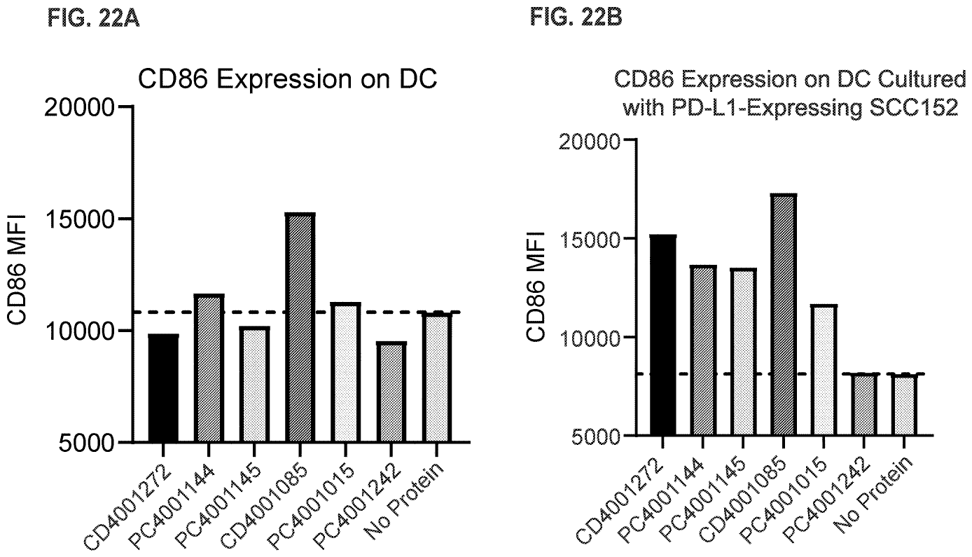

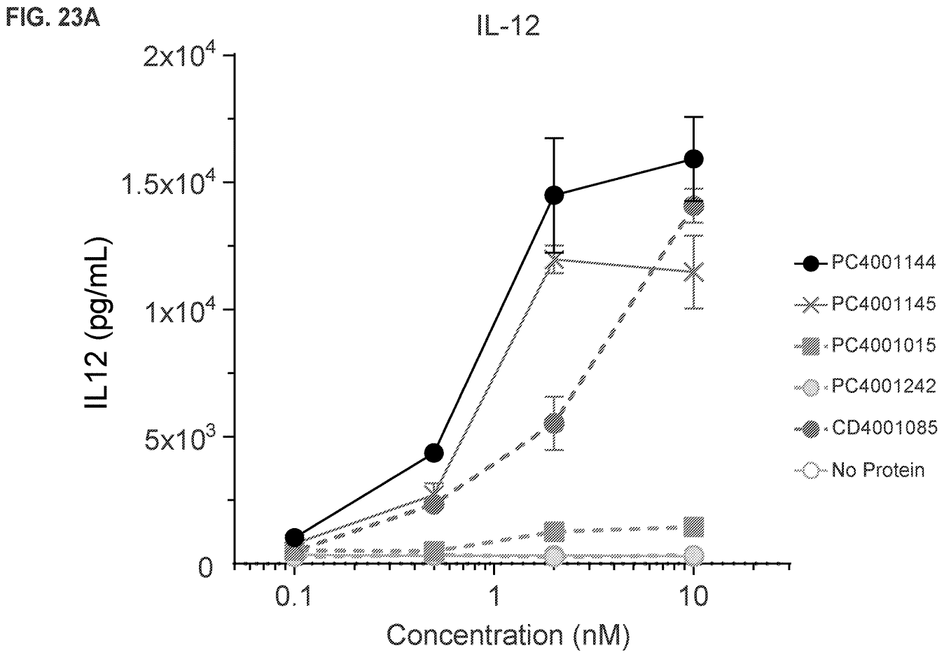

The effect of anti-PD-L1× anti-CD40 bispecific proteins with stabilized binding domains on DCs were assessed by culturing immature DC alone (FIG. 22A) or with PD-L1-expressing SCC152 tumor cells (FIG. 22B). Only the selicrelumab analogue (CD4001085) stimulated an increase in CD86 expression on DCs above background after 48 hours in cultures without PD-L1-expressing tumor cells to generate trimers (FIG. 22A). In contrast, all bispecifics stimulated DC resulting in an increased expression of CD86 with the addition of crosslinking cells.

### Example 37. In Vitro Evaluation of Anti-PD-L1× Anti-CD40 Bispecific Constructs with Stabilized Binding Domains on Cytokine Production

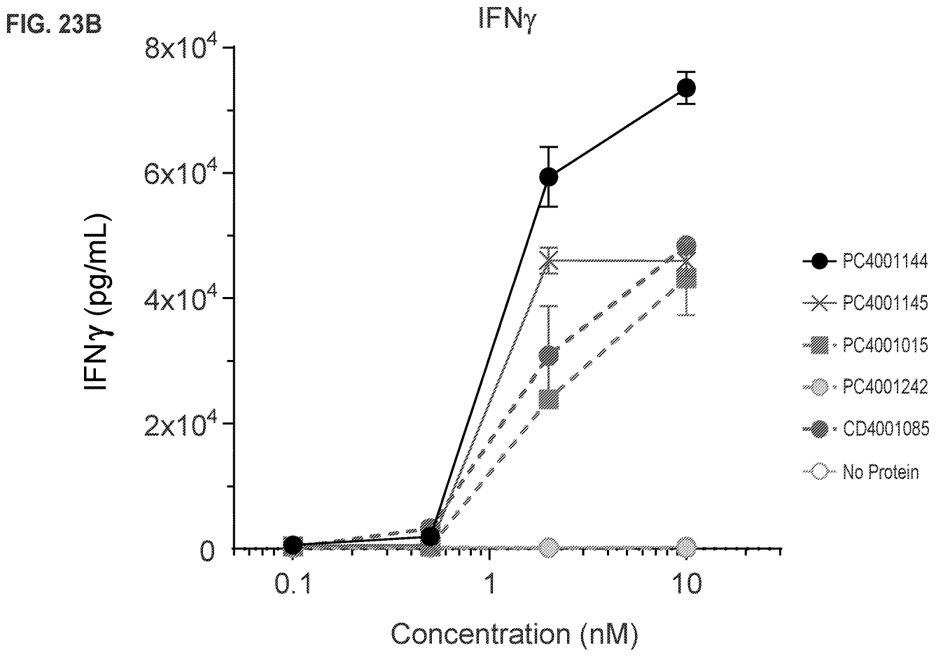

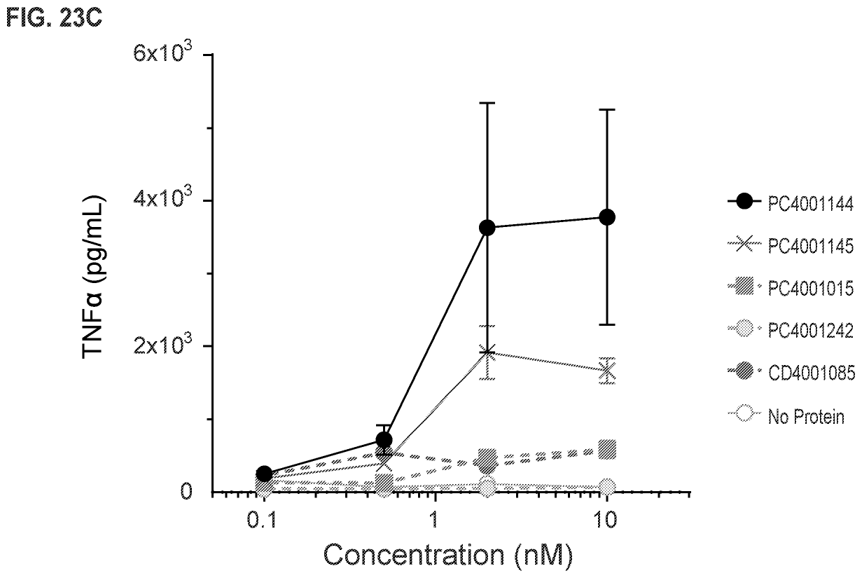

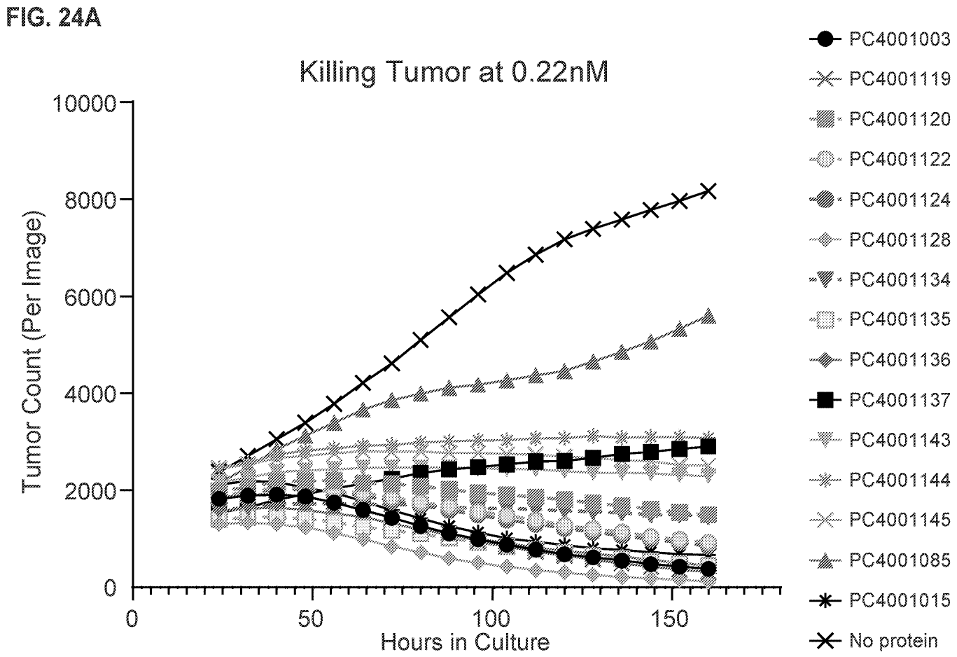

An outcome of APC and T cell activation is cytokine production and is correlated with inflammatory cells. Fifty thousand SCC152 tumor cells transduced with human PD-L1 and EBV protein (Vectorbuilder) were cocultured with 50000 immature DC and 100000 EBV-specific T cells. At 48 hours, supernatant was collected and analyzed for IL-12 (FIG. 23A), IFN-γ (FIG. 23B) and TNF-α (FIG. 23C) using a multiplex magnetic bead cytokine assay (Milliplex). As shown in FIGS. 23A-23C, the anti-PD-L1× anti-CD40 bispecific proteins induced a dose dependent secretion of the cytokines. Both bispecifics with stabilized binding domains (PC4001144 and PC4001145) had impacted cytokine secretion greater than controls.

### Example 38. In Vitro Evaluation of Anti-PD-L1× Anti-CD40 Bispecific Constructs with Stabilized Binding Domains on Tumor Cell Killing

To compare the activity of optimized anti-PD-L1×anti-CD40 bispecific proteins, live cell imaging was used to view tumor cell killing. The day prior to assay set up, 20.000 adherent SCC152 cells (expressing EBV, NLO and PD-L1) were plated and incubated ON. The following day, the media was then removed and replaced with 2× titrated αPD-L1×αCD40 test or control molecules. Immature DC, macrophages and EBV-specific T cells were mixed and transferred to the assay plate for a final cell number per well of 5000 and 10000 respectively. The plate was then transferred to the Incucyte reader (Sartorius) and imaged over the course of 6 days.

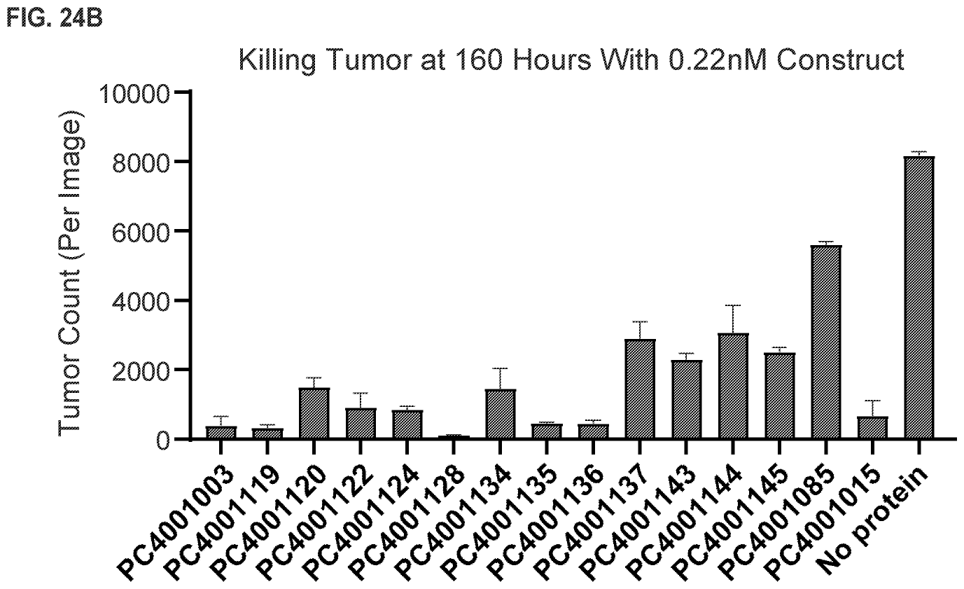

FIG. 24A graphically represents the reduced number of live SCC152 tumor cells at 0.22 nM of test or control molecules. All bispecifics with stabilized binding domains were able to enhance SCC152 killing but some were less effective than the parental bispecific (PC401003).

At the 160-hour timepoint, the tumor counts were plotted (FIG. 24B). Both the PD-L1 monospecific (PC401015) and parental bispecific (PC401003) from which all stabilized constructs were based were able to enhance SCC152 killing. All stabilized binding domain constructs were more effective at potentiating killing of SCC152 than the CD40 monoclonal antibody selicrelumab analogue (PC4001085).

Cells were harvested, fluorescently-labeled with APC/Cy7-αhCD5, BV605-αhCD56, BV650αhCD8 and BV510-α-hCD4 (Biolegend) and incubated for 30 minutes at 4° C. Cells were washed twice, and fixed and permeabilized for intracellular staining (Invitrogen). After permeabilization, cells were labeled with APC-α-granzyme B antibody and washed. Samples were resuspended and acquired on a BD FACSymphony™ flow cytometer. All samples were analyzed using FlowJo software to calculate the percentages of NK, CD8+, and CD4+ T cells expressing granzyme B.

1. A PD-L1 binding polypeptide that binds specifically to human PD-L1 wherein the PD-L1 binding polypeptide comprises, in order from amino terminus to carboxyl terminus or from carboxyl terminus to amino terminus,
(a) a first binding domain;
(b) a hinge region;
(c) an immunoglobulin constant region; and
(d) a second binding domain;
wherein the first binding domain is a PD-L1 binding domain and the second binding domain binds an immunostimulatory protein; or
wherein the first binding domain binds an immunostimulatory protein and the second binding domain is a PD-L1 binding domain.

2. The PD-L1 binding polypeptide of claim 1, wherein the PD-L1 binding domain comprises
(i) an immunoglobulin heavy chain variable region (VH) comprising HCDR1, HCDR2, and HCDR3; and
(ii) an immunoglobulin light chain variable region (VL) comprising LCDR1, LCDR2, and LCDR3.

3. The PD-L1 binding polypeptide of claim 2, wherein the HCDR1 comprises SEQ ID NO: 1, the HCDR2 comprises SEQ ID NO: 2, and the HCDR3 comprises SEQ ID NO: 3, the LCDR1 comprises SEQ ID NO:5, the LCDR2 comprises SEQ ID NO: 6, and the LCDR3 comprises SEQ ID NO: 7.

4. The PD-L1 binding polypeptide of claim 2 or 3, wherein the VH comprises SEQ ID NO: 4; and the VL comprises SEQ ID NO: 8.

5. The PD-L1 binding polypeptide ofany one of claims 1-4, wherein the PD-L1 binding domain comprises SEQ ID NO: 9.

6. The PD-L1 binding polypeptide of any one of claims 1-4, wherein the immunostimulatory protein is CD40, 4-1BB, CD3 or OX40.

7. A CD40 binding polypeptide that binds specifically to human CD40; wherein the CD40 binding polypeptide comprises, in order from amino terminus to carboxyl terminus or from carboxyl terminus to amino terminus,
(a) a first binding domain;
(b) a hinge region;
(c) an immunoglobulin constant region; and
(d) a second binding domain;
wherein the first binding domain is a CD40 binding domain and the second binding domain binds a tumor-associated antigen; or
wherein the first binding domain binds a tumor-associated antigen and the second binding domain is a CD40 binding domain.

8. The CD40 binding polypeptide of claim 7, wherein the CD40 binding domain comprises
(i) an immunoglobulin heavy chain variable region (VH) comprising HCDR1, HCDR2, and HCDR3; and
(ii) an immunoglobulin light chain variable region (VL) comprising LCDR1, LCDR2, and LCDR3.

9. The CD40 binding polypeptide of claim 8, wherein
(a) the HCDR1 comprises SEQ ID NO: 10, the HCDR2 comprises SEQ ID NO: 11, and the HCDR3 comprises SEQ ID NO: 12, the LCDR1 comprises SEQ ID NO: 14, the LCDR2 comprises SEQ ID NO: 15, and the LCDR3 comprises SEQ ID NO: 16;
(b) the HCDR1 comprises SEQ ID NO: 10, the HCDR2 comprises SEQ ID NO: 23, and the HCDR3 comprises SEQ ID NO: 12, the LCDR1 comprises SEQ ID NO: 14, the LCDR2 comprises SEQ ID NO: 15, and the LCDR3 comprises SEQ ID NO: 16;
(c) the HCDR1 comprises SEQ ID NO: 10, the HCDR2 comprises SEQ ID NO: 11, and the HCDR3 comprises SEQ ID NO: 31, the LCDR1 comprises SEQ ID NO: 14, the LCDR2 comprises SEQ ID NO: 15, and the LCDR3 comprises SEQ ID NO: 16;
(d) the HCDR1 comprises SEQ ID NO: 10, the HCDR2 comprises SEQ ID NO: 23, and the HCDR3 comprises SEQ ID NO: 31, the LCDR1 comprises SEQ ID NO: 14, the LCDR2 comprises SEQ ID NO: 15, and the LCDR3 comprises SEQ ID NO: 16;
(e) the HCDR1 comprises SEQ ID NO: 10, the HCDR2 comprises SEQ ID NO: 11, and the HCDR3 comprises SEQ ID NO: 12, the LCDR1 comprises SEQ ID NO: 37, the LCDR2 comprises SEQ ID NO: 15, and the LCDR3 comprises SEQ ID NO: 16;
(f) the HCDR1 comprises SEQ ID NO: 10, the HCDR2 comprises SEQ ID NO: 11, and the HCDR3 comprises SEQ ID NO: 12, the LCDR1 comprises SEQ ID NO: 41, the LCDR2 comprises SEQ ID NO: 15, and the LCDR3 comprises SEQ ID NO: 16;
(g) the HCDR1 comprises SEQ ID NO: 10, the HCDR2 comprises SEQ ID NO: 45, and the HCDR3 comprises SEQ ID NO: 12, the LCDR1 comprises SEQ ID NO: 14, the LCDR2 comprises SEQ ID NO: 15, and the LCDR3 comprises SEQ ID NO: 16;
(h) the HCDR1 comprises SEQ ID NO: 10, the HCDR2 comprises SEQ ID NO: 49, and the HCDR3 comprises SEQ ID NO: 12, the LCDR1 comprises SEQ ID NO: 14, the LCDR2 comprises SEQ ID NO: 15, and the LCDR3 comprises SEQ ID NO: 16; or
(i) the HCDR1 comprises SEQ ID NO: 10, the HCDR2 comprises SEQ ID NO: 23, and the HCDR3 comprises SEQ ID NO: 31, the LCDR1 comprises SEQ ID NO: 37, the LCDR2 comprises SEQ ID NO: 15, and the LCDR3 comprises SEQ ID NO: 16.

10. The CD40 binding polypeptide of claim 8 or 9, wherein
(a) the VH comprises SEQ ID NO: 13; and the VL comprises SEQ ID NO: 17;
(b) the VH comprises SEQ ID NO: 24; and the VL comprises SEQ ID NO: 17;
(c) the VH comprises SEQ ID NO: 27; and the VL comprises SEQ ID NO: 28;
(d) the VH comprises SEQ ID NO: 32; and the VL comprises SEQ ID NO: 17;
(e) the VH comprises SEQ ID NO:34; and the VL comprises SEQ ID NO: 17;
(f) the VH comprises SEQ ID NO: 13; and the VL comprises SEQ ID NO: 38;
(g) the VH comprises SEQ ID NO: 13; and the VL comprises SEQ ID NO: 42;
(h) the VH comprises SEQ ID NO: 46; and the VL comprises SEQ ID NO: 17;
(i) the VH comprises SEQ ID NO: 50; and the VL comprises SEQ ID NO: 17;
(j) the VH comprises SEQ ID NO: 484; and the VL comprises SEQ ID NO: 485; or
(k) the VH comprises SEQ ID NO: 484; and the VL comprises SEQ ID NO: 28.

11. The CD40 binding polypeptide of any one of claims 7-10, wherein the CD40 binding domain comprises SEQ ID NO: 18, SEQ ID NO: 25, SEQ ID NO: 29, SEQ ID NO: 33, SEQ ID NO: 35, SEQ ID NO: 39, SEQ ID NO: 43, SEQ ID NO: 47, SEQ ID NO: 51, SEQ ID NO: 486, or SEQ ID NO: 488.

12. The CD40 binding polypeptide of any one of claims 7-11, wherein the tumor-associated antigen is PD-L1, ROR1, or EGFR.

13. A binding polypeptide that binds specifically to human PD-L1 and human CD40; wherein the binding polypeptide comprises, in order from amino terminus to carboxyl terminus or from carboxyl terminus to amino terminus,
(a) a first binding domain;
(b) a hinge region;
(c) an immunoglobulin constant region; and
(d) a second binding domain;
wherein the first binding domain is a PD-L1 binding domain and the second binding domain is a CD40 binding domain; or
wherein the first binding domain is a CD40 binding domain and the second binding domain is a PD-L1 binding domain.

14. The binding polypeptide of claim 13, wherein
the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and
the CD40 binding domain comprises a VH comprising a HCDR1 comprising SEQ ID NO: 10, a HCDR2 comprising SEQ ID NO: 11, and a HCDR3 comprising SEQ ID NO: 12; and a VL comprising a LCDR1 comprising SEQ ID NO:14, a LCDR2 comprising SEQ ID NO: 15, and a LCDR3 comprising SEQ ID NO: 16.

15. The binding polypeptide of claim 13, wherein
the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and
the CD40 binding domain comprises a VH comprising a HCDR1 comprising SEQ ID NO:10, a HCDR2 comprising SEQ ID NO:23, and a HCDR3 comprising SEQ ID NO: 12; and a VL comprising a LCDR1 comprising SEQ ID NO:14, a LCDR2 comprising SEQ ID NO: 15, and a LCDR3 comprising SEQ ID NO: 16.

16. The binding polypeptide of claim 13, wherein
the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and
the CD40 binding domain comprises a VH comprising a HCDR1 comprising SEQ ID NO:10, a HCDR2 comprising SEQ ID NO: 11, and a HCDR3 comprising SEQ ID NO: 31; and a VL comprising a LCDR1 comprising SEQ ID NO: 14, a LCDR2 comprising SEQ ID NO: 15, and a LCDR3 comprising SEQ ID NO: 16.

17. The binding polypeptide of claim 13, wherein
the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and
the CD40 binding domain comprises a VH comprising a HCDR1 comprising SEQ ID NO:10, a HCDR2 comprising SEQ ID NO:23, and a HCDR3 comprising SEQ ID NO: 31; and a VL comprising a LCDR1 comprising SEQ ID NO:14, a LCDR2 comprising SEQ ID NO: 15, and a LCDR3 comprising SEQ ID NO: 16.

18. The binding polypeptide of claim 13, wherein
the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and
the CD40 binding domain comprises a VH comprising a HCDR1 comprising SEQ ID NO:10, a HCDR2 comprising SEQ ID NO: 11, and a HCDR3 comprising SEQ ID NO: 12; and a VL comprising a LCDR1 comprising SEQ ID NO:37, a LCDR2 comprising SEQ ID NO: 15, and a LCDR3 comprising SEQ ID NO: 16.

19. The binding polypeptide of claim 13, wherein
the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and
the CD40 binding domain comprises a VH comprising a HCDR1 comprising SEQ ID NO:10, a HCDR2 comprising SEQ ID NO: 11, and a HCDR3 comprising SEQ ID NO: 12; and a VL comprising a LCDR1 comprising SEQ ID NO:41, a LCDR2 comprising SEQ ID NO: 15, and a LCDR3 comprising SEQ ID NO: 16.

20. The binding polypeptide of claim 13, wherein
the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and
the CD40 binding domain comprises a VH comprising a HCDR1 comprising SEQ ID NO: 10, a HCDR2 comprising SEQ ID NO: 45, and a HCDR3 comprising SEQ ID NO: 12; and a VL comprising a LCDR1 comprising SEQ ID NO:14, a LCDR2 comprising SEQ ID NO: 15, and a LCDR3 comprising SEQ ID NO: 16.

21. The binding polypeptide of claim 13, wherein
the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7; and
the CD40 binding domain comprises a VH comprising a HCDR1 comprising SEQ ID NO:10, a HCDR2 comprising SEQ ID NO: 49, and a HCDR3 comprising SEQ ID NO: 12; and a VL comprising a LCDR1 comprising SEQ ID NO:14, a LCDR2 comprising SEQ ID NO: 15, and a LCDR3 comprising SEQ ID NO: 16.

22. The binding polypeptide of claim 13, wherein
the PD-L1 binding domain comprises an immunoglobulin heavy chain variable region (VH) comprising a HCDR1 comprising SEQ ID NO: 1, a HCDR2 comprising SEQ ID NO: 2, and a HCDR3 comprising SEQ ID NO: 3; and an immunoglobulin light chain variable region (VL) comprising a LCDR1 comprising SEQ ID NO:5, a LCDR2 comprising SEQ ID NO: 6, and a LCDR3 comprising SEQ ID NO: 7, and
the CD40 binding domain comprises a VH comprising a HCDR1 comprising SEQ ID NO:10, a HCDR2 comprising SEQ ID NO: 23, and a HCDR3 comprising SEQ ID NO: 31; and a VL comprising a LCDR1 comprising SEQ ID NO:37, a LCDR2 comprising SEQ ID NO: 15, and a LCDR3 comprising SEQ ID NO: 16.

23. The binding polypeptide of any one of claims 13-22, wherein
(a) the PD-L1 binding domain comprises a VH comprising SEQ ID NO: 4 and a VL comprising SEQ ID NO: 8; and the CD40 binding domain comprises a VH comprising SEQ ID NO: 13 and a VL comprising SEQ ID NO: 17;
(b) the PD-L1 binding domain comprises a VH comprising SEQ ID NO: 4 and a VL comprising SEQ ID NO: 8; and the CD40 binding domain comprises a VH comprising SEQ ID NO: 24 and a VL comprising SEQ ID NO: 17;
(c) the PD-L1 binding domain comprises a VH comprising SEQ ID NO: 4 and a VL comprising SEQ ID NO: 8; and the CD40 binding domain comprises a VH comprising SEQ ID NO: 27 and a VL comprising SEQ ID NO: 28;
(d) the PD-L1 binding domain comprises a VH comprising SEQ ID NO: 4 and a VL comprising SEQ ID NO: 8; and the CD40 binding domain comprises a VH comprising SEQ ID NO: 32 and a VL comprising SEQ ID NO: 17;
(e) the PD-L1 binding domain comprises a VH comprising SEQ ID NO: 4 and a VL comprising SEQ ID NO: 8; and the CD40 binding domain comprises a VH comprising SEQ ID NO: 34 and a VL comprising SEQ ID NO: 17;
(f) the PD-L1 binding domain comprises a VH comprising SEQ ID NO: 4 and a VL comprising SEQ ID NO: 8; and the CD40 binding domain comprises a VH comprising SEQ ID NO: 13 and a VL comprising SEQ ID NO: 38;
(g) the PD-L1 binding domain comprises a VH comprising SEQ ID NO: 4 and a VL comprising SEQ ID NO: 8; and the CD40 binding domain comprises a VH comprising SEQ ID NO: 13 and a VL comprising SEQ ID NO: 42;
(h) the PD-L1 binding domain comprises a VH comprising SEQ ID NO: 4 and a VL comprising SEQ ID NO: 8; and the CD40 binding domain comprises a VH comprising SEQ ID NO: 46 and a VL comprising SEQ ID NO: 17;
(i) the PD-L1 binding domain comprises a VH comprising SEQ ID NO: 4 and a VL comprising SEQ ID NO: 8; and the CD40 binding domain comprises a VH comprising SEQ ID NO: 50 and a VL comprising SEQ ID NO: 17;
(j) the PD-L1 binding domain comprises a VH comprising SEQ ID NO: 4 and a VL comprising SEQ ID NO: 8; and the CD40 binding domain comprises a VH comprising SEQ ID NO: 484 and a VL comprising SEQ ID NO: 485; or
(k) the PD-L1 binding domain comprises a VH comprising SEQ ID NO: 4 and a VL comprising SEQ ID NO: 8; and the CD40 binding domain comprises a VH comprising SEQ ID NO: 484 and a VL comprising SEQ ID NO: 28.

24. The binding polypeptide of any one of claims 13-23, wherein
(a) the PD-L1 binding domain comprises SEQ ID NO: 9; and the CD40 binding domain comprises SEQ ID NO: 18;
(b) the PD-L1 binding domain comprises SEQ ID NO: 9; and the CD40 binding domain comprises SEQ ID NO: 25;
(c) the PD-L1 binding domain comprises SEQ ID NO: 9; and the CD40 binding domain comprises SEQ ID NO: 29;
(d) the PD-L1 binding domain comprises SEQ ID NO: 9; and the CD40 binding domain comprises SEQ ID NO: 33;
(e) the PD-L1 binding domain comprises SEQ ID NO: 9; and the CD40 binding domain comprises SEQ ID NO: 35;
(f) the PD-L1 binding domain comprises SEQ ID NO: 9; and the CD40 binding domain comprises SEQ ID NO: 39;
(g) the PD-L1 binding domain comprises SEQ ID NO: 9; and the CD40 binding domain comprises SEQ ID NO: 43;
(h) the PD-L1 binding domain comprises SEQ ID NO: 9; and the CD40 binding domain comprises SEQ ID NO: 47;
(i) the PD-L1 binding domain comprises SEQ ID NO: 9; and the CD40 binding domain comprises SEQ ID NO: 51;
(j) the PD-L1 binding domain comprises SEQ ID NO: 9; and the CD40 binding domain comprises SEQ ID NO: 486; or
(k) the PD-L1 binding domain comprises SEQ ID NO: 9; and the CD40 binding domain comprises SEQ ID NO: 488.

25. The binding polypeptide of any one of claims 13-24, wherein the binding polypeptide comprises SEQ ID NO: 19, SEQ ID NO: 26, SEQ ID NO: 30, SEQ ID NO: 280, SEQ ID NO: 36, SEQ ID NO: 40, SEQ ID NO: 44, SEQ ID NO: 48, SEQ ID NO: 52: SEQ ID NO: 487, or SEQ ID NO: 489.

26. The binding polypeptide of any one of claims 1-25, wherein the hinge is an immunoglobulin hinge.

27. The binding polypeptide of any one of claims 1-26, wherein the immunoglobulin constant region comprises immunoglobulin CH2 and CH3 domains of IgG1, IgG2, IgG3, IgG4, IgA1, IgA2 or IgD.

28. The binding polypeptide of claim 27, wherein the immunoglobulin constant region comprises a human IgG1 CH2 domain comprising an amino acid substitution at one or more of the following residues, according to the EU numbering system: E233, L234, L235, G236, G237, E318, K320, K322.

29. The binding polypeptide of claim 28, wherein the amino acid substitution at residue E233 is E233P.

30. The binding polypeptide of claim 28, wherein the amino acid substitution at residue L234 is selected from the group consisting of L234A and L234V.

31. The binding polypeptide of claim 28, wherein the amino acid substitution at residue L235 is L235A.

32. The binding polypeptide of claim 28, wherein the amino acid substitution at residue G237 is G237A.

33. The binding polypeptide of claim 28, wherein the amino acid substitution at E318 is E318A.

34. The binding polypeptide of claim 28, wherein the amino acid substitution at K320 is K320A.

35. The binding polypeptide to claim 28, wherein the amino acid substitution at K322 is K322A.

36. The binding polypeptide of claim 27, wherein residue G236 according to the EU numbering system) is deleted.

37. A dimeric protein comprising the binding polypeptide of any one of claims 1-36.

38. The dimeric protein of claim 36, wherein the dimeric protein is a homodimer.

39. A composition comprising the binding polypeptide of any one of claims 1-36, and a pharmaceutically acceptable carrier, diluent or excipient.

40. A composition comprising the dimeric protein of claim 37 or 38, and a pharmaceutically acceptable carrier, diluent or excipient.

41. A nucleic acid molecule comprising a nucleotide sequence encoding the binding polypeptide of any one of claims 1-36.

42. The nucleic acid molecule of claim 41, wherein the nucleotide sequence comprises SEQ ID NO: 78, SEQ ID NO: 79, SEQ ID NO: 259, or SEQ ID NO: 260.

43. An expression vector comprising the nucleic acid molecule of claim 41 or 42.

44. A recombinant host cell comprising the nucleic acid molecule of claim 41 or 42 or the expression vector of claim 43.

45. A method of producing a binding polypeptide, the method comprising:
culturing the recombinant host cell of claim 44 under conditions whereby the nucleic acid molecule is expressed, thereby producing the binding polypeptide; and
isolating the binding polypeptide from the host cell or culture.

46. A method for treating cancer in a subject in need thereof, the method comprising administering to the subject a therapeutically effective amount of the binding polypeptide of any one of claims 1-36, the dimeric protein of claim 37 or 38, or the composition of claim 39 or 40.

47. A method for treating a symptom of cancer in a subject in need thereof, the method comprising administering to the subject a therapeutically effective amount of the binding polypeptide of any one of claims 1-36, the dimeric protein of claim 37 or 38, or the composition of claim 39 or 40.

48. The method of claim 46 or 47, wherein the cancer is a solid tumor.

49. The method of any one of claims 46-48, wherein the cancer expresses PD-L1.

50. The method of any one of claims 46-48, wherein the cancer is head and neck cancer, melanoma, lung cancer, brain cancer, thymus cancer, breast cancer, liver cancer, pancreatic cancer, kidney cancer, ovarian cancer, bladder cancer, gastrointestinal tract cancer or colon cancer.

51. The binding polypeptide of any one of claims 1-36, the dimeric protein of claim 37 or 38, or the composition of claim 39 or 40 for use as a medicament.
